(19) United States
(12) Patent Application Publication
(10) Pub. No.: US 2024/0268226 A1
(43) Pub. Date: Aug. 8, 2024

(54) COMPOUND, COMPOSITION, AND ORGANIC ELECTRONIC DEVICE

(71) Applicant: SHENZHEN CHINA STAR OPTOELECTRONICS SEMICONDUCTOR DISPLAY TECHNOLOGY CO., LTD., Shenzhen (CN)

(72) Inventors: Zeming XIA, Shenzhen (CN); Zhifei LONG, Shenzhen (CN); Ruifeng HE, Shenzhen (CN); Jingyao SONG, Shenzhen (CN)

(73) Assignee: SHENZHEN CHINA STAR OPTOELECTRONICS SEMICONDUCTOR DISPLAY TECHNOLOGY CO., LTD., Shenzhen (CN)

(21) Appl. No.: 18/175,544

(22) Filed: Feb. 28, 2023

(30) Foreign Application Priority Data
Dec. 29, 2022 (CN) 202211710113.1

(51) Int. Cl. H10K 85/60; C07D 307/91; C07D 405/10; C07D 405/14; C07D 409/10; C07D 409/14; C07F 7/08; H10K 85/40

(52) U.S. Cl. CPC H10K 85/6574; C07D 307/91; C07D 405/10; C07D 405/14; C07D 409/10; C07D 409/14; C07F 7/0812; H10K 85/40; H10K 85/615; H10K 85/622; H10K 85/626; H10K 85/654; H10K 85/6572; H10K 85/6576; C07B 2200/05; H10K 2102/301

(57) ABSTRACT

The disclosure provides a compound, a composition, and an organic electronic device. The compound is represented by formula (1):

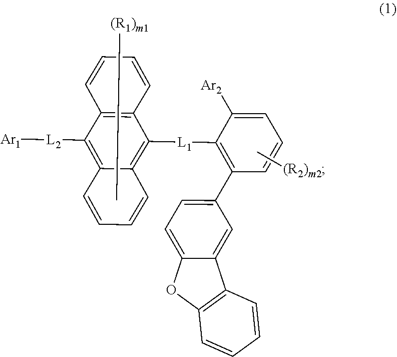

Ar₁ is selected from an aromatic group containing 6 to 18 ring atoms and a heteroaromatic group containing 6 to 13 ring atoms; Ar₂ is selected from an aromatic group containing 6 to 14 ring atoms and a heteroaromatic group containing 13 to 19 ring atoms; L₁ and L₂ are each independently selected from a single bond and a group containing 6 ring atoms; R₁ and R₂ are each independently selected from -D, a C₄ alkyl group, an aromatic group containing 6 to 10 ring atoms, and a heteroaromatic group containing 12 ring atoms; m1 is 0, 2, or 8, and m2 is 0 or 1.

### TECHNICAL FIELD

The disclosure relates to the technical field of display, in particular to a compound, a composition, and an organic electronic device.

### BACKGROUND

Organic light-emitting Diodes (OLEDs) have great potential for further development because of advantages of wide viewing angles, good responsiveness, low working voltages, and thin thicknesses in application of optoelectronic devices (such as flat panel displays and illumination).

In order to improve luminous efficiency of the organic light-emitting diodes, various light-emitting material systems based on fluorescence and phosphorescence have been developed. However, development of excellent blue light-emitting materials is a huge challenge for both fluorescent materials and phosphorescent materials. Compared with blue light phosphor materials, reliability of the organic light-emitting diodes using blue light phosphor materials is higher at present stage. In general, blue light-emitting layers adopt doping structures containing host materials and guest materials, and blue light host materials are mostly fused ring derivatives of anthracene. However, stability of the blue light host materials is poor, resulting in short life of the OLEDs, and luminous efficiency of the blue light host materials also needs to be improved.

### SUMMARY

The disclosure provides a compound, a composition, and an organic electronic device to improve luminous efficiency and service life of organic electronic devices.

To solve the above problems, technical solutions provided by the disclosure is as following:

The disclosure provides a compound represented by formula (1):

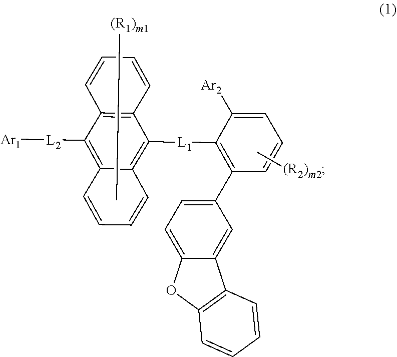

wherein Ar₁ is selected from an aromatic group containing 6 to 18 ring atoms and a heteroaromatic group containing 6 to 13 ring atoms;

Ar₂ is selected from an aromatic group containing 6 to 14 ring atoms and a heteroaromatic group containing 13 to 19 ring atoms;

L₁ and L₂ are each independently selected from a single bond and a group containing 6 ring atoms;

R₁ and R₂ are each independently selected from -D, a C₄ alkyl group, an aromatic group containing 6 to 10 ring atoms, and a heteroaromatic group containing 12 ring atoms;

m1 is 0, 2, or 8; and

m2 is 0 or 1.

In some embodiments of the disclosure, Ar₁ is selected from any of

following structures:

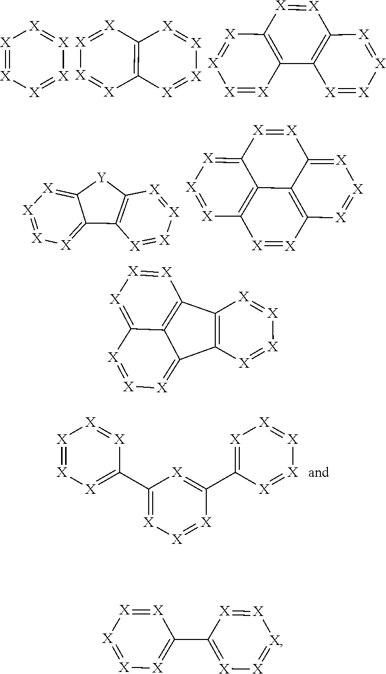

wherein X is independently selected from CR₃ and N at each occurrence;

Y is selected from CR₅R₆ and O;

R₃ is selected from —H and -D, R₅ and R₆ are each independently selected from a methyl group; and

when X is a linking site, X is C.

In some embodiments of the disclosure, Ar₁ is selected from any of following structures:

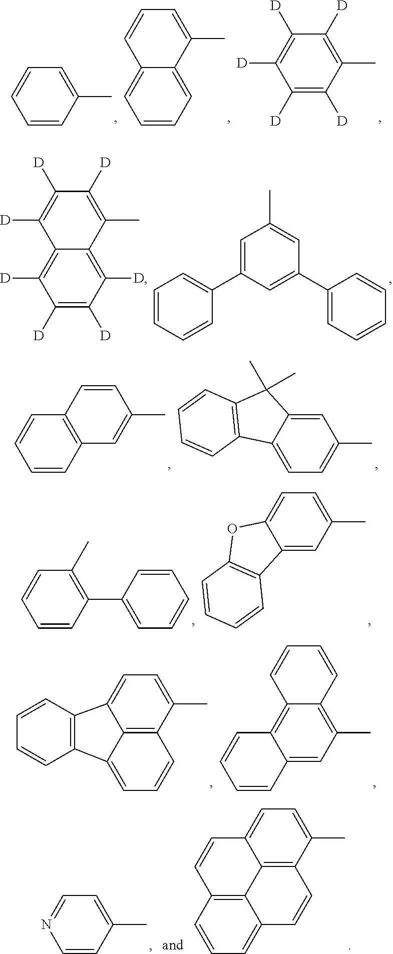

In some embodiments of the disclosure, Ar₂ is selected from any of following structures:

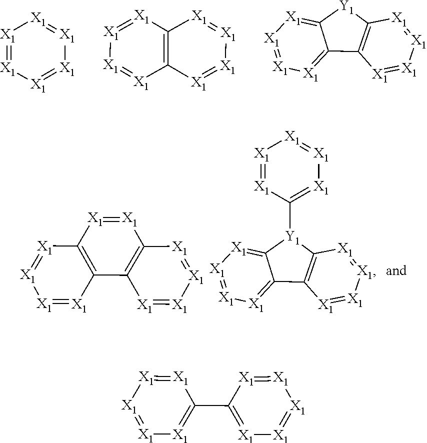

wherein X₁ is selected from CR₇;

Y₁ is selected from N and O; and

R is independently selected from —H at each occurrence.

In some embodiments of the disclosure, Ar₂ is selected from any of following structures:

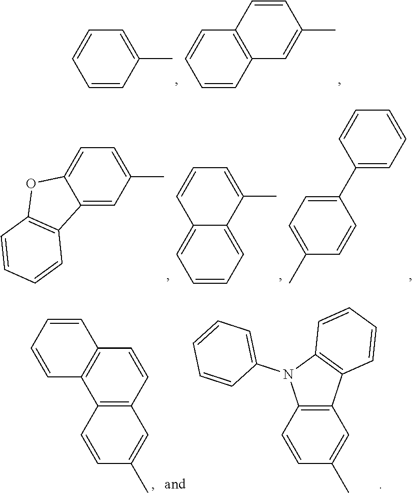

In some embodiments of the disclosure, L₁ is a single bond or selected from

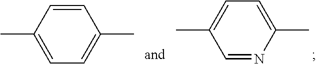

L₂ is a single bond or selected from

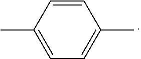

In some embodiments of the disclosure, R₁ is selected from -D and a tert-butyl group; R₂ is selected from

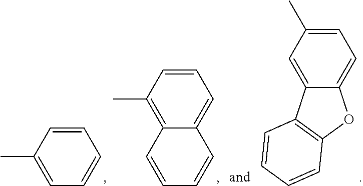

In some embodiments of the disclosure, the compound represented by formula (1) is selected from any of following structures:

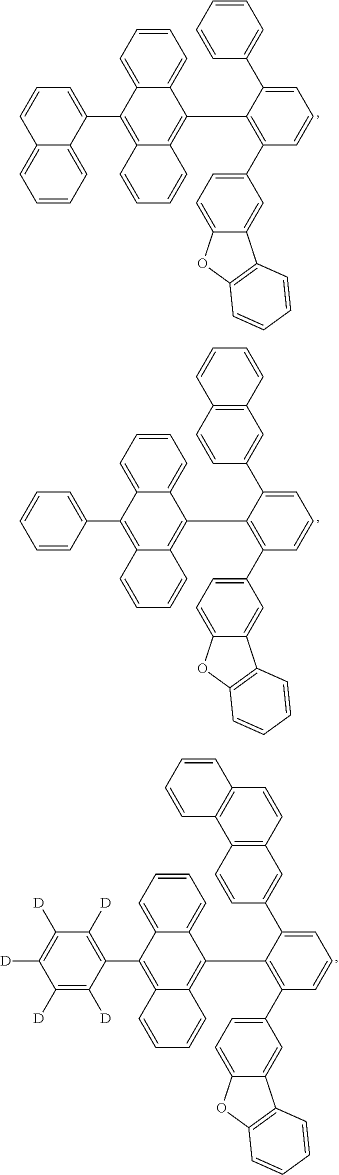

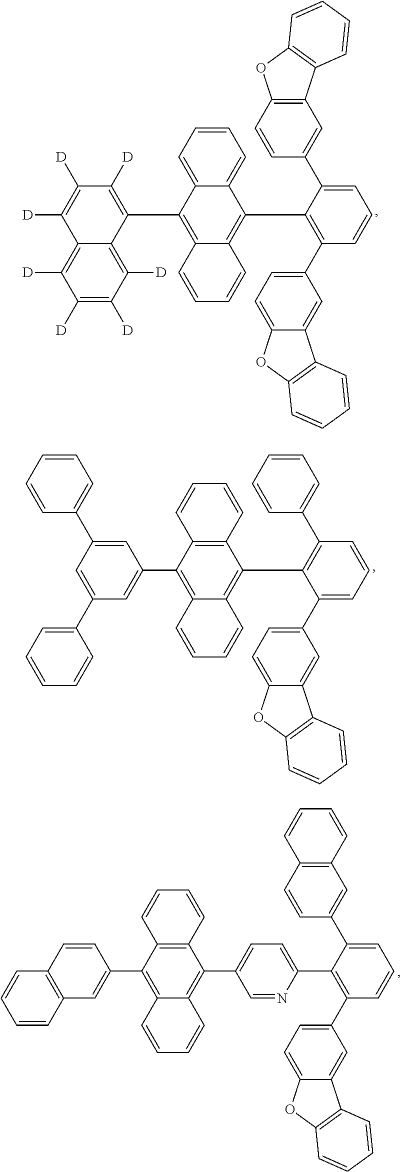

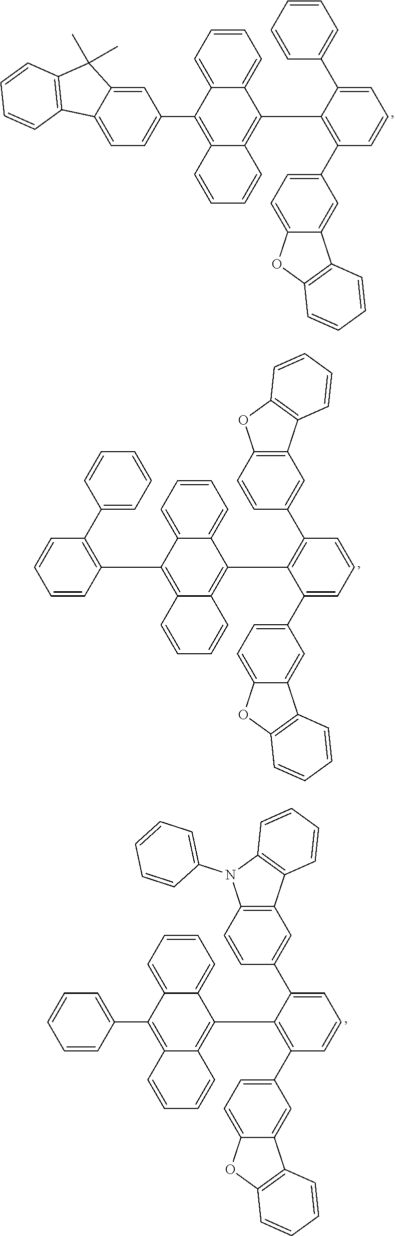

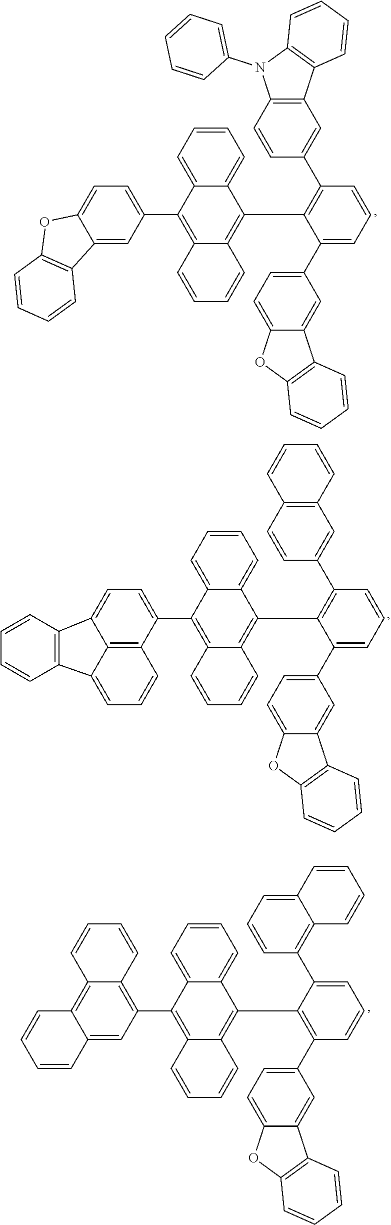

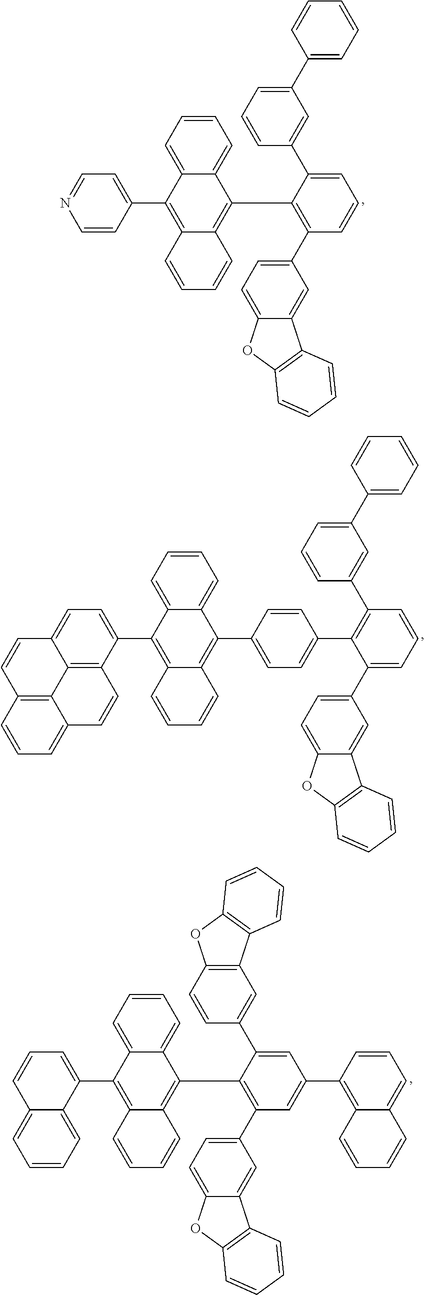 [SMILES: c1ccc(-c2cccc(-c3cccc(-c4ccc5oc6ccccc6c5c4)c3-c3c4ccccc4c(-c4ccncc4)c4ccccc34)c2)cc1 ; c1ccc(-c2cccc(-c3cccc(-c4ccc5oc6ccccc6c5c4)c3-c3ccc(-c4c5ccccc5c(-c5ccc6ccc7cccc8ccc5c6c78)c5ccccc45)cc3)c2)cc1 ; c1ccc2c(-c3cc(-c4ccc5oc6ccccc6c5c4)c(-c4c5ccccc5c(-c5cccc6ccccc56)c5ccccc45)c(-c4ccc5oc6ccccc6c5c4)c3)cccc2c1]

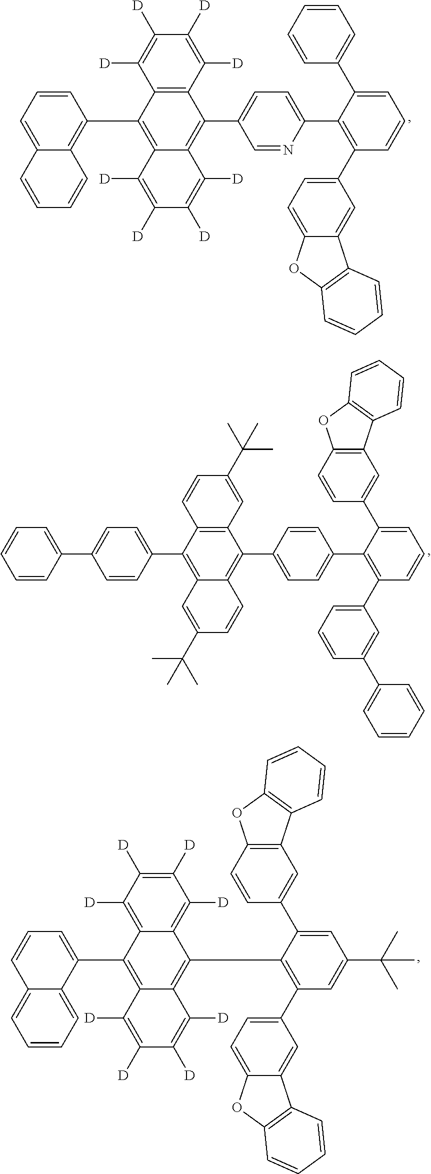

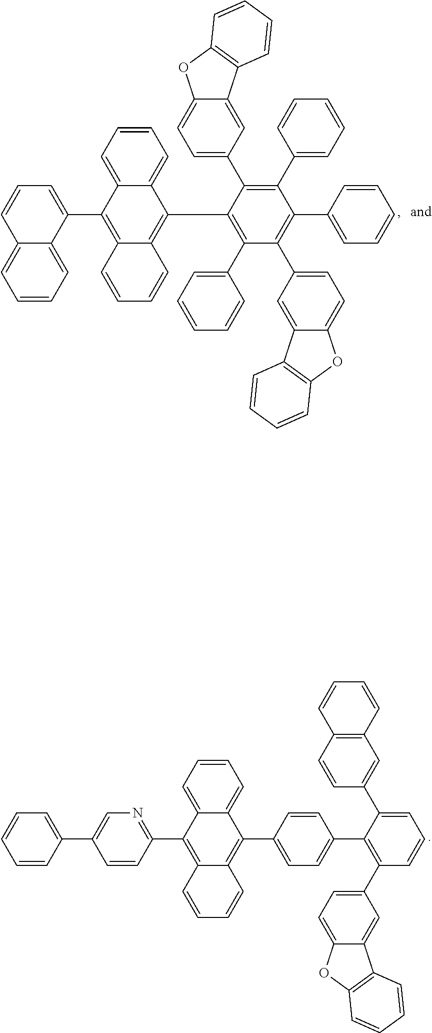 [SMILES: c1ccc(-c2c(-c3ccc4oc5ccccc5c4c3)c(-c3ccccc3)c(-c3c4ccccc4c(-c4cccc5ccccc45)c4ccccc34)c(-c3ccc4oc5ccccc5c4c3)c2-c2ccccc2)cc1 ; c1ccc(-c2ccc(-c3c4ccccc4c(-c4ccc(-c5c(-c6ccc7ccccc7c6)cccc5-c5ccc6oc7ccccc7c6c5)cc4)c4ccccc34)nc2)cc1]

The disclosure also provides a composition. The composition includes one or more compounds described in any of the embodiments of the disclosure and

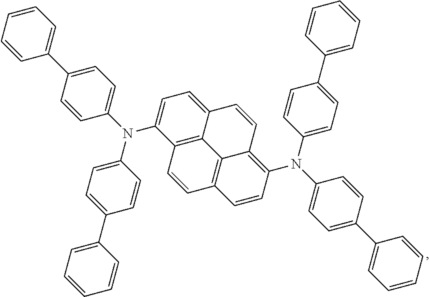 [SMILES: c1ccc(-c2ccc(N(c3ccc(-c4ccccc4)cc3)c3ccc4ccc5c(N(c6ccc(-c7ccccc7)cc6)c6ccc(-c7ccccc7)cc6)ccc6ccc3c4c65)cc2)cc1]

Moreover, the disclosure also provides an organic electronic device, the organic electronic device includes a first electrode, a second electrode, and an organic functional layer disposed between the first electrode and the second electrode. The organic functional layer includes one or more of the above-mentioned compounds or the mixture of the disclosure.

The disclosure provides the compound, the composition, and the organic electronic device. On one hand, the compound improves a hole transmission performance of a host material and luminous efficiency of a light-emitting layer by introducing a hole transport-typed group with large steric hindrance on an ortho-position of a benzene ring connected to a site of 9 of anthracene in the compound. On other hand, the compound increases the steric hindrance of an anthracene-based host material and reduces x-x interaction of the anthracene-based host material, thus reducing energy transfer between a host material and a guest material, improving a phenomena of triplet-triplet annihilation (TTA) and triplet polarity annihilation (TPA) in a reaction system, thereby improving the luminous efficiency of the light-emitting layer, and improving service life and stability of the organic electronic device. On another hand, the increase of steric hindrance of the anthracene-based host material reduces I-x accumulation of the anthracene-based host material, which makes solubility of the anthracene-based host material greater, thus making a film-forming performance of the organic functional layer including the anthracene-based host material better, further improving the luminous efficiency of the light-emitting layer, and improving service life and stability of the organic electronic device.

### BRIEF DESCRIPTION OF DRAWINGS

Technical solutions and other beneficial effects of the disclosure will be clear through detailed description of the specific implementation of the disclosure in combination with attached drawings.

FIG. 1 is a schematic structural diagram of an organic electronic device provided by an embodiment of the disclosure.

### DETAILED DESCRIPTION OF EMBODIMENT

Technical solutions in the disclosure will be illustrated clearly and completely below in combination with drawings in embodiments of the disclosure. It is apparant that the described embodiments are only a part of the embodiments of the disclosure, not all of them. Based on the embodiments in the disclosure, all other embodiments obtained by those skilled in the art without paying creative effort belong to a scope of the disclosure.

In the description of the disclosure, a term “include” refers to “include but not limited to”, and a term “more” refers to “two or more than two”. Various embodiments of the disclosure may exist in a form of a scope. It should be understood that description in a form of the scope is only for convenience and conciseness, and should not be understood as a rigid restriction on the scope of the disclosure. Therefore, it should be considered that the description of ranges has specifically disclosed all possible sub-ranges and single values within the sub-ranges. For example, it should be considered that the description of a range “from 1 to 6” has specifically disclosed a sub-range, such as from 1 to 3, from 1 to 4, from 1 to 5, from 2 to 4, from 2 to 6, from 3 to 6, or a single number within the sub-range, such as 1, 2, 3, 4, 5, or 6, which is applicable regardless of the scope. In addition, whenever a numerical range is indicated in this article, it refers to a number (fraction or integer) including any reference within the range.

Unless otherwise defined, all technical and scientific terms used herein have a same meaning as those commonly understood by those skilled in the technical field of the disclosure. The terms used in the specification of the disclosure are only for a purpose of describing specific embodiments, and are not intended to limit the disclosure. A term “and/or” as used herein includes any and all combinations of one or more related listed items.

In the disclosure, a composition, printing ink, and ink have a same meaning and may be interchanged.

In the disclosure, an aromatic group, an aromatic ring, and an aromatic ring system have a same meaning and may be interchanged.

In the disclosure, a heteroaromatic group, a heteroaromatic ring, and a heteroaromatic ring system have a same meaning and may be interchanged.

In the disclosure, “substituted” means that one or more hydrogen atoms in one substituted group are substituted by a substituent group.

In the disclosure, “substituted or unsubstituted” means that a defined group may be substituted or not be substituted. When the defined group is substituted, it should be understood that the defined group may be substituted by at least one substituent R. The substituent R is selected from, but not limited: -D, a cyano group, an isocyano group, a nitro group, a halogen group, a C₁-C₂₀ alkyl group, a heterocyclic group containing 3-20 ring atoms, an aromatic group containing 6-20 ring atoms, a heteroaromatic group containing 5-20 ring atoms, —NR′R″, a silyl group, a carbonyl group, an alkoxycarbonyl group, an aryloxycarbonyl group, a carbamoyl group, a haloformyl group, a formyl group, an isocyanate group, a thiocyanate group, an isothiocyanate group, a hydroxyl group, and a trifluoromethyl group. The above-mentioned groups may further be substituted by acceptable substituent groups in the art. It can be understood that R′ and R″ in the —NR′R″ are both independently selected from, but not limited: —H, -D, a cyanogen group, an isocyano group, a nitro group, a halogen group, a C₁-C₁₀ alkyl group, a heterocyclic group containing 3-20 ring atoms, an aromatic group containing 6-20 ring atoms, and a heteroaromatic group containing 5-20 ring atoms. Preferably, the R is selected from, but not limited: -D, a cyano group, an isocyano group, a nitro group, a halogen group, a C₁-C₁₀ alkyl group, a heterocyclic group containing 3-10 ring atoms, an aromatic group containing 6-20 ring atoms, a heteroaromatic group containing 5-20 ring atoms, a silyl group, a carbonyl group, an alkoxycarbonyl group, an aryloxycarbonyl group, a carbamoyl group, a haloformyl group, a formyl group, an isocyanate group, a thiocyanate group, an isothiocyanate group, a hydroxyl group, and a trifluoromethyl group, and the above-mentioned groups may further be substituted by acceptable substituent groups in the art.

In the disclosure, “a number of ring atoms” refers to a number of atoms constituting a ring of structural compounds (such as a monocyclic compound, a fused ring compound, a cross-linked compound, a carbon ring compound, and a heterocyclic compound) obtained by bonding between atoms. In a ring substituted by a substituent group, atoms contained in the substituent group is not included in the atoms forming the ring. The same applies to the “number of ring atoms” described below unless otherwise specified. For example, a number of ring atoms of a benzene ring is 6, a number of ring atoms of a naphthalene ring is 10, and a number of ring atoms of a thiophene group is 5.

“An aryl group or an aromatic group” refers to an aromatic hydrocarbon group derived from a basis of an aromatic ring compound removing an H. The aromatic ring compound may be a single ring aromatic group, a fused ring aromatic group, or a polycyclic aromatic group. For a polycyclic ring type, at least one ring is an aromatic ring system. For example, “a substituted or unsubstituted aryl group containing 6 to 40 ring atoms” refers to an aryl group containing 6 to 40 ring atoms. Preferably, “the substituted or unsubstituted aryl group containing 6 to 30 ring atoms” refers to a substituted or unsubstituted aryl group containing 6 to 30 ring atoms. Preferably, “the substituted or unsubstituted aryl group containing 6 to 18 ring atoms” refers to a substituted or unsubstituted aryl group containing 6 to 18 ring atoms. Preferably, “the substituted or unsubstituted aryl group containing 6 to 14 ring atoms” refers to a substituted or unsubstituted aryl group containing 6 to 14 ring atoms, and the aryl group is optionally further substituted. Suitable examples include, but are not limited: a phenyl group, a biphenyl group, a terphenyl group, a naphthyl group, an anthracyl group, a phenanthryl group, a fluoranthenyl group, a terphenylene group, a pyrenyl group, a perylene group, a tetraphenyl group, a fluorenyl group, a diphenyl group, an acenaphthenyl group, and derivatives of these groups. Understandably, multiple aryl groups may further be disconnected by short non-aromatic units (for example, non-hydrogen atoms, e.g., C, N, or O, is less than 10%). In particular, an acenaphthene group, a fluorene group, a 9,9-diarylfluorene group, a triarylamine group, and a diaryl ether system should be further included in the definition of the aryl groups.

“A heteroaryl group or a heteroaromatic group” refers a basis of an aryl group with at least one carbon atom substituted by a non-carbon atom, and the non-carbon atom may be N, O, S, and the like. For example, “a substituted or unsubstituted heteroaryl group containing 5 to 40 ring atoms” refers to a heteroaryl group containing 5 to 40 ring atoms. Preferably, “the substituted or unsubstituted heteroaryl group containing 6 to 30 ring atoms” refers to a substituted or unsubstituted heteroaryl group containing 6 to 30 ring atoms. Preferably, “the substituted or unsubstituted heteroaryl group containing 6 to 18 ring atoms” refers to a substituted or unsubstituted heteroaryl group containing 6 to 18 ring atoms. Preferably, “the substituted or unsubstituted heteroaryl group containing 6 to 14 ring atoms” refers to a substituted or unsubstituted heteroaryl group containing 6 to 14 ring atoms, and the heteroaryl group are optionally further substituted. Suitable examples include but are not limited: a thiophene group, a furan group, a pyrrolyl group, a diazo group, a triazole group, an imidazolyl group, a pyridinyl group, a bipyridyl group, a pyrimidinyl group, a triazinyl group, an acridine group, a pyridazinyl group, a pyrazinyl group, a quinolinyl group, an isoquinolinyl group, a quinazolinyl group, a quinoxalinyl group, a phthalazinyl group, a pyridino pyrimidinyl group, a pyridino pyrazinyl group, a benzo thienyl group, a benzofuranyl group, an indolyl group, a pyrrolo imidazolyl group, a pyrrolo pyrrolyl group, a thiophenopyrrolyl group, a thiophenothiophenyl group, a furanopyrrolyl group, a furanofuranyl group, a thiophenofuranyl group, a benzoisoxazolyl group, a benzoisothiazolyl group, a benzimidazolyl group, an o-diaznaphthyl group, a phenanthryl group, a pyridinyl group, a quinazolinketone group, a dibenzothiophenyl group, a dibenzofuranyl group, a carbazolyl group, and derivatives of these groups.

In the disclosure, “an alkyl group” may mean a linear alkyl group, a branched alkyl group, and/or a cyclic alkyl group. A number of carbon atoms in the alkyl group may range from 1 to 50, 1 to 30, 1 to 20, 1 to 10, or 1 to 6. Non-limiting examples of the alkyl group include a methyl group, an ethyl group, a n-propyl group, an isopropyl group, a n-butyl group, a sec-butyl group, a tert-butyl group, an isobutyl group, a 2-ethylbutyl group, a 3,3-dimethylbutyl group, a n-pentyl group, an isopentyl group, a neopentyl group, a tert-pentyl group, a cyclopentyl group, a 1-methylpentyl group, a 3-methylpentyl group, a 2-ethylpentyl group, a 4-methyl-2-pentyl group, a n-hexyl group, a 1-methylhexyl group, a 2-ethylhexyl group, a 2-butyl hexyl group, a cyclohexyl group, an adamantine group, and the like.

In the disclosure, “halogen” or “a halogen group” refers to —F, —Cl, —Br, or —I.

In the disclosure, a term “an alkoxy group” refers to a group containing a —O-alkyl group, that is, the alkyl group as defined above is connected to a parent structure via an oxygen atom. For phrases containing the above-mentioned terms, suitable examples include but are not limited: a methoxy group (—O—CH₃ or —OMe), an ethoxy group (—O—CH₂CH₃ or —OEt), and a tert-butoxy group (—O—C(CH₃)₃ or —OtBu).

In the disclosure, the “*” indicates a linking site.

In the disclosure, when one group contains a plurality of substituent groups having a same symbol, the substituent groups may be the same or be different, such as

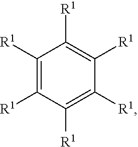

six R₁ groups of a benzene ring may be the same or different.

In the disclosure, a single bond connected to a substituent group and penetrated a corresponding ring/bond indicates that the substituent group may be connected to any site of the ring. For example,

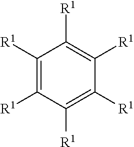

means that R₁ is connected to any substituent site of the benzene ring, and

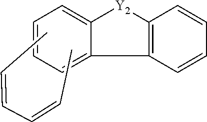

means that

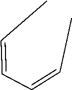

may be connected to any substituent site of the benzene ring of the

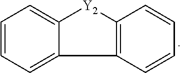

A cyclic alkyl group or a naphthyl group described in the disclosure have a same meaning and may be interchanged.

Embodiments of the disclosure provide a compound, a mixture, and an organic electronic device to improve luminous efficiency and service life of organic electronic devices.

In an embodiment, the compound is represented by formula (1):

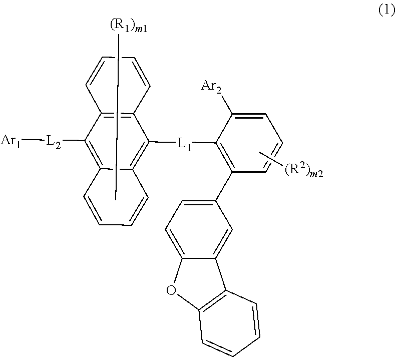

wherein Ar₁ and Ar₂ are each independently selected from an aromatic group containing 6 to 60 ring atoms, a heteroaromatic group containing 5 to 60 ring atoms, and combinations of these groups;

L₁ and L₂ are each independently selected from a single bond, an aromatic group containing 6 to 60 ring atoms, and a heteroaromatic group containing 5 to 60 ring atoms;

R₁ and R₂ are each independently selected from -D, a C₁-C₁₀ linear alkyl group, a C₁-C₁₀ linear alkoxy group, a C₁-C₁₀ linear thioalkoxy group, a C₃-C₁₀ branched alkyl group, a C₃-C₁₀ branched alkoxy group, a C₃-C₁₀ branched thioalkoxy group, a C₃-C₁₀ cyclic alkyl group, a C₃-C₁₀ cyclic alkoxy group, a C₃-C₁₀ cyclic thioalkoxy group, a methylsilyl group, a C₁-C₁₀ ketone group, a C₂-C₁₀ alkoxycarbonyl group, a C₇-C₁₀ aryloxycarbonyl group, a cyano group, an aminoformyl group, a haloformyl group, a formyl group, an isocyano group, an isocyanate group, a thiocyanate group, an isothiocyanate group, a hydroxy group, a nitro group, an amino group, —CF₃, —Cl, —Br, —F, —I, an aromatic group containing 6 to 30 ring atoms, a heteroaromatic group containing 5 to 30 ring atoms, an aryloxy group containing 5 to 30 ring atoms, and a heteroaryloxy group containing 5 to 30 ring atoms at each occurrence;

m1 is 0, 1, 2, 3, 4, 5, 6, 7, or 8; and

m2 is 0, 1, 2, or 3.

In some embodiments, R₁ and R₂ are each independently selected from -D, a C₁-C₂₀ linear alkyl group, a C₁-C₂₀ linear alkoxy group, a C₁-C₂₀ linear thioalkoxy group, a C₃-C₂₀ branched alkyl group, a C₃-C₂₀ branched alkoxy group, a C₃-C₂₀ branched thioalkoxy group, a C₃-C₂₀ cyclic alkyl group, a C₃-C₂₀ cyclic alkoxy group, a C₃-C₂₀ cyclic thioalkoxy group, a methylsilyl group, a C₁-C₂₀ ketone group, a C₂-C₂₀ alkoxycarbonyl group, a C₇-C₂₀ aryloxycarbonyl group, a cyano group, an aminoformyl group, a haloformyl group, a formyl group, an isocyano group, an isocyanate group, a thiocyanate group, an isothiocyanate group, a hydroxy group, a nitro group, an amino group, —CF₃, —Cl, —Br, —F, —I, an aromatic group containing 6 to 60 ring atoms, a heteroaromatic group containing 5 to 60 ring atoms, an aryloxy group containing 5 to 60 ring atoms, and a heteroaryloxy group containing 5 to 60 ring atoms at each occurrence.

In some embodiments, Ar₁ is selected from a substituted or unsubstituted aromatic group containing 6 to 16 ring atoms and a substituted or unsubstituted heteroaromatic group containing 6 to 16 ring atoms.

Further, Ar₁ is selected from any of following structures:

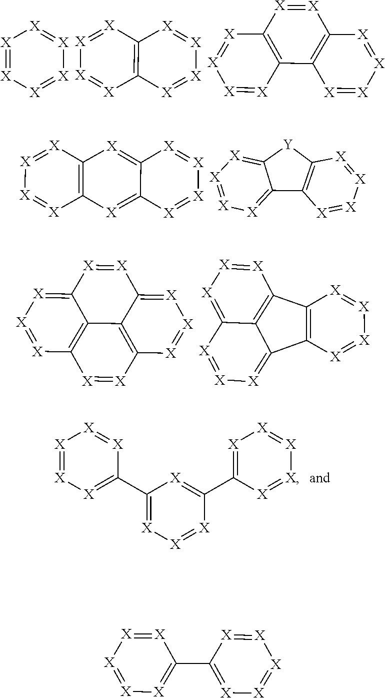

wherein X is independently selected from CR₃ and N at each occurrence;

Y is selected from NR₄, CR₅R₆, SiR₅R₆, O, S, S═O, and SO₂; and

R₃, R₄, R₅, and R₆ are each independently selected from —H, -D, a C₁-C₂₀ linear alkyl group, a C₁-C₂₀ linear alkoxy group, a C₁-C₂₀ linear thioalkoxy group, a C₃-C₂₀ branched alkyl group, a C₃-C₂₀ branched alkoxy group, a C₃-C₂₀ branched thioalkoxy group, a C₃-C₂₀ cyclic alkyl group, a C₃-C₂₀ cyclic alkoxy group, a C₃-C₂₀ cyclic thioalkoxy group, a methylsilyl group, a C₁-C₂₀ ketone group, a C₂-C₂₀ alkoxycarbonyl group, a C₇-C₂₀ aryloxycarbonyl group, a cyano group, an aminoformyl group, a haloformyl group, a formyl group, an isocyano group, an isocyanate group, a thiocyanate group, an isothiocyanate group, a hydroxy group, a nitro group, an amino group, —CF₃, —Cl, —Br, —F, —I, a substituted or unsubstituted aromatic group containing 6 to 60 ring atoms, a substituted or unsubstituted heteroaromatic group containing 5 to 60 ring atoms, a substituted or unsubstituted aryloxy group containing 5 to 60 ring atoms, and a substituted or unsubstituted heteroaryloxy group containing 5 to 60 ring atoms at each occurrence; a ring is formed or no ring is formed by an interconnection of R₅ and R₆.

In the disclosure, when X is a linking site, X is C; when Y is a linking site, Y is N.

In some specific embodiments, R₃ is independently selected from —H, -D, a C₁-C₁₀ linear alkyl group, a C₃-C₁₀ branched alkyl group, a C₃-C₁₀ cyclic alkyl group, a silyl group, a cyano group, an isocyano group, a nitro group, —CF₃, —Cl, —Br, —F, —I, a substituted or unsubstituted aromatic group containing 6 to 20 ring atoms, and a substituted or unsubstituted heteroaromatic group containing 5 to 20 ring atoms at each occurrence.

Further, R₃ is independently selected from —H, -D, a C₁-C₈ linear alkyl group, a C₃-C₈ branched alkyl group, a C₃-C₈ cyclic alkyl group, a silyl group, a substituted or unsubstituted aromatic group containing 6 to 10 ring atoms, and a substituted or unsubstituted heteroaromatic group containing 5 to 10 ring atoms at each occurrence.

A substituent group of R₃ is preferably selected from -D, a C₁-C₄ linear alkyl group, a C₃-C₄ branched alkyl group, a phenyl group, and a pyridyl group.

In some specific embodiments, R₃ is independently selected from a C₁-C₁₀ linear alkyl group, a C₃-C₁₀ branched alkyl group, a C₃-C₁₀ cyclic alkyl group, a substituted or unsubstituted aromatic group containing 6 to 20 ring atoms, and a substituted or unsubstituted heteroaromatic group containing 5 to 20 ring atoms.

Further, R₄ is selected from a methyl group, an ethyl group, an isopropyl group, a tert-butyl group, a substituted or unsubstituted aromatic group containing 6 to 13 ring atoms, and a substituted or unsubstituted heteroaromatic group containing 6 to 13 ring atoms.

Furthermore, R₄ is selected from a methyl group, an ethyl group, an isopropyl group, a tert-butyl group, a phenyl group, a pyridyl group, a pyrimidinyl group, a triazine group, a biphenyl group, a terphenyl group, and a naphthyl group.

In some specific embodiments, R₅ and R₆ are selected from —H, -D, a C₁-C₈ linear alkyl group, a C₃-C₈ branched alkyl group, a C₃-C₈ cyclic alkyl group, a substituted or unsubstituted aromatic group containing 6 to 10 ring atoms, and a substituted or unsubstituted heteroaromatic group containing 5 to 10 ring atoms.

Further, R₅ and R₆ are selected from —H, -D, a methyl group, an ethyl group, an isopropyl group, a tert-butyl group, a phenyl group, a pyridyl group, a pyrimidinyl group, a triazine group, a biphenyl group, a terphenyl group, and a naphthyl group.

In particular, Ar₁ is selected from any of following structures:

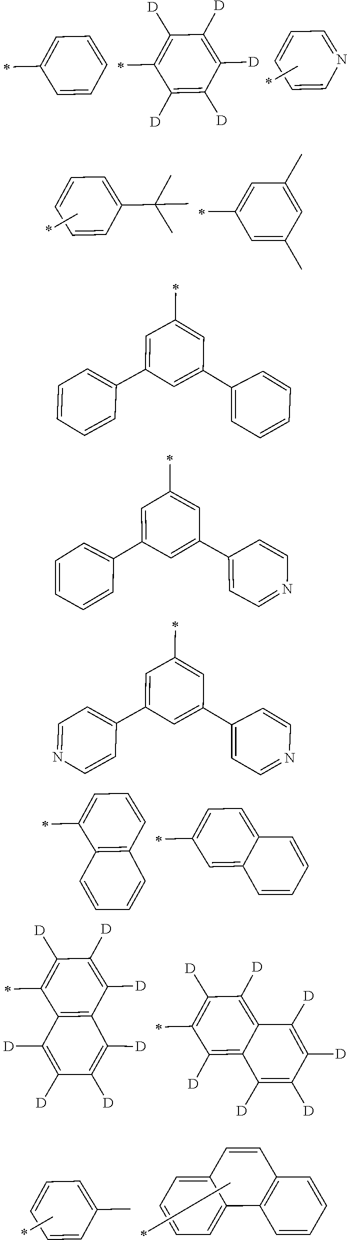

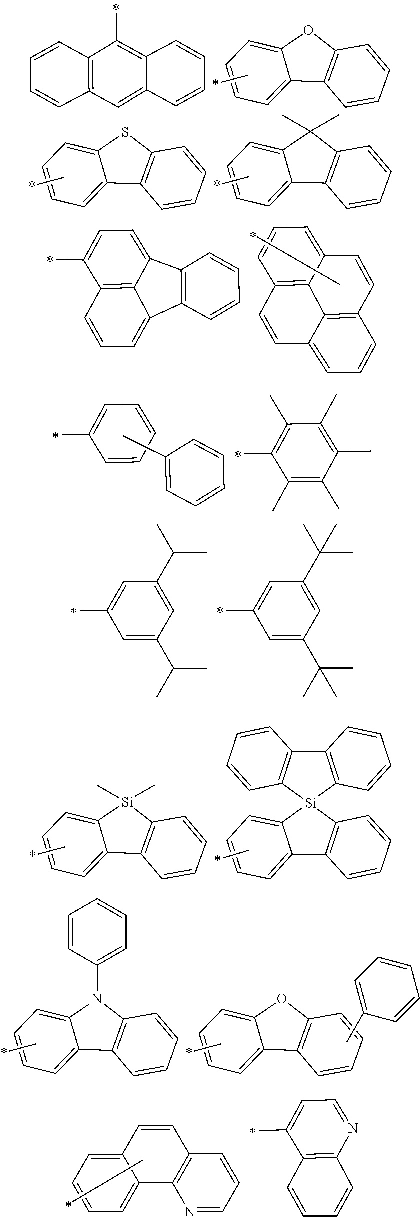

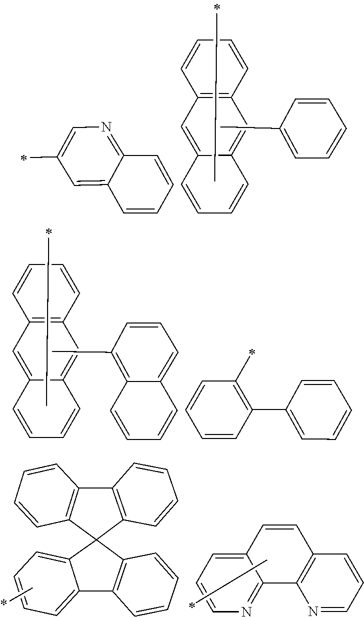

wherein * indicates a linking site.

In some embodiments, Ar₂ is selected from a substituted or unsubstituted aromatic group containing 6 to 16 ring atoms and a substituted or unsubstituted heteroaromatic group containing 6 to 16 ring atoms.

Further, Ar₂ is selected from any of following structures:

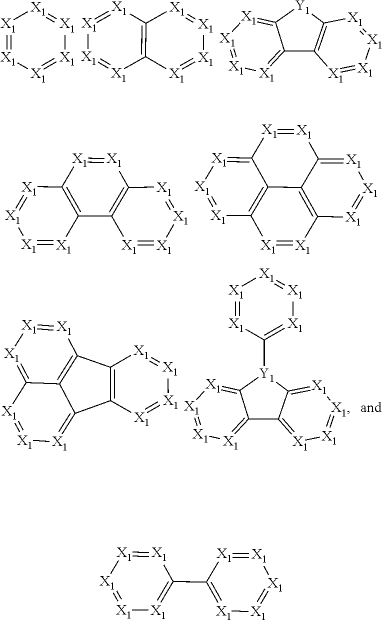

wherein X₁ is independently selected from CR₇ and N at each occurrence;

Y₁ is selected from NR₈, CR₉R₁₀, SiR₁₁R₁₂, O, S, S═O, and SO₂; and

R₇, R₈, R₉ and R₁₀ are each independently selected from —H, -D, a C₁-C₂₀ linear alkyl group, a C₁-C₂₀ linear alkoxy group, a C₁-C₂₀ linear thioalkoxy group, a C₃-C₂₀ branched alkyl group, a C₃-C₂₀ branched alkoxy group, a C₃-C₂₀ branched thioalkoxy group, a C₃-C₂₀ cyclic alkyl group, a C₃-C₂₀ cyclic alkoxy group, a C₃-C₂₀ cyclic thioalkoxy group, a methylsilyl group, a C₁-C₂₀ ketone group, a C₂-C₂₀ alkoxycarbonyl group, a C₇-C₂₀ aryloxycarbonyl group, a cyano group, an aminoformyl group, a haloformyl group, a formyl group, an isocyano group, an isocyanate group, a thiocyanate group, an isothiocyanate group, a hydroxy group, a nitro group, an amino group, —CF₃, —Cl, —Br, —F, —I, a substituted or unsubstituted aromatic group containing 6 to 60 ring atoms, a substituted or unsubstituted heteroaromatic group containing 5 to 60 ring atoms, a substituted or unsubstituted aryloxy group containing 5 to 60 ring atoms, and a substituted or unsubstituted heteroaryloxy group containing 5 to 60 ring atoms at each occurrence; a ring is formed or no ring is formed by an interconnection of R₉ and R₁₀.

In the disclosure, when X₁ is a linking site, X₁ is C; when Y₁ is a linking site, Y₁ is N.

In some specific embodiments, R₇ is independently selected from —H, -D, a C₁-C₁₀ linear alkyl group, a C₃-C₁₀ branched alkyl group, a C₃-C₁₀ cyclic alkyl group, a silyl group, a cyano group, an isocyano group, a nitro group, —CF₃, —Cl, —Br, —F, —I, a substituted or unsubstituted aromatic group containing 6 to 20 ring atoms, and a substituted or unsubstituted heteroaromatic group containing 5 to 20 ring atoms at each occurrence.

Further, R₇ is independently selected from —H, -D, a C₁-C₈ linear alkyl group, a C₃-C₈ branched alkyl group, a C₃-C₈ cyclic alkyl group, a silyl group, a substituted or unsubstituted aromatic group containing 6 to 10 ring atoms, and a substituted or unsubstituted heteroaromatic group containing 5 to 10 ring atoms at each occurrence.

A substituent group of R₇ is preferably selected from -D, a C₁-C₄ linear alkyl group, a C₃-C₄ branched alkyl group, a phenyl group, and a pyridyl group.

In some specific embodiments, R₈ is independently selected from a C₁-C₁₀ linear alkyl group, a C₃-C₁₀ branched alkyl group, a C₃-C₁₀ cyclic alkyl group, a substituted or unsubstituted aromatic group containing 6 to 20 ring atoms, and a substituted or unsubstituted heteroaromatic group containing 5 to 20 ring atoms.

Further, R₈ is selected from a methyl group, an ethyl group, an isopropyl group, a tert-butyl group, a substituted or unsubstituted aromatic group containing 6 to 13 ring atoms, and a substituted or unsubstituted heteroaromatic group containing 6 to 13 ring atoms, which is substituted or unsubstituted.

Furthermore, R₈ is selected from a methyl group, an ethyl group, an isopropyl group, a tert-butyl group, a phenyl group, a pyridyl group, a pyrimidinyl group, a triazine group, a biphenyl group, a terphenyl group, and a naphthyl group.

In some specific embodiments, R₉ and R₁₀ are selected from —H, -D, a C₁-C₈ linear alkyl group, a C₃-C₈ branched alkyl group, a C₃-C₈ cyclic alkyl group, a substituted or unsubstituted aromatic group containing 6 to 10 ring atoms, and a substituted or unsubstituted heteroaromatic group containing 5 to 10 ring atoms.

Further, R₉ and R₁₀ are selected from —H, -D, a methyl group, an ethyl group, an isopropyl group, a phenyl group, a pyridyl group, a pyrimidinyl group, a triazine group, a biphenyl group, a terphenyl group, and a naphthyl group.

In particular, Ar₂ is selected from any of following structures:

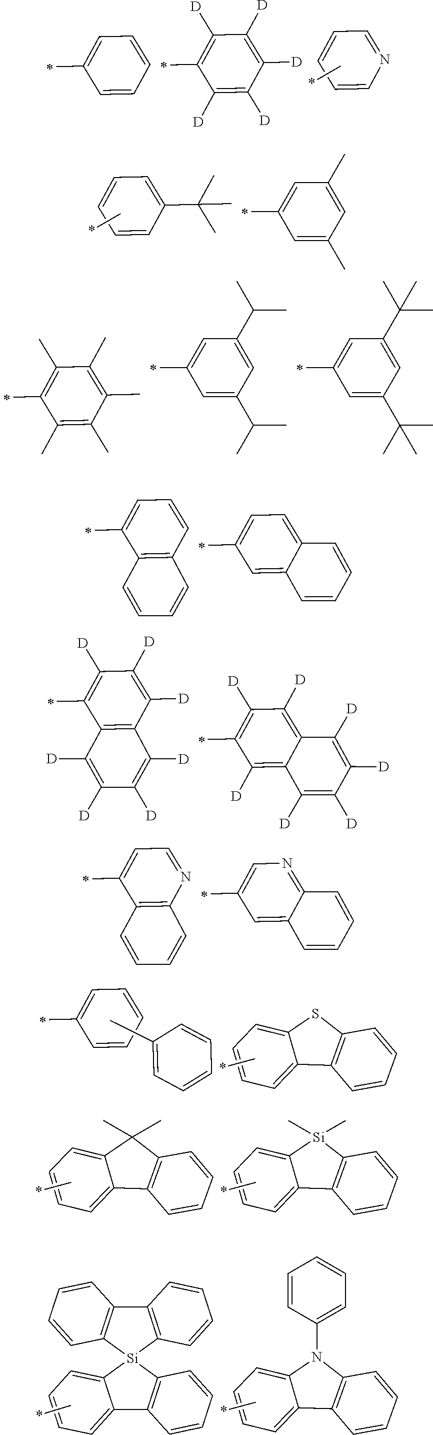

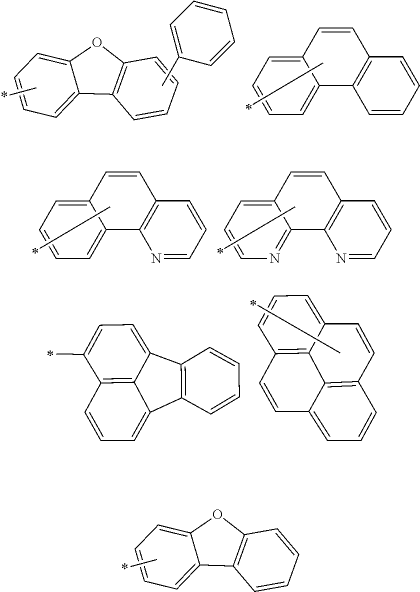

In some specific embodiments, L₁ and L₂ are each independently selected from a single bond and any of following structures:

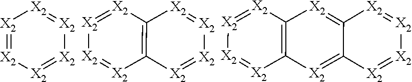

wherein X₂ is independently selected from CR₁₃ and N at each occurrence; and

R₁₃ is independently selected from —H, -D, a C₁-C₂₀ linear alkyl group, a C₁-C₂₀ linear alkoxy group, a C₁-C₂₀ linear thioalkoxy group, a C₃-C₂₀ branched alkyl group, a C₃-C₂₀ branched alkoxy group, a C₃-C₂₀ branched thioalkoxy group, a C₃-C₂₀ cyclic alkyl group, a C₃-C₂₀ cyclic alkoxy group, a C₃-C₂₀ cyclic thioalkoxy group, a methylsilyl group, a C₁-C₂₀ ketone group, a C₂-C₂₀ alkoxycarbonyl group, a C₇-C₂₀ aryloxycarbonyl group, a cyano group, an aminoformyl group, a haloformyl group, a formyl group, an isocyano group, an isocyanate group, a thiocyanate group, an isothiocyanate group, a hydroxy group, a nitro group, an amino group, —CF₃, —Cl, —Br, —F, —I, a substituted or unsubstituted aromatic group containing 6 to 60 ring atoms, a substituted or unsubstituted heteroaromatic group containing 5 to 60 ring atoms, a substituted or unsubstituted aryloxy group containing 5 to 60 ring atoms, and a substituted or unsubstituted heteroaryloxy group containing 5 to 60 ring atoms at each occurrence.

Further, R₁₃ is selected from —H, -D, a C₁-C₄ linear alkyl group, a C₃-C₄ branched alkyl group, an aromatic group containing 6 to 14 ring atoms, a heteroaromatic group containing 6 to 14 ring atoms, an aromatic group containing 6 to 14 ring atoms substituted by a C₁-C₄ linear alkyl group or a C₃-C₄ branched alkyl group, and a heteroaromatic group containing 6 to 14 ring atoms substituted by a C₁-C₄ linear alkyl group or a C₃-C₆ branched alkyl group.

In the disclosure, when X₂ is a linking site, X₂ is C.

In particular, L₁ and L₂ are each independently selected from a single bond and any of following structures:

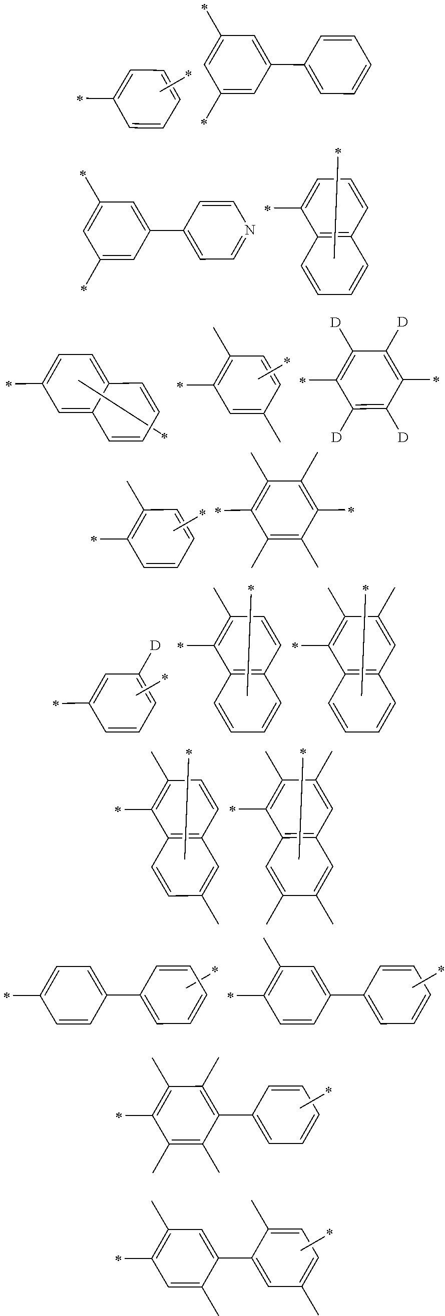

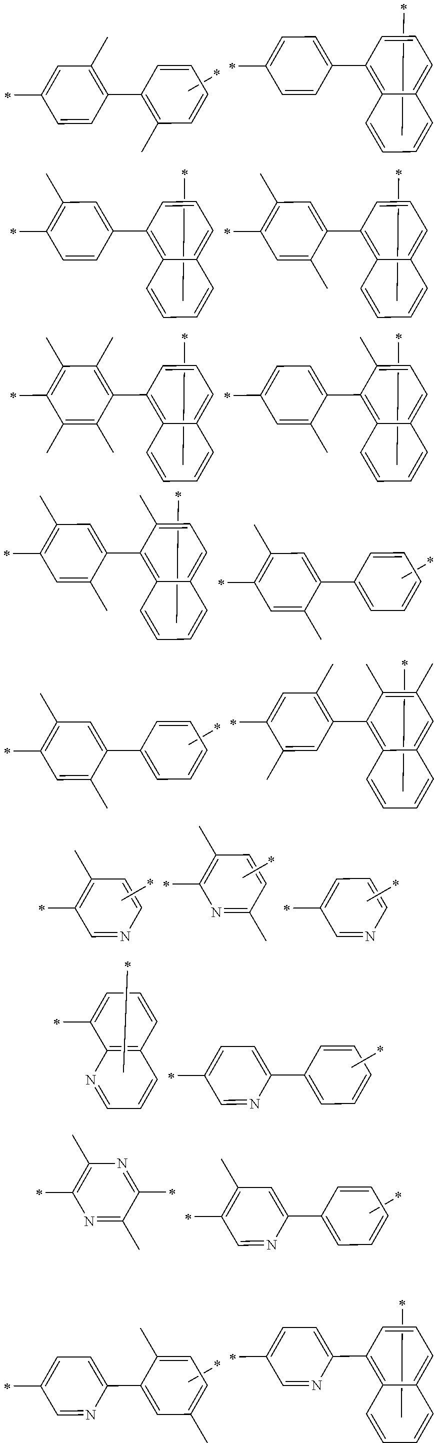

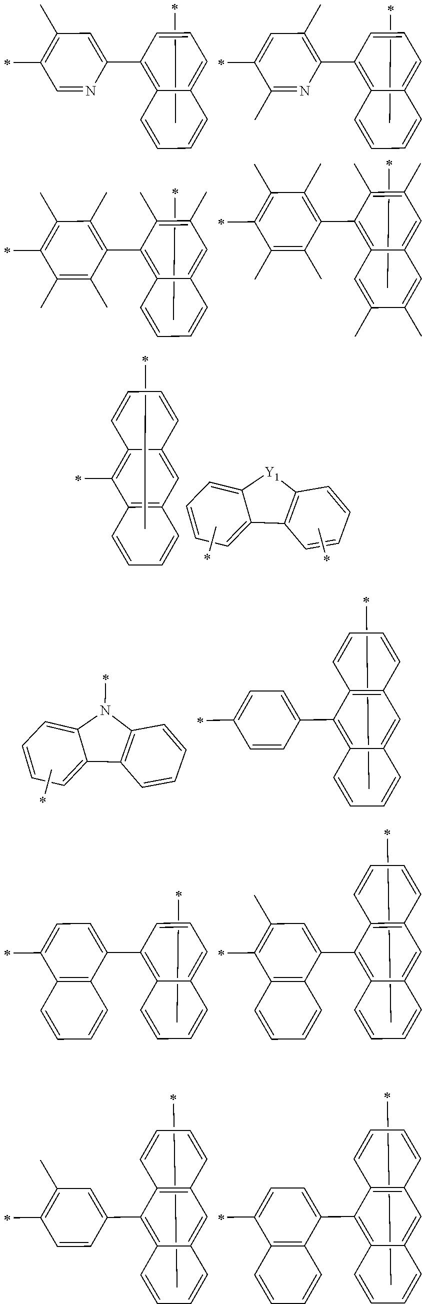

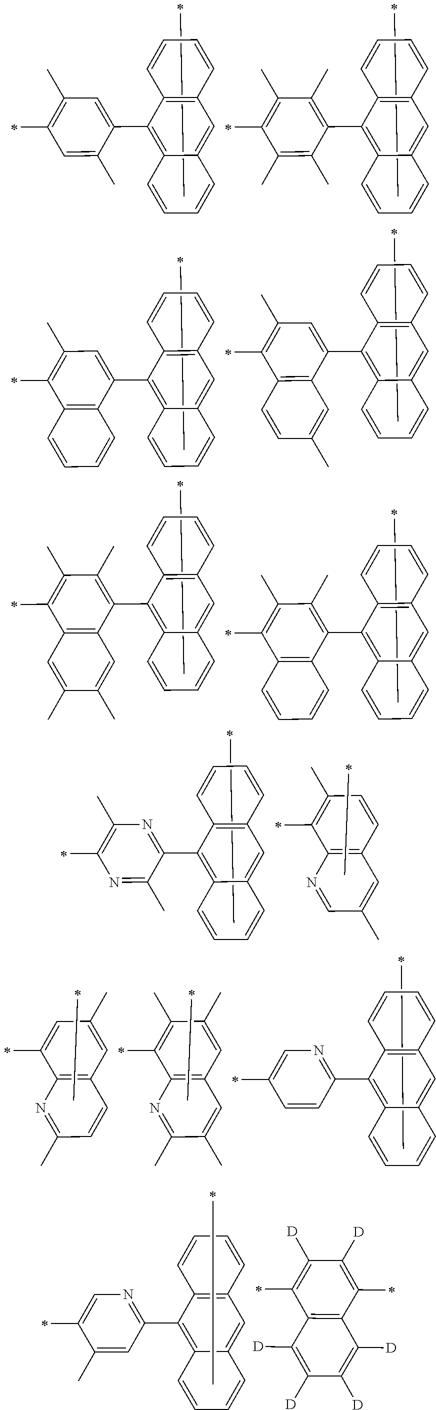

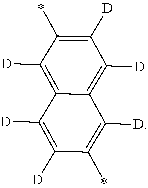

In an embodiment, the compound represented by formula (1) is specifically selected from but is not limited to any of following structures:

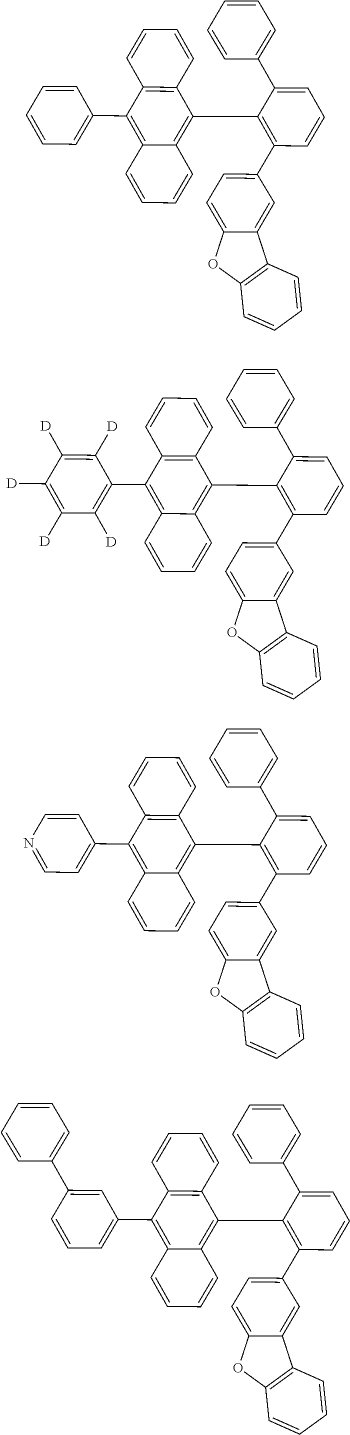

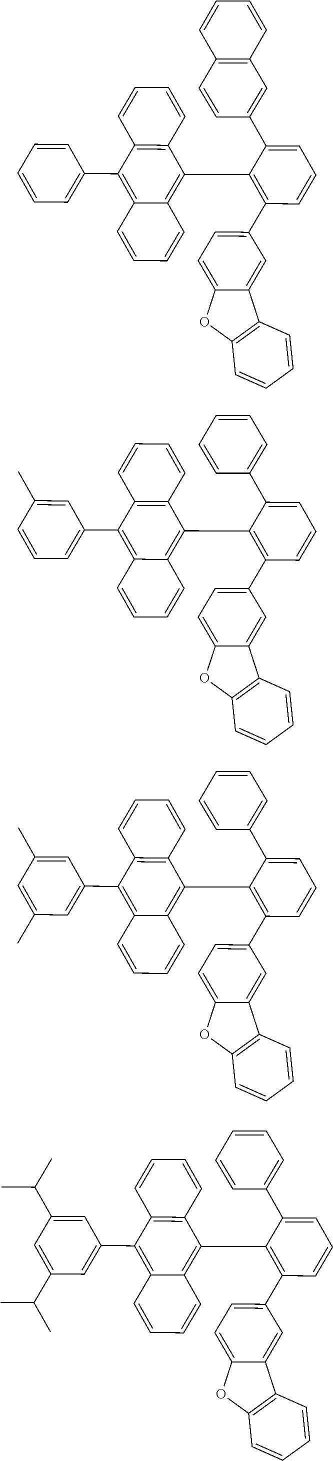

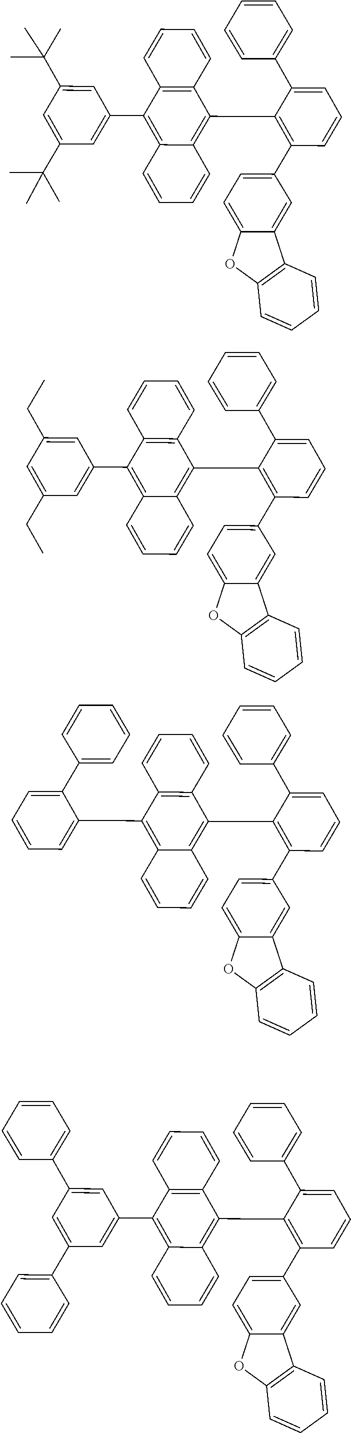

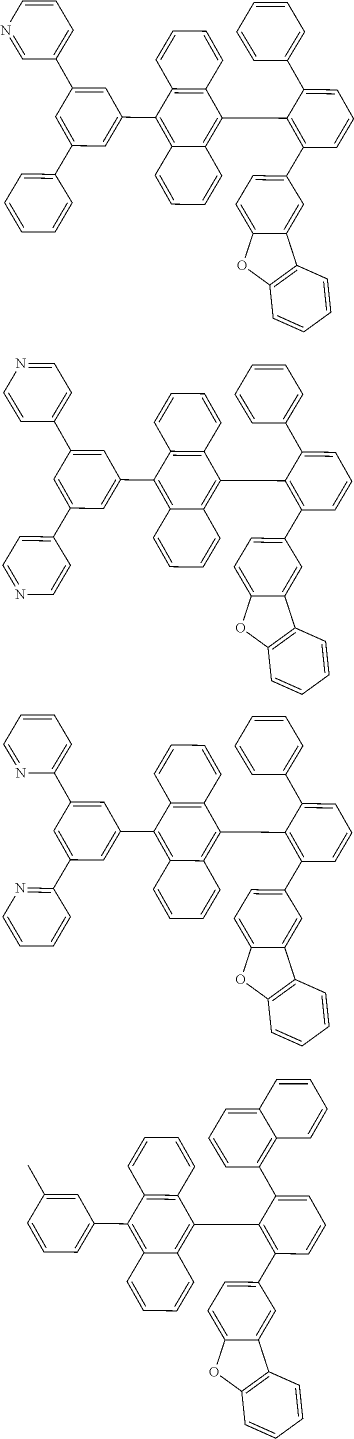

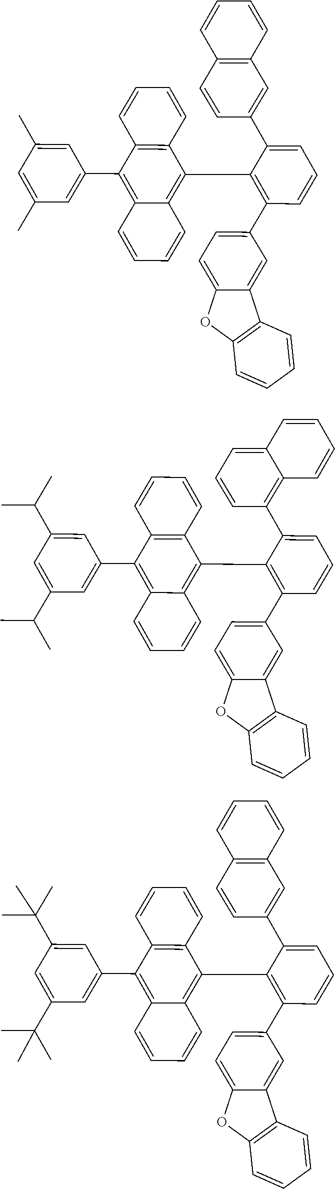

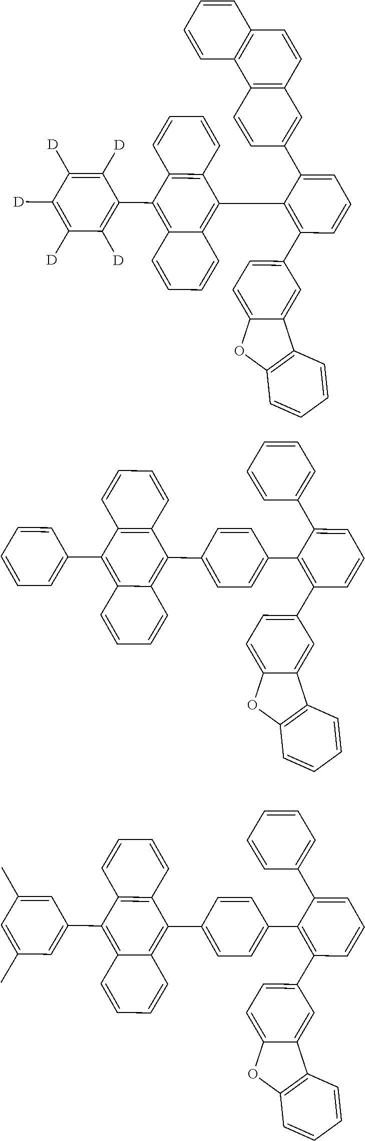

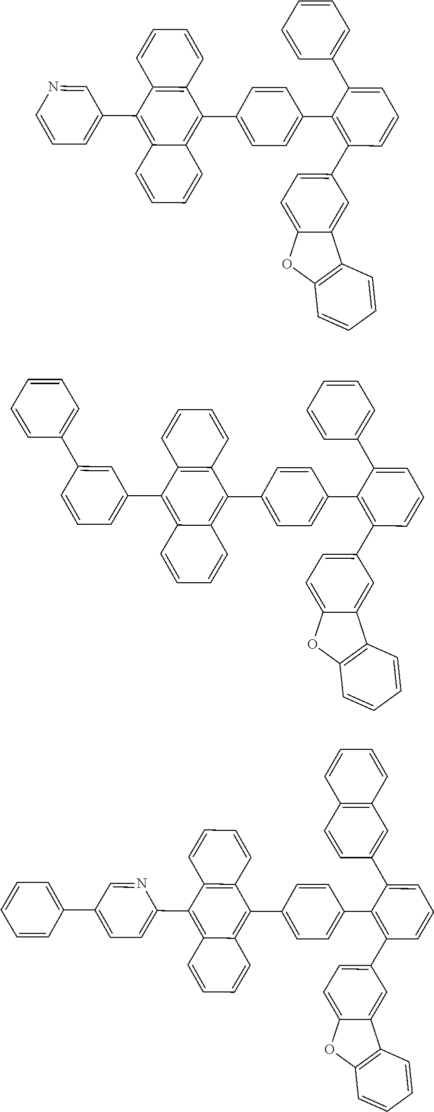

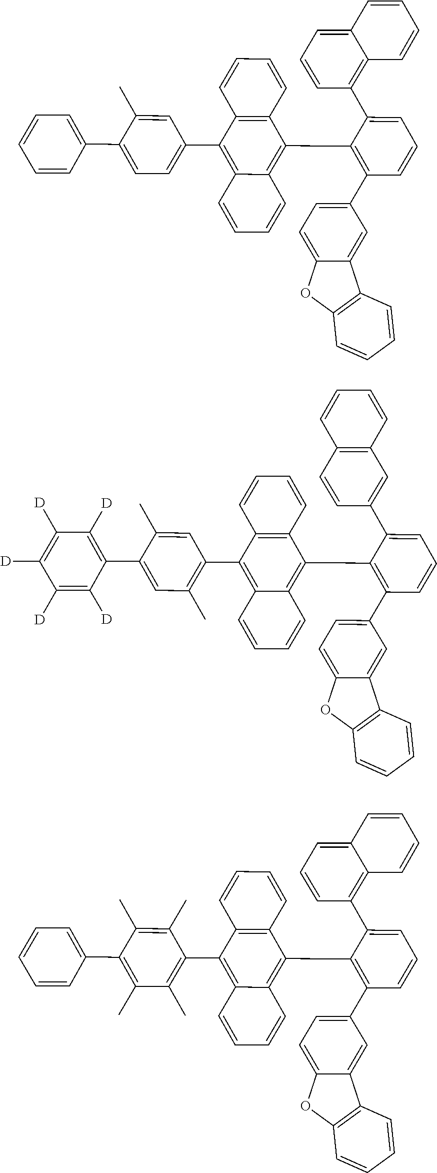

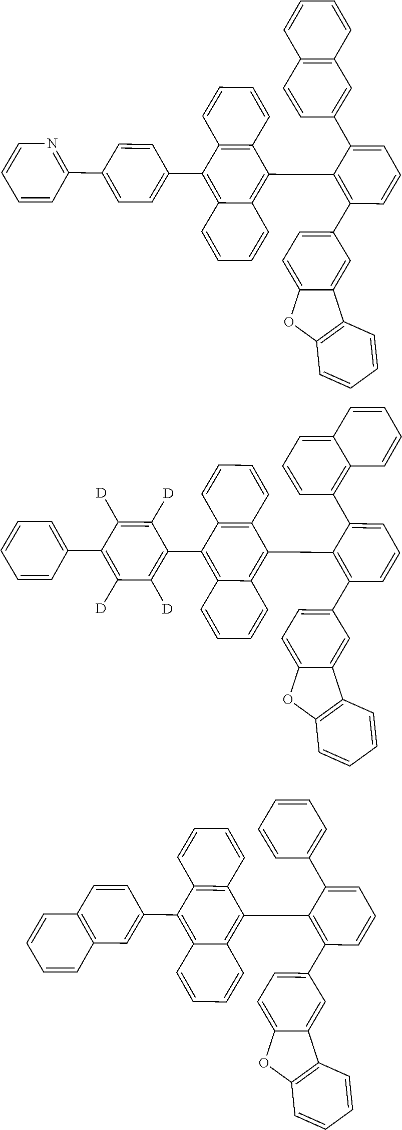

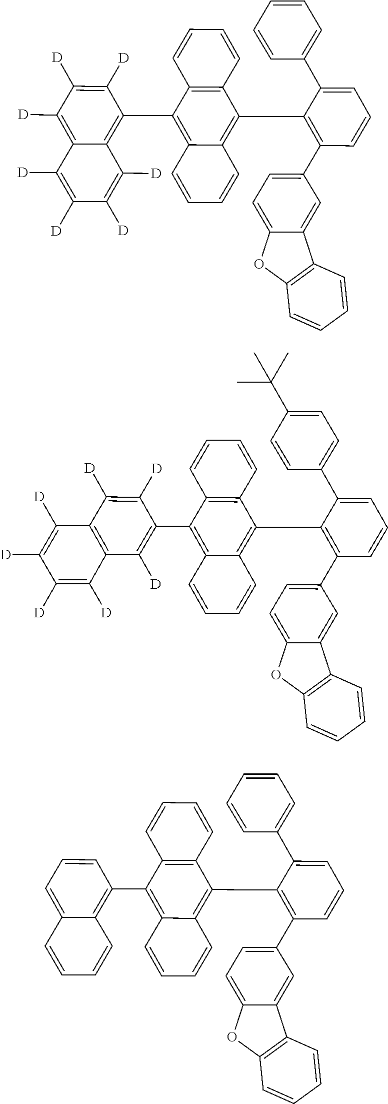

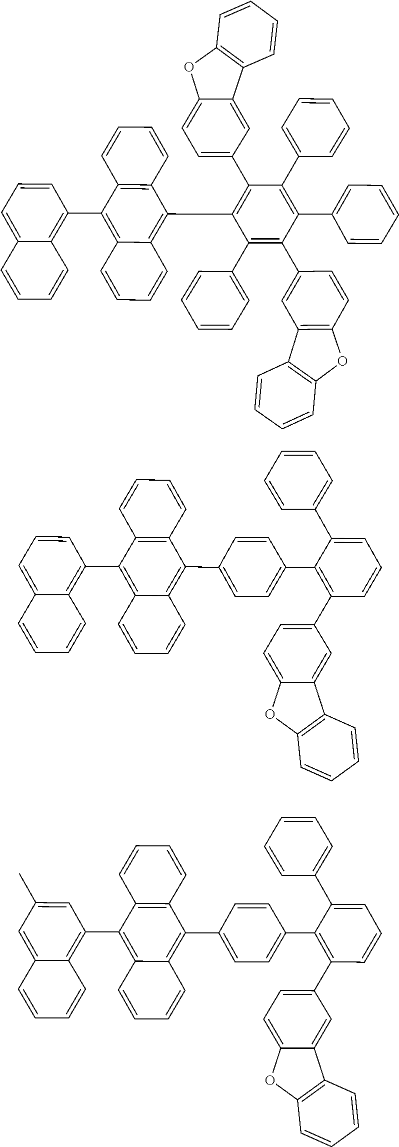

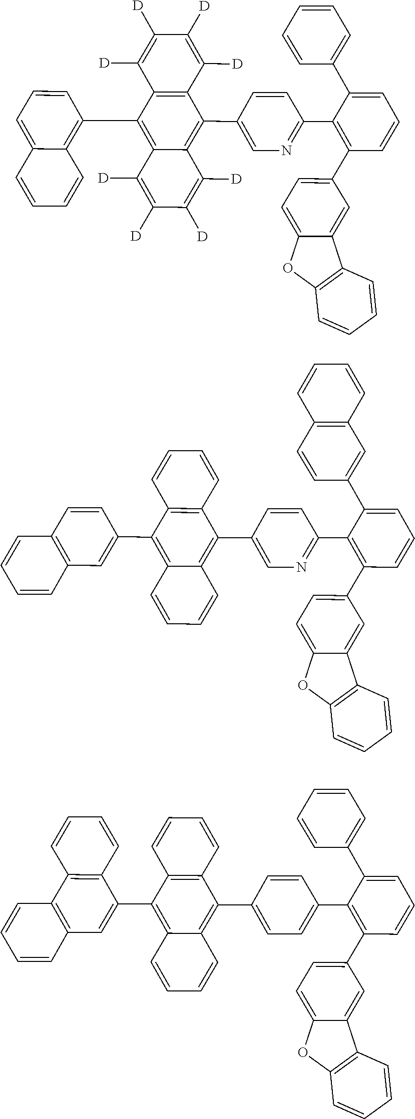

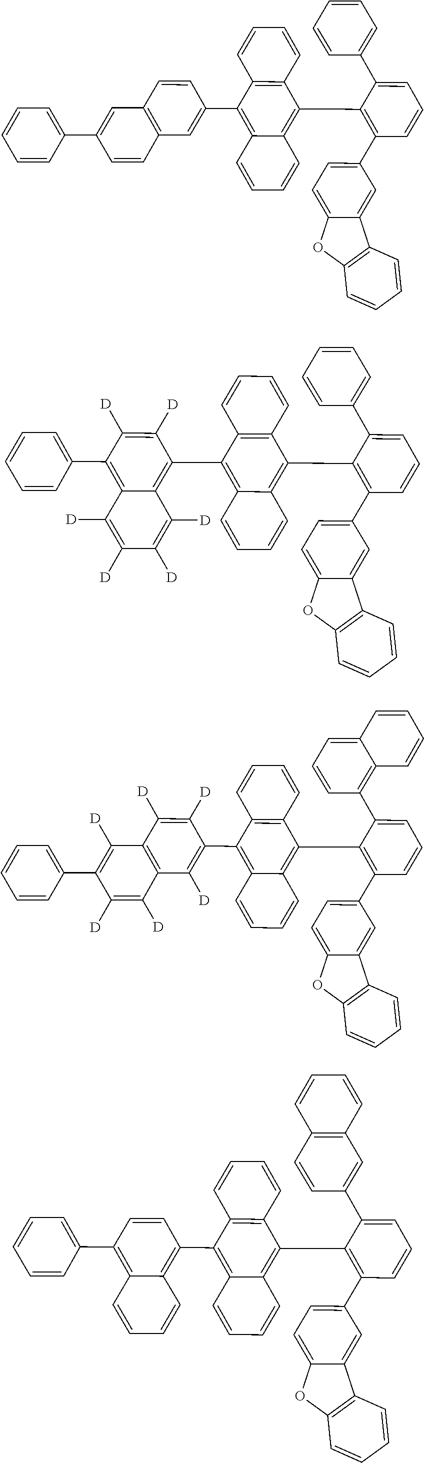

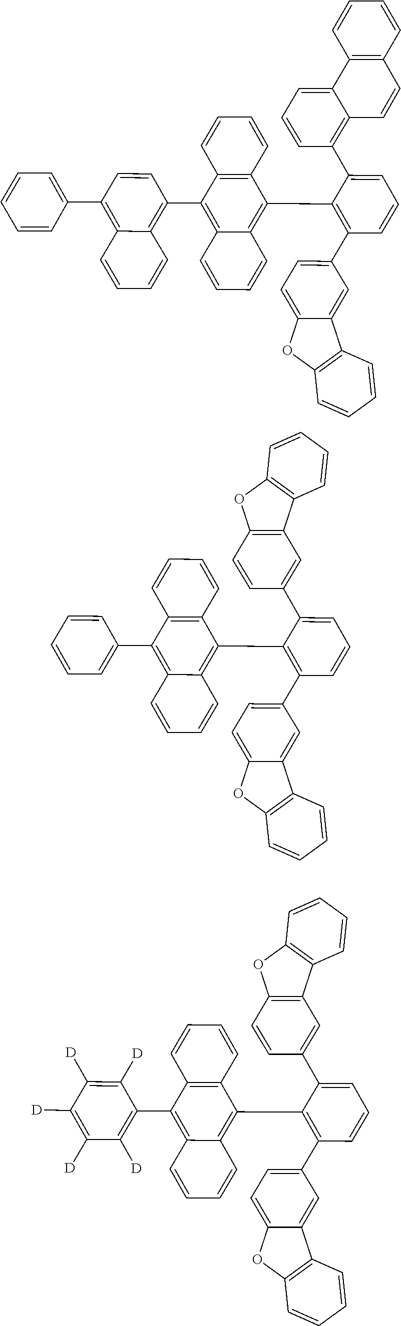

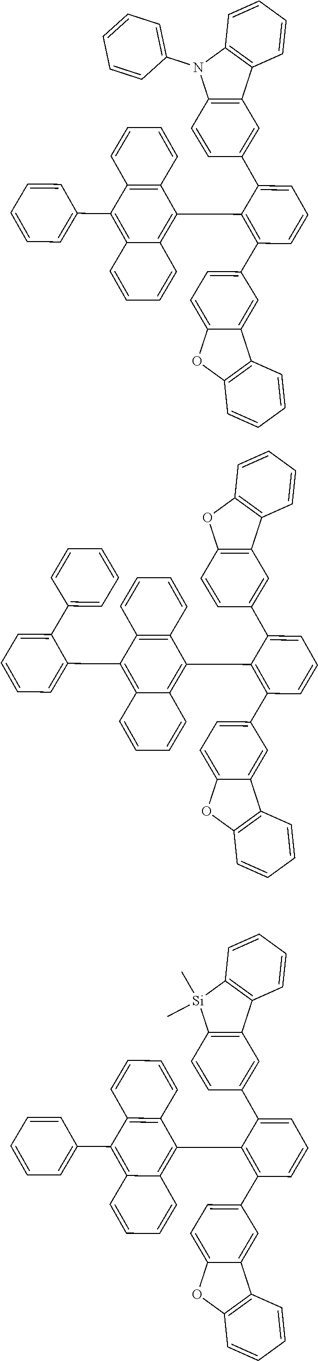

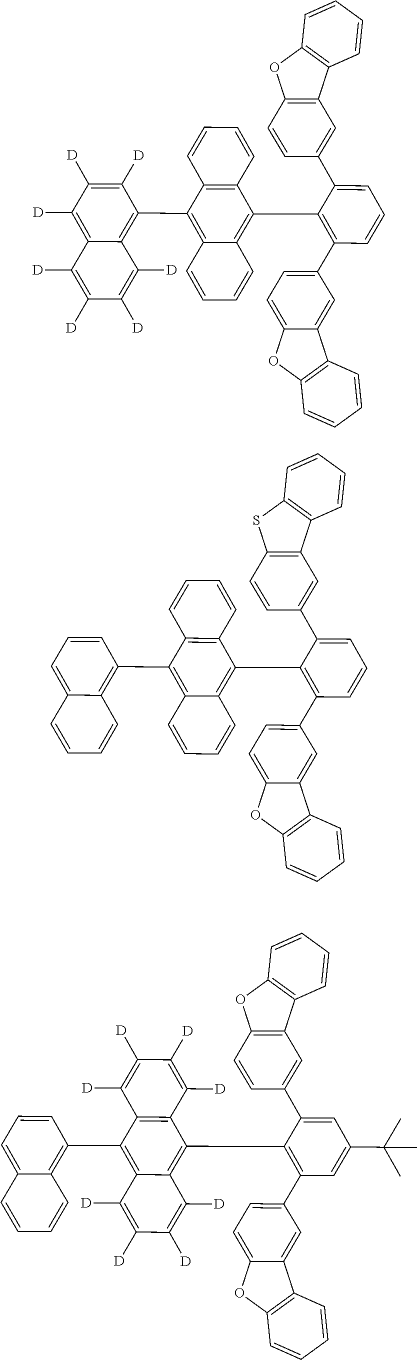

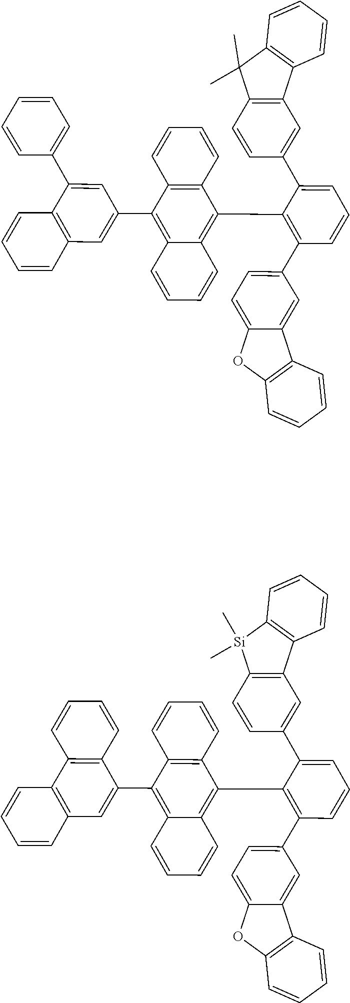 [SMILES: CC1(C)c2ccccc2-c2cc(-c3cccc(-c4ccc5oc6ccccc6c5c4)c3-c3c4ccccc4c(-c4cc(-c5ccccc5)c5ccccc5c4)c4ccccc34)ccc21 ; C[Si]1(C)c2ccccc2-c2cc(-c3cccc(-c4ccc5oc6ccccc6c5c4)c3-c3c4ccccc4c(-c4cc5ccccc5c5ccccc45)c4ccccc34)ccc21]

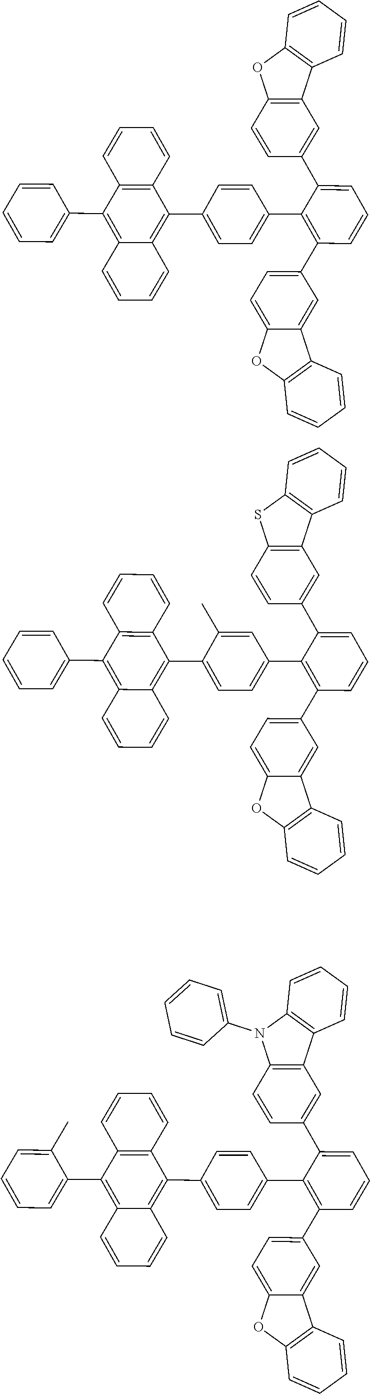

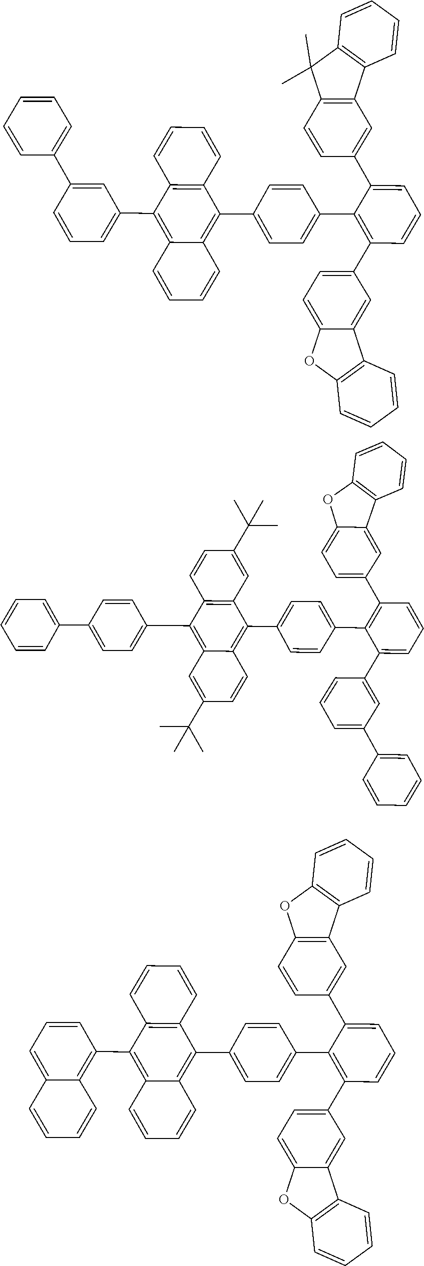

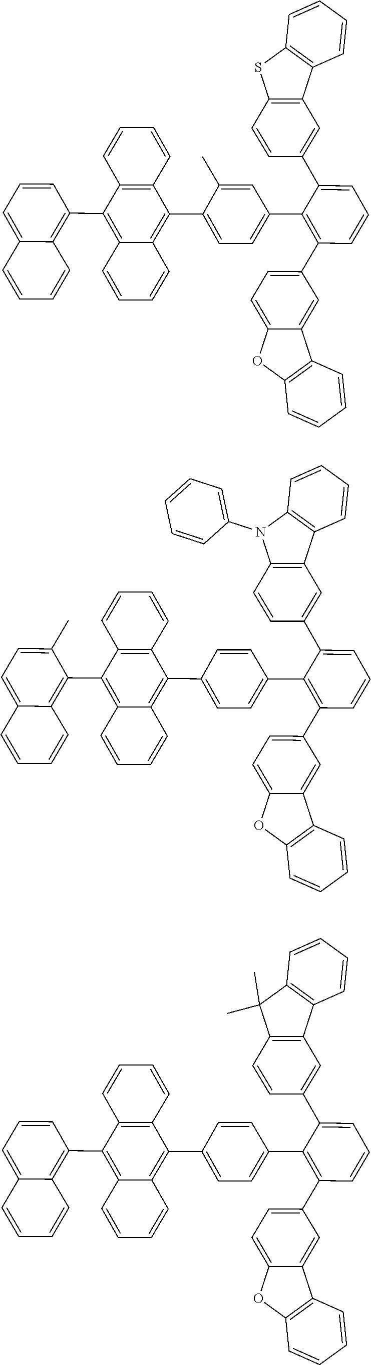

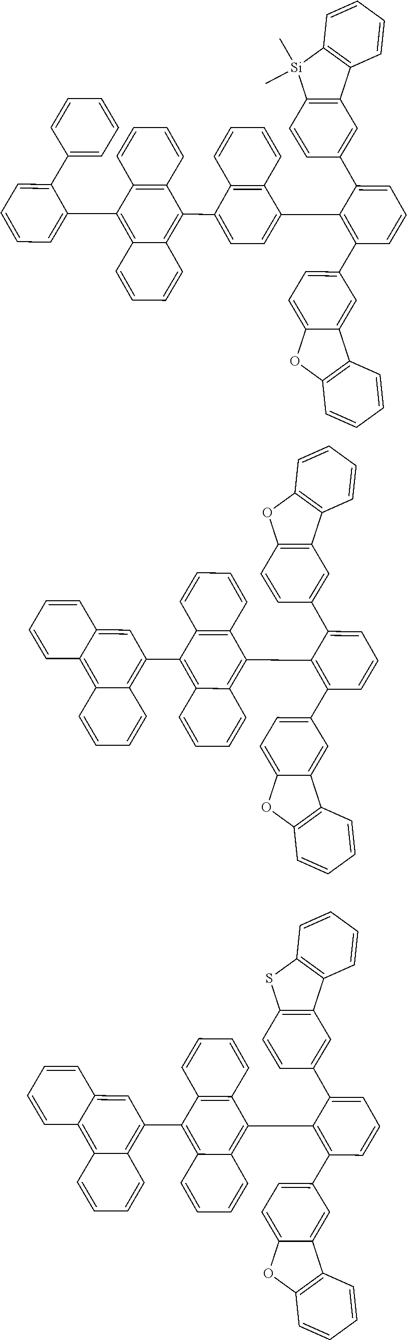

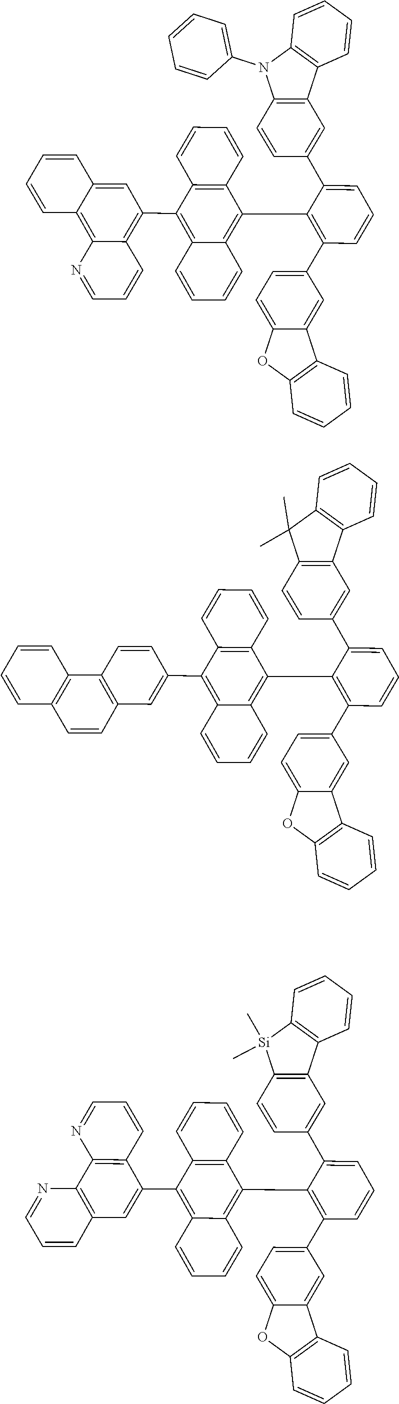

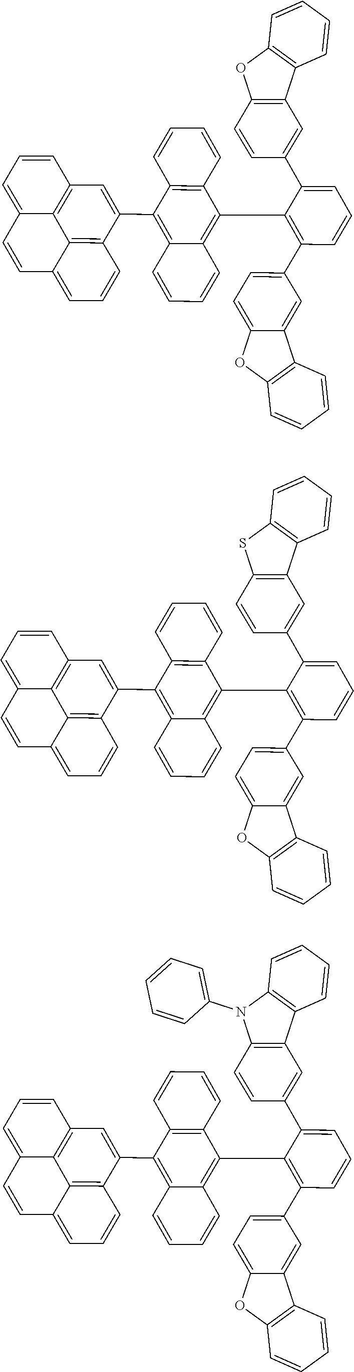

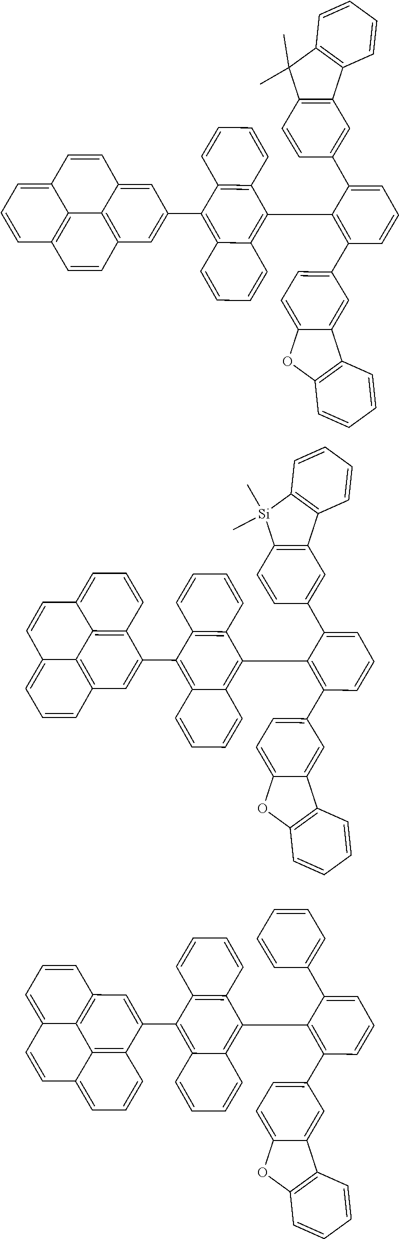

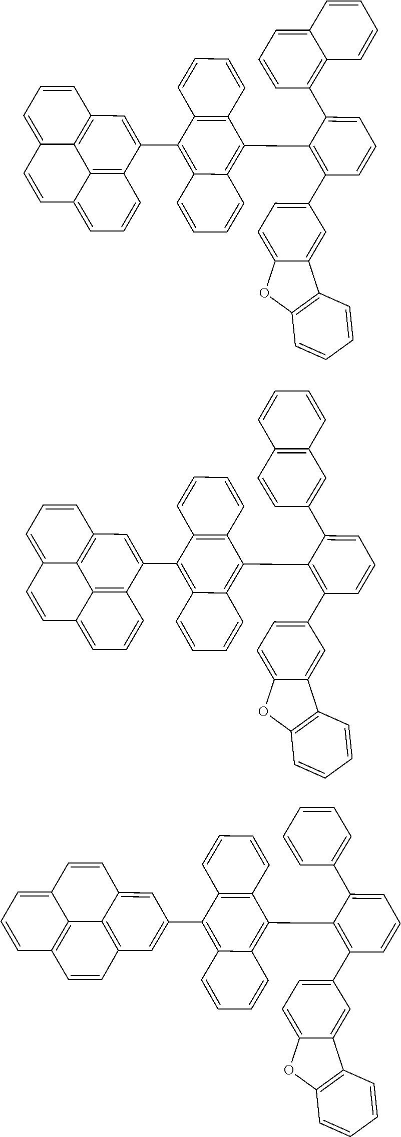

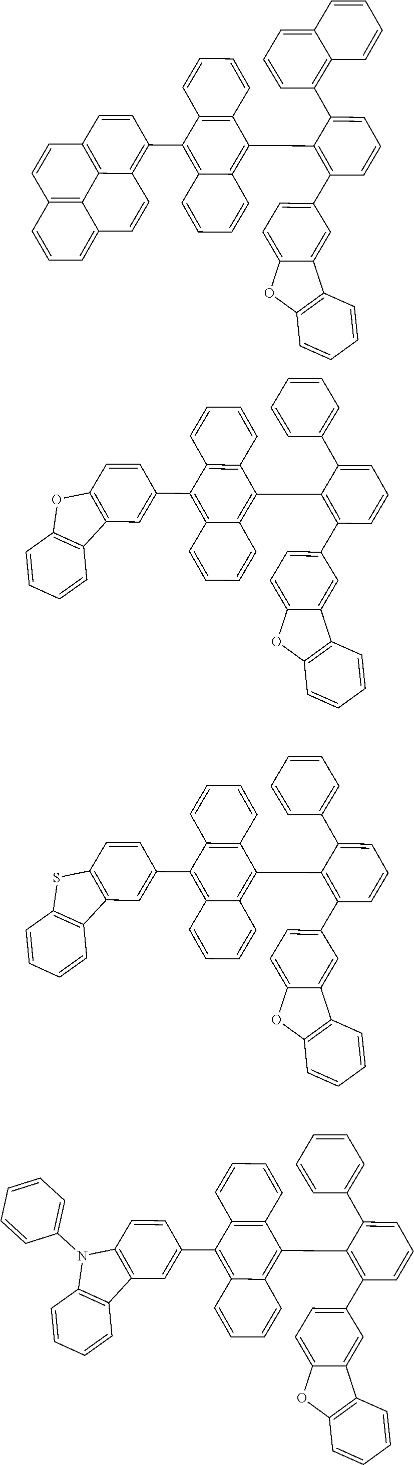

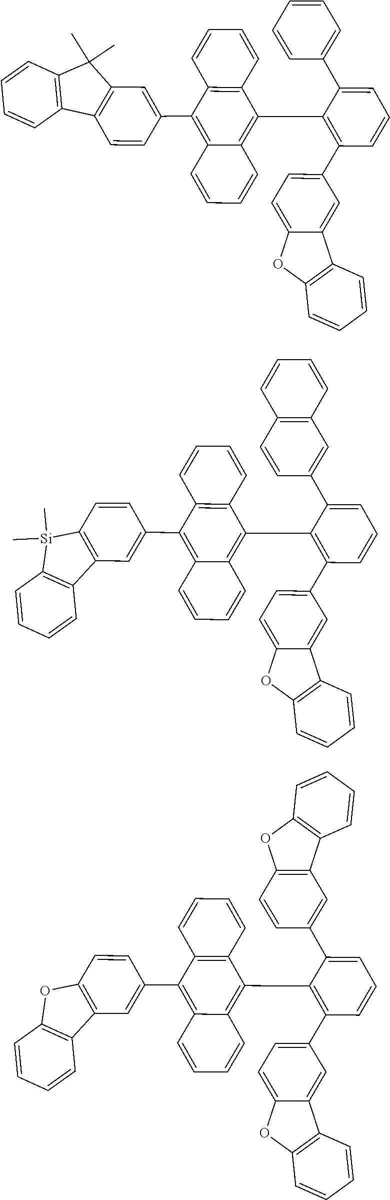

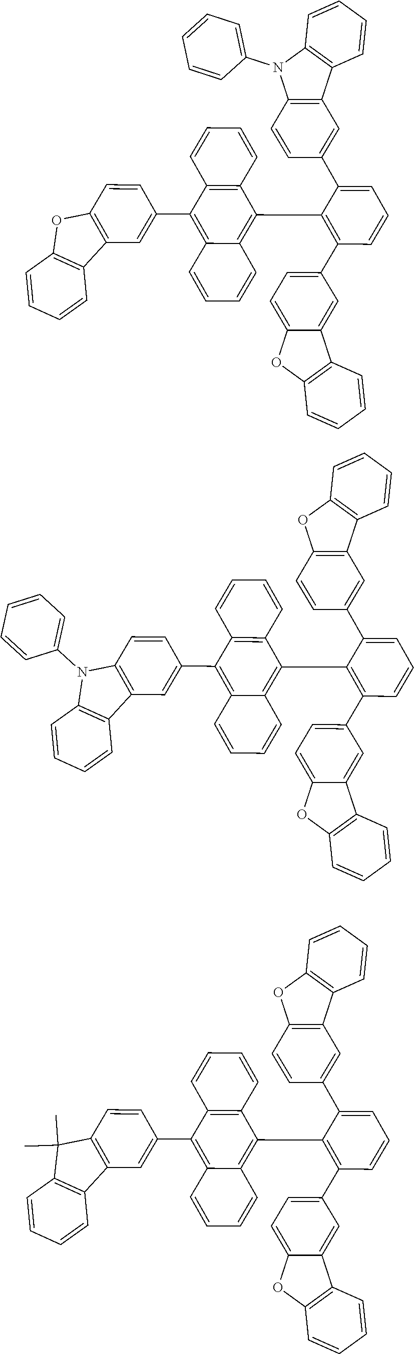

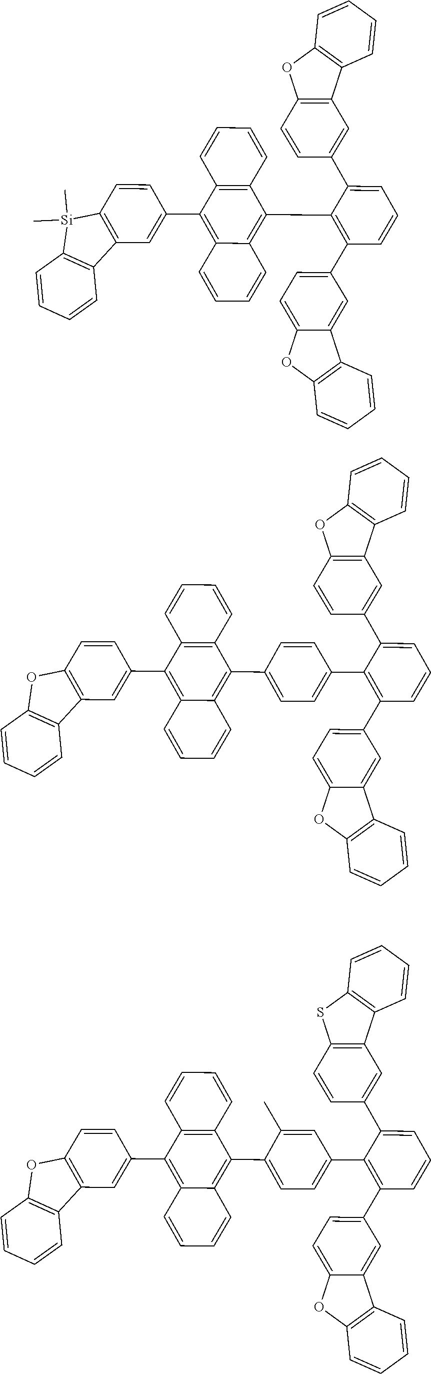

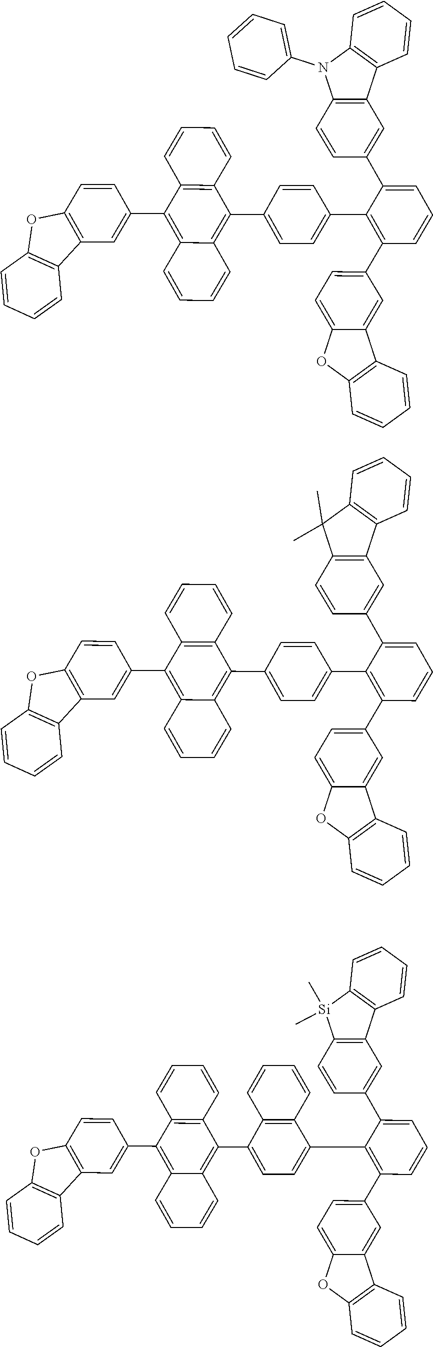

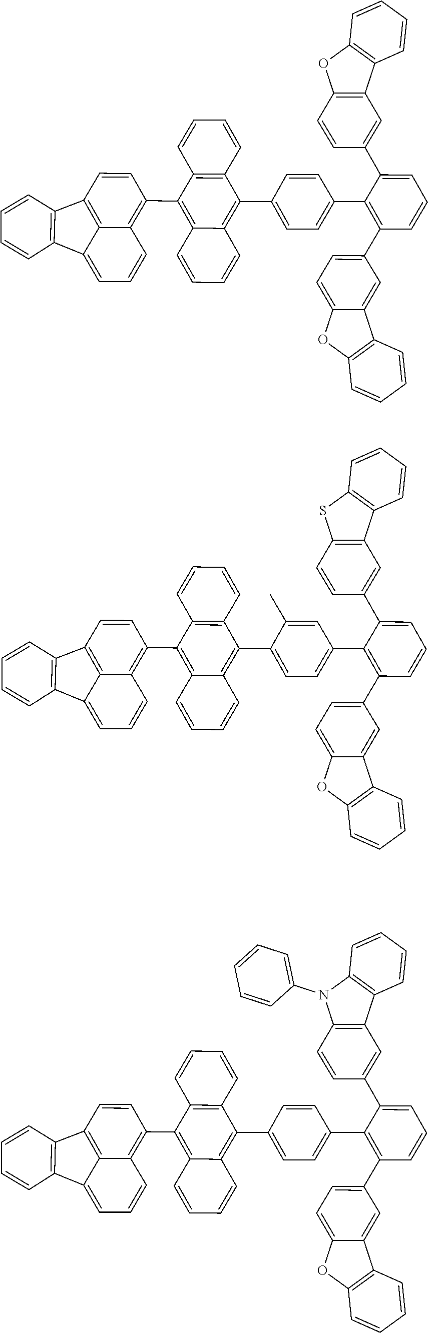 [SMILES: c1ccc2c(c1)-c1cccc3c(-c4c5ccccc5c(-c5ccc(-c6c(-c7ccc8oc9ccccc9c8c7)cccc6-c6ccc7oc8ccccc8c7c6)cc5)c5ccccc45)ccc-2c13 ; Cc1cc(-c2c(-c3ccc4oc5ccccc5c4c3)cccc2-c2ccc3sc4ccccc4c3c2)ccc1-c1c2ccccc2c(-c2ccc3c4c(cccc24)-c2ccccc2-3)c2ccccc12 ; c1ccc(-n2c3ccccc3c3cc(-c4cccc(-c5ccc6oc7ccccc7c6c5)c4-c4ccc(-c5c6ccccc6c(-c6ccc7c8c(cccc68)-c6ccccc6-7)c6ccccc56)cc4)ccc32)cc1]

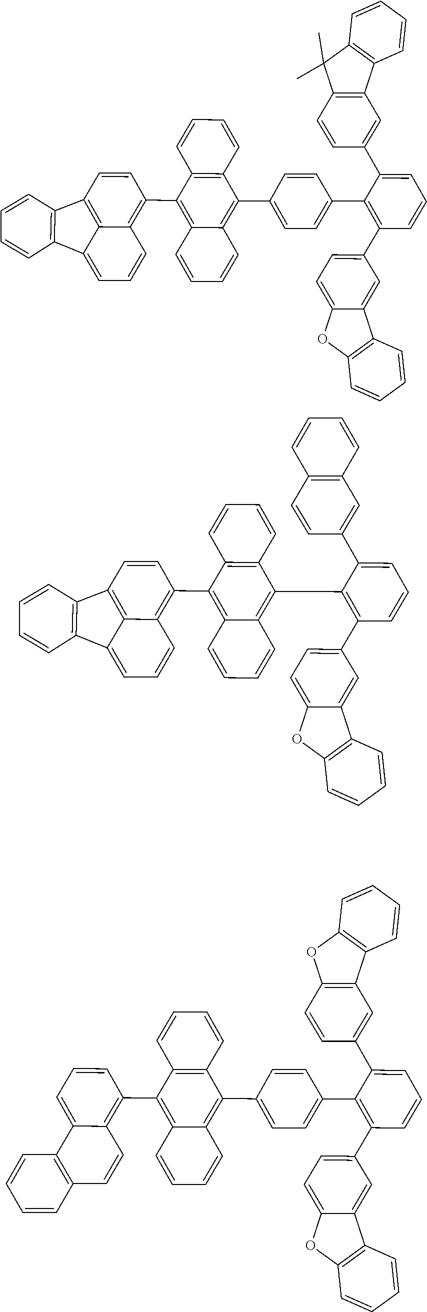

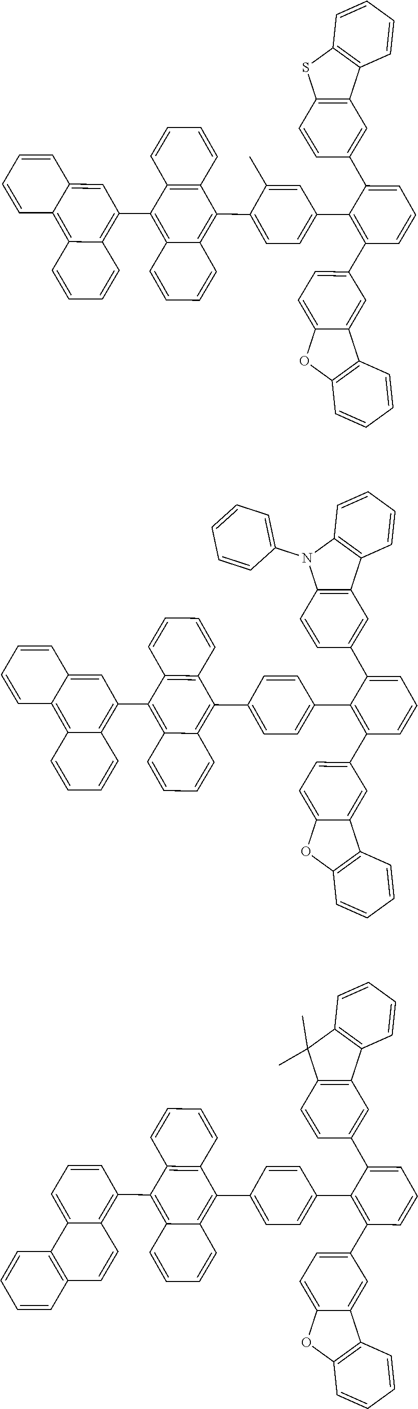 [SMILES: Cc1cc(-c2c(-c3ccc4oc5ccccc5c4c3)cccc2-c2ccc3sc4ccccc4c3c2)ccc1-c1c2ccccc2c(-c2cc3ccccc3c3ccccc23)c2ccccc12 ; c1ccc(-n2c3ccccc3c3cc(-c4cccc(-c5ccc6oc7ccccc7c6c5)c4-c4ccc(-c5c6ccccc6c(-c6cc7ccccc7c7ccccc67)c6ccccc56)cc4)ccc32)cc1 ; CC1(C)c2ccccc2-c2cc(-c3cccc(-c4ccc5oc6ccccc6c5c4)c3-c3ccc(-c4c5ccccc5c(-c5cccc6c5ccc5ccccc56)c5ccccc45)cc3)ccc21]

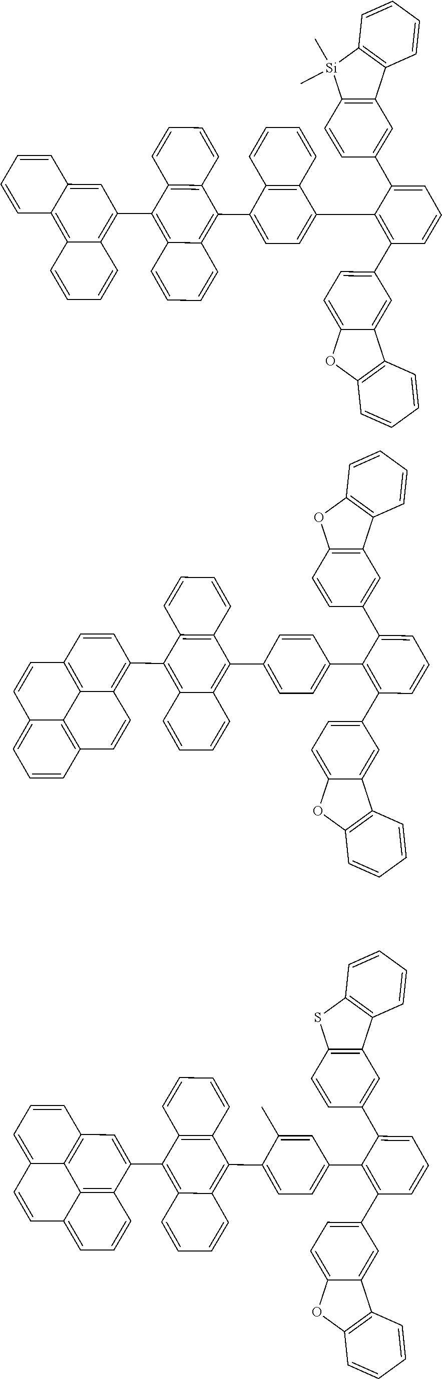

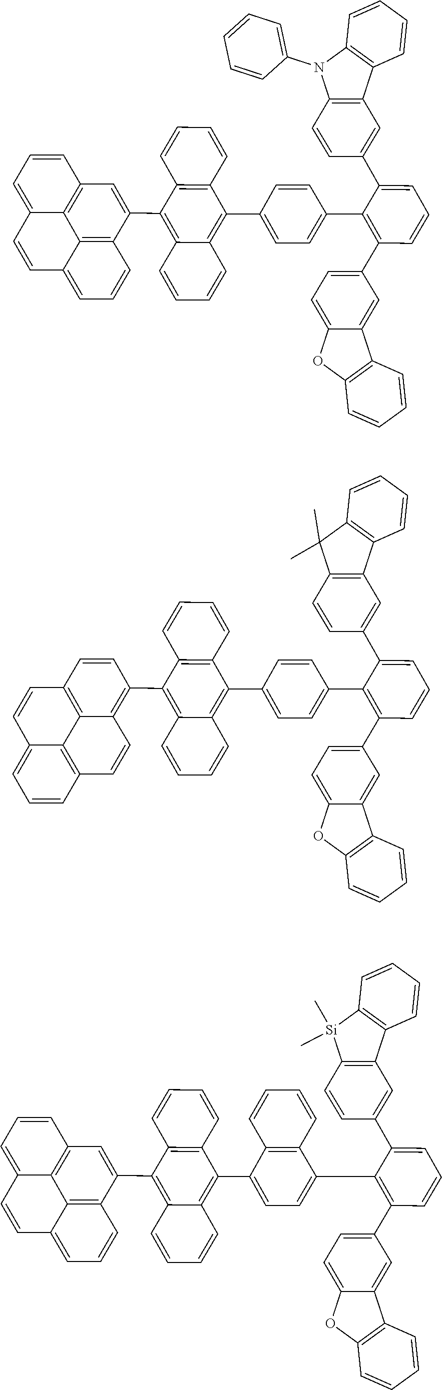 [SMILES: c1ccc(-n2c3ccccc3c3cc(-c4cccc(-c5ccc6oc7ccccc7c6c5)c4-c4ccc(-c5c6ccccc6c(-c6cc7cccc8ccc9cccc6c9c87)c6ccccc56)cc4)ccc32)cc1 ; CC1(C)c2ccccc2-c2cc(-c3cccc(-c4ccc5oc6ccccc6c5c4)c3-c3ccc(-c4c5ccccc5c(-c5ccc6ccc7cccc8ccc5c6c78)c5ccccc45)cc3)ccc21 ; C[Si]1(C)c2ccccc2-c2cc(-c3cccc(-c4ccc5oc6ccccc6c5c4)c3-c3ccc(-c4c5ccccc5c(-c5cc6cccc7ccc8cccc5c8c76)c5ccccc45)c4ccccc34)ccc21]

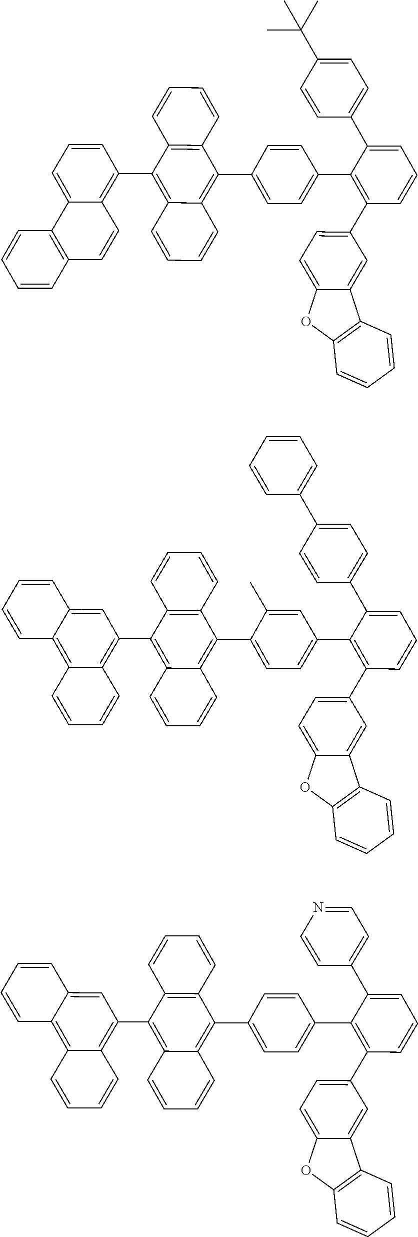 [SMILES: CC(C)(C)c1ccc(-c2cccc(-c3ccc4oc5ccccc5c4c3)c2-c2ccc(-c3c4ccccc4c(-c4cccc5c4ccc4ccccc45)c4ccccc34)cc2)cc1 ; Cc1cc(-c2c(-c3ccc(-c4ccccc4)cc3)cccc2-c2ccc3oc4ccccc4c3c2)ccc1-c1c2ccccc2c(-c2cc3ccccc3c3ccccc23)c2ccccc12 ; c1cc(-c2ccncc2)c(-c2ccc(-c3c4ccccc4c(-c4cc5ccccc5c5ccccc45)c4ccccc34)cc2)c(-c2ccc3oc4ccccc4c3c2)c1]

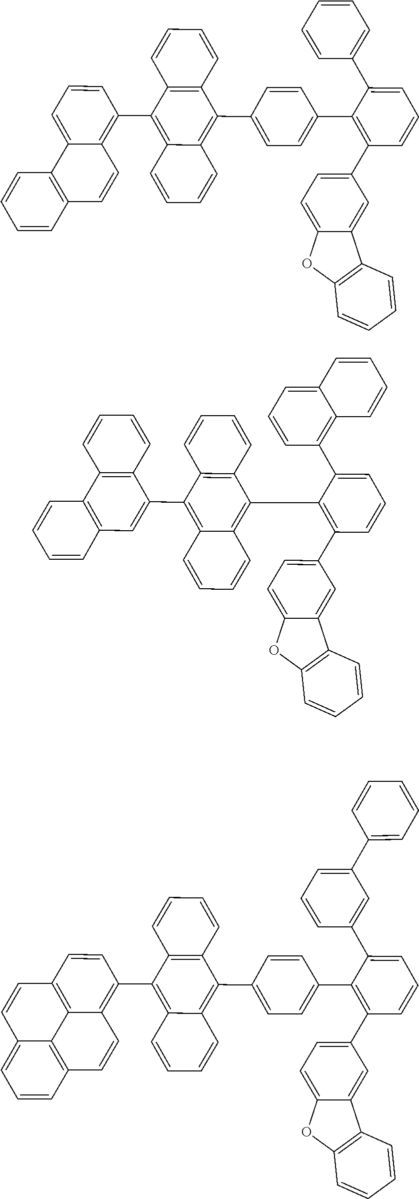

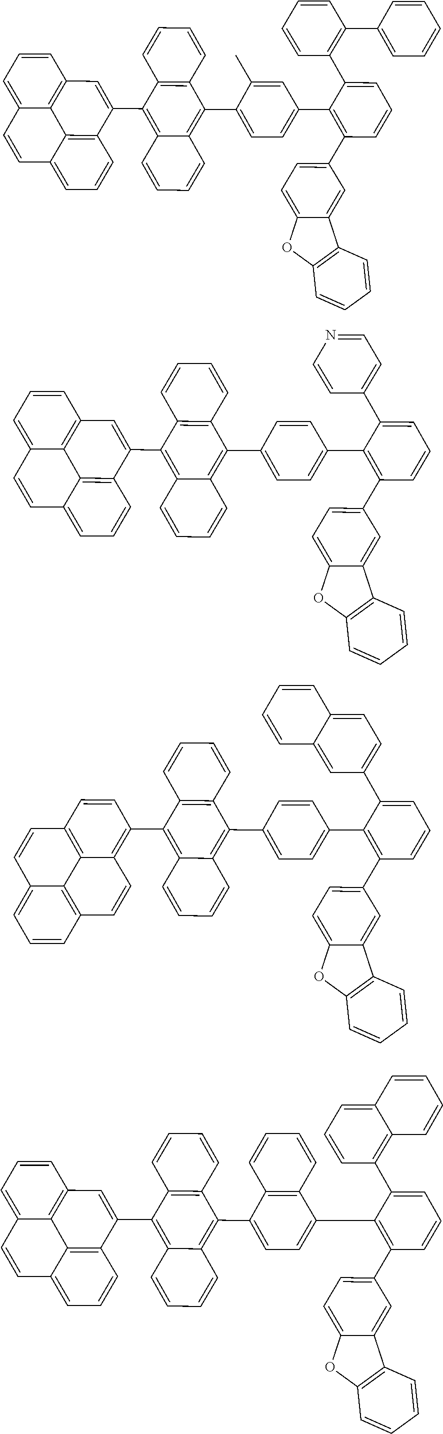

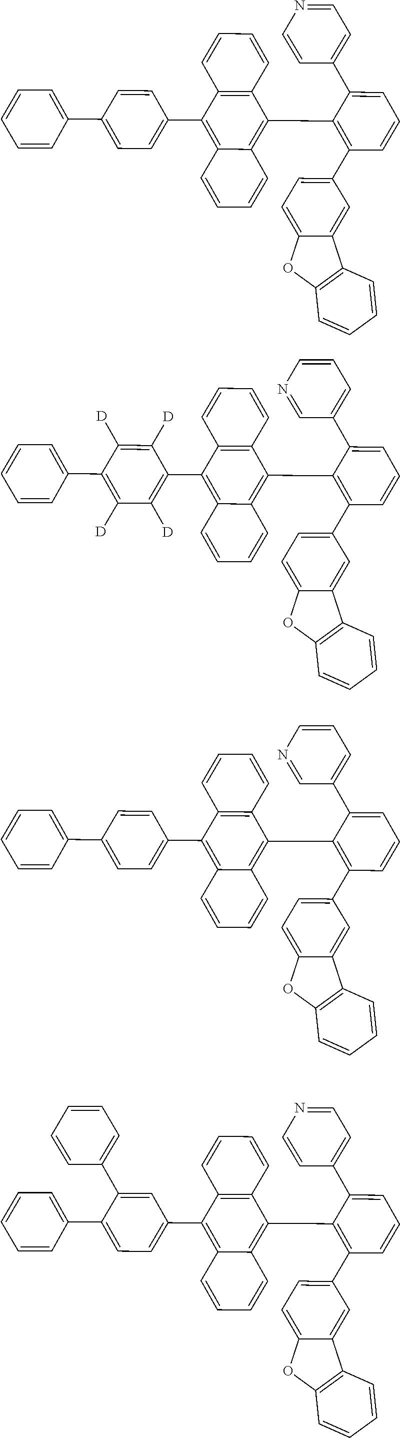

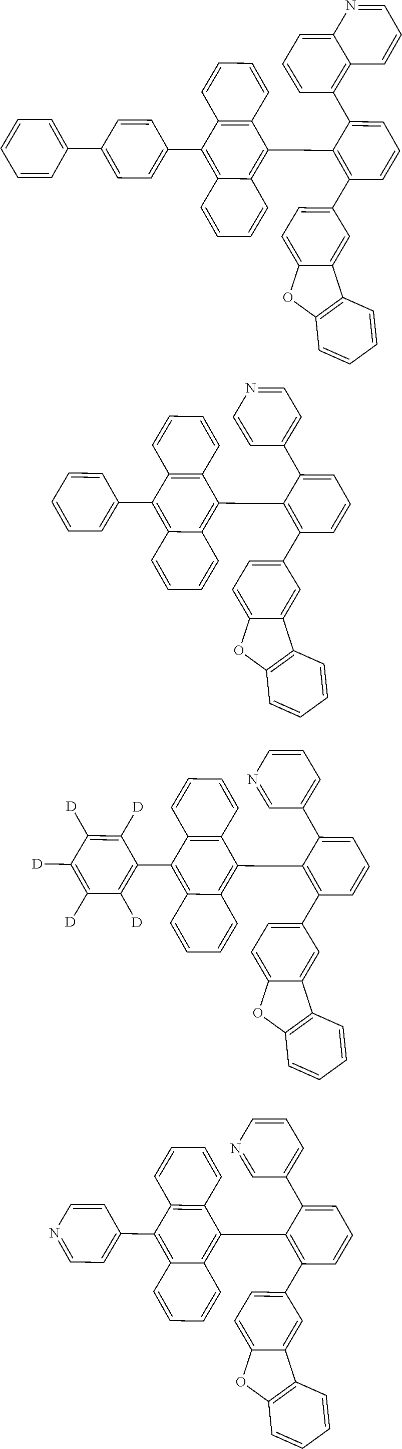

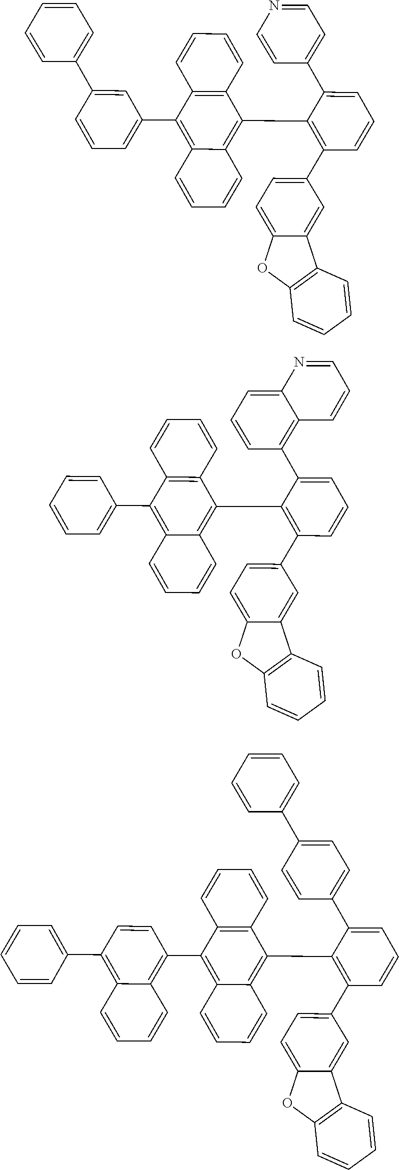

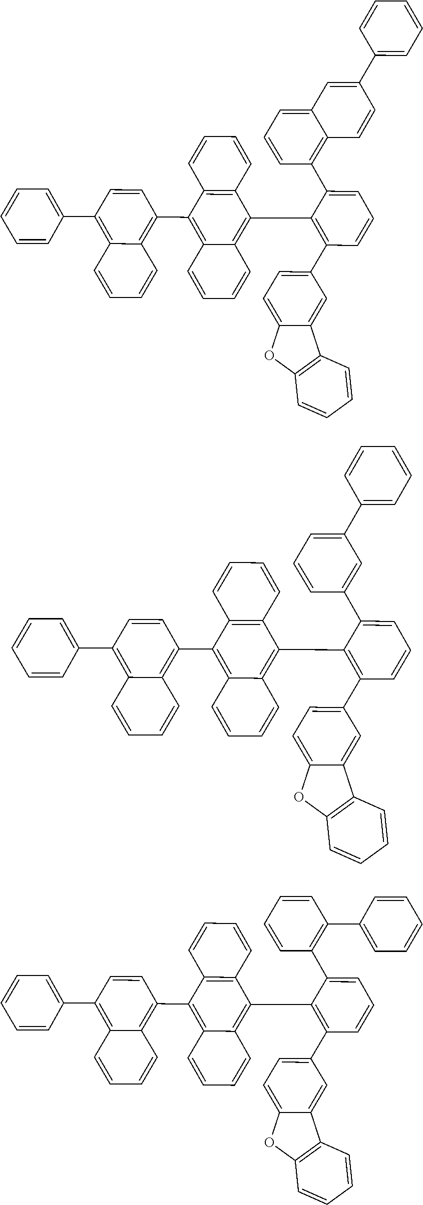

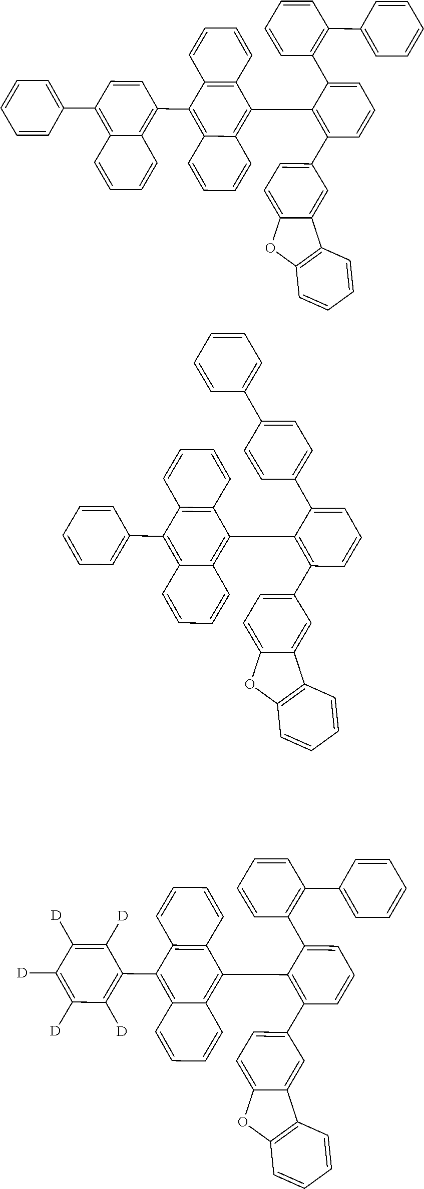 [SMILES: c1ccc(-c2ccccc2-c2cccc(-c3ccc4oc5ccccc5c4c3)c2-c2c3ccccc3c(-c3ccc(-c4ccccc4)c4ccccc34)c3ccccc23)cc1 ; c1ccc(-c2ccc(-c3cccc(-c4ccc5oc6ccccc6c5c4)c3-c3c4ccccc4c(-c4ccccc4)c4ccccc34)cc2)cc1 ; [2H]c1c([2H])c([2H])c(-c2c3ccccc3c(-c3c(-c4ccc5oc6ccccc6c5c4)cccc3-c3ccccc3-c3ccccc3)c3ccccc23)c([2H])c1[2H]]

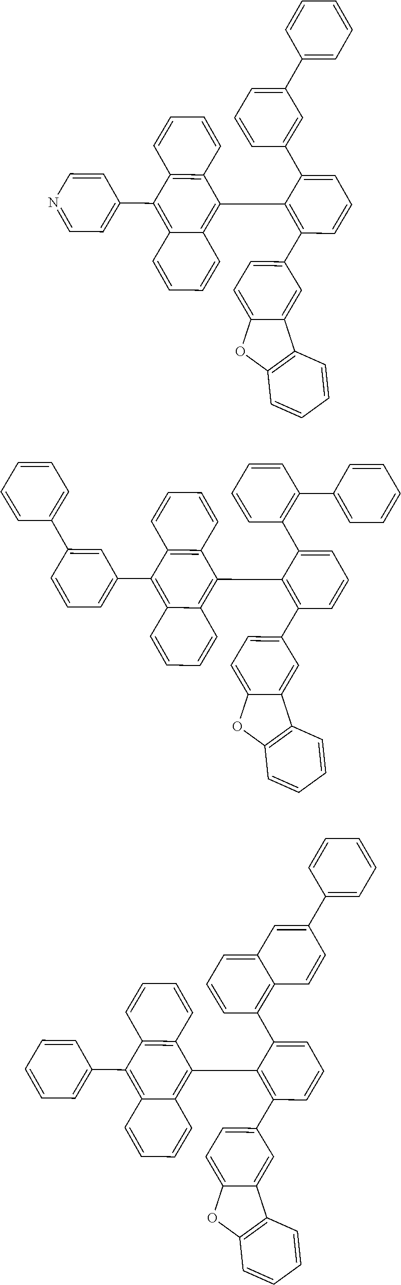 [SMILES: c1ccc(-c2cccc(-c3cccc(-c4ccc5oc6ccccc6c5c4)c3-c3c4ccccc4c(-c4ccncc4)c4ccccc34)c2)cc1 ; c1ccc(-c2cccc(-c3c4ccccc4c(-c4c(-c5ccc6oc7ccccc7c6c5)cccc4-c4ccccc4-c4ccccc4)c4ccccc34)c2)cc1 ; c1ccc(-c2ccc3c(-c4cccc(-c5ccc6oc7ccccc7c6c5)c4-c4c5ccccc5c(-c5ccccc5)c5ccccc45)cccc3c2)cc1]

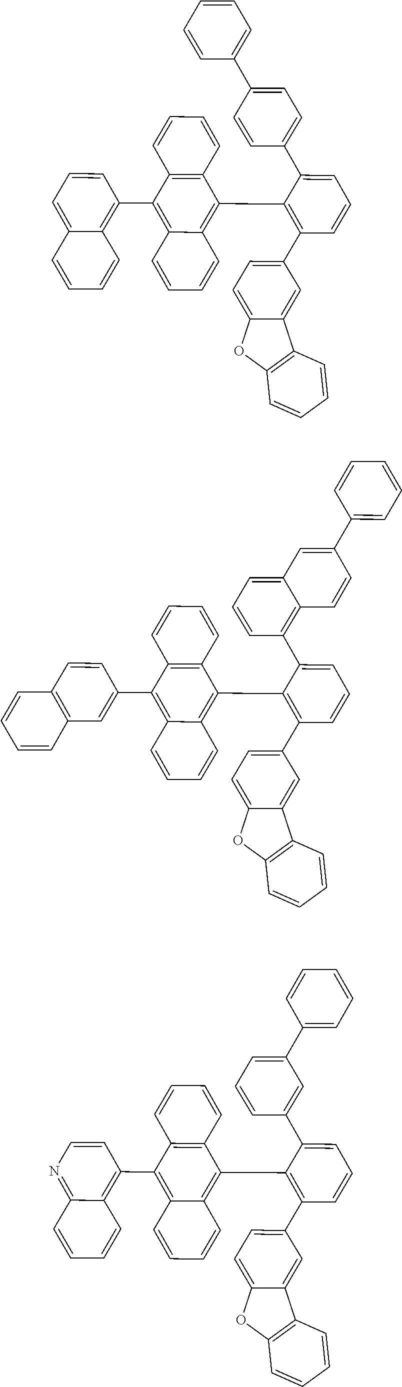 [SMILES: c1ccc(-c2ccc(-c3cccc(-c4ccc5oc6ccccc6c5c4)c3-c3c4ccccc4c(-c4cccc5ccccc45)c4ccccc34)cc2)cc1 ; c1ccc(-c2ccc3c(-c4cccc(-c5ccc6oc7ccccc7c6c5)c4-c4c5ccccc5c(-c5ccc6ccccc6c5)c5ccccc45)cccc3c2)cc1 ; c1ccc(-c2cccc(-c3cccc(-c4ccc5oc6ccccc6c5c4)c3-c3c4ccccc4c(-c4ccnc5ccccc45)c4ccccc34)c2)cc1]

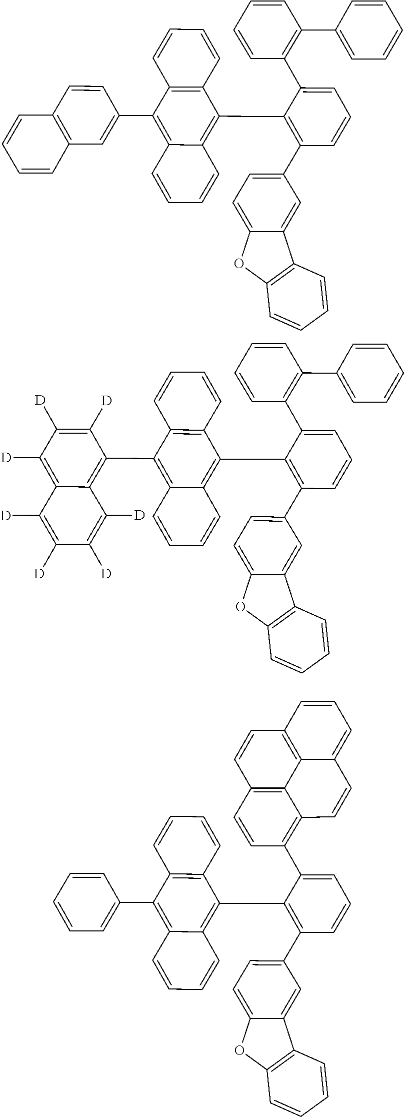

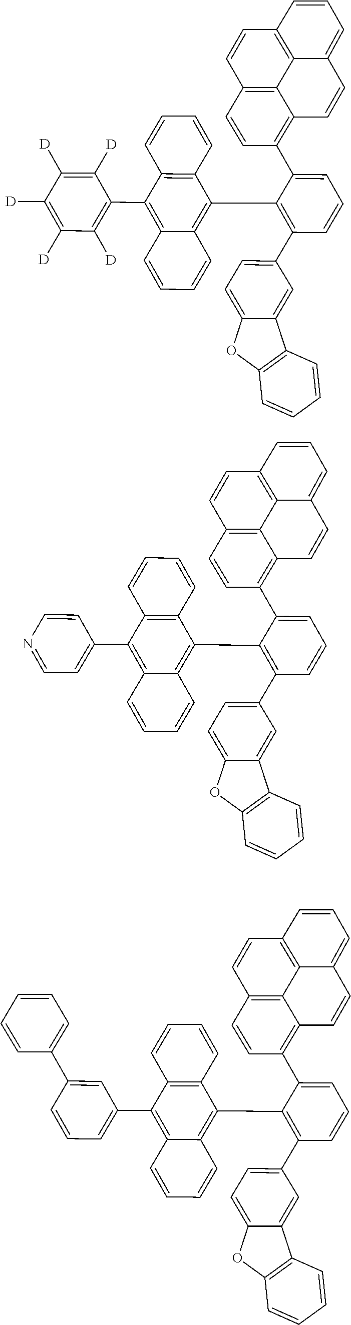

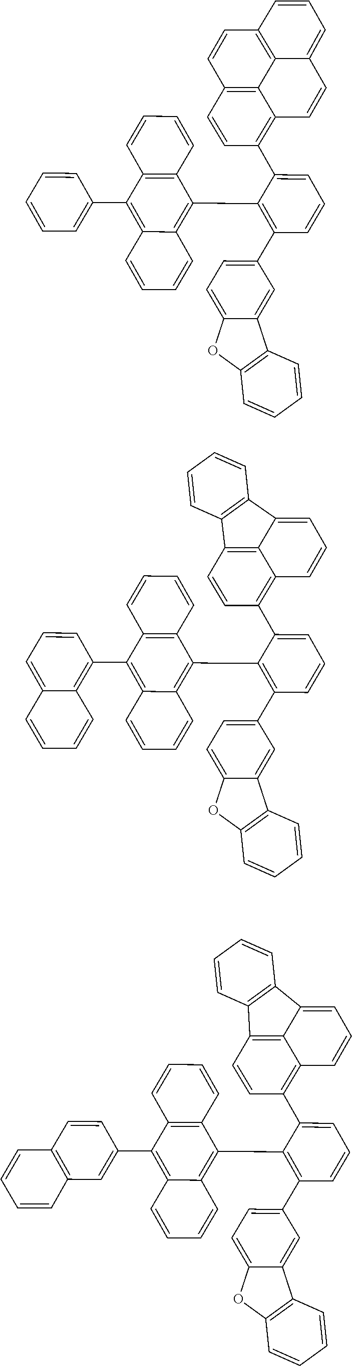 [SMILES: c1ccc(-c2c3ccccc3c(-c3c(-c4ccc5oc6ccccc6c5c4)cccc3-c3ccc4ccc5cccc6ccc3c4c56)c3ccccc23)cc1 ; c1ccc2c(c1)-c1cccc3c(-c4cccc(-c5ccc6oc7ccccc7c6c5)c4-c4c5ccccc5c(-c5cccc6ccccc56)c5ccccc45)ccc-2c13 ; c1ccc2c(c1)-c1cccc3c(-c4cccc(-c5ccc6oc7ccccc7c6c5)c4-c4c5ccccc5c(-c5ccc6ccccc6c5)c5ccccc45)ccc-2c13]

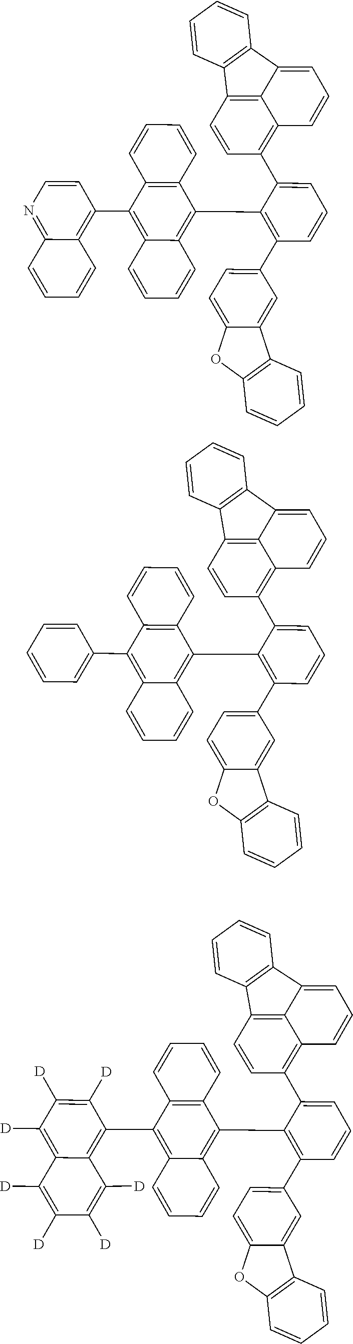

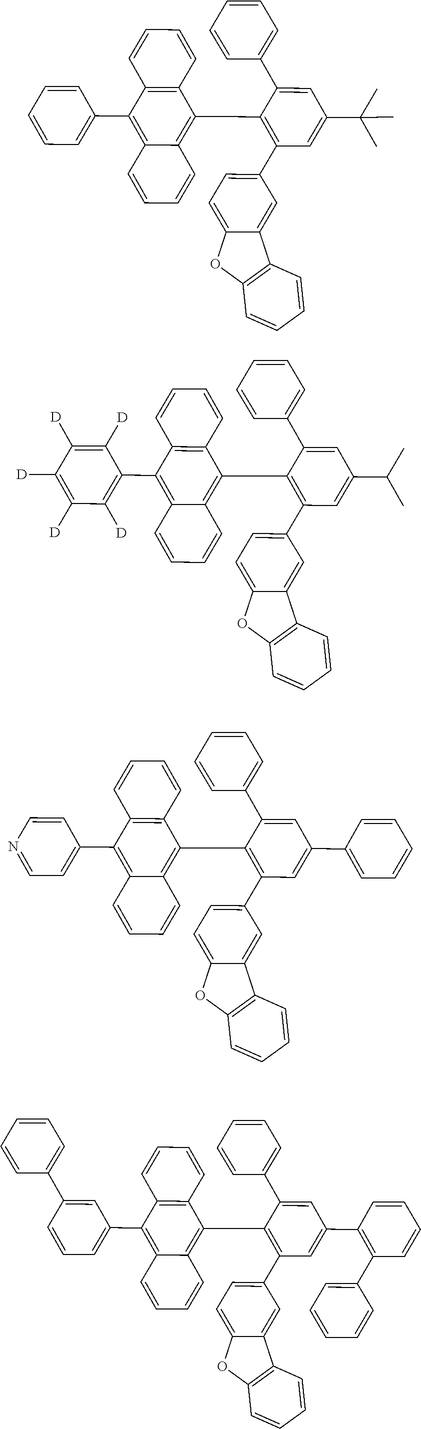

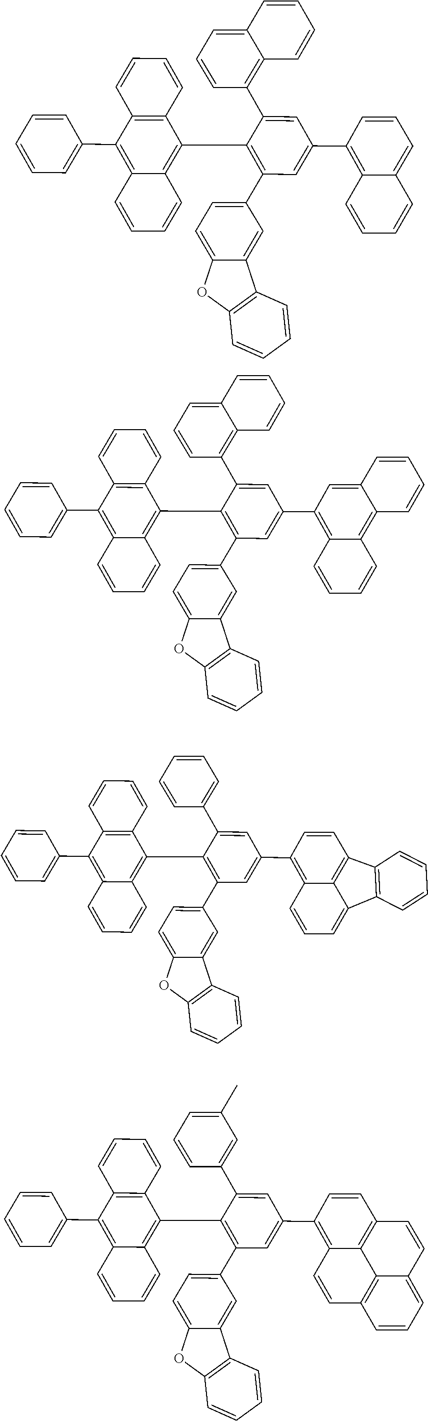

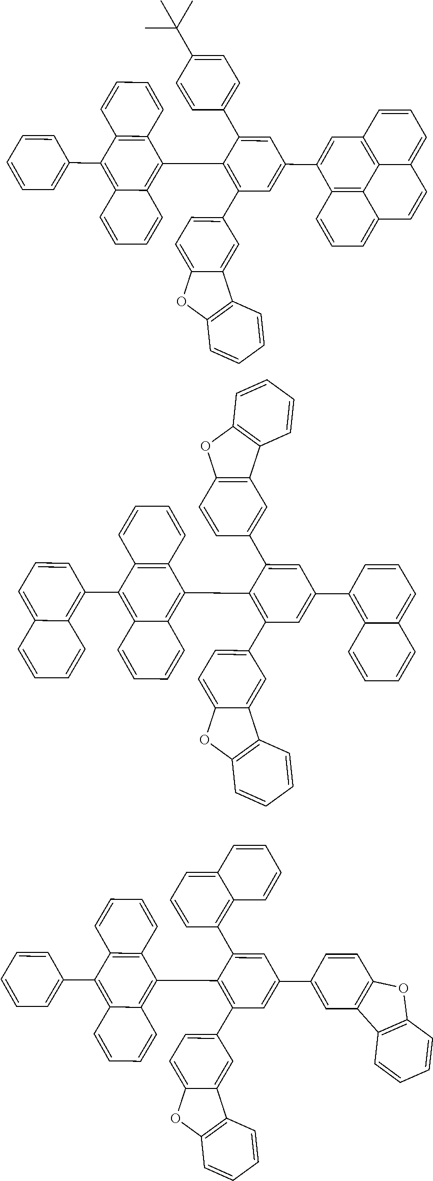

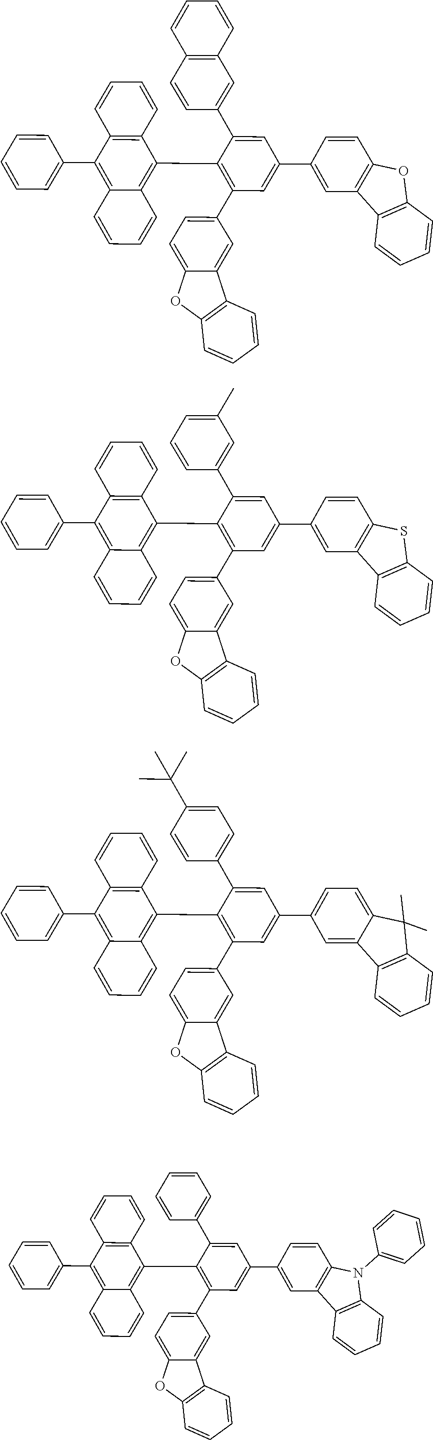

### Example 1

The compound represented by formula (1) is compound 1, and the compound 1 is synthesized through a following synthetic route:

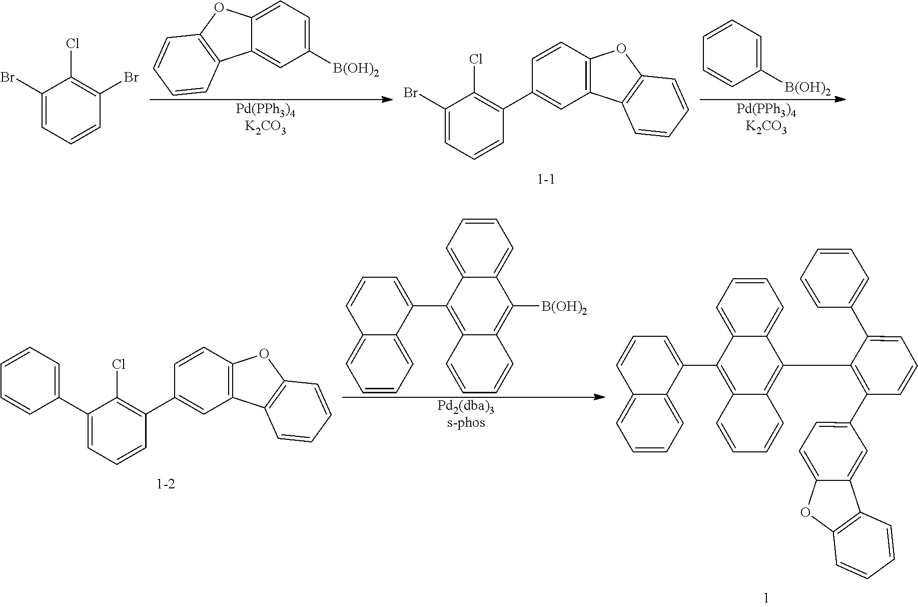

### Synthesis of Intermediate 1-1

2-chloro-1,3-dibromobenzene (27 g, 100 mmol) and dibenzofuran-2-boric acid (21 g, 100 mmol) were dissolved in a mixed solvent of 1,4-dioxane and water (500 mL:50 mL), and tetrakis(triphenylphosphine)palladium (Pd(PPh₃)₄ (1.8 g, 1.5 mmol)) and potassium carbonate (62 g, 450 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is petroleum ether (PE)) to obtain a white solid, namely the intermediate 1-1 with yield of 74%. MS (ASAP)=356.0.

### Synthesis of Intermediate 1-2

The intermediate 1-1 (17.8 g, 50 mmol) and phenylboric acid (6.1 g, 50 mmol) were dissolved in a mixed solvent of 1,4-dioxane and water (250 mL:50 mL), and Pd(PPh₃)₄ (0.88 g, 0.75 mmol) and potassium carbonate (28 g, 200 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of dichloromethane (DCM) and PE with a volume ratio of 1:8) to obtain a white solid, namely the intermediate 1-2 with yield of 90%. MS (ASAP)=354.1.

### Synthesis of the Compound 1

The intermediate 1-2 (10 g, 28.2 mmol), 10-(1-naphthyl)-9-anthraceneboric acid (9.8 g, 28.2 mmol) were dissolved in a mixed solvent of 1,4-dioxane and water (200 mL:20 mL), and tris(dibenzylideneacetone)dipalladium (Pd₂(dba)₃, (0.52 g, 0.56 mmol)), di(1-adamantyl)-n-butylphosphine hydriodide (s-phos, (0.46 g, 1.12 mmol)), and potassium carbonate (19.5 g, 141 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:5) and recrystallized to obtain the compound 1 with yield of 67%. MS (ASAP)=622.2.

### Example 2

The compound represented by formula (1) is compound 2, and the compound 2 is synthesized through a following synthetic route:

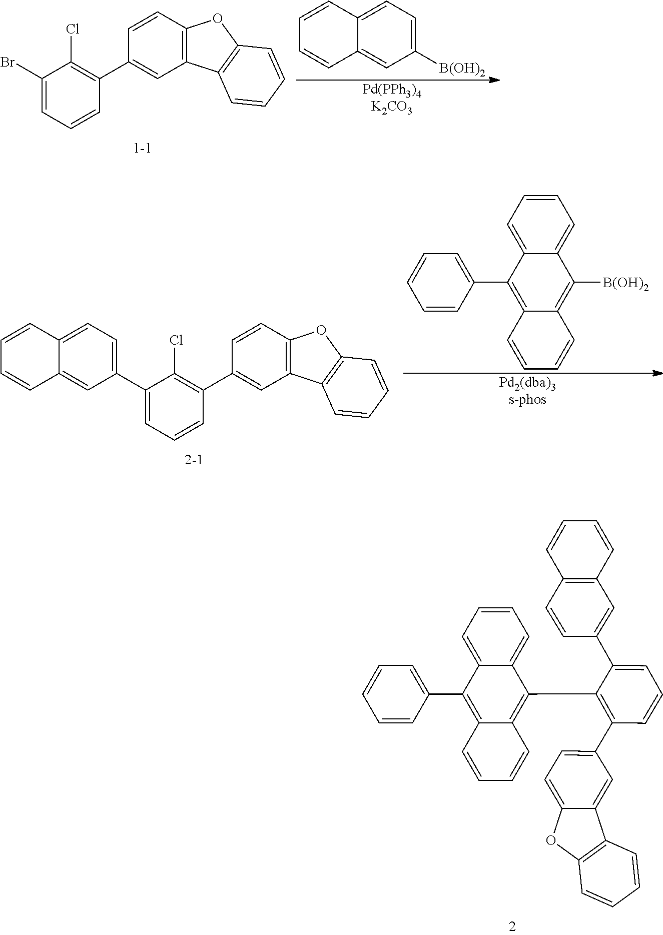

### Synthesis of Intermediate 2-1

The intermediate 1-1 (7.1 g, 20 mmol) and 2-naphthyl boric acid (3.5 g, 20 mmol) were dissolved in a mixed solvent of 1,4-dioxane and water (250 mL:50 mL), and Pd(PPh₃)₄ (0.46 g, 0.4 mmol) and potassium carbonate (13.8 g, 100 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:8) to obtain a white solid, namely the intermediate 2-1 with yield of 84%. MS (ASAP)=404.1.

### Synthesis of the Compound 2

The intermediate 2-1 (10 g, 24.8 mmol) and 10-phenyl-9-anthroboronic acid (7.4 g, 24.8 mmol) were dissolved in a mixed solvent of 1,4-dioxane and water (200 mL:20 mL), and Pd₂(dba)₃ (0.46 g, 0.5 mmol), s-phos (0.41 g, 1.0 mmol), and potassium carbonate (17 g, 124 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:4) and recrystallized to obtain the compound 2 with yield of 72%. MS (ASAP)=622.2.

### Example 3

The compound represented by formula (1) is compound 3, and the compound 3 is synthesized through a following synthetic route:

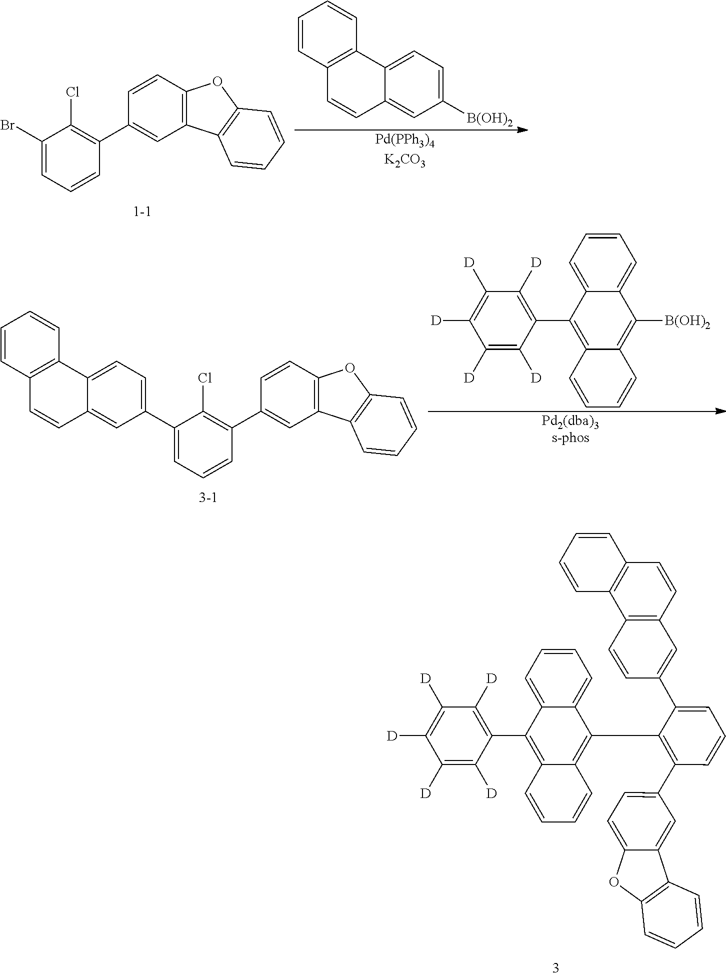

### Synthesis of Intermediate 3-1:

The intermediate 1-1 (7.1 g, 20 mmol) and 2-bromophenanthrene (4.5 g, 20 mmol) were dissolved in a mixed solvent of 1,4-dioxane and water (250 mL:50 mL), and Pd(PPh₃)₄ (0.46 g, 0.4 mmol) and potassium carbonate (13.8 g, 100 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:8) to obtain a white solid, namely the intermediate 3-1 with yield of 82%. MS (ASAP)=454.1.

### Synthesis of the Compound 3

The intermediate 3-1 (10 g, 22 mmol) and 10-deuteriophenyl-9-anthroboronic acid (6.7 g, 22 mmol) were dissolved in a mixed solvent of 1,4-dioxane and water (200 mL:20 mL), and Pd₂(dba)₃ (0.41 g, 0.45 mmol), s-phos (0.37 g, 0.9 mmol), and potassium carbonate (15 g, 110 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:4) and recrystallized to obtain the compound 3 with yield of 78%. MS (ASAP)=677.3.

### Example 4

The compound represented by formula (1) is compound 4, and the compound 4 is synthesized through a following synthetic route:

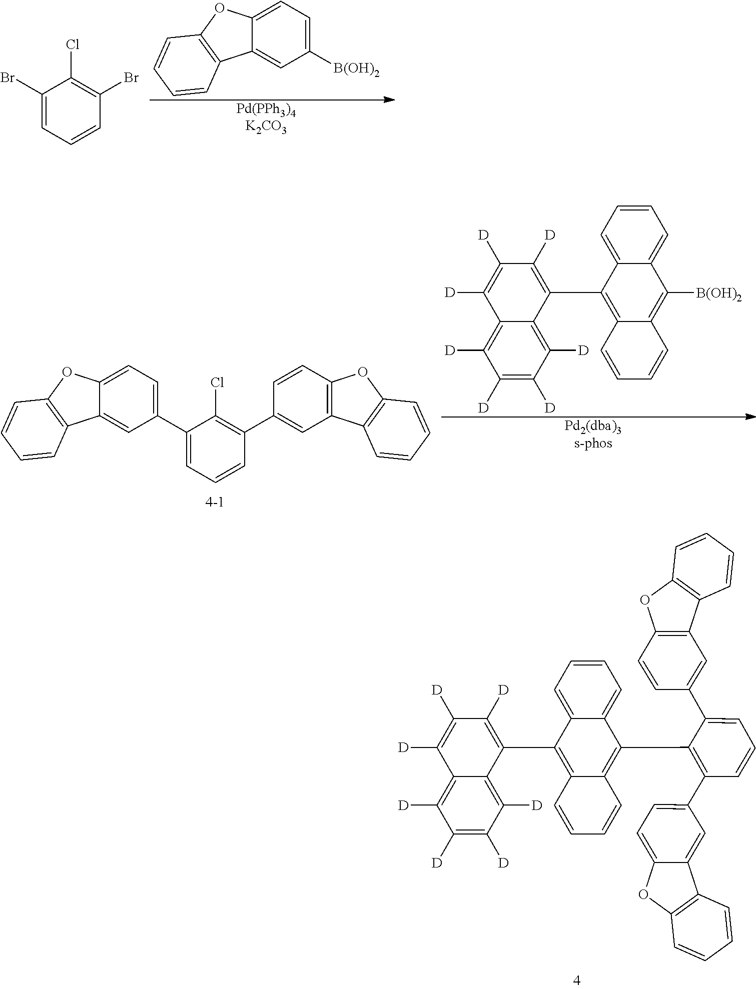

### Synthesis of Intermediate 4-1

2-chloro-1,3-dibromobenzene (27 g, 100 mmol) and dibenzofuran-2-boric acid (42 g, 200 mmol) were dissolved in a mixed solvent of 1,4-dioxane and water (500 mL:50 mL), and Pd(PPh₃)₄ (1.8 g, 1.5 mmol) and potassium carbonate (62 g, 450 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:8) to obtain a white solid, namely the intermediate 4-1 with yield of 68%. MS (ASAP)=444.1.

### Synthesis of the Compound 4

The intermediate 4-1 (10 g, 22.5 mmol) and 10-(deuteronaphthyl)-9-anthraceneboric acid (8.0 g, 22.5 mmol) were dissolved in a mixed solvent of 1,4-dioxane and water (200 mL:20 mL), and Pd₂(dba)₃ (0.41 g, 0.45 mmol), s-phos (0.37 g, 0.9 mmol), and potassium carbonate (15.5 g, 113 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:4) and recrystallized to obtain the compound 4 with yield of 73%. MS (ASAP)=719.3.

### Example 5

The compound represented by formula (1) is compound 5, and the compound 5 is synthesized through a following synthetic route:

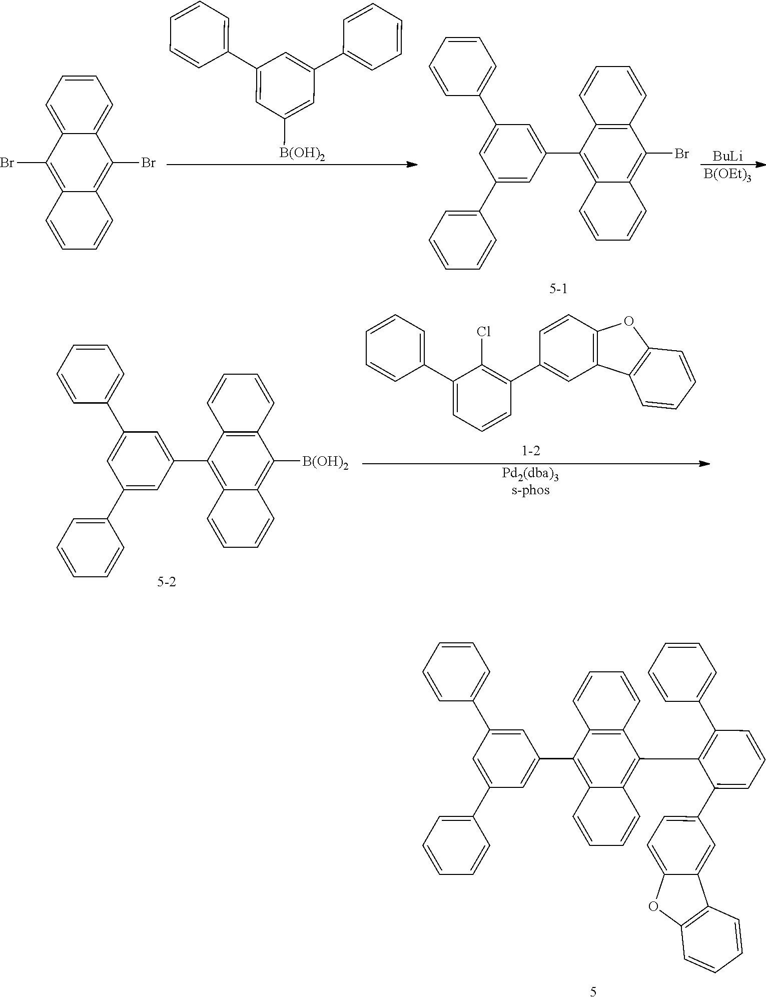

### Synthesis of Intermediate 5-1

9,10-dibromoanthrene (3.4 g, 10 mmol) and 3,5-diphenyl-phenylboric acid (2.8 g, 10 mmol) were dissolved in a mixed solvent of 1,4-dioxane and water (100 mL:10 mL), and Pd(PPh₃)₄ (1.15 g, 1 mmol) and potassium carbonate (8.3 g, 60 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography and recrystallized to obtain the intermediate 5-1 with yield of 69%. MS (ASAP)=484.1.

### Synthesis of Intermediate 5-2

A dry three neck-flask (250 mL) was provided, a reaction system was set up, and the three neck-flask was vacuumed and nitrogen was injected. Nitrogen was kept flowing in the three neck-flask, THF (100 mL) was added to the intermediate 5-1 (5.0 g, 10.3 mmol), the three neck-flask was vacuumized, and nitrogen was circulated for three times. Then, the solution was cooled to a temperature of −78° C. A n-butyl lithium solution (4.1 mL, 10.3 mmol) was slowly added into the three neck-flask dropwise. After the reaction lasted at a temperature of −78° C.for 60 minutes, triethyl borate (1.5 g, 10.3 mmol) was slowly added dropwise. Then, the temperature of the reaction system was slowly raised to room temperature, and the reaction lasted for 12 hours. Diluted hydrochloric acid was added to the solution and stirred for 30 minutes. The solution was extracted with ethanol (EA) and then was rotationally evaporated to remove the solvent, PE was used to obtain a white solid, namely the intermediate 5-2 with yield of 83%. MS(ASAP)=450.2.

### Synthesis of the Compound 5

The intermediate 5-2 (4.5 g, 10 mmol) and the intermediate 1-2 (3.6 g, 10 mmol) were dissolved in a mixed solvent of 1,4-dioxane and water (100 mL:10 mL), and Pd₂(dba)₃ (0.18 g, 0.2 mmol), s-phos (0.16 g, 0.4 mmol), and potassium carbonate (6.9 g, 50 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:4) and recrystallized to obtain the compound 5 with yield of 77%. MS (ASAP)=724.3.

### Example 6

The compound represented by formula (1) is compound 6, and the compound 6 is synthesized through a following synthetic route:

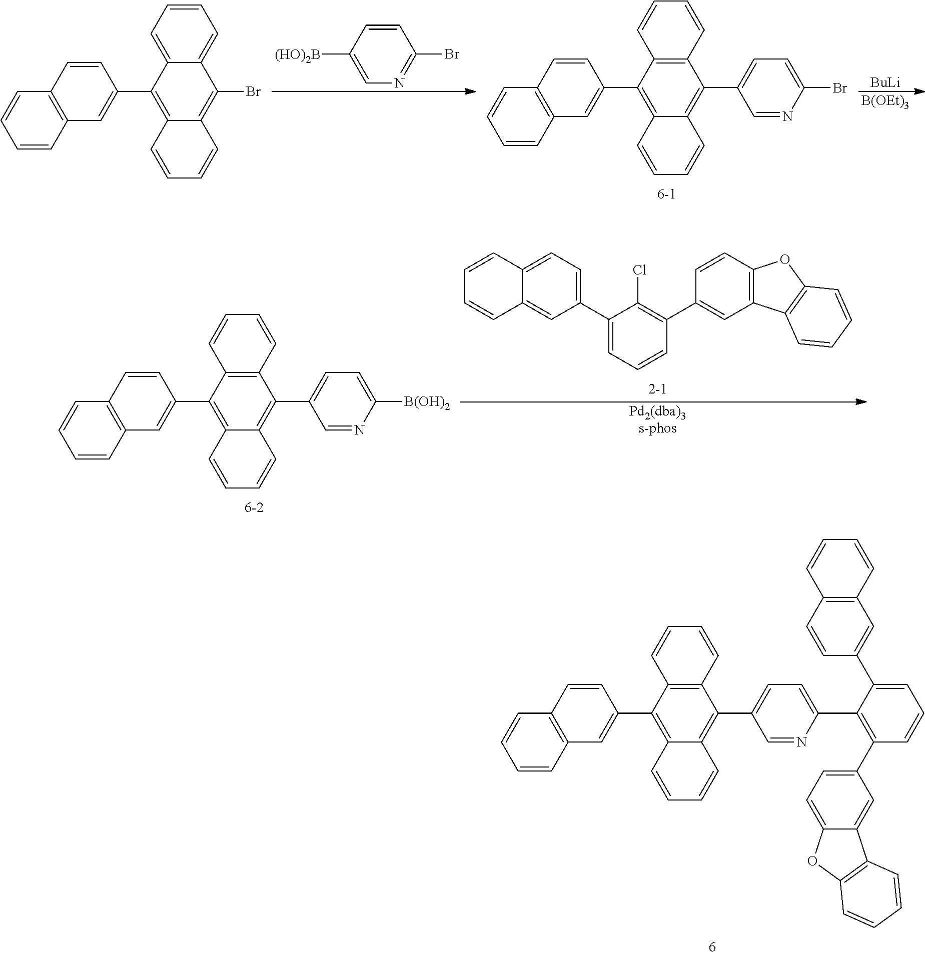

### Synthesis of Intermediate 6-1

9-bromo-10-(2-naphthyl) anthracene (3.8 g, 10 mmol) and 2-bromo-pyridine-5-boric acid (2.0 g, 10 mmol) were dissolved in a mixed solvent of 1,4-dioxane and water (100 mL:10 mL), and Pd(PPh₃)₄ (1.15 g, 1 mmol) and potassium carbonate (4.1 g, 30 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography and recrystallized to obtain the intermediate 6-1 with yield of 83%. MS (ASAP)=459.1.

### Synthesis of Intermediate 6-2

A dry three neck-flask (250 mL) was provided, a reaction system was set up, and the three neck-flask was vacuumed and nitrogen was injected. Nitrogen was kept flowing in the three neck-flask, THF (100 mL) was added to the intermediate 6-1 (5.0 g, 10.9 mmol), the three neck-flask was vacuumized, and nitrogen was circulated for three times. Then, the solution was cooled to a temperature of −78° C. A n-butyl lithium solution (4.4 mL, 10.9 mmol) was slowly added into the three neck-flask dropwise. After the reaction lasted at a temperature of −78° C. for 60 minutes, triethyl borate (1.6 g, 10.9 mmol) was slowly added dropwise. Then, the temperature of the reaction system was slowly raised to room temperature, and the reaction lasted for 12 hours. Diluted hydrochloric acid was added to the solution and stirred for 30 minutes. The solution was extracted with EA and then was rotationally evaporated to remove the solvent, PE was used to obtain a white solid, namely the intermediate 6-2 with yield of 84%. MS(ASAP)=425.2.

### Synthesis of the Compound 6

The intermediate 6-2 (4.3 g, 10 mmol) and the intermediate 2-1 (4.1 g, 10 mmol) were dissolved in a mixed solvent of 1,4-dioxane and water (100 mL:10 mL), and Pd₂(dba)₃ (0.18 g, 0.2 mmol), s-phos (0.16 g, 0.4 mmol), and potassium carbonate (6.9 g, 50 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:4) and recrystallized to obtain the compound 6 with yield of 81%. MS (ASAP)=749.3.

### Example 7

The compound represented by formula (1) is compound 7, and the compound 7 is synthesized through a following synthetic route:

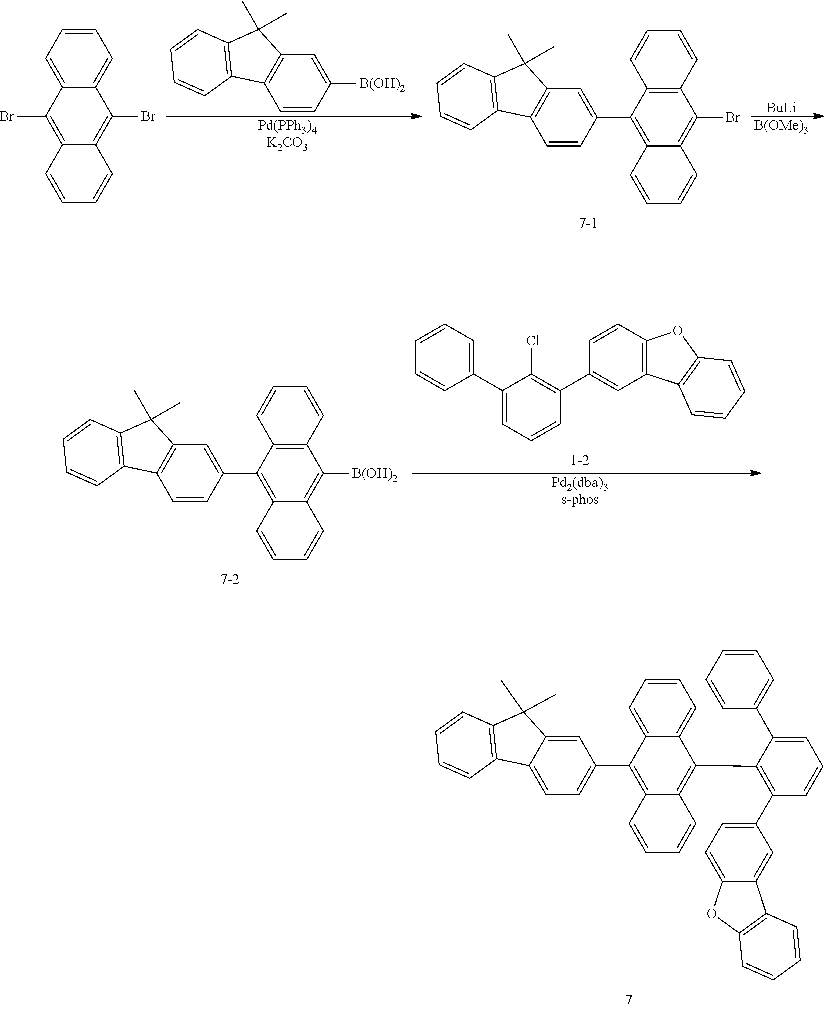

### Synthesis of Intermediate 7-1

9,10-dibromoanthrene (6.8 g, 20 mmol) and 9,9-dimethylfluoren-2-boric acid (4.8 g, 20 mmol) were dissolved in a mixed solvent of 1,4-dioxane and water (100 mL:10 mL), and Pd(PPh₃)₄ (2.3 g, 2 mmol) and potassium carbonate (14 g, 100 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography and recrystallized to obtain the intermediate 7-1 with yield of 69%. MS (ASAP)=448.1.

### Synthesis of Intermediate 7-2

A dry three neck-flask (250 mL) was provided, a reaction system was set up, and the three neck-flask was vacuumed and nitrogen was injected. Nitrogen was kept flowing in the three neck-flask, THF (100 mL) was added to the intermediate 7-1 (5.0 g, 11.1 mmol), the three neck-flask was vacuumized, and nitrogen was circulated for three times. Then, the solution was cooled to a temperature of −78° C. A n-butyl lithium solution (4.5 mL, 11.1 mmol) was slowly added into the three neck-flask dropwise. After the reaction lasted at a temperature of −78° C. for 60 minutes, triethyl borate (1.6 g, 11.1 mmol) was slowly added dropwise. Then, the temperature of the reaction system was slowly raised to room temperature, and the reaction lasted for 12 hours. Diluted hydrochloric acid was added to the solution and stirred for 30 minutes. The solution was extracted with EA and then was rotationally evaporated to remove the solvent, PE was used to obtain a white solid, namely the intermediate 7-2 with yield of 94%. MS(ASAP)=414.2.

### Synthesis of the Compound 7

The intermediate 7-2 (8.3 g, 20 mmol) and the intermediate 1-2 (7.1 g, 20 mmol) were dissolved in a mixed solvent of 1,4-dioxane and water (100 mL:10 mL), and Pd₂(dba)₃ (0.46 g, 0.5 mmol), s-phos (0.41 g, 1 mmol), and potassium carbonate (8.3 g, 60 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:4) and recrystallized to obtain the compound 7 with yield of 64%. MS (ASAP)-688.3.

### Example 8

The compound represented by formula (1) is compound 8, and the compound 8 is synthesized through a following synthetic route:

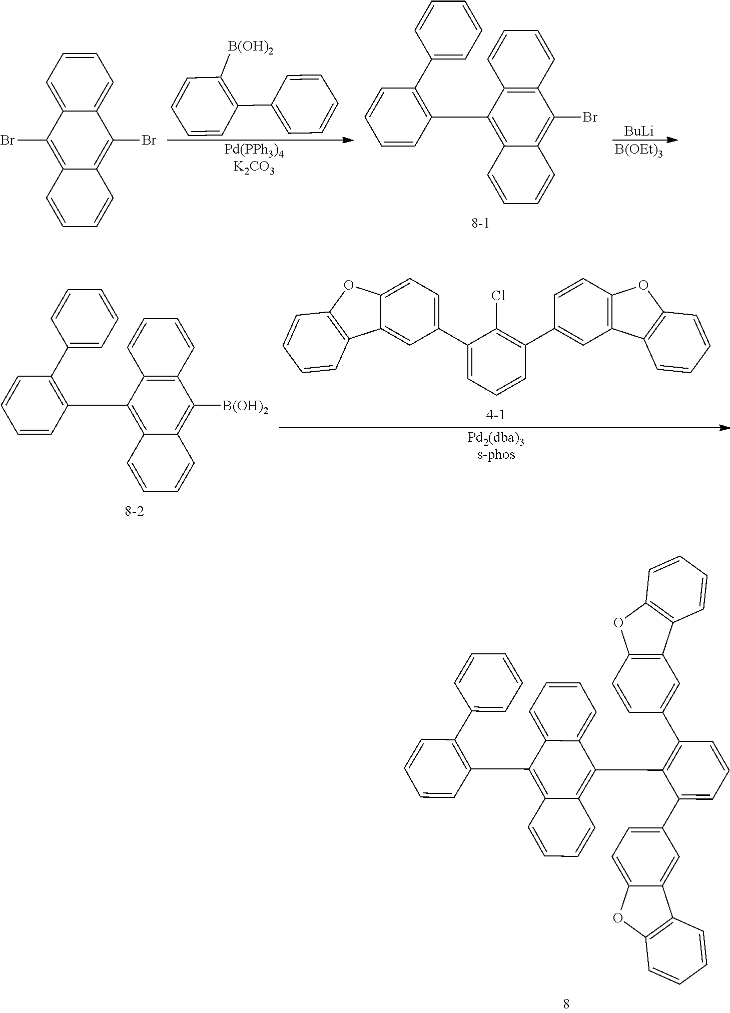

### Synthesis of Intermediate 8-1

9,10-dibromoanthrene (6.8 g, 20 mmol) and 9,9-dimethylfluoren-2-boric acid (4.8 g, 20 mmol) were dissolved in a mixed solvent of 1,4-dioxane and water (100 mL:10 mL), and Pd(PPh₃)₄ (1.2 g, 1 mmol) and potassium carbonate (6.9 g, 50 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography and recrystallized to obtain the intermediate 8-1 with yield of 76%. MS (ASAP)=408.1.

### Synthesis of Intermediate 8-2

A dry three neck-flask (250 mL) was provided, a reaction system was set up, and the three neck-flask was vacuumed and nitrogen was injected. Nitrogen was kept flowing in the three neck-flask, THF (100 mL) was added to the intermediate 8-1 (5.0 g, 12 mmol), the three neck-flask was vacuumized, and nitrogen was circulated for three times. Then, the solution was cooled to a temperature of −78° C. A n-butyl lithium solution (4.9 mL, 12 mmol) was slowly added into the three neck-flask dropwise. After the reaction lasted at a temperature of −78° C. for 60 minutes, triethyl borate (1.8 g, 12 mmol) was slowly added dropwise. Then, the temperature of the reaction system was slowly raised to room temperature, and the reaction lasted for 12 hours. Diluted hydrochloric acid was added to the solution and stirred for 30 minutes. The solution was extracted with EA and then was rotationally evaporated to remove the solvent, PE was used to obtain a white solid, namely the intermediate 8-2 with yield of 82%. MS(ASAP)=374.2.

### Synthesis of the Compound 8

The intermediate 8-2 (3.7 g, 10 mmol) and the intermediate 4-1 (4.4 g, 10 mmol) were dissolved in the mixed solvent of 1,4-dioxane and water (100 mL: 10 mL), and Pd₂(dba)₃ (0.18 g, 0.2 mmol), s-phos (0.16 g, 0.4 mmol), and potassium carbonate (6.2 g, 45 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography and recrystallized to obtain the compound 8 with yield of 53%. MS (ASAP)=738.3.

### Example 9

The compound represented by formula (1) is compound 9, and the compound 9 is synthesized through a following synthetic route:

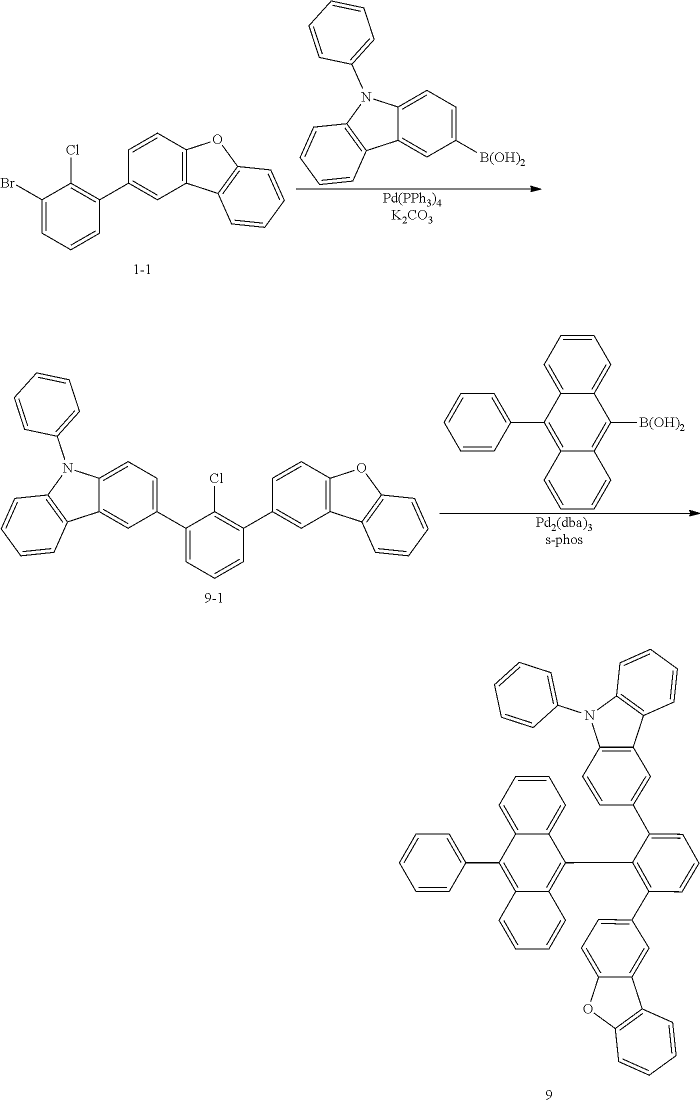

### Synthesis of Intermediate 9-1

The intermediate 1-1 (7.1 g, 20 mmol) and (9-phenyl-9H-carbazol-3-yl) boric acid (5.7 g, 20 mmol) were dissolved in a mixed solvent of 1,4-dioxane and water (250 mL:50 mL), and Pd(PPh₃)₄ (0.46 g, 0.4 mmol) and potassium carbonate (13.8 g, 100 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:8) to obtain a white solid, namely the intermediate 9-1 with yield of 81%. MS (ASAP)=519.1.

### Synthesis of the Compound 9

The intermediate 9-1 (10 g, 19.3 mmol) and 10-phenyl-9-anthroboronic acid (5.7 g, 19.3 mmol) were dissolved in a mixed solvent of 1,4-dioxane and water (200 mL:20 mL), and Pd₂(dba)₃ (0.55 g, 0.6 mmol), s-phos (0.49 g, 1.2 mmol), and potassium carbonate (13.8 g, 100 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:4) and recrystallized to obtain the compound 9 with yield of 71%. MS (ASAP)=737.3.

### Example 10

The compound represented by formula (1) is compound 10, and the compound 10 is synthesized through a following synthetic route:

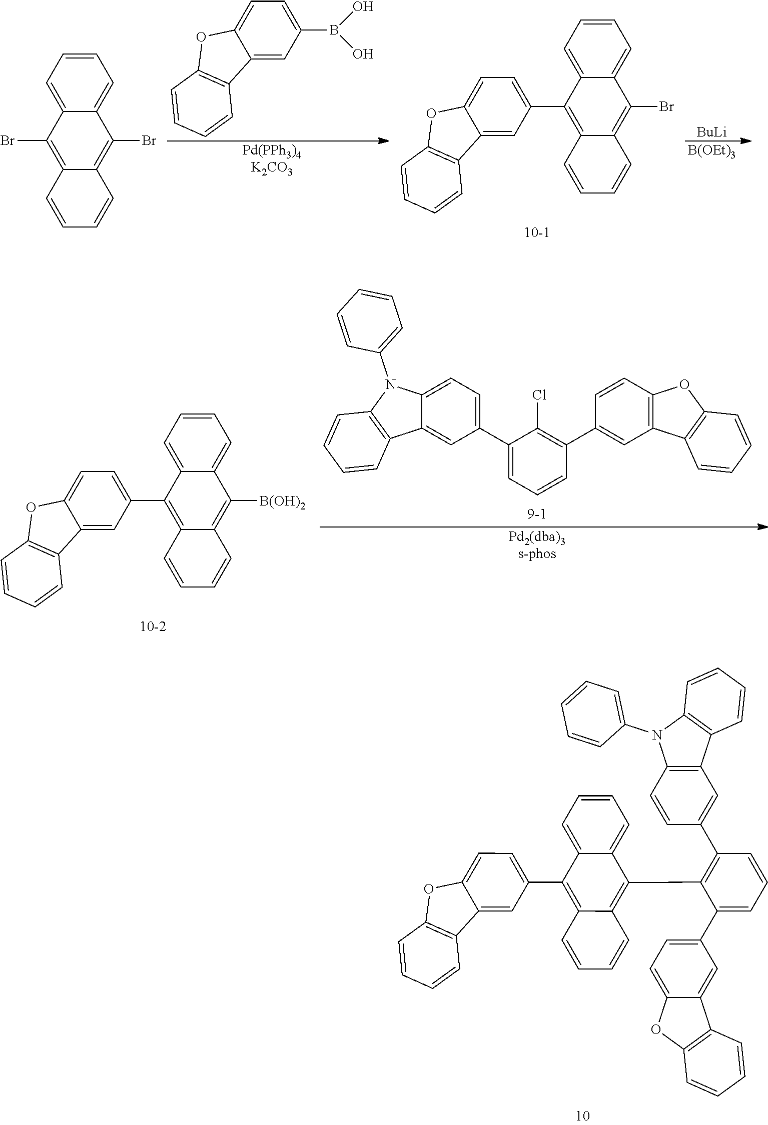

### Synthesis of Intermediate 10-1

9,10-dibromoanthrene (6.7 g, 20 mmol) and dibenzo [b, d] furan-2-boric acid (4.3 g, 20 mmol) were dissolved in a mixed solvent of 1,4-dioxane and water (100 mL:10 mL), and Pd(PPh₃)₄ (1.2 g, 1 mmol) and potassium carbonate (14 g, 100 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography and recrystallized to obtain the intermediate 10-1 with yield of 58%. MS (ASAP)=422.0.

### Synthesis of Intermediate 10-2

A dry three neck-flask (250 mL) was provided, a reaction system was set up, and the three neck-flask was vacuumed and nitrogen was injected. Nitrogen was kept flowing in the three neck-flask, THF (100 mL) was added to the intermediate 10-1 (5.0 g, 11.8 mmol), the three neck-flask was vacuumized, and nitrogen was circulated for three times. Then, the solution was cooled to a temperature of −78° C. A n-butyl lithium solution (4.7 mL, 11.8 mmol) was slowly added into the three neck-flask dropwise. After the reaction lasted at a temperature of −78° C. for 60 minutes, triethyl borate (1.7 g, 11.8 mmol) was slowly added dropwise. Then, the temperature of the reaction system was slowly raised to room temperature, and the reaction lasted for 12 hours. Diluted hydrochloric acid was added to the solution and stirred for 30 minutes. The solution was extracted with EA and then was rotationally evaporated to remove the solvent, PE was used to obtain a white solid, namely the intermediate 10-2 with yield of 88%. MS(ASAP)-388.1.

### Synthesis of the Compound 10

The intermediate 10-2 (3.9 g, 10 mmol) and the intermediate 9-1 (5.2 g, 10 mmol) were dissolved in a mixed solvent of 1,4-dioxane and water (100 mL:10 mL), and Pd₂(dba)₃ (0.18 g, 0.2 mmol), s-phos (0.16 g, 0.4 mmol), and potassium carbonate (4.1 g, 30 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography and recrystallized to obtain the compound 10 with yield of 68%. MS (ASAP)=827.3.

### Example 11

The compound represented by formula (1) is compound 11, and the compound 11 is synthesized through a following synthetic route:

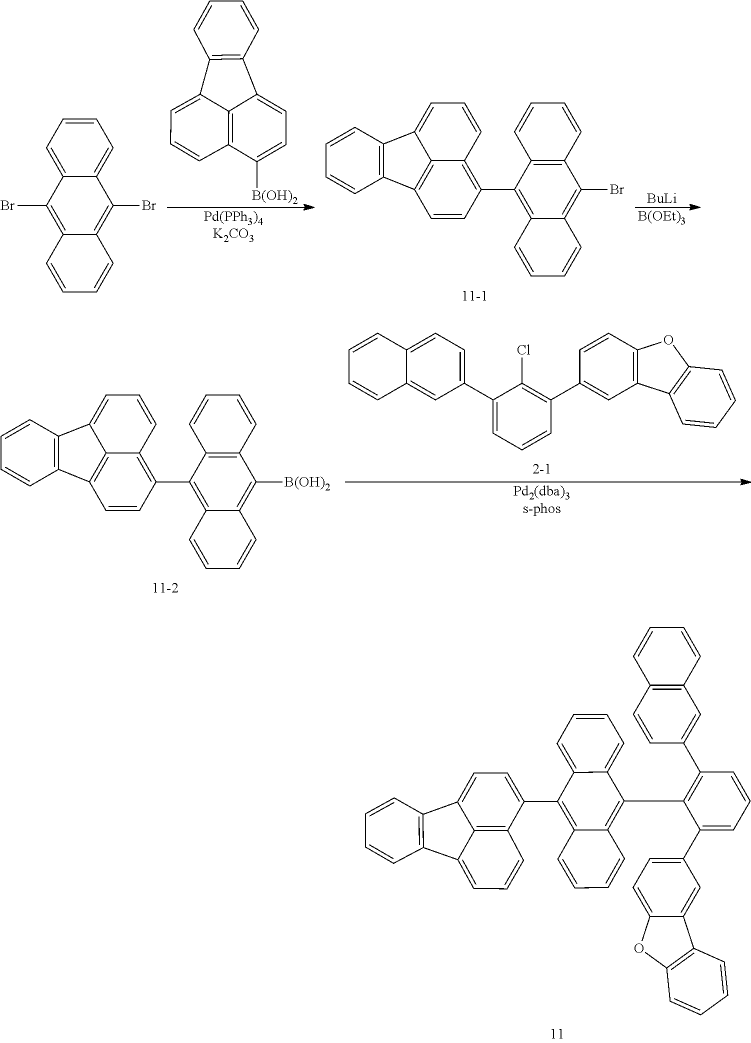

### Synthesis of Intermediate 11-1

9,10-dibromoanthrene (3.4 g, 10 mmol) and fluoranthene-3-boric acid (2.5 g, 10 mmol) were dissolved in a mixed solvent of 1,4-dioxane and water (100 mL:10 mL), and Pd(PPh₃)₄ (1.15 g, 1 mmol) and potassium carbonate (8.3 g, 60 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography and recrystallized to obtain the intermediate 11-1 with yield of 62%. MS (ASAP)=456.1.

### Synthesis of Intermediate 11-2

A dry three neck-flask (250 mL) was provided, a reaction system was set up, and the three neck-flask was vacuumed and nitrogen was injected. Nitrogen was kept flowing in the three neck-flask, THF (100 mL) was added to the intermediate 11-1 (10 g, 22 mmol), the three neck-flask was vacuumized, and nitrogen was circulated for three times, Then, the solution was cooled to a temperature of −78° C. A n-butyl lithium solution (8.8 mL, 22 mmol) was slowly added into the three neck-flask dropwise. After the reaction lasted at a temperature of −78° C. for 60 minutes, triethyl borate (3.2 g, 22 mmol) was slowly added dropwise. Then, the temperature of the reaction system was slowly raised to room temperature, and the reaction lasted for 12 hours. Diluted hydrochloric acid was added to the solution and stirred for 30 minutes. The solution was extracted with EA and then was rotationally evaporated to remove the solvent, PE was used to obtain a white solid, namely the intermediate 11-2 with yield of 89%. MS(ASAP)=422.2.

### Synthesis of the Compound 11

The intermediate 11-2 (4.2 g, 10 mmol) and the intermediate 2-1 (4.1 g, 10 mmol) were dissolved in a mixed solvent of 1,4-dioxane and water (100 mL:10 mL), and Pd₂(dba)₃ (0.18 g, 0.2 mmol), s-phos (0.16 g, 0.4 mmol), and potassium carbonate (4.1 g, 30 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography and recrystallized to obtain the compound 11 with yield of 61%. MS (ASAP)=746.3.

### Example 12

The compound represented by formula (1) is compound 12, and the compound 12 is synthesized through a following synthetic route:

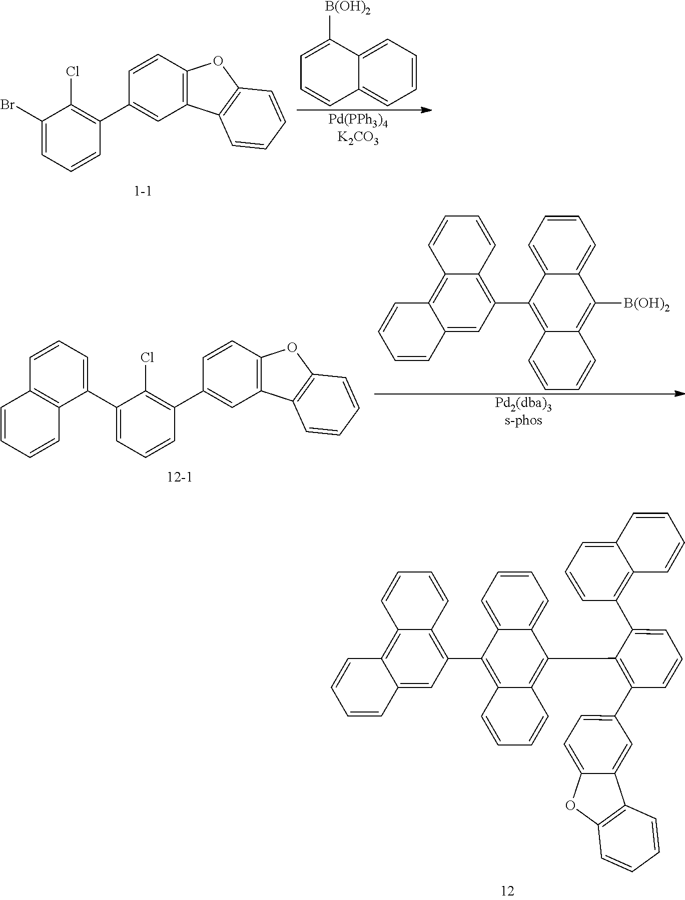

### Synthesis of Intermediate 12-1

The intermediate 1-1 (14.2 g, 40 mmol) and 1-naphthyl boric acid (6.9 g, 40 mmol) were dissolved in a mixed solvent of 1,4-dioxane and water (250 mL:50 mL), and Pd(PPh₃)₄ (0.92 g, 0.8 mmol) and potassium carbonate (27.6 g, 200 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:8) to obtain a white solid, namely the intermediate 12-1 with yield of 82%. MS (ASAP)=404.1.

### Synthesis of the Compound 12

The intermediates 12-1 (10 g, 24.8 mmol) and 10-(9-phenyl)-9-anthraceneboric acid (9.9 g, 24.8 mmol) were dissolved in a mixed solvent of 1,4-dioxane and water (200 mL:20 mL), and Pd₂(dba)₃ (0.46 g, 0.5 mmol), s-phos (0.41 g, 1.0 mmol), and potassium carbonate (17 g, 124 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:4) and recrystallized to obtain the compound 12 with yield of 66%. MS (ASAP)=722.3.

### Example 13

The compound represented by formula (1) is compound 13, and the compound 13 is synthesized through a following synthetic route:

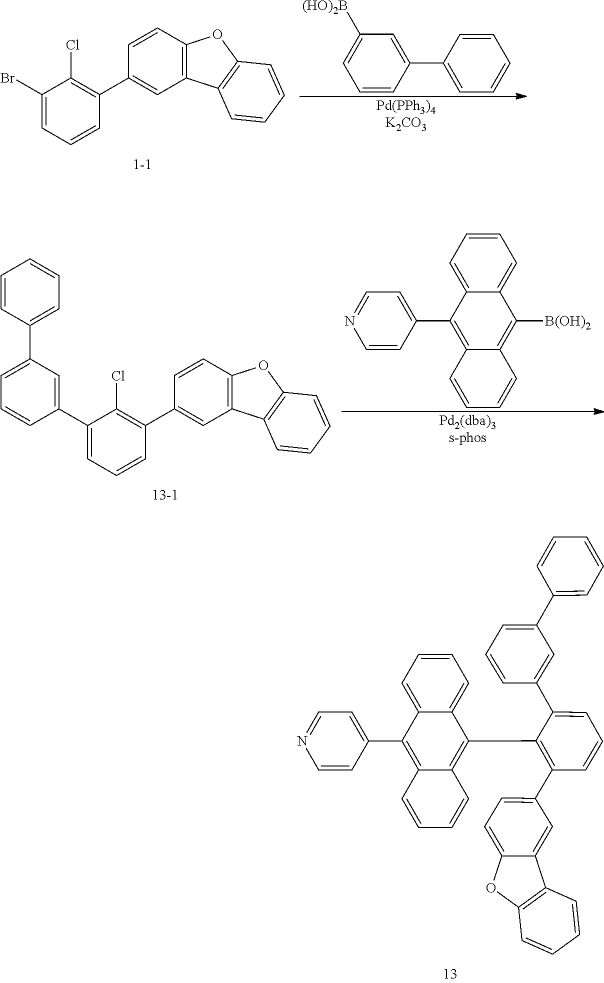

### Synthesis of Intermediate 13-1

The intermediate 1-1 (17.8 g, 50 mmol) and 2-biphenylboric acid (9.9 g, 50 mmol) were dissolved in a mixed solvent of 1,4-dioxane and water (250 mL:50 mL), and Pd(PPh₃)₄ (1.2 g, 1 mmol) and potassium carbonate (27.6 g, 200 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:8) to obtain a white solid, namely the intermediate 13-1 with yield of 77%. MS (ASAP)=430.1.

### Synthesis of the Compound 13

The intermediate 13-1 (10 g, 23.3 mmol) and 10-(3-pyridyl)-9-anthraceneboric acid (7.0 g, 23.3 mmol) were dissolved in a mixed solvent of 1,4-dioxane and water (200 mL:20 mL), and Pd₂(dba)₃ (0.42 g, 0.46 mmol), s-phos (0.38 g, 0.92 mmol), and potassium carbonate (16 g, 116 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:4) and recrystallized to obtain the compound 13 with yield of 62%. MS (ASAP)=649.2.

### Example 14

The compound represented by formula (1) is compound 14, and the compound 14 is synthesized through a following synthetic route:

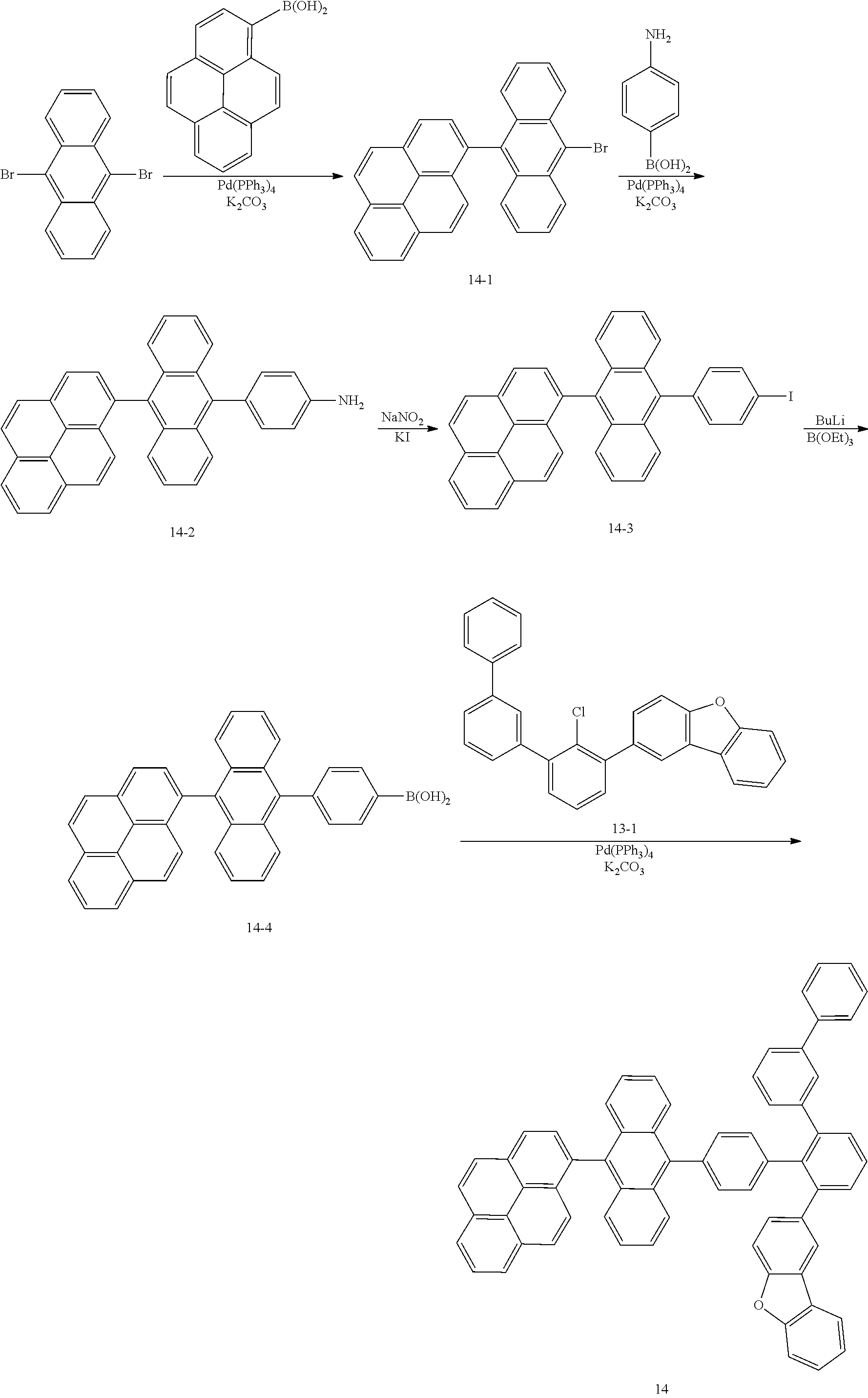

### Synthesis of Intermediate 14-1

9,10-dibromoanthrene (3.4 g, 10 mmol) and 1-pyrene boric acid (2.5 g, 10 mmol) were dissolved in a mixed solvent of 1,4-dioxane and (100 mL:10 mL), and Pd(PPh₃)₄ (1.15 g, 1 mmol) and potassium carbonate (8.3 g, 60 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography and recrystallized to obtain the intermediate 14-1 with yield of 71%. MS (ASAP)=456.1.

### Synthesis of Intermediate 14-2

The intermediate 14-1 (3.4 g, 10 mmol) and 4-aminophenylboric acid (1.4 g, 10 mmol) were dissolved in a mixed solvent of 1,4-dioxane and water (100 mL:10 mL), and Pd(PPh₃)₄ (1.15 g, 1 mmol) and potassium carbonate (8.3 g, 60 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography and recrystallized to obtain the intermediate 14-2 with yield of 71%. MS (ASAP)=469.2.

### Synthesis of Intermediate 14-3

The intermediate 14-2 (8.0 g, 17 mmol) was added into a three neck-flask (250 mL), and 100 mL of tetrahydrofuran and 20 mL of dilute hydrochloric acid were added into the three neck-flask. The three neck-flask was putted in an ice bath. NaNO₂ (1.4 g, 20 mmol) was dissolved in 20 mL of water, and then was slowly added into a tetrahydrofuran solution of the intermediate 14-2 dropwise, and stirred for 1 hour under the ice bath. KI (3.4 g, 20 mmol) was dissolved in 50 mL of water, and was slowly added into the above reaction solution. The reaction lasted at room temperature for 12 hours. After the reaction was completed, sodium thiosulfate aqueous solution was added into the solution, and was extracted with dichloromethane, rotationally evaporated, and washed with water. The product was purified by column chromatography (an eluent is a mixture of PE and DCM with a volume ratio of 2:1) to obtain a white solid, namely the intermediate 14-3 with yield of 81%. MS(ASAP)=580.1.

### Synthesis of Intermediate 14-4

A dry three neck-flask (250 mL) was provided, a reaction system was set up, and the three neck-flask was vacuumed and nitrogen was injected. Nitrogen was kept flowing in the three neck-flask, THF (100 mL) was added to the intermediate 14-3 (10 g, 17 mmol), the three neck-flask was vacuumized, and nitrogen was circulated for three times. Then, the solution was cooled to a temperature of −78° C. A n-butyl lithium solution (6.9 mL, 17 mmol) was slowly added into the three neck-flask dropwise. After the reaction lasted at a temperature of −78° C. for 60 minutes, triethyl borate (2.5 g, 17 mmol) was slowly added dropwise. Then, the temperature of the reaction system was slowly raised to room temperature, and the reaction lasted for 12 hours. Diluted hydrochloric acid was added to the solution and stirred for 30 minutes. The solution was extracted with EA and then was rotationally evaporated to remove the solvent, PE was used to obtain a white solid, namely the intermediate 14-4 with yield of 89%. MS(ASAP)=498.2.

### Synthesis of the Compound 14

The intermediate 14-4 (5.0 g, 10 mmol) and the intermediate 13-1 (4.3 g, 10 mmol) were dissolved in a mixed solvent of 1,4-dioxane and water (200 mL:20 mL), and Pd₂(dba)₃ (0.18 g, 0.2 mmol), s-phos (0.16 g, 0.4 mmol), and potassium carbonate (6.9 g, 50 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:4) and recrystallized to obtain the compound 14 with yield of 58%. MS (ASAP)=848.3.

### Example 15

The compound represented by formula (1) is compound 15, and the compound 15 is synthesized through a following synthetic route:

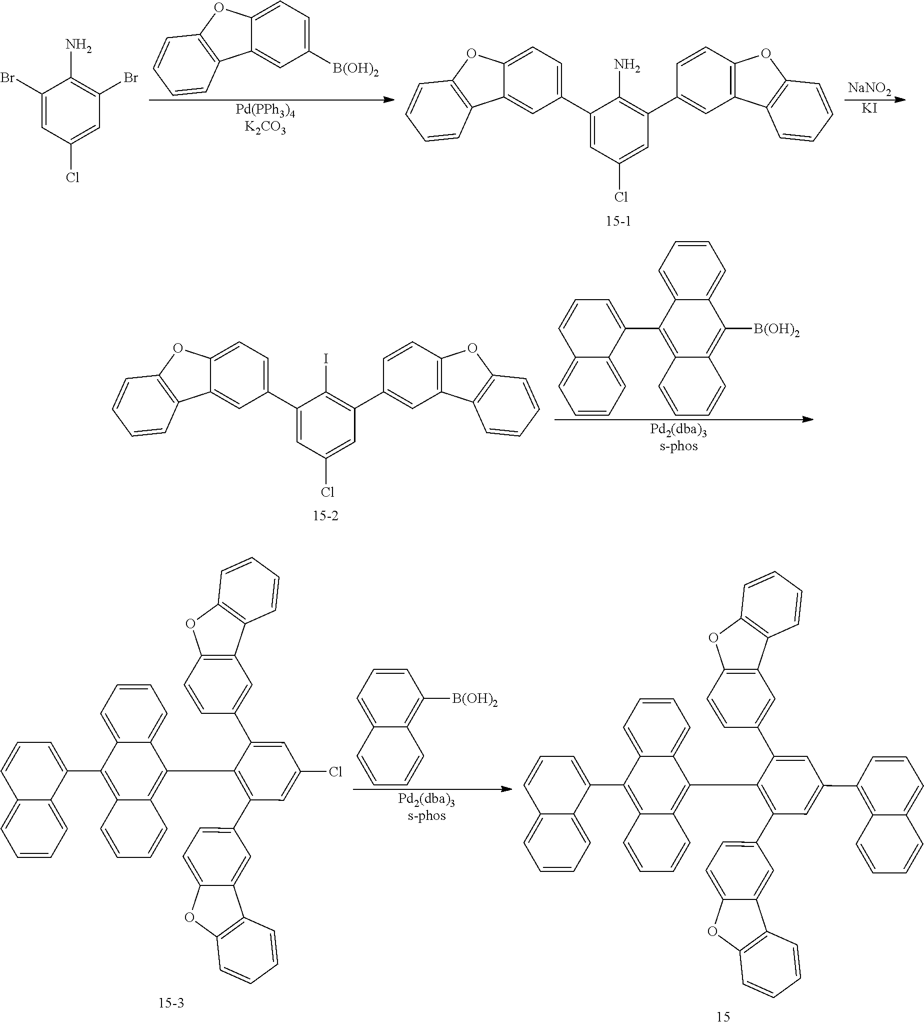

### Synthesis of Intermediate 15-1

4-chloro-2,6-dibromoaniline (28.5 g, 100 mmol) and dibenzofuran-2-boric acid (42 g, 200 mmol) were dissolved in a mixed solvent of 1,4-dioxane and water (500 mL:50 mL), and Pd(PPh₃)₄ (2.3 g, 2 mmol) and potassium carbonate (55 g, 400 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:8) to obtain a white solid, namely the intermediate 15-1 with yield of 82%. MS(ASAP)=459.1.

### Synthesis of Intermediate 15-2

The intermediate 15-1 (10 g, 21.8 mmol) was added into a three neck-flask (250 mL), and 100 mL of tetrahydrofuran and 20 mL of dilute hydrochloric acid were added into the three neck-flask. The three neck-flask was putted in an ice bath. NaNO₂ (3 g, 43.6 mmol) was dissolved in 20 mL of water, and then was slowly added into a tetrahydrofuran solution of the intermediate 15-1 dropwise, and stirred for 1 hour under the ice bath. KI (7.2 g, 43.6 mmol) was dissolved in 50 mL of water, and was slowly added into the above reaction solution. The reaction lasted at room temperature for 12 hours. After the reaction was completed, sodium thiosulfate aqueous solution was added into the solution, and was extracted with dichloromethane, rotationally evaporated, and washed with water. The product was purified by column chromatography (an eluent is a mixture of PE and DCM with a volume ratio of 2:1) to obtain a white solid, namely the intermediate 15-2 with yield of 43%. MS(ASAP)=570.0.

### Synthesis of Intermediate 15-3

The intermediate 15-2 (10 g, 17.5mmol) and 10-(1-naphthyl)-9-anthraceneboric acid (6.1 g, 17.5 mmol) were dissolved in a mixed solvent of 1,4-dioxane and water (200 mL:20 mL), and Pd(PPh₃)₄ (0.32 g, 0.35 mmol), s-phos (0.29 g, 0.7 mmol), and potassium carbonate (12 g, 88 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:5) and recrystallized to obtain the intermediate 15-3 with yield of 62%. MS (ASAP)=746.2.

### Synthesis of the Compound 15

The intermediate 15-3 (7.5 g, 10 mmol) and 1-naphthyl boric acid (1.7 g, 10 mmol) were dissolved in a mixed solvent of 1,4-dioxane and water (100 mL: 10 mL), and Pd₂(dba)₃ (0.18 g, 0.2 mmol), s-phos (0.16 g, 0.4 mmol), and potassium carbonate (6.9 g, 50 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography and recrystallized to obtain the compound 15 with yield of 82%. MS (ASAP)=838.3.

### Example 16

The compound represented by formula (1) is compound 16, and the compound 16 is synthesized through a following synthetic route:

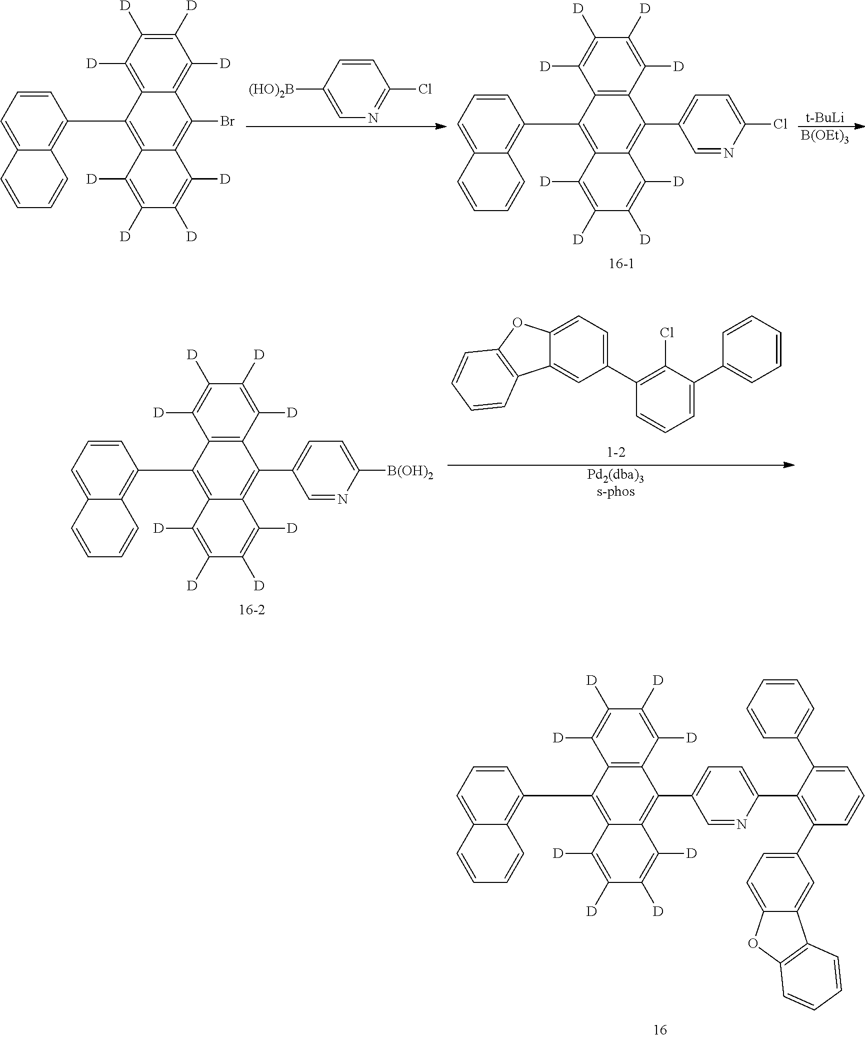

### Synthesis of Intermediate 16-1

9-bromo-10-(1-naphthyl) anthracene-1,2,3,4,5,6,7,8-d8 (3.9 g, 10 mmol) and 2-chloro-pyridine-5-boric acid (1.6 g, 10 mmol) were dissolved in a mixed solvent of 1,4-dioxane and water (100 mL:10 mL), and Pd(PPh₃)₄ (1.15 g, 1 mmol) and potassium carbonate (4.1 g, 30 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography and recrystallized to obtain the intermediate 16-1 with yield of 82%. MS (ASAP)=423.2.

### Synthesis of Intermediate 16-2

A dry three neck-flask (250 mL) was provided, a reaction system was set up, and the three neck-flask was vacuumed and nitrogen was injected. Nitrogen was kept flowing in the three neck-flask, THF (100 mL) was added to the intermediate 16-1 (4.2 g, 10 mmol), the three neck-flask was vacuumized, and nitrogen was circulated for three times, then, the solution was cooled to a temperature of −78° C. A n-butyl lithium solution (4.0 mL, 10 mmol) was slowly added into the three neck-flask dropwise. After the reaction lasted at a temperature of −78° C. for 60 minutes, triethyl borate (2.2 g, 15 mmol) was slowly added dropwise. Then, the temperature of the reaction system was slowly raised to room temperature, and the reaction lasted for 12 hours. Dilute hydrochloric acid was added to the solution and stirred for 30 minutes. The solution was extracted with EA and then was rotationally evaporated to remove the solvent, PE was used to obtain a white solid, namely the intermediate 16-2 with yield of 84%. MS(ASAP)=433.2.

### Synthesis of the Compound 16

The intermediate 16-2 (4.3 g, 10 mmol) and the intermediate 1-2 (3.5 g, 10 mmol) were dissolved in a mixed solvent of 1,4-dioxane and water (100 mL:10 mL), and Pd₂(dba)₃ (0.18 g, 0.2 mmol), s-phos (0.16 g, 0.4 mmol), and potassium carbonate (6.9 g, 50 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:4) and recrystallized to obtain the compound 16 with yield of 81%. MS (ASAP)=707.3.

### Example 17

The compound represented by formula (1) is compound 17, and the compound 17 is synthesized through a following synthetic route:

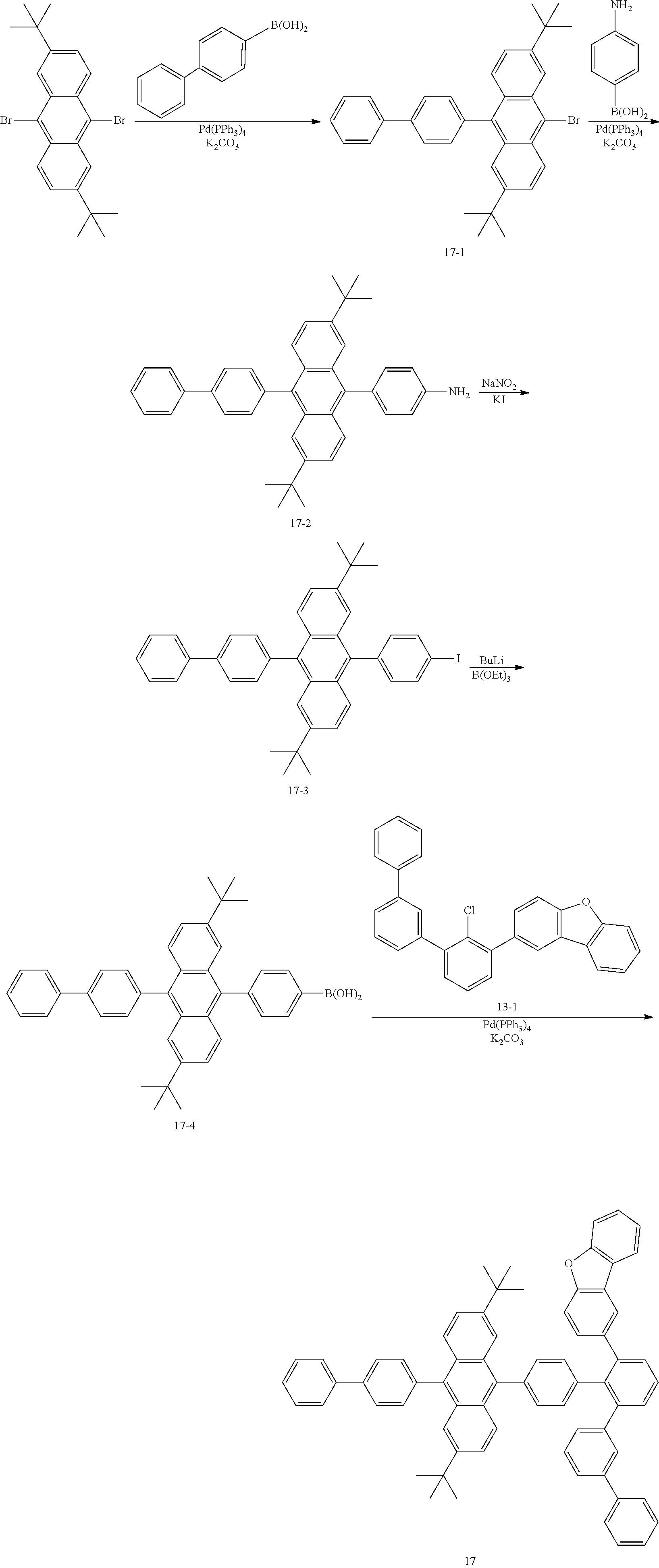

### Synthesis of Intermediate 17-1

9,10-dibromo-2,6-di-tert-butyl-anthracene (4.5 g, 10 mmol) and 1-biphenylboric acid (2.0 g, 10 mmol) were dissolved in a mixed solvent of 1,4-dioxane and water (100 mL:10 mL), and Pd(PPh₃)₄ (1.15 g, 1 mmol) and potassium carbonate (8.3 g, 60 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography and recrystallized to obtain the intermediate 17-1 with yield of 73%. MS (ASAP)=520.2.

### Synthesis of Intermediate 17-2

The intermediate 17-1 (5.2 g, 10 mmol) and 4-aminophenylboric acid (1.4 g, 10 mmol) were dissolved in a mixed solvent of 1,4-dioxane and water (100 mL:10 mL), and Pd(PPh₃)₄ (1.15 g, 1 mmol) and potassium carbonate (8.3 g, 60 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography and recrystallized to obtain the intermediate 17-2 with yield of 76%. MS (ASAP)=533.3.

### Synthesis of Intermediate 17-3

The intermediate 17-2 (5.3 g, 10 mmol) was added into a three neck-flask (250 mL), and 100 mL of tetrahydrofuran and 20 mL of dilute hydrochloric acid were added into the three neck-flask. The three neck-flask was putted in an ice bath. NaNO₂ (1.4 g, 20 mmol) was dissolved in 20 mL of water, and then was slowly added into a tetrahydrofuran solution of the intermediate 17-2 dropwise, and stirred for 1 hour under the ice bath. KI (3.4 g, 20 mmol) was dissolved in 50 mL of water, and was slowly added into the above reaction solution. The reaction lasted at room temperature for 12 hours. After the reaction was completed, sodium thiosulfate aqueous solution was added into the solution, and was extracted with dichloromethane, rotationally evaporated, and washed with water. The product was purified by column chromatography (an eluent is a mixture of PE and DCM with a volume ratio of 2:1) to obtain a white solid, namely the intermediate 17-3 with yield of 85%. MS(ASAP)=644.2.

### Synthesis of Intermediate 17-4

A dry three neck-flask (250 mL) was provided, a reaction system was set up, and the three neck-flask was vacuumed and nitrogen was injected. Nitrogen was kept flowing in the three neck-flask, THF (100 mL) was added to the intermediate 17-3 (6.4 g, 10 mmol), the three neck-flask was vacuumized, and nitrogen was circulated for three times. Then, the solution was cooled to a temperature of −78° C. A n-butyl lithium solution (4.0 mL, 10 mmol) was slowly added into the three neck-flask dropwise. After the reaction lasted at a temperature of −78° C. for 60 minutes, triethyl borate (2.5 g, 17 mmol) was slowly added dropwise. Then, the temperature of the reaction system was slowly raised to room temperature, and the reaction lasted for 12 hours. Diluted hydrochloric acid was added to the solution and stirred for 30 minutes. The solution was extracted with EA and then was rotationally evaporated to remove the solvent, PE was used to obtain a white solid, namely the intermediate 17-4 with yield of 84%. MS(ASAP)=562.3.

### Synthesis of the Compound 17

The intermediate 17-4 (5.6 g, 10 mmol) and the intermediate 13-1 (4.3 g, 10 mmol) were dissolved in a mixed solvent of 1,4-dioxane and water (200 mL:20 mL), and Pd₂(dba)₃ (0.18 g, 0.2 mmol), s-phos (0.16 g, 0.4 mmol), and potassium carbonate (6.9 g, 50 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:4) and recrystallized to obtain the compound 17 with yield of 52%. MS (ASAP)=912.4.

### Example 18

The compound represented by formula (1) is compound 18, and the compound 18 is synthesized through a following synthetic route:

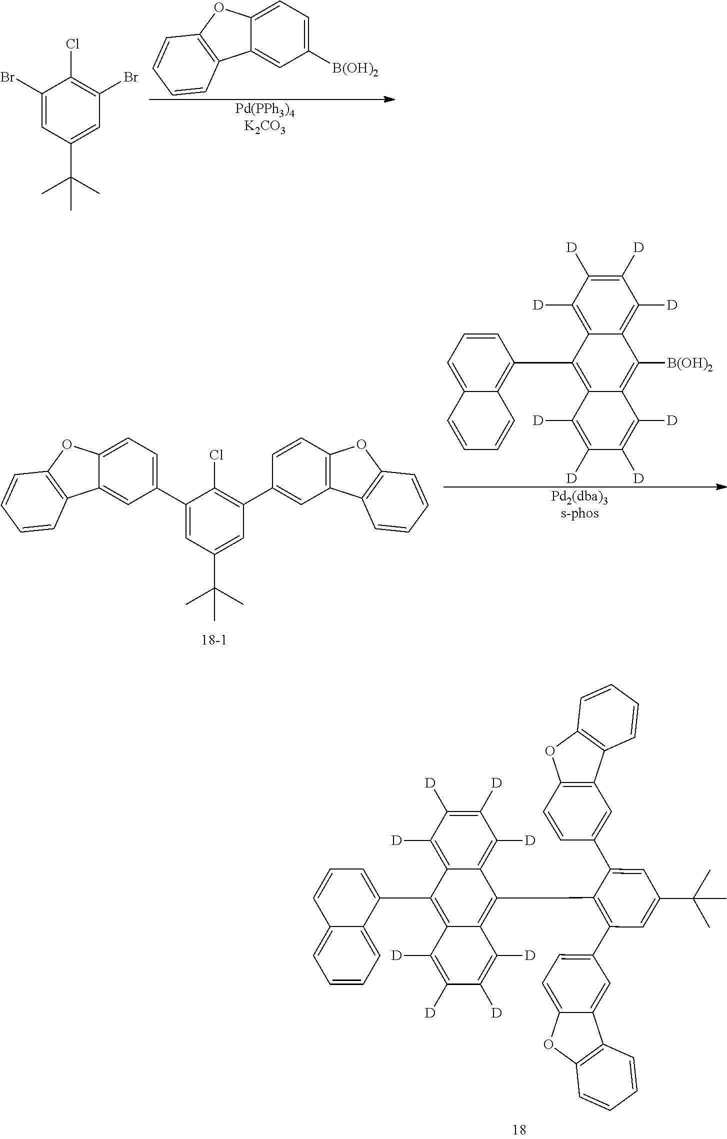

### Synthesis of Intermediate 18-1

2-chloro-1,3-dibromo-5-tert-butylbenzene (32.6 g, 100 mmol) and dibenzofuran-2-boric acid (42.4 g, 200 mmol) were dissolved in a mixed solvent of 1,4-dioxane and water (500 mL:50 mL), and Pd(PPh₃)₄ (1.8 g, 1.5 mmol) and potassium carbonate (62 g, 450 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:8) to obtain a white solid, namely the intermediate 18-1 with yield of 61%. MS(ASAP)=500.2.

### Synthesis of the Compound 18

The intermediate 18-1 (5.0 g, 10 mmol) and 9-boric acid-10-(1-naphthyl) anthracene-1,2,3,4,5,6,7,8-d8 (3.6 g, 10 mmol) were dissolved in a mixed solvent of 1,4-dioxane and water (200 mL:20 mL), and Pd₂(dba)₃ (0.41 g, 0.45 mol), s-phos (0.37 g, 0.9 mmol), and potassium carbonate (6.9 g, 50 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:4) and recrystallized to obtain the compound 18 with yield of 68%. MS (ASAP)=776.4.

### Example 19

The compound represented by formula (1) is compound 19, and the compound 19 is synthesized through a following synthetic route:

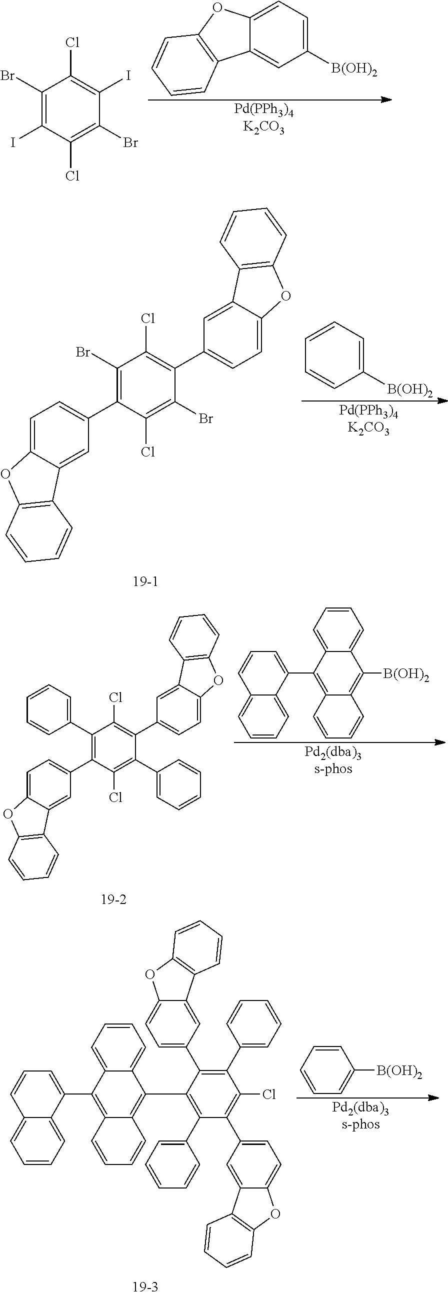

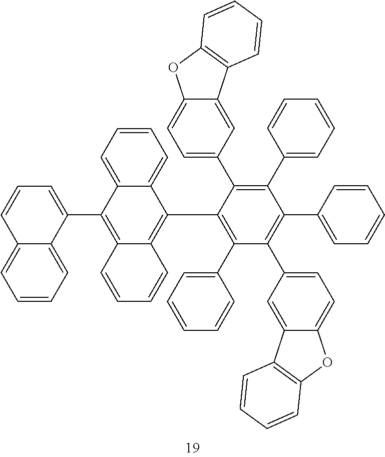 [SMILES: c1ccc(-c2c(-c3ccc4oc5ccccc5c4c3)c(-c3ccccc3)c(-c3c4ccccc4c(-c4cccc5ccccc45)c4ccccc34)c(-c3ccc4oc5ccccc5c4c3)c2-c2ccccc2)cc1]

### Synthesis of Intermediate 19-1

2,5-dichloro-1,4-dibromo-3,6-diiodiobenzene (55.7 g, 100 mmol) and dibenzofuran-2-boric acid (42.4 g, 200 mmol) were dissolved in a mixed solvent of 1,4-dioxane and water (500 mL:50 mL), and Pd(PPh₃)₄ (1.8 g, 1.5 mmol) and potassium carbonate (69 g, 500 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is PE) to obtain a white solid, namely the intermediate 19-1 with yield of 43%. MS (ASAP)=637.2.

### Synthesis of Intermediate 19-2

The intermediate 19-1 (31.9 g, 50 mmol) and phenylboric acid (24.4 g, 100 mmol) were dissolved in a mixed solvent of 1,4-dioxane and water (250 mL:50 mL), and Pd(PPh₃)₄ (0.88 g, 0.75 mmol) and potassium carbonate (28 g, 200 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:8) to obtain a white solid, namely the intermediate 19-2 with yield of 92%. MS (ASAP)=630.1.

### Synthesis of Intermediate 19-3

The intermediate 19-2 (63 g, 100 mmol) and 10-(1-naphthyl)-9-anthraceneboric acid (34.8 g, 100 mmol) were dissolved in a mixed solvent of 1,4-dioxane and water (200 mL:00 mL), and Pd₂(dba)₃ (0.92 g, 1 mmol), s-phos (0.82 g, 2 mmol), and potassium carbonate (69 g, 500 mmol) were added into the solution. In a nitrogen atmosphere, the solution was stirred at a temperature of 100° C.for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:5) and recrystallized to obtain the intermediate 19-3 with yield of 64%. MS (ASAP)=898.3.

### Synthesis of the Compound 19

The intermediate 19-3 (89.8 g, 100 mmol) and phenylboric acid (12.2 g, 100 mmol) were dissolved in a mixed solvent of 1,4-dioxane and water (200 mL:20 mL), and Pd₂(dba)₃ (0.92 g, 1 mmol), s-phos (0.82 g, 2 mmol), and potassium carbonate (69 g, 500 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:5) and recrystallized to obtain the compound 19 with yield of 67%. MS (ASAP)=940.3.

### Example 20

The compound represented by formula (1) is compound 20, and the compound 20 is synthesized through a following synthetic route:

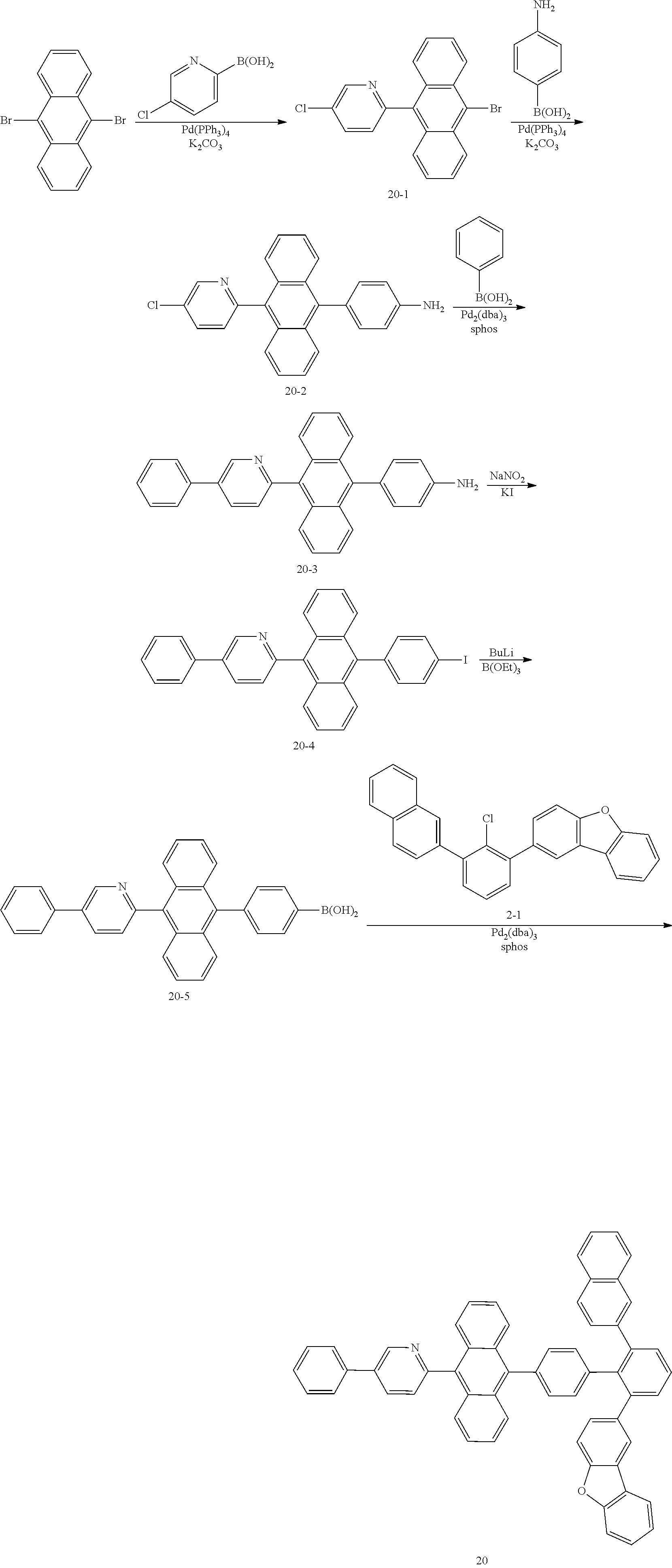

### Synthesis of Intermediate 20-1

9,10-dibromoanthrene (3.4 g, 10 mmol) and 5-chloro-2-pyridine boric acid (1.6 g, 10 mmol) were dissolved in a mixed solvent of 1,4-dioxane and water (100 mL:10 mL), and Pd(PPh₃)₄ (1.15 g, 1 mmol) and potassium carbonate (8.3 g, 60 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography and recrystallized to obtain the intermediate 20-1 with yield of 73%. MS (ASAP)=369.0.

### Synthesis of Intermediate 20-2

The intermediate 20-1 (3.7 g, 10 mmol) and 4-aminophenylboric acid (1.4 g, 10 mmol) were dissolved in a mixed solvent of 1,4-dioxane and water (100 mL:10 mL), and Pd(PPh₃)₄ (1.15 g, 1 mmol) and potassium carbonate (8.3 g, 60 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography and recrystallized to obtain the intermediate 20-2 with yield of 67%. MS (ASAP)=380.1.

### Synthesis of Intermediate 20-3

The intermediate 20-2 (3.8 g, 10 mmol) and phenylboric acid (1.2 g, 10 mmol) were dissolved in a mixed solvent of 1,4-dioxane and water (100 mL:10 mL), and Pd(PPh₃)₄ (1.15 g, 1 mmol) and potassium carbonate (8.3 g, 60 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography and recrystallized to obtain the intermediate 20-3 with yield of 76%. MS (ASAP)=422.2.

### Synthesis of Intermediate 20-4

The intermediate 20-3 (4.2 g, 10 mmol) was added into a three neck-flask (250 mL), and 100 mL of tetrahydrofuran and 20 mL of dilute hydrochloric acid were added into the three neck-flask. The three neck-flask was putted in an ice bath. NaNO₂ (1.4 g, 20 mmol) was dissolved in 20 mL of water, and then was slowly added into a tetrahydrofuran solution of the intermediate 14-2 dropwise, and stirred for 1 hour under the ice bath. KI (3.4 g, 20 mmol) was dissolved in 50 mL of water, and was slowly added into the above reaction solution. The reaction lasted at room temperature for 12 hours. After the reaction was completed, sodium thiosulfate aqueous solution was added into the solution, and was extracted with dichloromethane, rotationally evaporated, and washed with water. The product was purified by column chromatography (an eluent is a mixture of PE and DCM with a volume ratio of 2:1) to obtain a white solid, namely the intermediate 20-4 with yield of 79%. MS(ASAP)=533.1.

### Synthesis of Intermediate 20-5

A dry three neck-flask (250 mL) was provided, a reaction system was set up, and the three neck-flask was vacuumed and nitrogen was injected. Nitrogen was kept flowing in the three neck-flask, THF (100 mL) was added to the intermediate 20-4 (5.3 g, 10 mmol), the three neck-flask was vacuumized, and nitrogen was circulated for three times. Then, the solution was cooled to a temperature of −78° C. A n-butyl lithium solution (4.0 mL, 10 mmol) was slowly added into the three neck-flask dropwise. After the reaction lasted at a temperature of −78° C. for 60 minutes, triethyl borate (2.5 g, 17 mmol) was slowly added dropwise. Then, the temperature of the reaction system was slowly raised to room temperature, and the reaction lasted for 12 hours. Diluted hydrochloric acid was added to the solution and stirred for 30 minutes. The solution was extracted with EA and then was rotationally evaporated to remove the solvent, PE was used to obtain a white solid, namely the intermediate 20-5 with yield of 82%. MS(ASAP)=451.2.

### Synthesis of the Compound 20

The intermediate 20-5 (4.5 g, 10 mmol) and the intermediate 2-1 (4.0 g, 10 mmol) were dissolved in a mixed solvent of 1,4-dioxane and water (200 mL:20 mL), and Pd₂(dba)₃ (0.18 g, 0.2 mmol), s-phos (0.16 g, 0.4 mmol), and potassium carbonate (6.9 g, 50 mmol) were added into the solution. In a nitrogen atmosphere, the solution was stirred at a temperature of 100° C. for 12 hours. After the reaction was completed, the reaction solution was cooled and rotationally evaporated to remove most of the solvent, and then was extracted and washed with water. The organic solution was collected and then was purified by column chromatography (an eluent is a mixture of DCM and PE with a volume ratio of 1:4) and recrystallized to obtain the compound 20 with yield of 53%. MS (ASAP)=775.3.

The disclosure also provides a mixture. The mixture includes one or more compounds represented by formula (1) and one or more organic functional materials. The organic functional materials are selected from hole injection materials (HIM), hole transfer materials (HTM), electron transfer materials (ETM), electron injection materials (EIM), electron barrier materials (EBM), hole barrier materials (HBM), and light-emitting materials, host materials, or organic dye, and the like.

In an embodiment, the organic functional materials are selected from guest materials. Further, the organic functional materials are selected from blue light guest materials.

In a specific embodiment, the guest materials include a compound represented by formula (2):

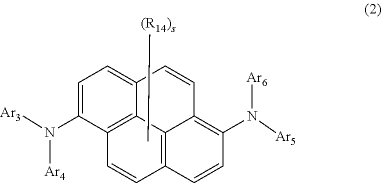

Ar₃ to Ar₆ are each independently selected from a substituted or unsubstituted alkyl aromatic group containing 6 to 60 ring atoms and a substituted or unsubstituted heteroaromatic group containing 6 to 60 ring atoms;

R₁₄ is independently selected from —H, -D, a C₁-C₂₀ linear alkyl group, a C₁-C₂₀ linear alkoxy group, a C₁-C₂₀ linear thioalkoxy group, a C₃-C₂₀ branched alkyl group, a C₃-C₂₀ branched alkoxy group, a C₃-C₂₀ branched thioalkoxy group, a C₃-C₁₀ cyclic alkyl group, a C₃-C₂₀ cyclic alkoxy group, a C₃-C₂₀ cyclic thioalkoxy group, a methylsilyl group, a C₁-C₂₀ ketone group, a C₂-C₂₀ alkoxycarbonyl group, a C₇-C₂₀ aryloxycarbonyl group, a cyano group, an aminoformyl group, a haloformyl group, a formyl group, an isocyano group, an isocyanate group, a thiocyanate group, an isothiocyanate group, a hydroxy group, a nitro group, an amino group, —CF₃, —Cl, —Br, —F, —I, an aromatic group containing 6 to 60 ring atoms, a heteroaromatic group containing 5 to 60 ring atoms, an aryloxy group containing 5 to 60 ring atoms, and a heteroaryloxy group containing 5 to 60 ring atoms at each occurrence; and

s is 0, 1, 2, 3, 4, 5, 6, 7, or 8.

Preferably, Ar₃ to Ar₆ are each independently selected from a substituted or unsubstituted aromatic group containing 6 to 14 ring atoms and a substituted or unsubstituted heteroaromatic group containing 6 to 14 ring atoms.

In particular, Ar₃ to Ar₆ are each independently selected from any of following structures:

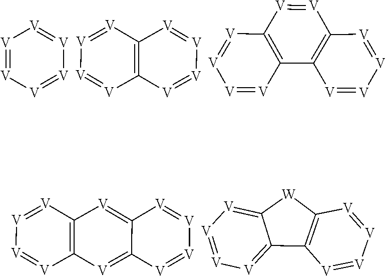

V is independently selected from CR₁₅ and N at each occurrence;

W is selected from NR₁₆, CR₁₆R₁₇, SiR₁₆R₁₇, O, S, S—O, and SO₂; and

R₁₅ to R₁₇ are each independently selected from —H, -D, a C₁-C₂₀ linear alkyl group, a C₁-C₂₀ linear alkoxy group, a C₁-C₂₀ linear thioalkoxy group, a C₃-C₂₀ branched alkyl group, a C₃-C₂₀ branched alkoxy group, a C₃-C₂₀ branched thioalkoxy group, a C₃-C₂₀ cyclic alkyl group, a C₃-C₂₀ cyclic alkoxy group, a C₃-C₂₀ cyclic thioalkoxy group, a methylsilyl group, a C₁-C₂₀ ketone group, a C₂-C₂₀ alkoxycarbonyl group, a C₇-C₂₀ aryloxycarbonyl group, a cyano group, an aminoformyl group, a haloformyl group, a formyl group, an isocyano group, an isocyanate group, a thiocyanate group, an isothiocyanate group, a hydroxy group, a nitro group, an amino group, —CF₃, —Cl, —Br, —F, —I, a substituted or unsubstituted aromatic group containing 6 to 60 ring atoms, a substituted or unsubstituted heteroaromatic group containing 5 to 60 ring atoms, a substituted or unsubstituted aryloxy group containing 5 to 60 ring atoms, and a substituted or unsubstituted heteroaryloxy group containing 5 to 60 ring atoms at each occurrence.

Preferably, R₁₅ to R₁₇ are each independently selected from —H, -D, a C₁-C₁₀ linear alkyl group, a C₃-C₁₀ branched alkyl group, a C₃-C₁₀ cyclic alkyl group, a substituted or unsubstituted aromatic group containing 6 to 30 ring atoms, and a substituted or unsubstituted heteroaromatic group containing 5 to 30 ring atoms at each occurrence.

More preferably, R₁₅ to R₁₇ are each independently selected from —H, -D, a C₁-C₁₀ linear alkyl group, a C₃-C₁₀ branched alkyl group, a cyclic alkyl group, and a phenyl group at each occurrence.

In particular, the compound represented by formula (2) is a compound represented by following formula:

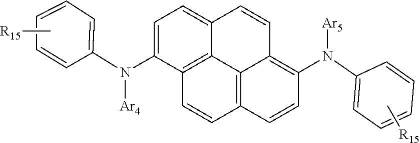

Preferably, the compound represented by formula (2) is selected from compounds represented by following formulas:

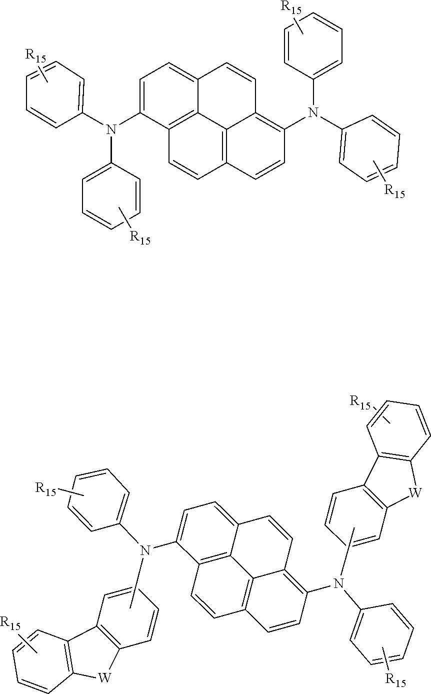

More specifically, the compound represented by formula (2) is selected from any of following structures:

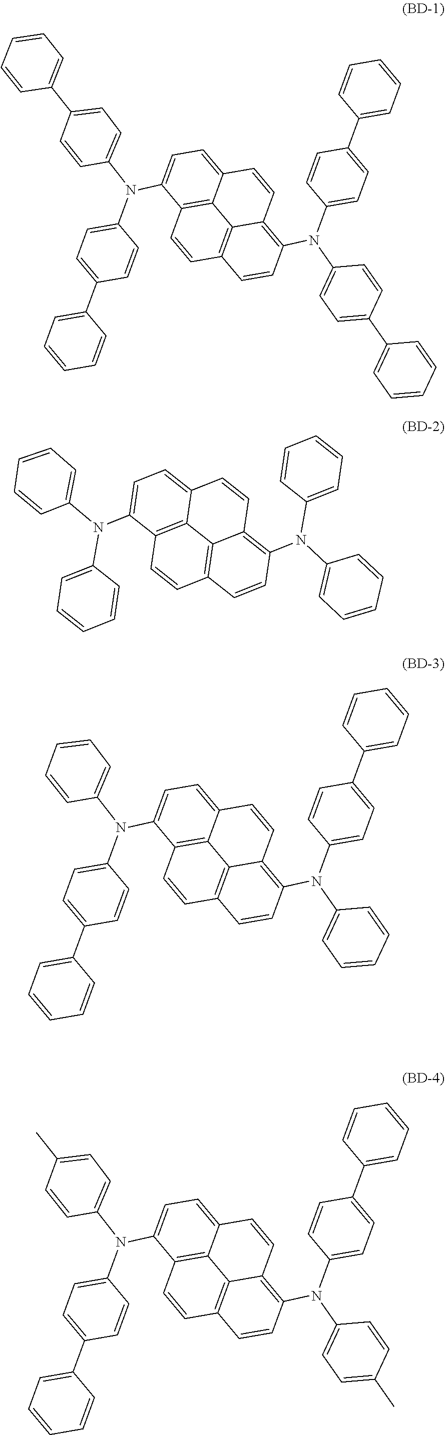 [SMILES: c1ccc(-c2ccc(N(c3ccc(-c4ccccc4)cc3)c3ccc4ccc5c(N(c6ccc(-c7ccccc7)cc6)c6ccc(-c7ccccc7)cc6)ccc6ccc3c4c65)cc2)cc1 ; c1ccc(N(c2ccccc2)c2ccc3ccc4c(N(c5ccccc5)c5ccccc5)ccc5ccc2c3c54)cc1 ; c1ccc(-c2ccc(N(c3ccccc3)c3ccc4ccc5c(N(c6ccccc6)c6ccc(-c7ccccc7)cc6)ccc6ccc3c4c65)cc2)cc1 ; Cc1ccc(N(c2ccc(-c3ccccc3)cc2)c2ccc3ccc4c(N(c5ccc(C)cc5)c5ccc(-c6ccccc6)cc5)ccc5ccc2c3c54)cc1]

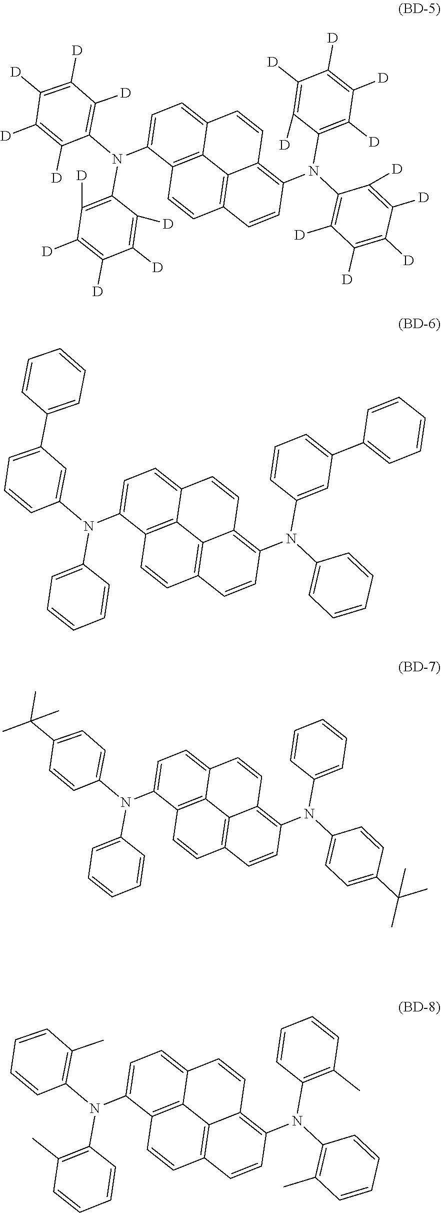 [SMILES: [2H]c1c([2H])c([2H])c(N(c2c([2H])c([2H])c([2H])c([2H])c2[2H])c2ccc3ccc4c(N(c5c([2H])c([2H])c([2H])c([2H])c5[2H])c5c([2H])c([2H])c([2H])c([2H])c5[2H])ccc5ccc2c3c54)c([2H])c1[2H] ; c1ccc(-c2cccc(N(c3ccccc3)c3ccc4ccc5c(N(c6ccccc6)c6cccc(-c7ccccc7)c6)ccc6ccc3c4c65)c2)cc1 ; CC(C)(C)c1ccc(N(c2ccccc2)c2ccc3ccc4c(N(c5ccccc5)c5ccc(C(C)(C)C)cc5)ccc5ccc2c3c54)cc1 ; Cc1ccccc1N(c1ccccc1C)c1ccc2ccc3c(N(c4ccccc4C)c4ccccc4C)ccc4ccc1c2c43]

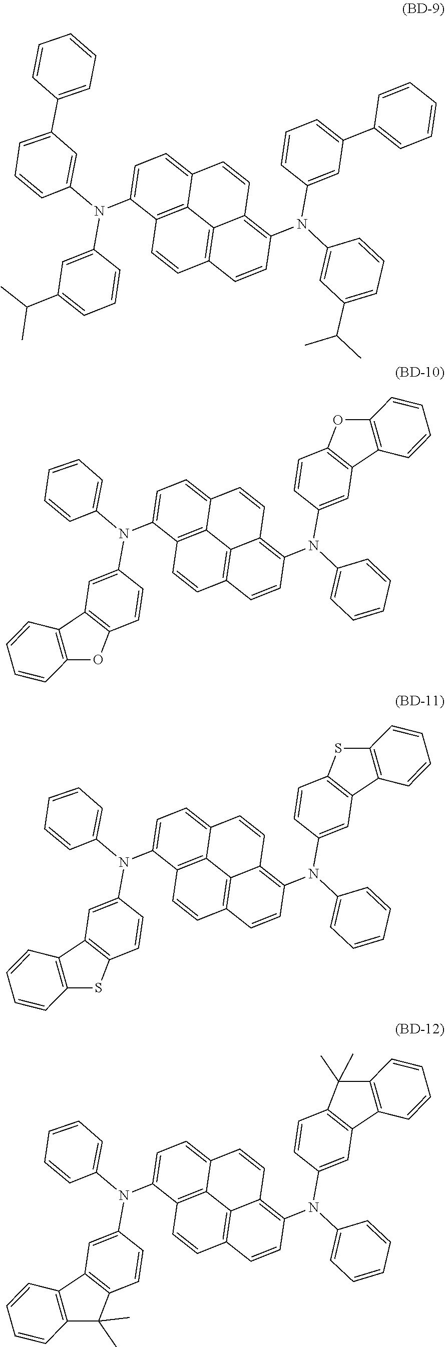 [SMILES: CC(C)c1cccc(N(c2cccc(-c3ccccc3)c2)c2ccc3ccc4c(N(c5cccc(-c6ccccc6)c5)c5cccc(C(C)C)c5)ccc5ccc2c3c54)c1 ; c1ccc(N(c2ccc3oc4ccccc4c3c2)c2ccc3ccc4c(N(c5ccccc5)c5ccc6oc7ccccc7c6c5)ccc5ccc2c3c54)cc1 ; c1ccc(N(c2ccc3sc4ccccc4c3c2)c2ccc3ccc4c(N(c5ccccc5)c5ccc6sc7ccccc7c6c5)ccc5ccc2c3c54)cc1 ; CC1(C)c2ccccc2-c2cc(N(c3ccccc3)c3ccc4ccc5c(N(c6ccccc6)c6ccc7c(c6)-c6ccccc6C7(C)C)ccc6ccc3c4c65)ccc21]

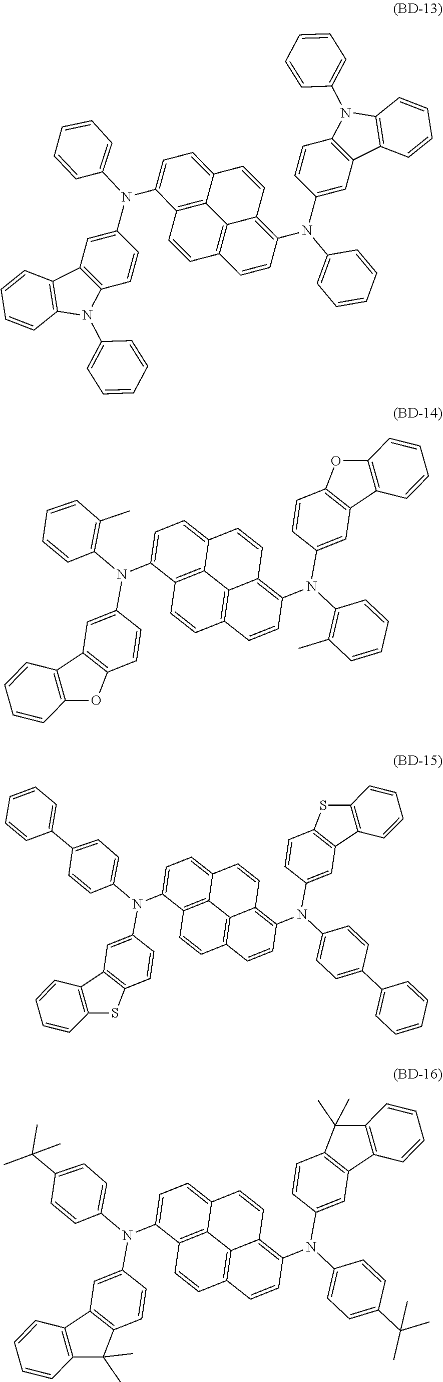 [SMILES: c1ccc(N(c2ccc3c(c2)c2ccccc2n3-c2ccccc2)c2ccc3ccc4c(N(c5ccccc5)c5ccc6c(c5)c5ccccc5n6-c5ccccc5)ccc5ccc2c3c54)cc1 ; Cc1ccccc1N(c1ccc2oc3ccccc3c2c1)c1ccc2ccc3c(N(c4ccc5oc6ccccc6c5c4)c4ccccc4C)ccc4ccc1c2c43 ; c1ccc(-c2ccc(N(c3ccc4sc5ccccc5c4c3)c3ccc4ccc5c(N(c6ccc(-c7ccccc7)cc6)c6ccc7sc8ccccc8c7c6)ccc6ccc3c4c65)cc2)cc1 ; CC(C)(C)c1ccc(N(c2ccc3c(c2)-c2ccccc2C3(C)C)c2ccc3ccc4c(N(c5ccc(C(C)(C)C)cc5)c5ccc6c(c5)-c5ccccc5C6(C)C)ccc5ccc2c3c54)cc1]

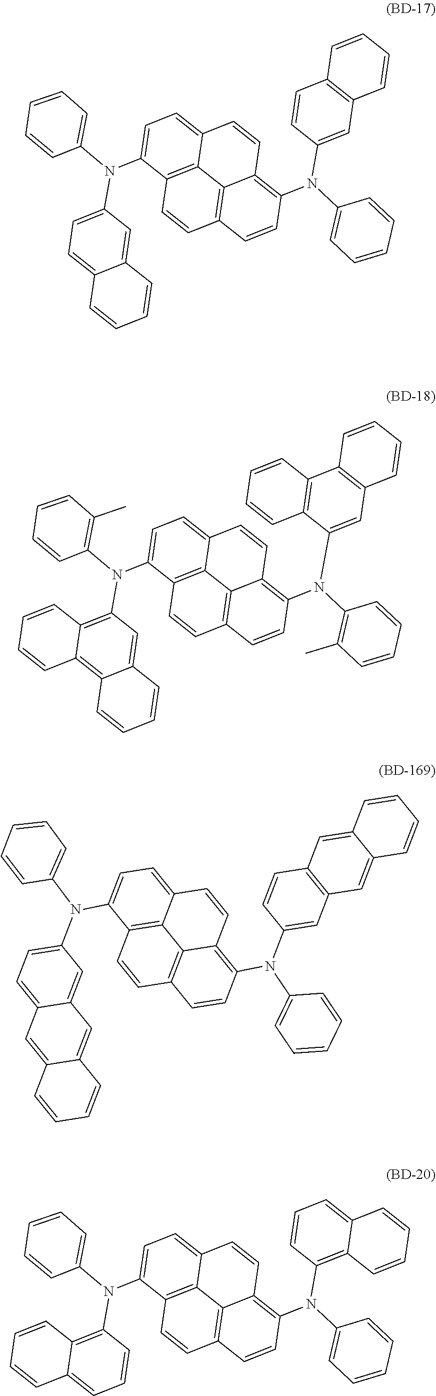 [SMILES: c1ccc(N(c2ccc3ccccc3c2)c2ccc3ccc4c(N(c5ccccc5)c5ccc6ccccc6c5)ccc5ccc2c3c54)cc1 ; Cc1ccccc1N(c1cc2ccccc2c2ccccc12)c1ccc2ccc3c(N(c4ccccc4C)c4cc5ccccc5c5ccccc45)ccc4ccc1c2c43 ; c1ccc(N(c2ccc3cc4ccccc4cc3c2)c2ccc3ccc4c(N(c5ccccc5)c5ccc6cc7ccccc7cc6c5)ccc5ccc2c3c54)cc1 ; c1ccc(N(c2cccc3ccccc23)c2ccc3ccc4c(N(c5ccccc5)c5cccc6ccccc56)ccc5ccc2c3c54)cc1]

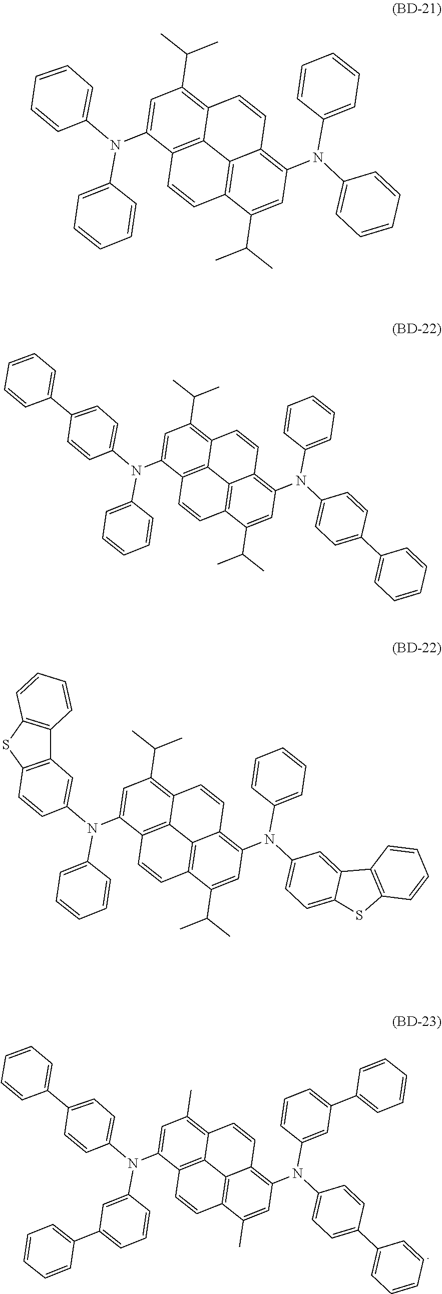 [SMILES: CC(C)c1cc(N(c2ccccc2)c2ccccc2)c2ccc3c(C(C)C)cc(N(c4ccccc4)c4ccccc4)c4ccc1c2c34 ; CC(C)c1cc(N(c2ccccc2)c2ccc(-c3ccccc3)cc2)c2ccc3c(C(C)C)cc(N(c4ccccc4)c4ccc(-c5ccccc5)cc4)c4ccc1c2c34 ; CC(C)c1cc(N(c2ccccc2)c2ccc3sc4ccccc4c3c2)c2ccc3c(C(C)C)cc(N(c4ccccc4)c4ccc5sc6ccccc6c5c4)c4ccc1c2c34 ; Cc1cc(N(c2ccc(-c3ccccc3)cc2)c2cccc(-c3ccccc3)c2)c2ccc3c(C)cc(N(c4ccc(-c5ccccc5)cc4)c4cccc(-c5ccccc5)c4)c4ccc1c2c34]

The disclosure also provides a composition, and the composition includes one or more compounds represented by formula (1) or one or more mixtures, and organic solvents. The one or more compounds or the one or more mixtures are present at a concentration of from 0.01% to 10% by mass, preferably 0.1% to 8% by mass, more preferably 0.2% to 5% by mass, and most preferably 0.25% to 3% by mass.

The organic solvents are selected from aromatic, heteroaromatic, ester, aromatic ketone, aromatic ether, aliphatic ketone, aliphatic ether, alicyclic, olefin compound, borate ester, and phosphate ester.

Aromatic or heteroaromatic solvents suitable for the disclosure include, but are not limited: p-diisopropylbenzene, pentyl benzene, tetrahydronaphthalene, cyclohexylbenzene, chloronaphthalene, 1,4-dimethylnaphthalene, 3-isopropylbiphenyl, p-methylisopropyl benzene, dipentyl benzene, tripentyl benzene, pentyl toluene, o-diethylbenzene, m-diethylbenzene, p-diethylbenzene, 1,2,3,4-tetratoluene, 1,2,3,5-tetratoluene, 1,2,4,5-tetratoluene, butadiene benzene, dodecylbenzene, dihexylbenzene, dibutylbenzene, p-diisopropylbenzene, cyclohexylbenzene, benzylbutylbenzene, dimethylnaphthalene, 3-isopropylbiphenyl, p-methylcumene, 1-methylnaphthalene, 1,2,4-trichlorobenzene, 4,4-difluorodiphenylmethane, 1,2-dimethoxy-4-(1-propenyl) benzene, diphenylmethane, 2-phenylpyridine, 3-phenylpyridine, N-methyldiphenylamine, 4-isopropylbiphenyl, α, α-dichlorodiphenylmethane, 4-(3-phenylpropyl) pyridine, benzyl benzoate, 1,1-bis(3,4-dimethylphenyl) ethane, 2-isopropylnaphthalene, quinoline, isoquinoline, methyl 2-furanoate, ethyl 2-furanoate, and the like.

Aromatic ketone solvents suitable for the disclosure include but are not limited: 1-tetrahydronaphthalenone, 2-tetrahydronaphthalenone, 2-(phenyl epoxy) tetrahydronaphthalenone, 6-(methoxy) tetrahydronaphthalenone, acetophenone, phenylacetone, benzophenone, and derivatives of these compounds, such as 4-methylacetophenone, 3-methylacetophenone, 2-methylacetophenone, 4-methylphenylacetone, 3-methylphenylacetone, 2-methylphenylacetone, and the like.

Aromatic ether solvents suitable for the disclosure include but are not limited: 3-phenoxytoluene, butoxybenzene, p-anisaldehyde dimethyl acetal, tetrahydro-2-phenoxy-2H-pyran, 1,2-dimethoxy-4-(1-propenyl) benzene, 1,4-benzodioxane, 1,3-dipropylbenzene, 2,5-dimethoxytoluene, 4-ethylbasic ether, 1,3-dipropoxybenzene, 1,2,4-trimethoxybenzene, 4-(1-propenyl)-1,2-dimethoxybenzene, 1,3-dimethoxybenzene, glycidyl phenyl ether, dibenzyl ether, 4-tert-butyl anisole, trans-p-propenylanisole, 1,2-dimethoxybenzene, 1-methoxynaphthalene, diphenyl ether, 2-phenoxymethyl ether, 2-phenoxytetrahydrofuran, and ethyl-2-naphthyl ether.

Aliphatic ketone solvents suitable for the disclosure include but are not limited: 2-nonone, 3-nonone, 5-nonone, 2-decanone, 2,5-hexanedione, 2,6,8-trimethyl-4-nonone, fenone, phorone, isophorone, and di-n-pentyl ketone, and aliphatic ether, such as amyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, and tetraethylene glycol dimethyl ether, and the like.

Ester-based solvents suitable for the disclosure include but are not limited: octanoate, sebacate, stearate, benzoate, phenylacetate, cinnamate, oxalate, maleate, alkyl lactone, oleate, and the like. In some embodiments, the ester-based solvents may be selected from octyl octanoate, diethyl sebacate, diallyl phthalate, and isononyl isononanoate.

The organic solvents may be one organic solvent or as a mixture of two or more organic solvents.

In some embodiments, the organic solvents also include one or more of following compounds: methanol, ethanol, 2-methoxyethanol, dichloromethane, chloroform, chlorobenzene, o-dichlorobenzene, tetrahydrofuran, anisole, morpholine, toluene, o-xylene, m-xylene, p-xylene, 1,4-dioxane, acetone, methyl ethyl ketone, 1,2-dichloroethane, 3-phenoxytoluene, 1,1,1-trichloroethane, 1,1,2,2-tetrachloroethane, ethyl acetate, butyl acetate, dimethylformamide, dimethylacetamide, dimethyl sulfoxide, tetralin, naphthane, and indene.

Preferably, the organic solvents are those with Hansen solubility parameters in following ranges:

δd (dispersion force) of the organic solvents range from 17.0 MPa^{1/2} to 23.2 MPa^{1/2}, and further ranges from 18.5 MPa^{1/2} to 21.0 MPa^{1/2};

δp (polarity force) of the organic solvents range from 0.2 MPa^{1/2} to 12.5 MPa^{1/2}, and further ranges from 2.0 MPa^{1/2} to 6.0 MPa^{1/2}; and

δh (hydrogen bonding force) of the organic solvents range from 0.9 MPa^{1/2} to 14.2 MPa^{1/2}, and further ranges from 2.0 MPa^{1/2} to 6.0 MPa^{1/2}

Boiling points of the organic solvents are greater than or equal to 150° C. In some embodiments, the boiling points are greater than or equal to 180° C. In some embodiments, the boiling points are greater than or equal to 200° C. In some embodiments, the boiling points are greater than or equal to 250° C. In some embodiments, the boiling points are greater than or equal to 300° C. The boiling points within these ranges are beneficial to prevent nozzles of inkjet printing heads from clogging. The organic solvents can be evaporated from solvent system to form films containing functional materials.

The composition may be a solution or a suspension.

The composition may be used as a coating or printing ink to prepare organic electronic devices. The methods for preparing the composition include but are not limited to inkjet printing, letterpress printing, screen printing, dip coating, rotating coating, scraper coating, roller printing, rotary roller printing, lithographic printing, flexographic printing brush, rotary printing, spray coating, brush coating or pad printing, slit extrusion coating, and the like. A first choice is intaglio printing, jet printing, and inkjet printing. The solution or the suspension may also include one or more components such as surfactant, lubricant, wetting agent, dispersant, hydrophobic agent, adhesive, which may be used for adjusting viscosity, forming performance of films, and improving adhesion, and the like.

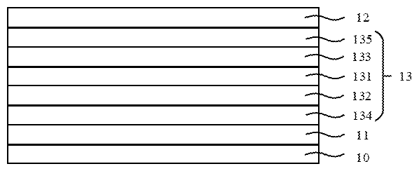

Referring to FIG. 1, the disclosure also provides an organic electronic device. The organic electronic device includes a first electrode 11, a second electrode 12, and an organic functional layer 13 disposed between the first electrode 11 and the second electrode 12. The organic functional layer 13 includes a film layer structure prepared by the compound, the mixture, or the composition described in any of the embodiments of the disclosure.

The organic electronic device includes but is not limited to an organic light-emitting diode, an organic photovoltaic battery, an organic light-emitting battery, an organic field-effect tube, an organic light-emitting field-effect tube, an organic laser, an organic spin electron device, an organic sensor, or an organic plasmon emission diode, and the like. The organic electronic device is preferably the organic light-emitting diode. The organic electronic device may be applied to various electronic devices, including but not limited to a display panel, an illuminating device, and a lighting source, and the like.

In a specific embodiment, the organic functional layer 13 includes a light-emitting layer (EML) 131, a hole transport layer (HTL) 132, and an electron transport layer (ETL) 133. Further, the organic functional layer 13 also includes a hole injection layer (HIL) 134 and/or an electron injection layer (EIL) 135. The first electrode 11 is an anode, and the second electrode 12 is a cathode.

The anode may include a conductive metal, a conductive metal oxide, or a conductive polymer. Holes in the anode may be easily injected into the HIL, the HTL, or the light-emitting layer. In an embodiment, absolute value of a difference between work function of the anode and highest occupied molecular orbital (HOMO) energy level or valence band energy level of emitters of the light-emitting layer, or a p-type semiconductor material of the HIL, the HTL, or the electron blocking layer (EBL) is less than 0.5 eV. In some embodiments, the above-mentioned absolute value is less than 0.3 eV. In some embodiments, the above-mentioned absolute value is less than 0.2 eV. Examples of materials of the anode include but are not limited: aluminum (Al), copper (Cu), aurum (Au), argentum (Ag), magnesium (Mg), ferrum (Fe), cobalt (Co), nickel (Ni), manganese (Mn), palladium (Pd), platinum (Pt), indium tin oxide (ITO), and aluminum doped zinc oxide (AZO), and the like. The materials of the anode may be applied by any suitable technology, such as a suitable physical vapor deposition method including RF magnetron sputtering, vacuum thermal evaporation, electron beam (e-beam), and the like.

The cathode may include a conductive metal or a conductive metal oxide. Electrons in the cathode may be easily injected into the EIL, the ETL, or directly the light-emitting layer. In an embodiment, absolute value of a difference between work function of the cathode and lowest unoccupied molecular orbital (LUMO) energy level or valence band energy level of emitters in the light-emitting layer, or a n-type semiconductor material of the EIL, the ETL, or the HBL is less than 0.5 eV. In some embodiments, the above-mentioned absolute value is less than 0.3 eV. In some embodiments, the above-mentioned absolute value is less than 0.2 eV. Materials of the cathode include, but are not limited: Al, Au, Ag, calcium (Ca), barium (Ba), Mg, LiF/Al, MgAg alloy, BaF₂/Al, Cu, Fe, Co, Ni, Mn, Pd, Pt, ITO, and the like. The materials of the cathode may be applied to by any suitable technology, such as a suitable physical vapor deposition method including RF magnetron sputtering, vacuum thermal evaporation, or electron beam (e-beam), and the like.

In a specific embodiment, as shown in FIG. 1, the organic electronic device includes a substrate 10, an anode (ITO) 11, a hole injection layer (HIL) 134, a hole transfer layer (HTL) 132, a light-emitting layer (EML) 131, an electron transfer layer (ETL) 133, and a cathode 12. Preparation steps of the organic electronic device are as following:

a, a conductive glass substrate was provided and cleaned with a solvent (such as one or more of chloroform, ketone, and isopropyl alcohol), then, an ultraviolet ozone plasma treatment was performed on the conductive glass substrate;

b, polyethylene dioxythiophene (PEDOT, Clevios™ AI4083) was provided as a HIL material, a thin film with a thickness of 60 nm was deposited on the ITO conductive glass substrate by a rotating coating method in an ultra-clean room, and then treated on a hot plate at a temperature of 180° C. for 10 minutes to obtain the hole injection layer with a thickness of 40 nm;

c, polyvinylcarbazole (PVK, Sigma Aldrich, with an average molecular weight of 25000-50000) was provided as an HTL material, a film with a thickness of 20 nm was deposited on the HIL by a rotating coating method in a glove box full of nitrogen, and then treated on a hot plate at a temperature of 180° C.for 60 minutes; a solution used for rotating coating was the PVK added to a toluene solvent, and a concentration of the PVK in the solution was 5 mg/ml;

d, a methyl benzoate solution including a host material and a guest material was provided as an EML material, a film was deposited on the HTL by a rotating coating method in a glove box full of nitrogen, and then treated on a hot plate at a temperature of 140° C. for 10 minutes; the host material is the compound represented by formula (1) provided by the embodiment of the disclosure, the guest material is the compound represented by formula (2) provided by the embodiment of the disclosure, a mass ratio of the host material and the guest material is 95:5, and a concentration of the methyl benzoate in the solution was 15 mg/mL;

e, the heat-treated substrate was transferred to a vacuum chamber, and then

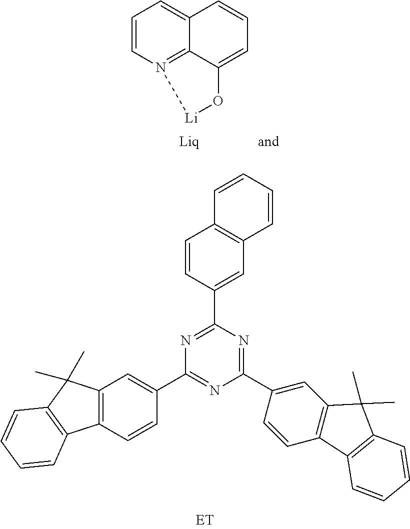

were placed in different evaporation units and co-deposited on the light-emitting layer at a ratio of 50 wt % respectively under high vacuum (1×10⁻⁶ mbar), to form the electron transport layer with a thickness of 20 nm; then, a cathode using a material of Al with a thickness of 100 nm was deposited; and

f, the above-mentioned device was encapsulated with UV curing resin in a glove box full of nitrogen.

The above-mentioned compounds 1 to 20 and following compounds BH-ref-01 and BH-ref-02 below are used as host materials, and the above-mentioned compound BD-1 are used guest materials. The above-mentioned preparation steps are used to prepare corresponding organic electronic devices OLED-1 to OLED-20, and OLED-ref1 and OLED-ref2.

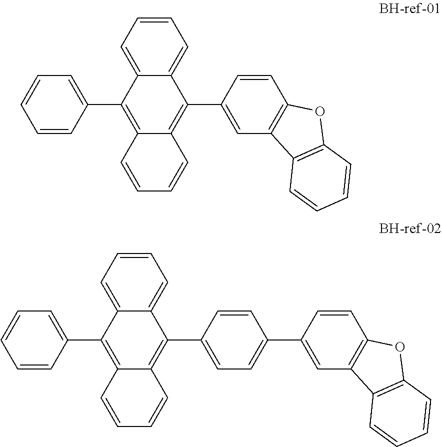 [SMILES: c1ccc(-c2c3ccccc3c(-c3ccc4oc5ccccc5c4c3)c3ccccc23)cc1 ; c1ccc(-c2c3ccccc3c(-c3ccc(-c4ccc5oc6ccccc6c5c4)cc3)c3ccccc23)cc1]

Performances of the OLED-1 to OLED-20 and the OLED-ref1 and OLED-ref2 are characterized, and performance parameters of each organic electronic device are as shown in Table 1.

TABLE 1

| Example of | Host | CE@1knits | LT90@1knits |
|---|---|---|---|
| device | material | [cd/A] | [h] |
| OLED-1 | Compound 1 | 6.4 | 319 |
| OLED-2 | Compound 2 | 6.2 | 324 |
| OLED-3 | Compound 3 | 6.8 | 348 |
| OLED-4 | Compound 4 | 7.2 | 399 |
| OLED-5 | Compound 5 | 5.2 | 337 |
| OLED-6 | Compound 6 | 5.9 | 353 |
| OLED-7 | Compound 7 | 6.3 | 387 |
| OLED-8 | Compound 8 | 6.5 | 326 |
| OLED-9 | Compound 9 | 7.1 | 347 |
| OLED-10 | Compound 10 | 7.7 | 386 |
| OLED-11 | Compound 11 | 5.2 | 358 |
| OLED-12 | Compound 12 | 5.7 | 391 |
| OLED-13 | Compound 13 | 6.5 | 360 |
| OLED-14 | Compound 14 | 6.6 | 326 |
| OLED-15 | Compound 15 | 6.3 | 319 |
| OLED-16 | Compound 16 | 5.8 | 312 |
| OLED-17 | Compound 17 | 6.0 | 301 |
| OLED-18 | Compound 18 | 5.8 | 306 |
| OLED-19 | Compound 19 | 6.1 | 309 |
| OLED-20 | Compound 20 | 6.5 | 321 |
| OLED-Ref1 | BH-ref-01 | 4.2 | 254 |
| OLED-Ref2 | BH-ref-02 | 4.8 | 287 |

It can be seen from Table 1 that color coordinates of blue light devices prepared by using the compounds 1 to 20 as host materials of light-emitting layers are better than color coordinates of blue light devices prepared by using comparative compounds BH-ref-01 and BH-ref-02 as host materials of light-emitting layers. In addition, luminous efficiency of the blue light devices prepared by using the compounds 1 to 20 as the host materials of the light-emitting layers all range from 5 cd/A to 8 cd/A, which means that the blue light devices prepared by using the compounds 1 to 20 as the host materials of the light-emitting layers have more excellent luminous efficiency than the blue light devices prepared by using the comparative compounds BH-ref-01 and BH-ref-02 as the host materials of the light-emitting layers. Moreover, the time for the brightness of the blue light device prepared by using the compounds 1 to 20 as the host materials in the light-emitting layers to be decayed to 90% is more than 300 hours, resulting in a longer service life

Both anthracene and pyrene have large rigid planes, and there are large conjugated π electron systems in anthracene and the pyrene. When fused ring-based derivatives of anthracene are used as host materials and fused ring-based derivatives of pyrene are used as guest materials of organic electronic devices, there is a large interaction between the host materials and the guest materials, which makes triplet-state energy levels of the host materials transfer to the guest materials, and ultimately affects service life of the organic electronic devices. In addition, materials of existing organic functional layers are generally prepared by a coating or an ink-jet printing method, therefore, solubility of the host materials affects film-forming uniformity of the organic functional materials, thus affecting luminous efficiency and service life of the organic electronic devices.

On one hand, the compound improves a hole transmission performance of a host material and luminous efficiency of a light-emitting layer by introducing a hole transport-typed group with large steric hindrance on an ortho-position of a benzene ring connected to a site of 9 of anthracene in the compound of the disclosure. On other hand, the compound increases the steric hindrance of an anthracene-based host material and reduces π-π interaction of the anthracene-based host material, thus reducing energy transfer between a host material and a guest material, improving a phenomena of triplet-triplet annihilation (TTA) and triplet polarity annihilation (TPA) in a reaction system, thereby improving the luminous efficiency of the light-emitting layer, and improving service life and stability of the organic electronic device. On another hand, the increase of steric hindrance of the anthracene-based host material reduces π-π accumulation of the anthracene-based host material, which makes solubility of the anthracene-based host material greater, thus making a film-forming performance of the organic functional layer including the anthracene-based host material better, further improving the luminous efficiency of the light-emitting layer, and improving service life and stability of the organic electronic device.

The compound, the composition, and the organic electronic device provided by the embodiments of the disclosure are described in detail. In this context, specific embodiments are adopted to illustrate a principle and implementation modes of the disclosure. The description of the above-mentioned embodiments is only used to help understand methods and a core idea of the disclosure. At the same time, for those skilled in the art, according to the idea of the disclosure, there might be changes in specific implementation modes and a scope of the disclosure, which falls within the scope of the protection of the disclosure. In conclusion, contents of the specification should not be interpreted as a limitation of the disclosure.

What is claimed is:

1. A compound represented by formula (1): 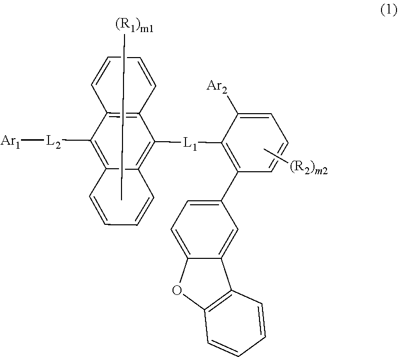
wherein Ar₁ and Ar₂ are each independently selected from an aromatic group containing 6 to 60 ring atoms, a heteroaromatic group containing 5 to 60 ring atoms, and combinations of these groups;
L₁ and L₂ are each independently selected from a single bond, an aromatic group containing 6 to 60 ring atoms, and a heteroaromatic group containing 5 to 60 ring atoms;
R₁ and R₂ are each independently selected from -D, a C₁-C₁₀ linear alkyl group, a C₁-C₁₀ linear alkoxy group, a C₁-C₁₀ linear thioalkoxy group, a C₃-C₁₀ branched alkyl group, a C₃-C₁₀ branched alkoxy group, a C₃-C₁₀ branched thioalkoxy group, a C₃-C₁₀ cyclic alkyl group, a C₃-C₁₀ cyclic alkoxy group, a C₃-C₁₀ cyclic thioalkoxy group, a methylsilyl group, a C₁-C₁₀ ketone group, a C₂-C₁₀ alkoxycarbonyl group, a C₇-C₁₀ aryloxycarbonyl group, a cyano group, an aminoformyl group, a haloformyl group, a formyl group, an isocyano group, an isocyanate group, a thiocyanate group, an isothiocyanate group, a hydroxy group, a nitro group, an amino group, —CF₃, —Cl, —Br, —F, —I, an aromatic group containing 6 to 30 ring atoms, a heteroaromatic group containing 5 to 30 ring atoms, an aryloxy group containing 5 to 30 ring atoms, and a heteroaryloxy group containing 5 to 30 ring atoms at each occurrence;
m1 is 0, 1, 2, 3, 4, 5, 6, 7, or 8; and
m2 is 0, 1, 2, or 3.

2. The compound of claim 1, wherein
Ar₁ is selected from an aromatic group containing 6 to 18 ring atoms and a heteroaromatic group containing 6 to 13 ring atoms;
Ar₂ is selected from an aromatic group containing 6 to 14 ring atoms and a heteroaromatic group containing 13 to 19 ring atoms;
L₁ and L₂ are each independently selected from a single bond and a group containing 6 ring atoms;
R₁ and R₂ are each independently selected from -D, a C₄ alkyl group, an aromatic group containing 6 to 10 ring atoms, and a heteroaromatic group containing 12 ring atoms;
m1 is 0, 2, or 8; and
m2 is 0 or 1.

3. The compound of claim 2, wherein Ar₁ is selected from any of following structures: 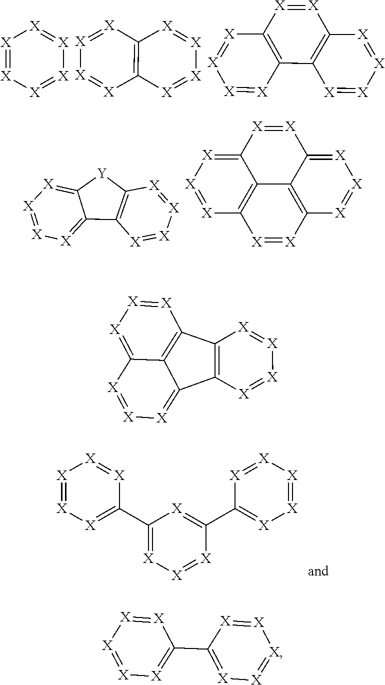
wherein X is independently selected from CR₃ and N at each occurrence;
Y is selected from CR₅R₆ and O;
R₃ is selected from —H and -D; R₅ and R₆ are each independently selected from a methyl group; and
when X is a linking site, X is C.

4. The compound of claim 3, wherein Ar₁ is selected from any of following structures:

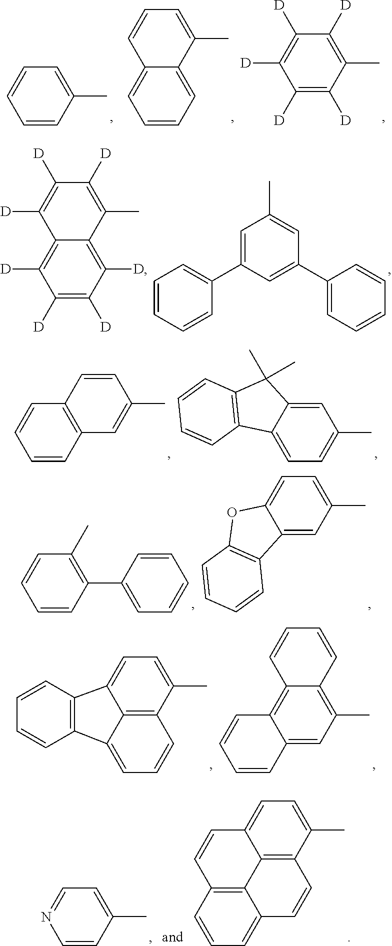

5. The compound of claim 2, wherein Ar₂ is selected from any of following structures: 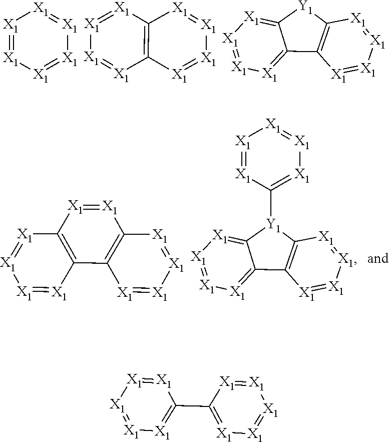
wherein X₁ is selected from CR₇;
Y₁ is selected from N and O; and
R₇ is independently selected from —H at each occurrence.

6. The compound of claim 5, wherein Ar₂ is selected from any of following structures:

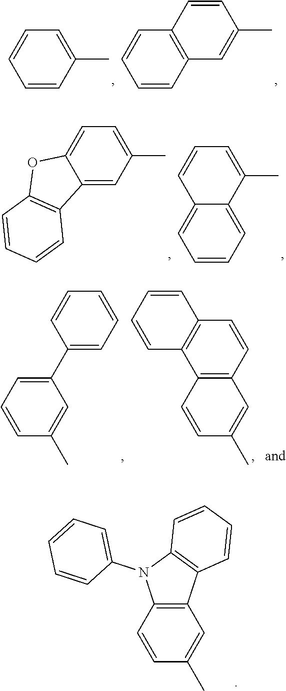

7. The compound of claim 2, wherein L₁ is a single bond or selected from 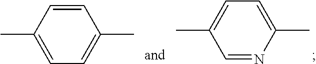
L₂ is a single bond or selected from

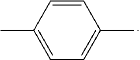

8. The compound of claim 2, wherein R₁ is selected from -D and a tert-butyl group;
R₂ is selected from

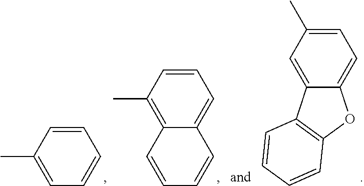

9. The compound of claim 2, wherein the compound represented by formula (1) is selected from any of following structures:

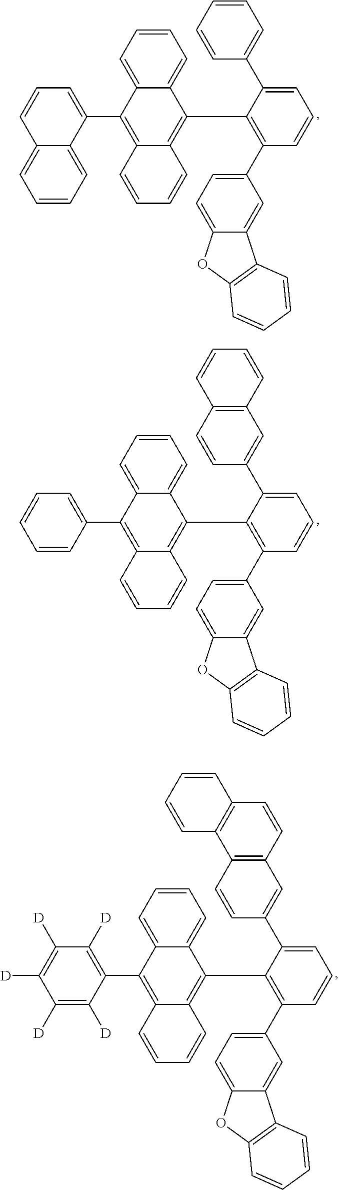

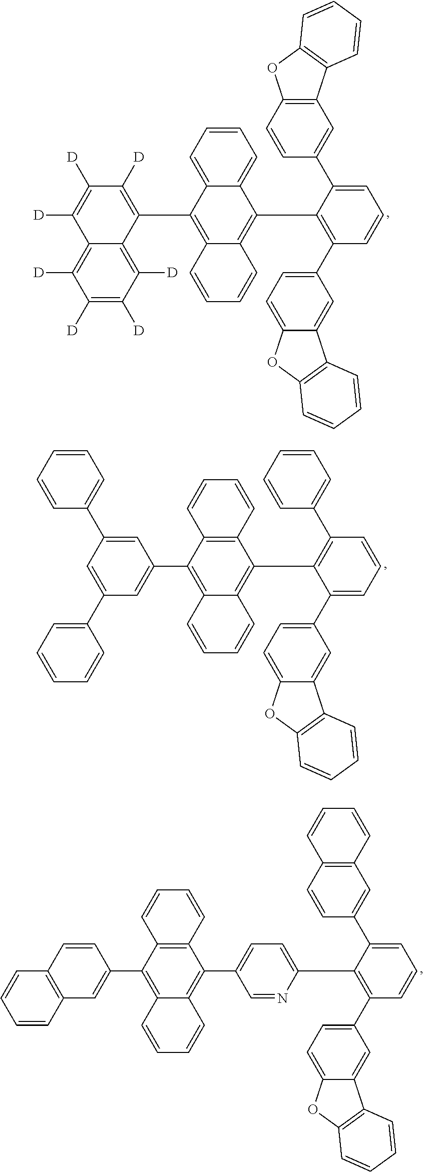

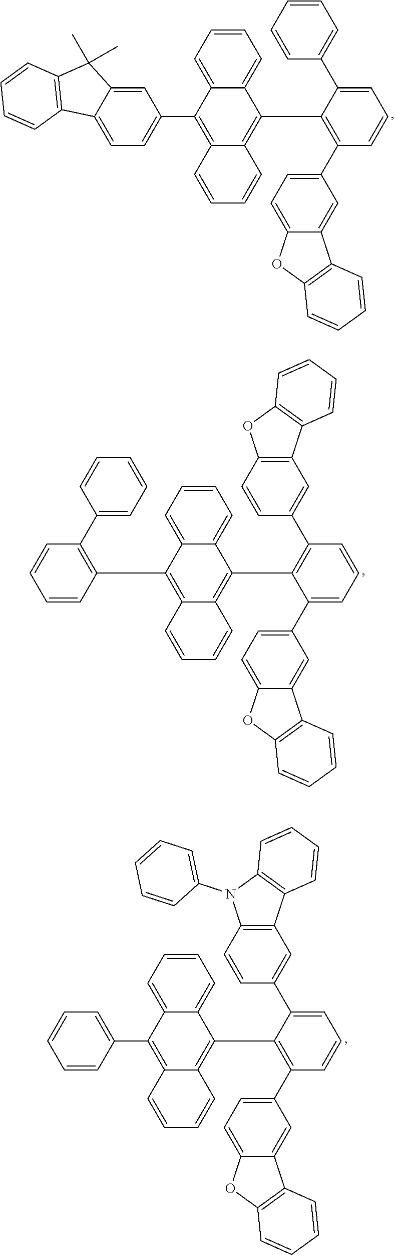

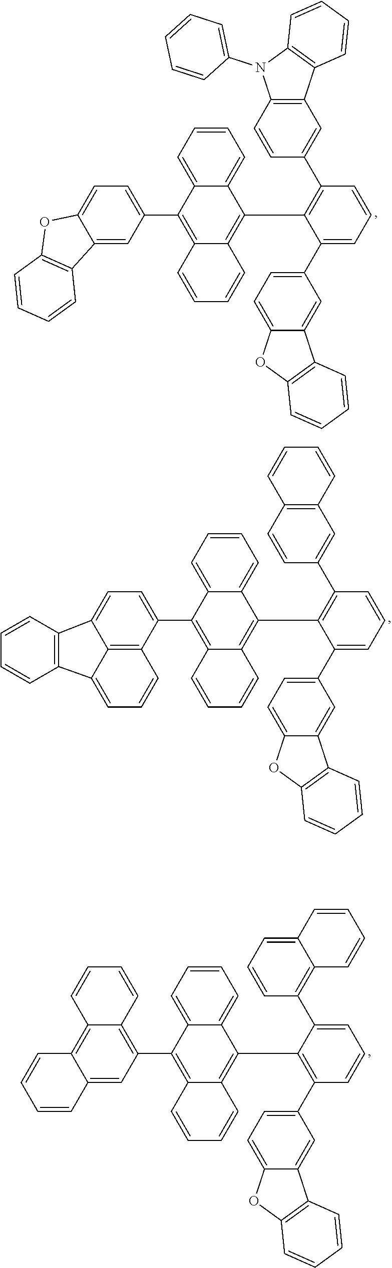

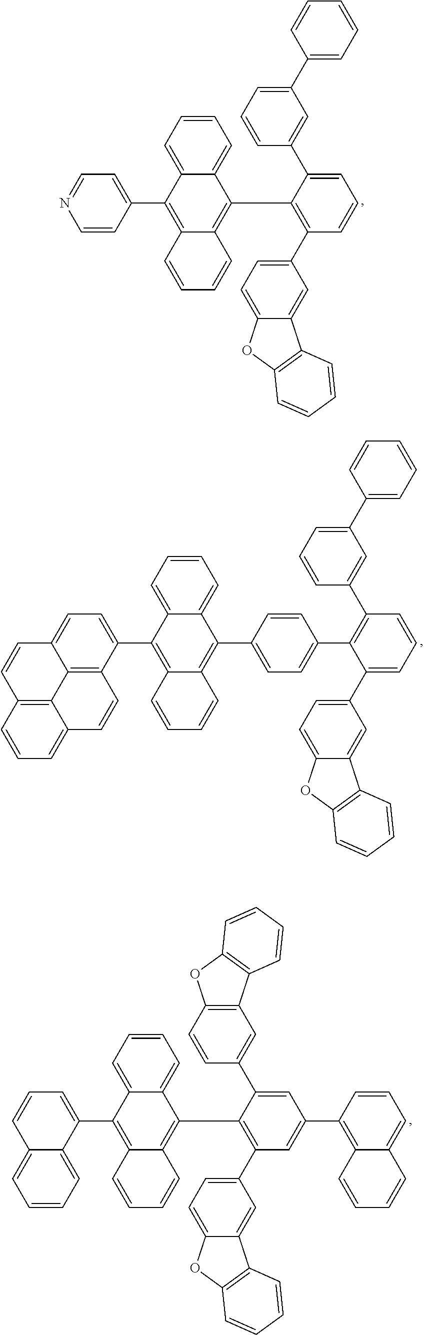 [SMILES: c1ccc(-c2cccc(-c3cccc(-c4ccc5oc6ccccc6c5c4)c3-c3c4ccccc4c(-c4ccncc4)c4ccccc34)c2)cc1 ; c1ccc(-c2cccc(-c3cccc(-c4ccc5oc6ccccc6c5c4)c3-c3ccc(-c4c5ccccc5c(-c5ccc6ccc7cccc8ccc5c6c78)c5ccccc45)cc3)c2)cc1 ; c1ccc2c(-c3cc(-c4ccc5oc6ccccc6c5c4)c(-c4c5ccccc5c(-c5cccc6ccccc56)c5ccccc45)c(-c4ccc5oc6ccccc6c5c4)c3)cccc2c1]

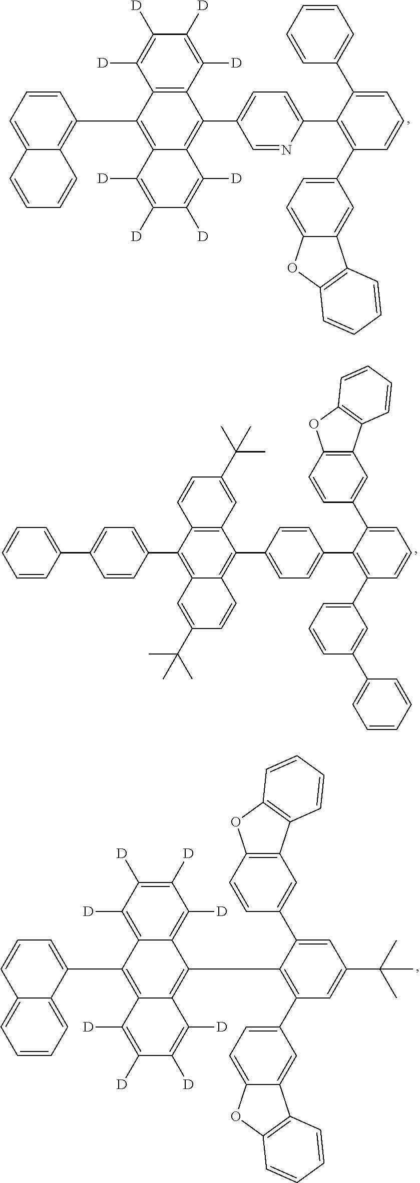

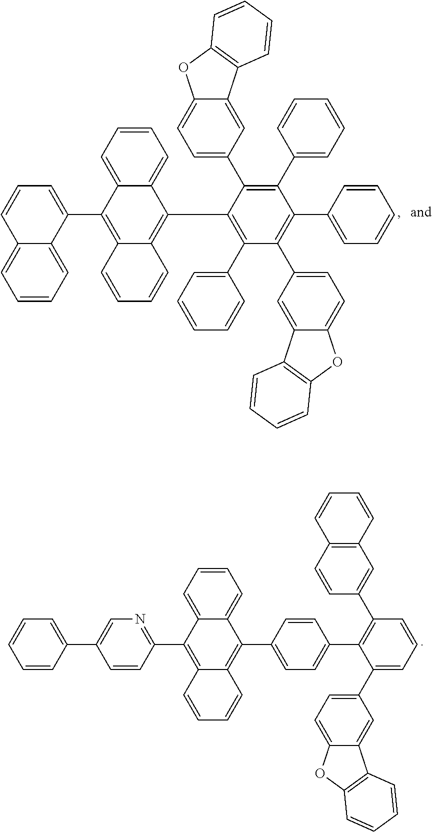 [SMILES: c1ccc(-c2c(-c3ccc4oc5ccccc5c4c3)c(-c3ccccc3)c(-c3c4ccccc4c(-c4cccc5ccccc45)c4ccccc34)c(-c3ccc4oc5ccccc5c4c3)c2-c2ccccc2)cc1 ; c1ccc(-c2ccc(-c3c4ccccc4c(-c4ccc(-c5c(-c6ccc7ccccc7c6)cccc5-c5ccc6oc7ccccc7c6c5)cc4)c4ccccc34)nc2)cc1]

10. The compound of claim 1, wherein Ar₁ is selected from any of following structures: 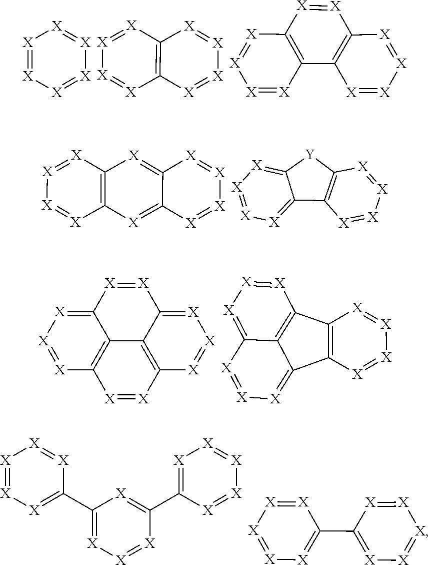
wherein X is independently selected from CR₃ and N at each occurrence;
Y is selected from NR₄, CR₅R₆, SiR₅R₆, O, S, S═O, and SO₂; and
R₃, R₄, R₅, and R₆ are each independently selected from —H, -D, a C₁-C₂₀ linear alkyl group, a C₁-C₂₀ linear alkoxy group, a C₁-C₂₀ linear thioalkoxy group, a C₃-C₂₀ branched alkyl group, a C₃-C₂₀ branched alkoxy group, a C₃-C₂₀ branched thioalkoxy group, a C₃-C₂₀ cyclic alkyl group, a C₃-C₂₀ cyclic alkoxy group, a C₃-C₂₀ cyclic thioalkoxy group, a methylsilyl group, a C₁-C₂₀ ketone group, a C₂-C₂₀ alkoxycarbonyl group, a C₇-C₂₀ aryloxycarbonyl group, a cyano group, an aminoformyl group, a haloformyl group, a formyl group, an isocyano group, an isocyanate group, a thiocyanate group, an isothiocyanate group, a hydroxy group, a nitro group, an amino group, —CF₃, —Cl, —Br, —F, —I, a substituted or unsubstituted aromatic group containing 6 to 60 ring atoms, a substituted or unsubstituted heteroaromatic group containing 5 to 60 ring atoms, a substituted or unsubstituted aryloxy group containing 5 to 60 ring atoms, and a substituted or unsubstituted heteroaryloxy group containing 5 to 60 ring atoms at each occurrence; and a ring is formed or no ring is formed by an interconnection of R₅ and R₆.

11. The compound of claim 10, wherein when X is a linking site, X is C; when Y is a linking site, Y is N.

12. The compound of claim 1, wherein Ar₁ is selected from any of following structures: 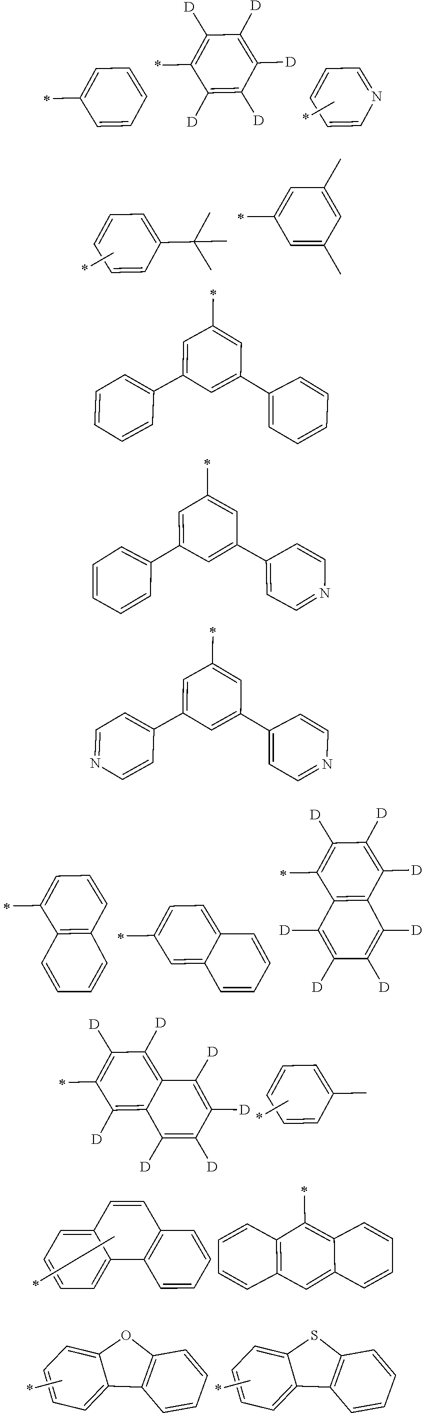 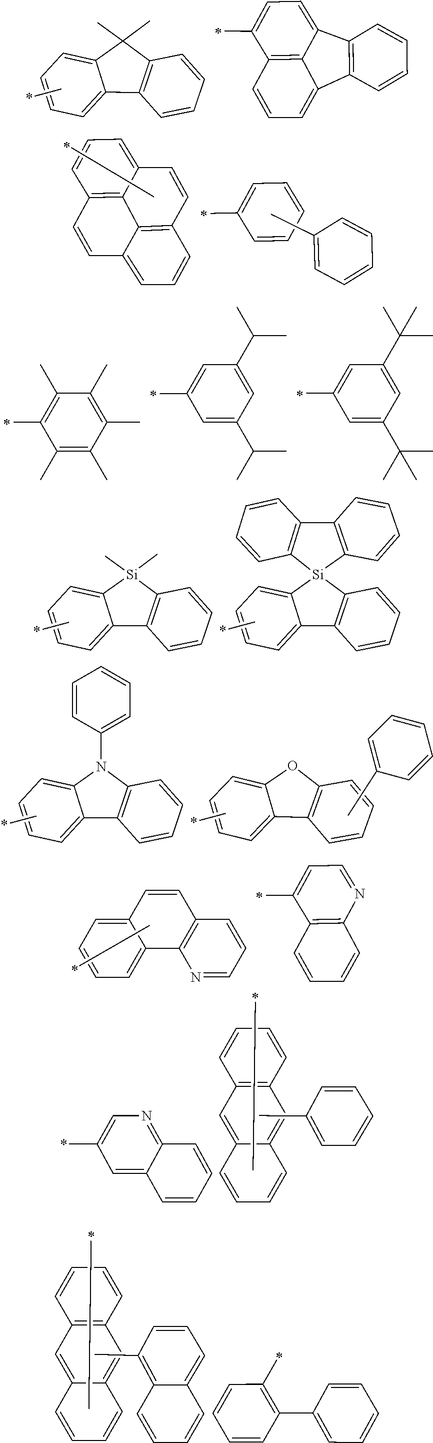 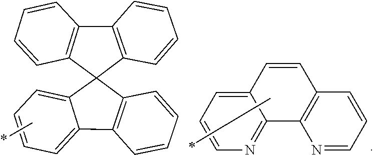
wherein * indicates a linking site.

13. The compound of claim 1, wherein Ar₂ is selected from any of following structures: 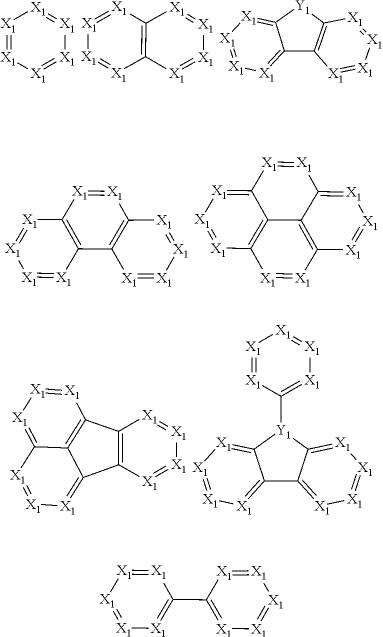
wherein X₁ is independently selected from CR₇ and N at each occurrence;
Y₁ is selected from NR₈, CR₉R₁₀, SiR₁₁R₁₂, O, S, S═O, and SO₂; and
R₇, R₈, R₉, and R₁₀ are each independently selected from —H, -D, a C₁-C₂₀ linear alkyl group, a C₁-C₂₀ linear alkoxy group, a C₁-C₂₀ linear thioalkoxy group, a C₃-C₂₀ branched alkyl group, a C₃-C₂₀ branched alkoxy group, a C₃-C₂₀ branched thioalkoxy group, a C₃-C₂₀ cyclic alkyl group, a C₃-C₂₀ cyclic alkoxy group, a C₃-C₂₀ cyclic thioalkoxy group, a methylsilyl group, a C₁-C₂₀ ketone group, a C₂-C₂₀ alkoxycarbonyl group, a C₇-C₂₀ aryloxycarbonyl group, a cyano group, an aminoformyl group, a haloformyl group, a formyl group, an isocyano group, an isocyanate group, a thiocyanate group, an isothiocyanate group, a hydroxy group, a nitro group, an amino group, —CF₃, —Cl, —Br, —F, —I, a substituted or unsubstituted aromatic group containing 6 to 60 ring atoms, a substituted or unsubstituted heteroaromatic group containing 5 to 60 ring atoms, a substituted or unsubstituted aryloxy group containing 5 to 60 ring atoms, and a substituted or unsubstituted heteroaryloxy group containing 5 to 60 ring atoms at each occurrence; a ring is formed or no ring is formed by an interconnection of R₉ and R₁₀.

14. The compound of claim 13, wherein when X₁ is a linking site, X₁ is C; when Y₁ is a linking site, Y₁ is N.

15. The compound of claim 1, wherein Ar₂ is selected from any of following structures:

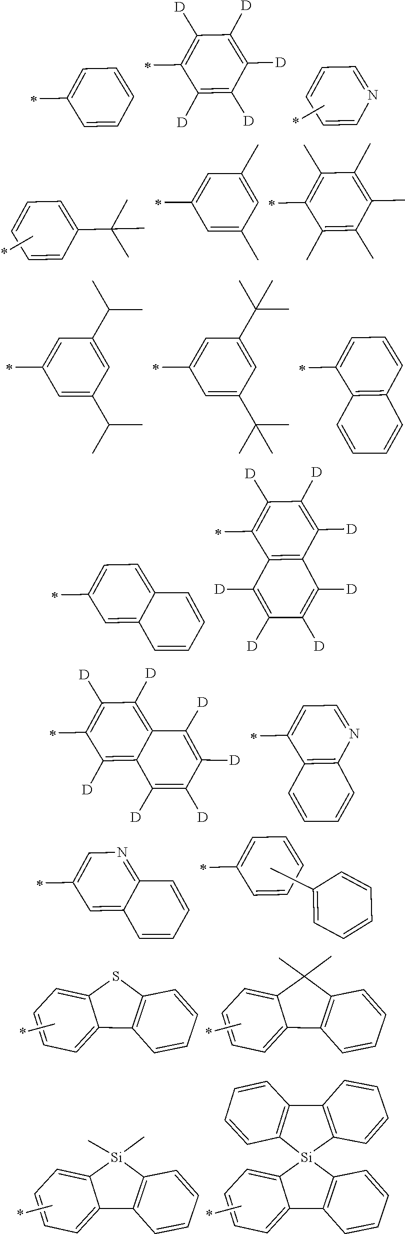

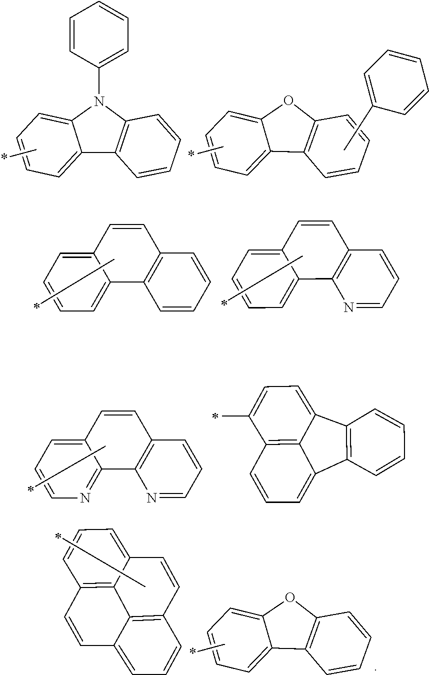

16. The compound of claim 1, wherein L₁ and L₂ are each independently selected from a single bond and any of following structures: 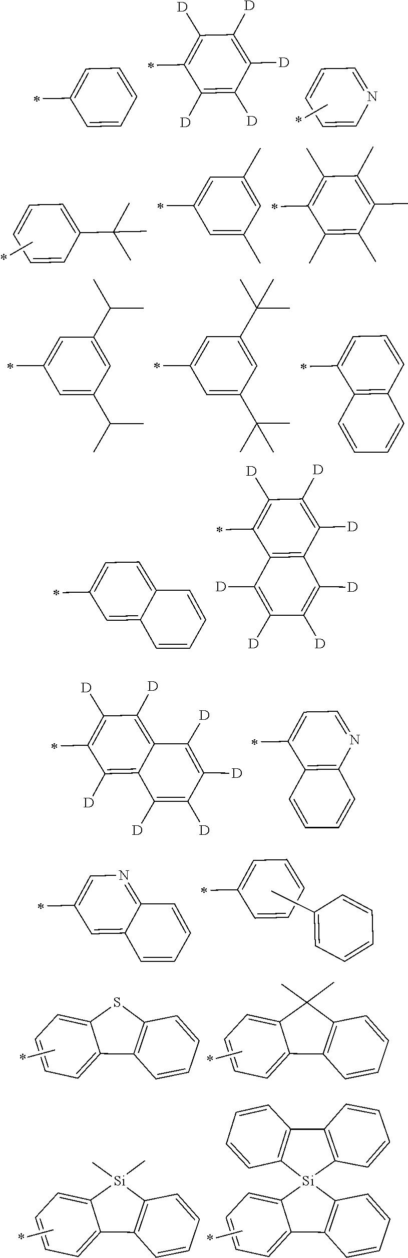 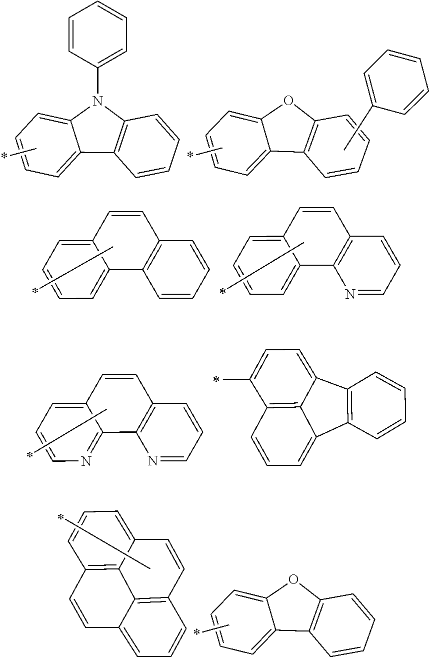
wherein X₂ is independently selected from CR₁₃ and N at each occurrence; and
R₁₃ is independently selected from —H, -D, a C₁-C₂₀ linear alkyl group, a C₁-C₂₀ linear alkoxy group, a C₁-C₂₀ linear thioalkoxy group, a C₃-C₂₀ branched alkyl group, a C₃-C₂₀ branched alkoxy group, a C₃-C₂₀ branched thioalkoxy group, a C₃-C₂₀ cyclic alkyl group, a C₃-C₂₀ cyclic alkoxy group, a C₃-C₂₀ cyclic thioalkoxy group, a methylsilyl group, a C₁-C₂₀ ketone group, a C₂-C₂₀ alkoxycarbonyl group, a C₇-C₂₀ aryloxycarbonyl group, a cyano group, an aminoformyl group, a haloformyl group, a formyl group, an isocyano group, an isocyanate group, a thiocyanate group, an isothiocyanate group, a hydroxy group, a nitro group, an amino group, —CF₃, —Cl, —Br, —F, —I, a substituted or unsubstituted aromatic group containing 6 to 60 ring atoms, a substituted or unsubstituted heteroaromatic group containing 5 to 60 ring atoms, a substituted or unsubstituted aryloxy group containing 5 to 60 ring atoms, and a substituted or unsubstituted heteroaryloxy group containing 5 to 60 ring atoms at each occurrence.

17. The compound of claim 1, wherein L₁ and L₂ are each independently selected from a single bond and any of following structures:

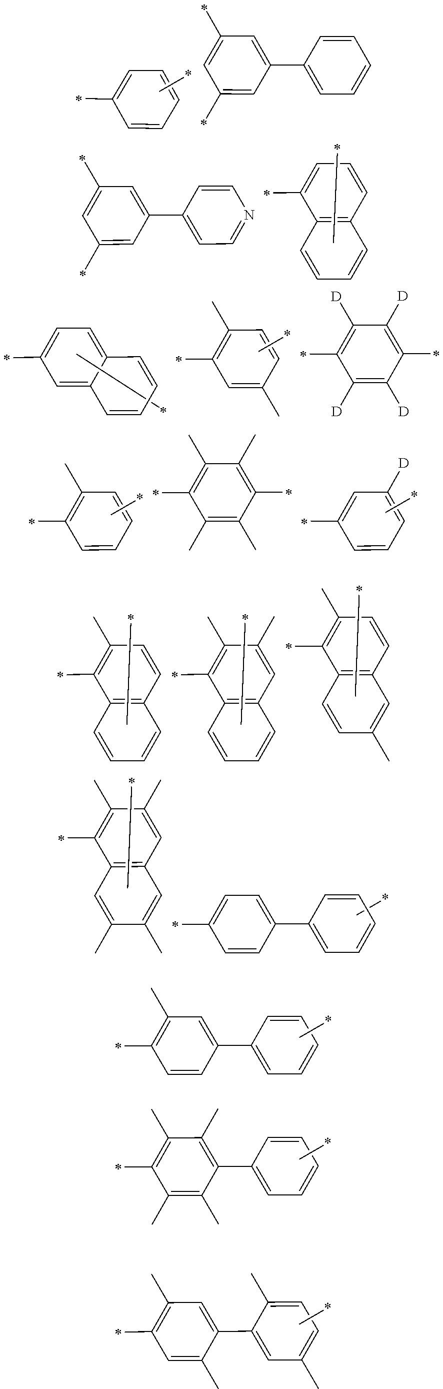

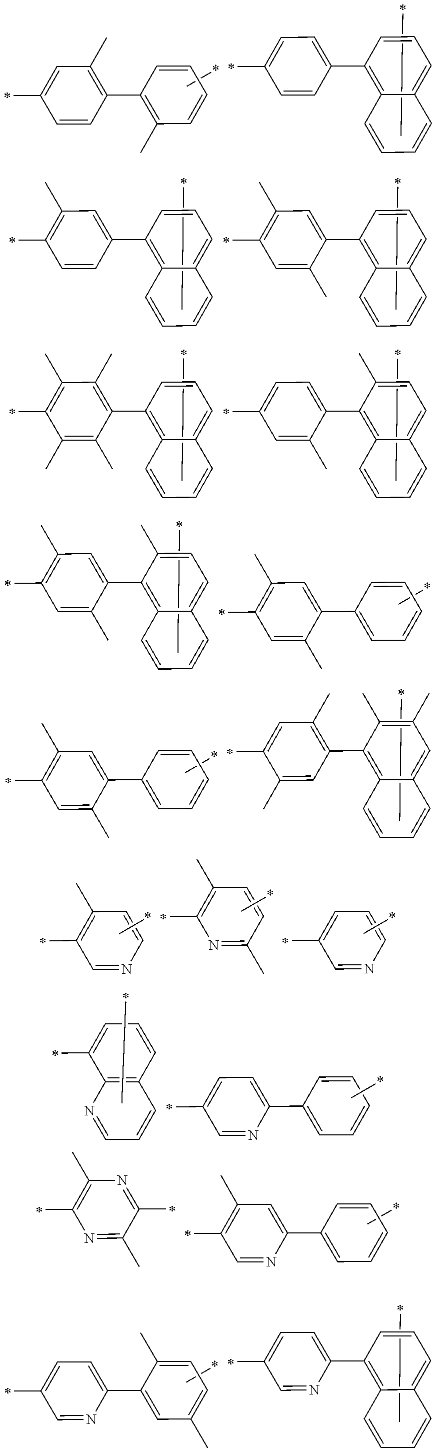

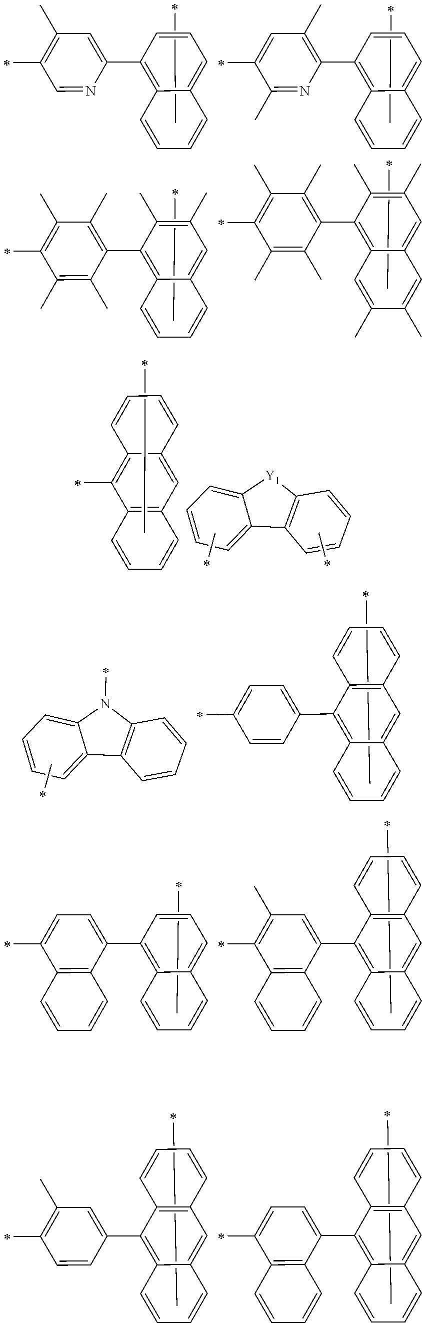

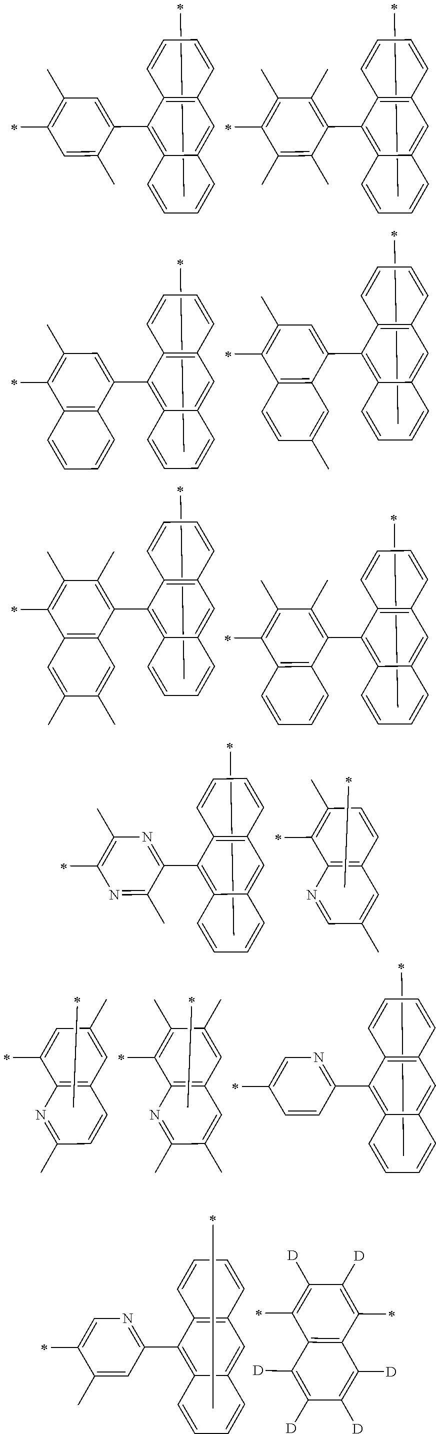

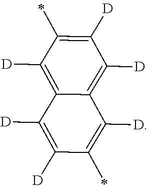

18. The compound of claim 1, wherein the compound represented by formula (1) is selected from any of following structures:

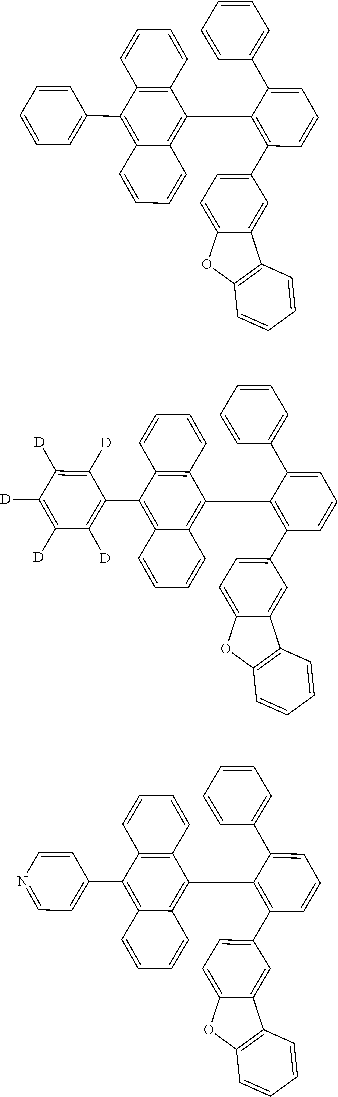 [SMILES: c1ccc(-c2cccc(-c3ccc4oc5ccccc5c4c3)c2-c2c3ccccc3c(-c3ccccc3)c3ccccc23)cc1 ; [2H]c1c([2H])c([2H])c(-c2c3ccccc3c(-c3c(-c4ccccc4)cccc3-c3ccc4oc5ccccc5c4c3)c3ccccc23)c([2H])c1[2H] ; c1ccc(-c2cccc(-c3ccc4oc5ccccc5c4c3)c2-c2c3ccccc3c(-c3ccncc3)c3ccccc23)cc1]

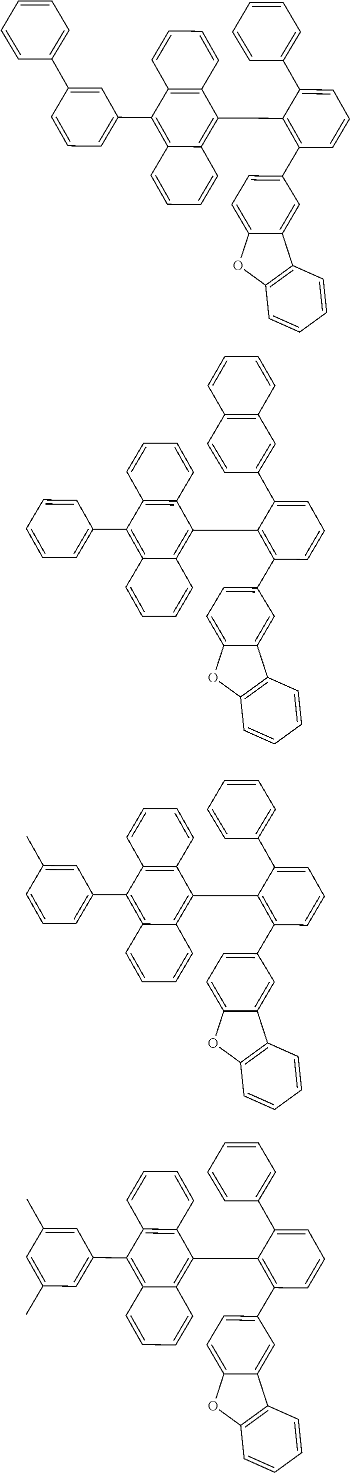

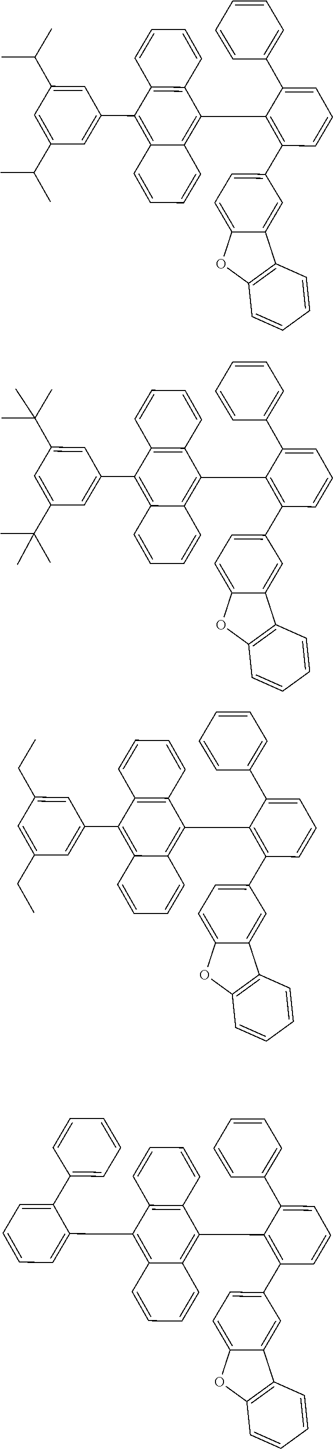

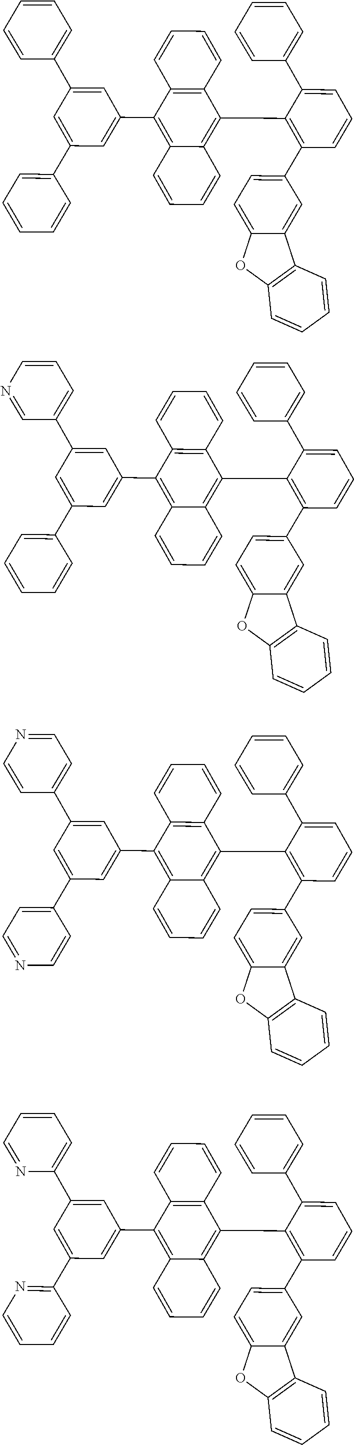

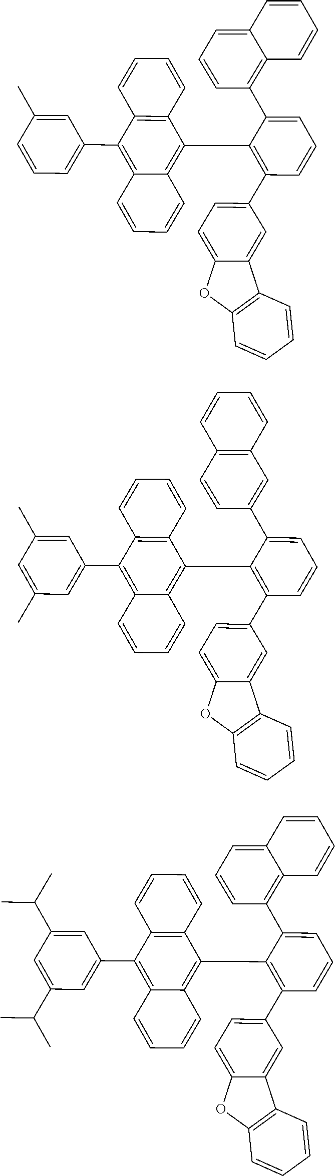

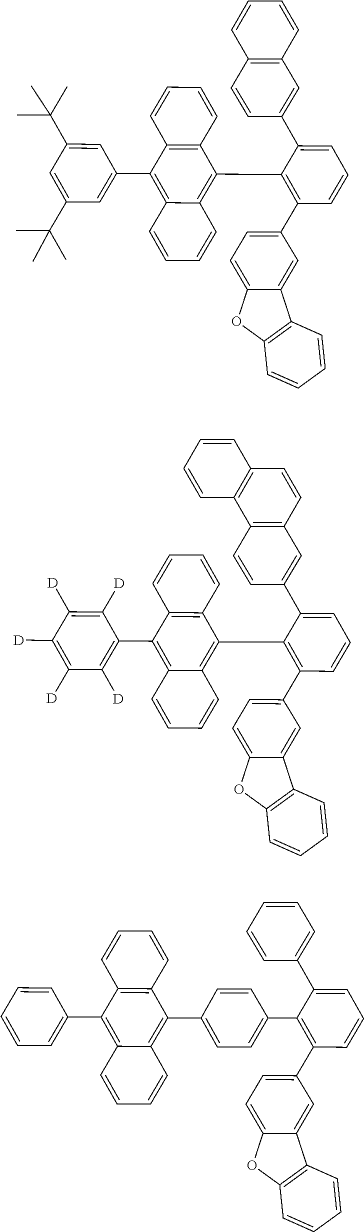 [SMILES: CC(C)(C)c1cc(-c2c3ccccc3c(-c3c(-c4ccc5ccccc5c4)cccc3-c3ccc4oc5ccccc5c4c3)c3ccccc23)cc(C(C)(C)C)c1 ; [2H]c1c([2H])c([2H])c(-c2c3ccccc3c(-c3c(-c4ccc5c(ccc6ccccc65)c4)cccc3-c3ccc4oc5ccccc5c4c3)c3ccccc23)c([2H])c1[2H] ; c1ccc(-c2cccc(-c3ccc4oc5ccccc5c4c3)c2-c2ccc(-c3c4ccccc4c(-c4ccccc4)c4ccccc34)cc2)cc1]

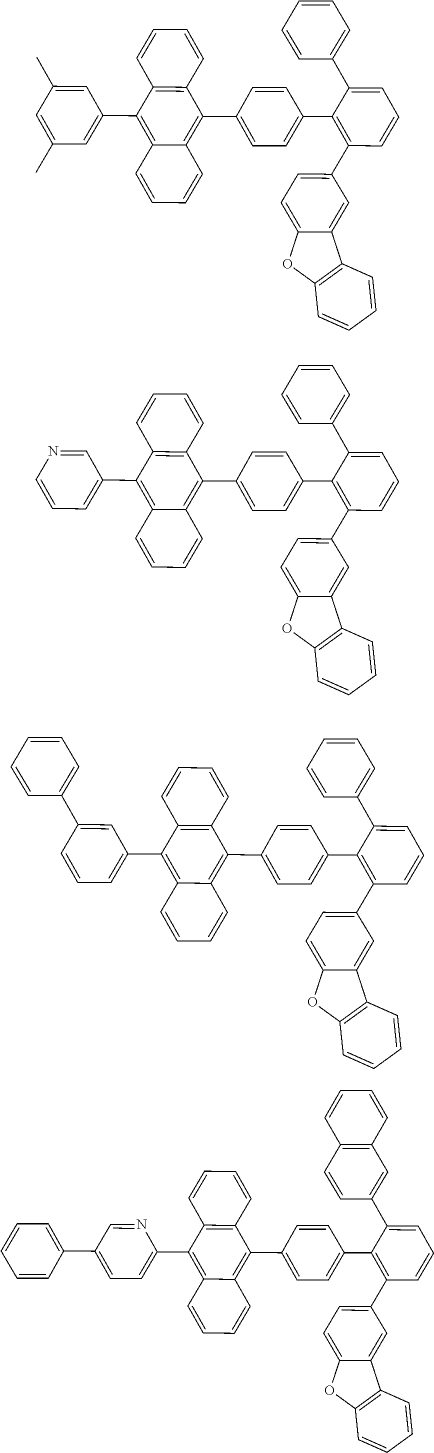

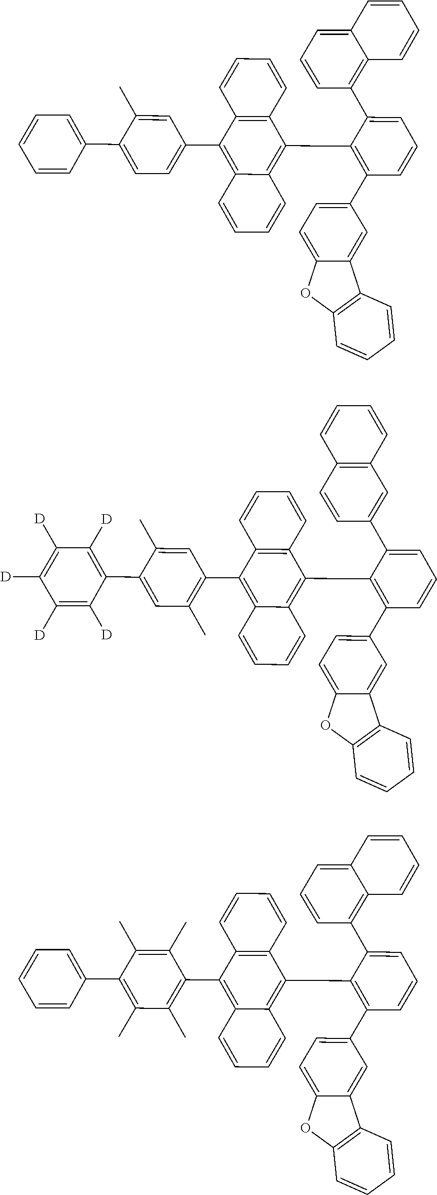

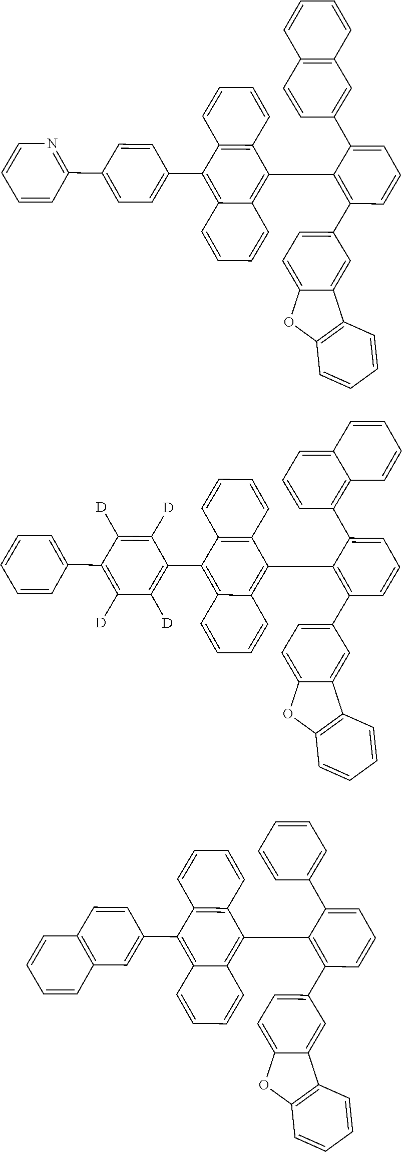

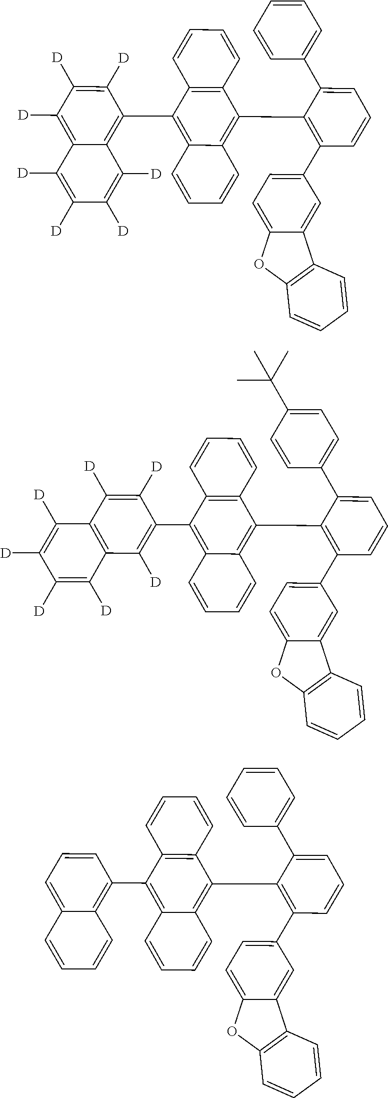

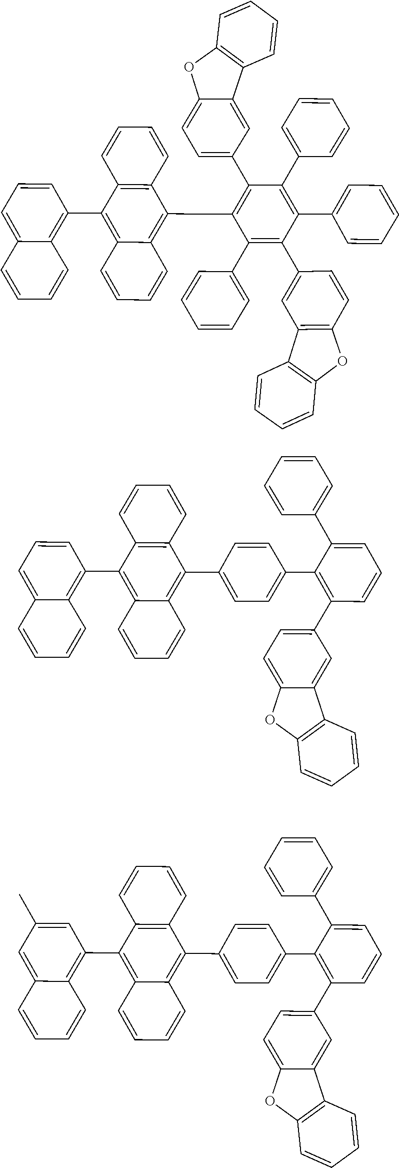

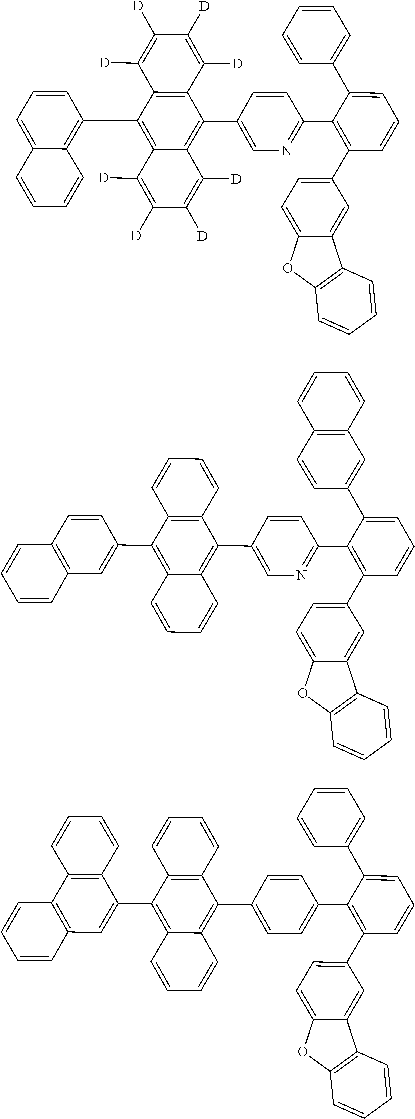

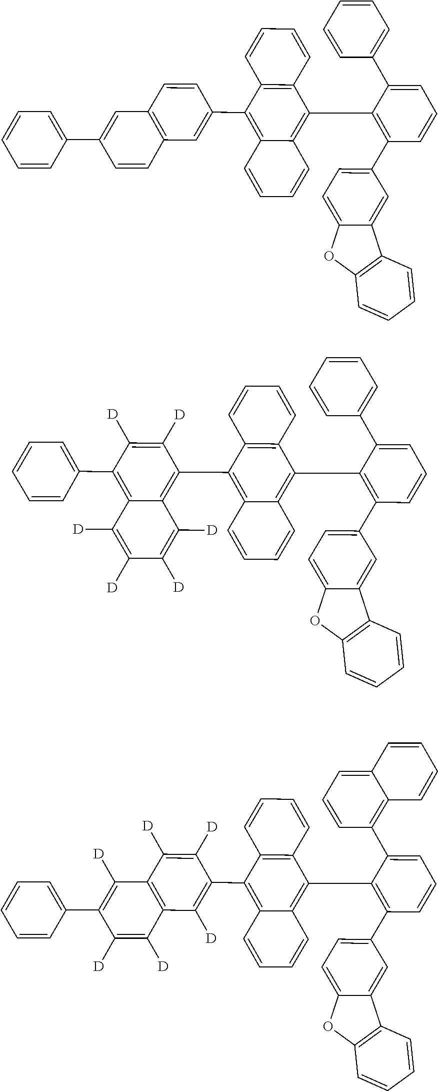 [SMILES: c1ccc(-c2ccc3cc(-c4c5ccccc5c(-c5c(-c6ccccc6)cccc5-c5ccc6oc7ccccc7c6c5)c5ccccc45)ccc3c2)cc1 ; [2H]c1c([2H])c([2H])c2c(-c3c4ccccc4c(-c4c(-c5ccccc5)cccc4-c4ccc5oc6ccccc6c5c4)c4ccccc34)c([2H])c([2H])c(-c3ccccc3)c2c1[2H] ; [2H]c1c(-c2c3ccccc3c(-c3c(-c4ccc5oc6ccccc6c5c4)cccc3-c3cccc4ccccc34)c3ccccc23)c([2H])c2c([2H])c([2H])c(-c3ccccc3)c([2H])c2c1[2H]]

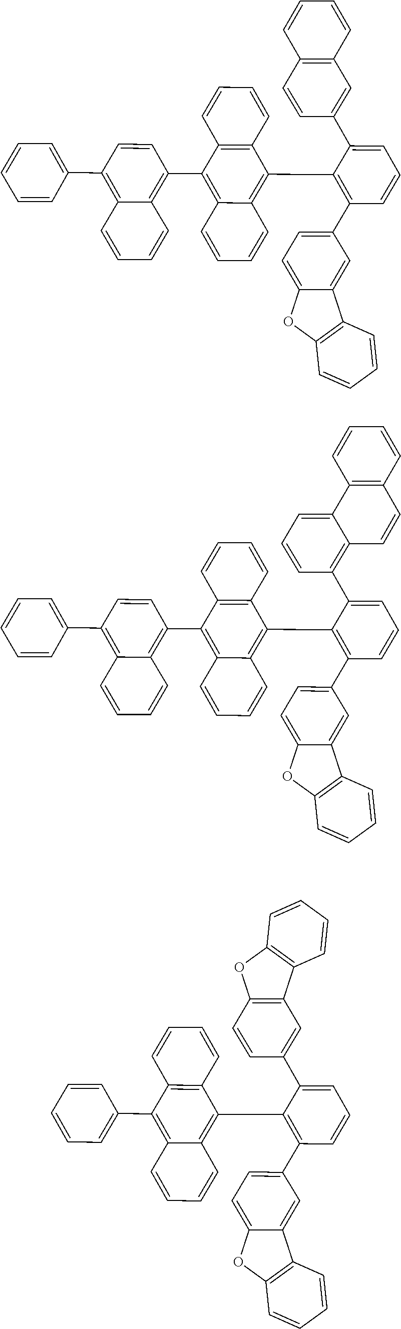

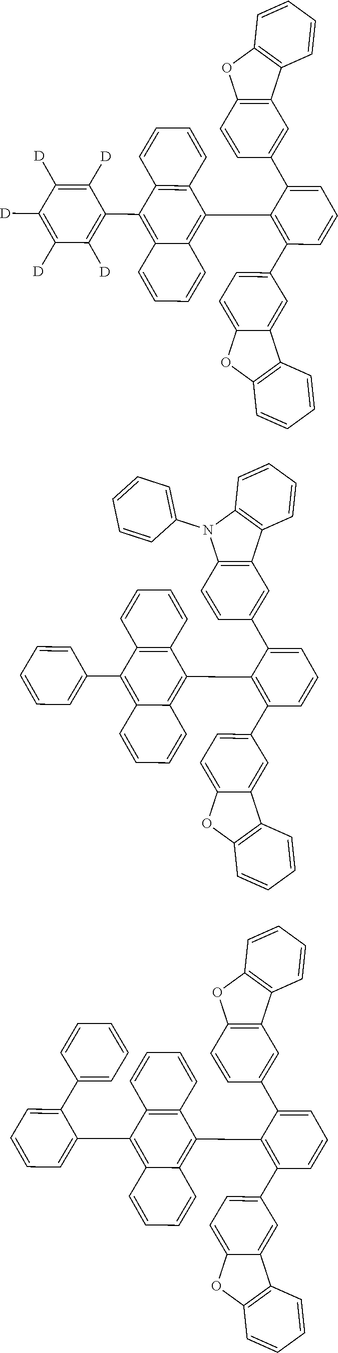

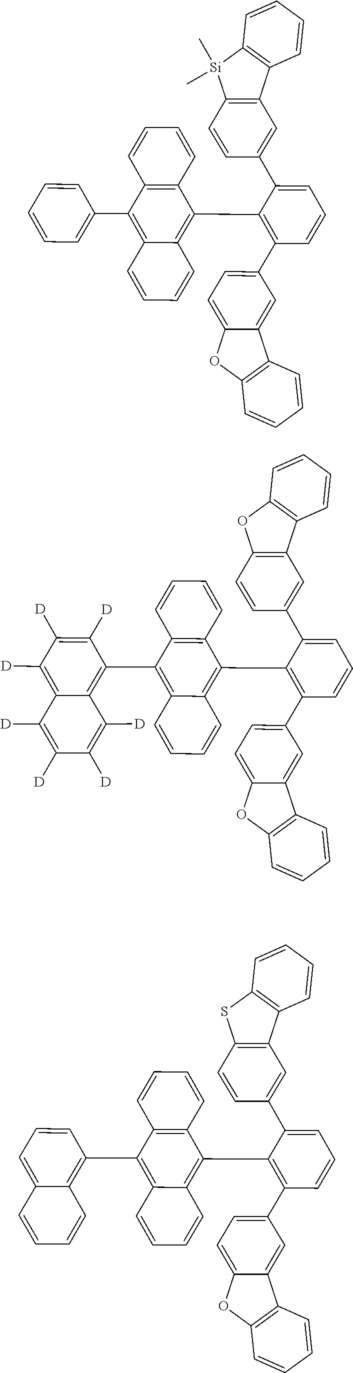

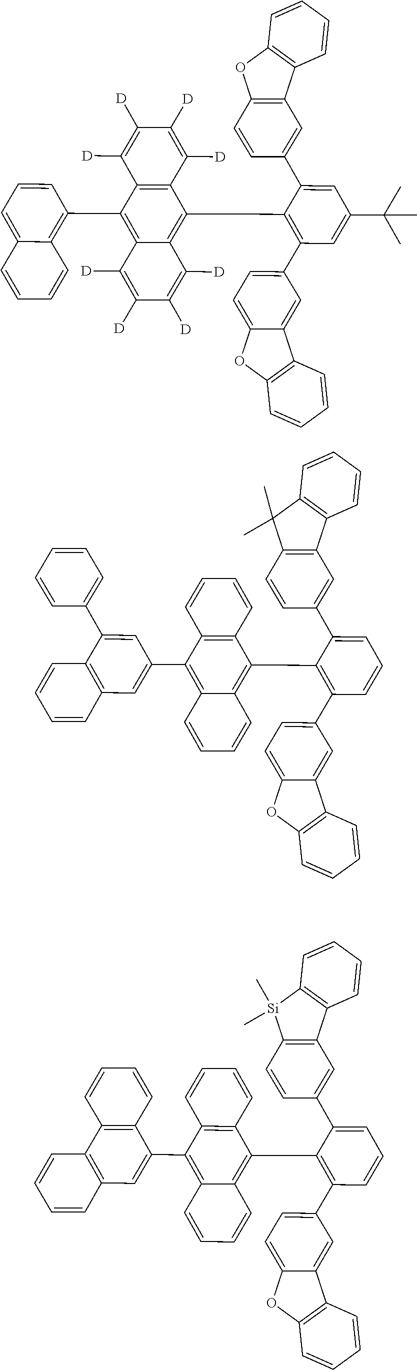

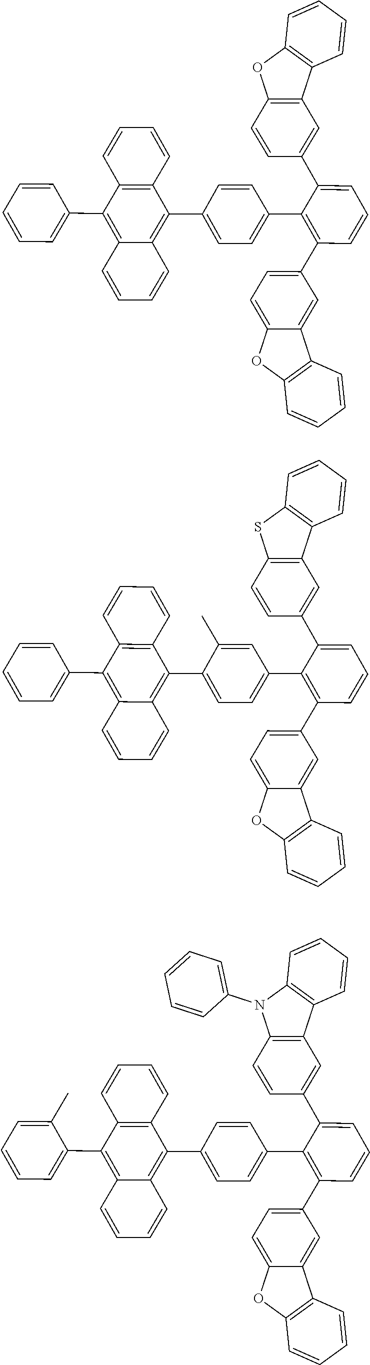

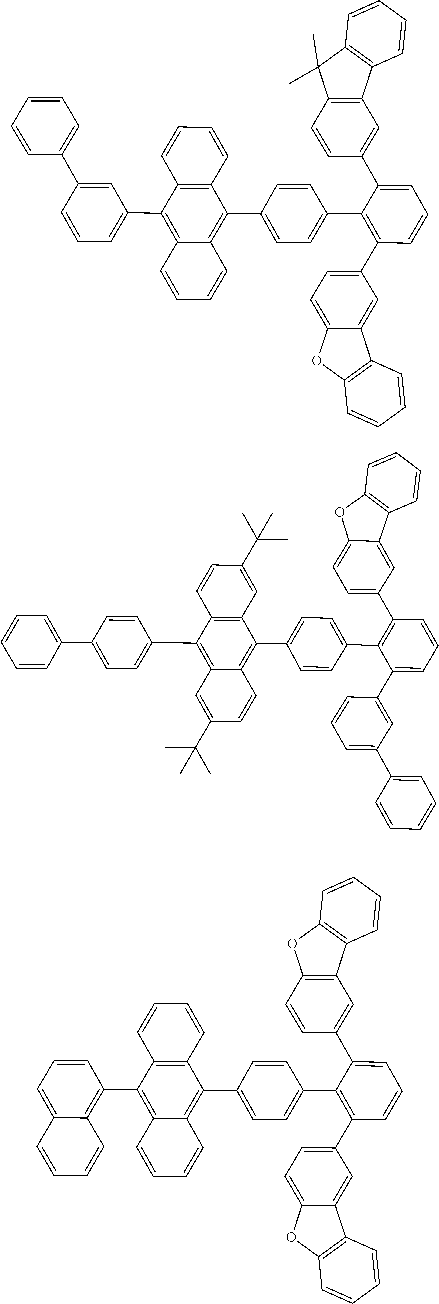

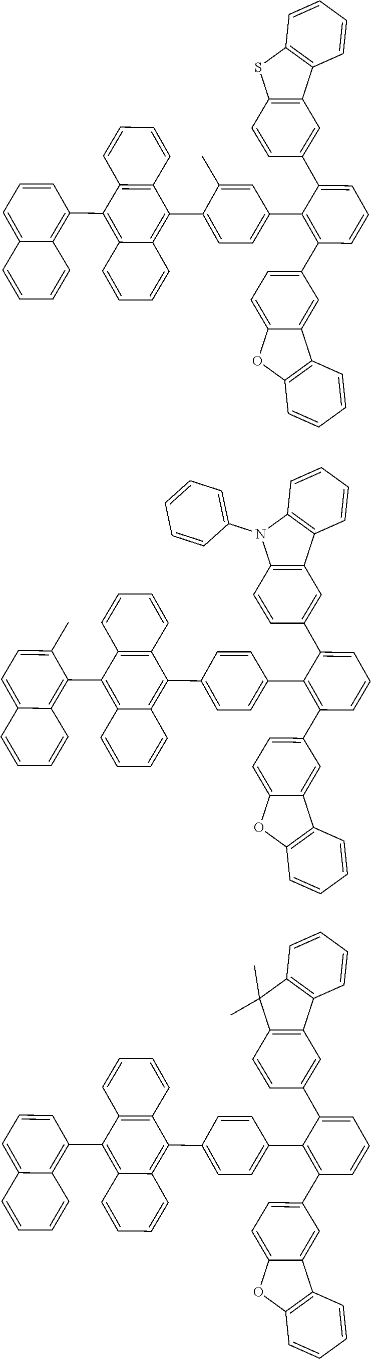

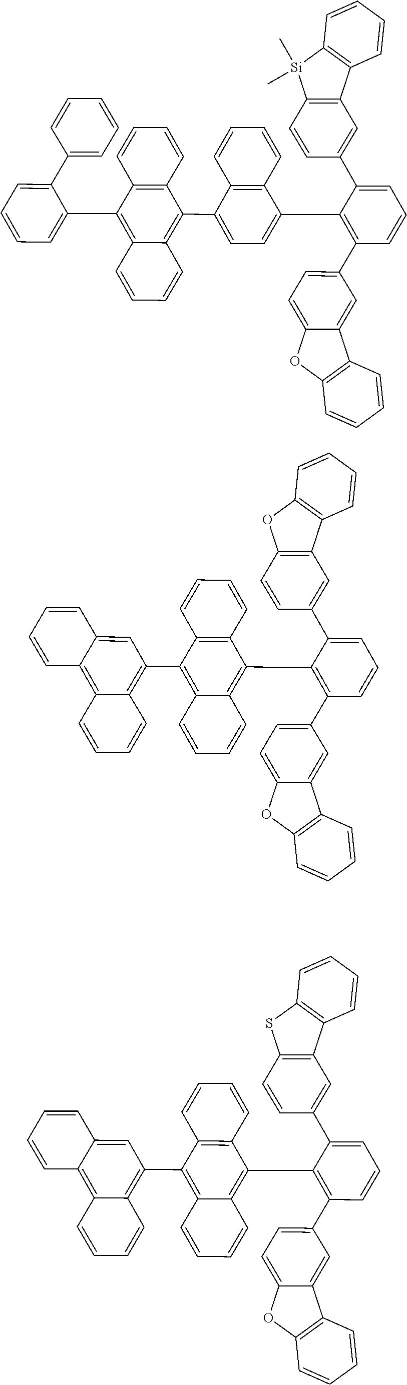

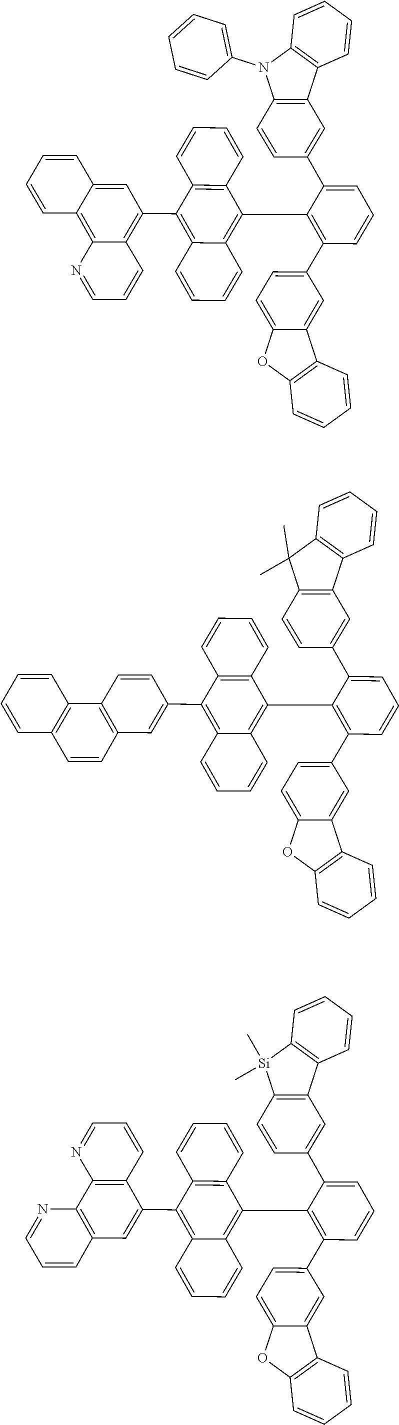 [SMILES: c1ccc(-n2c3ccccc3c3cc(-c4cccc(-c5ccc6oc7ccccc7c6c5)c4-c4c5ccccc5c(-c5cc6ccccc6c6ncccc56)c5ccccc45)ccc32)cc1 ; CC1(C)c2ccccc2-c2cc(-c3cccc(-c4ccc5oc6ccccc6c5c4)c3-c3c4ccccc4c(-c4ccc5c(ccc6ccccc65)c4)c4ccccc34)ccc21 ; C[Si]1(C)c2ccccc2-c2cc(-c3cccc(-c4ccc5oc6ccccc6c5c4)c3-c3c4ccccc4c(-c4cc5cccnc5c5ncccc45)c4ccccc34)ccc21]

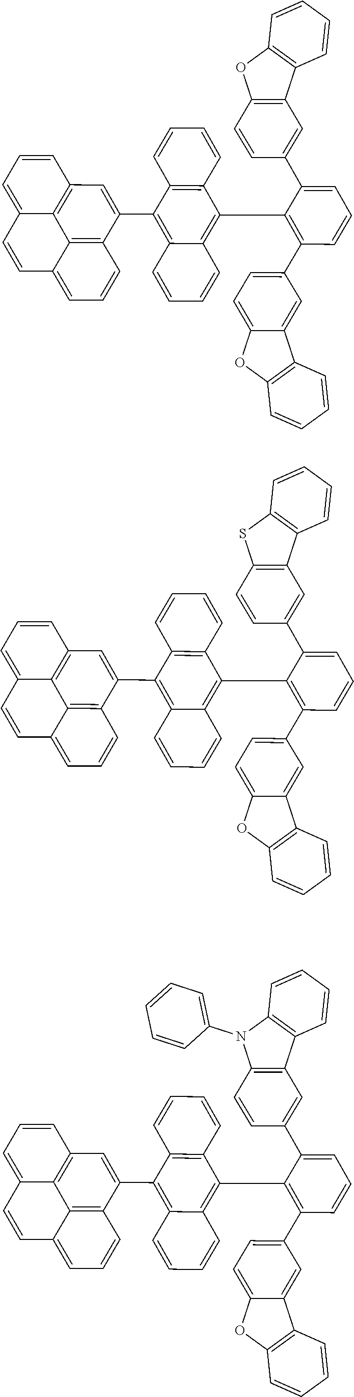

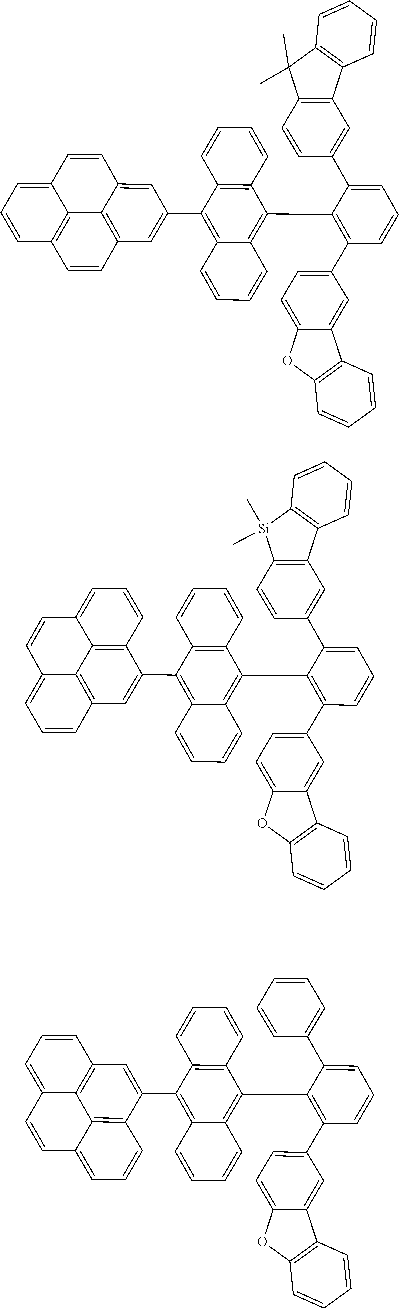

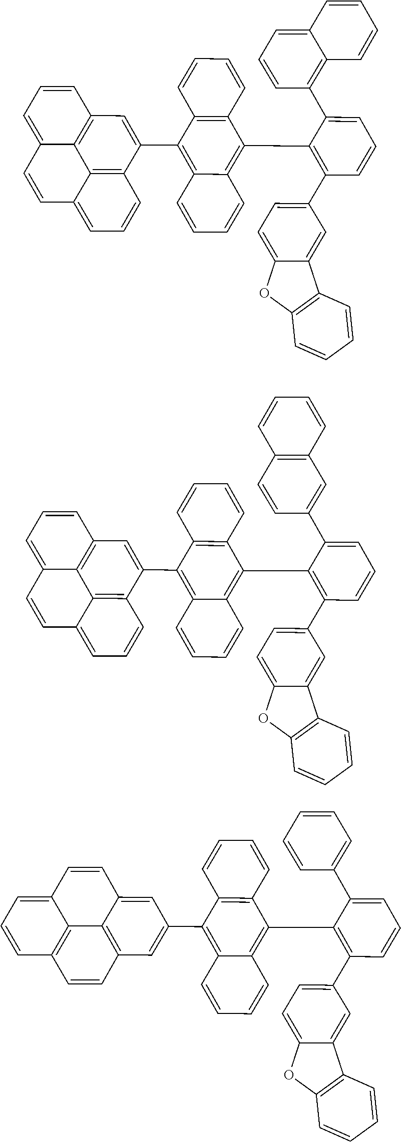

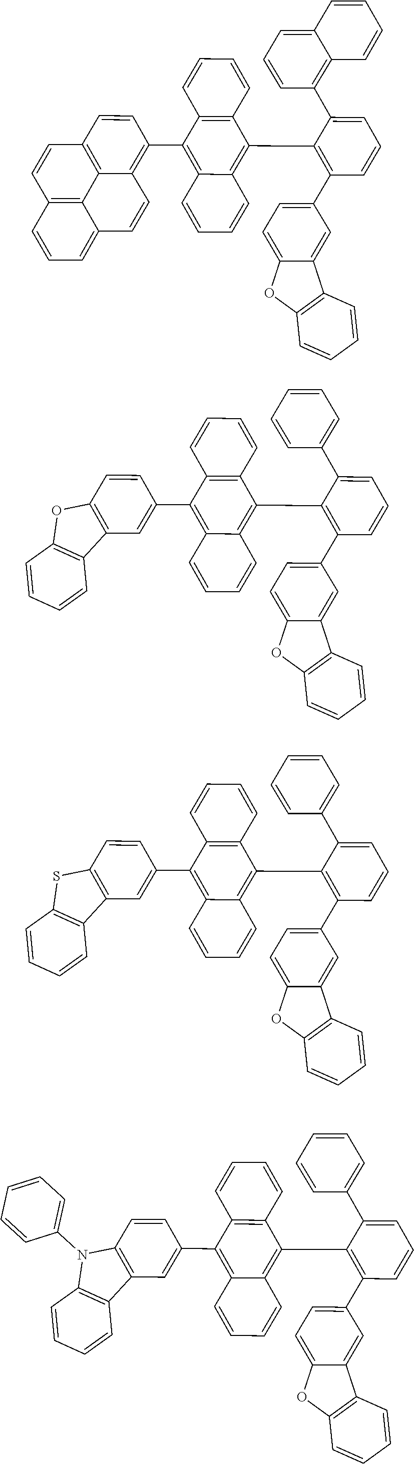

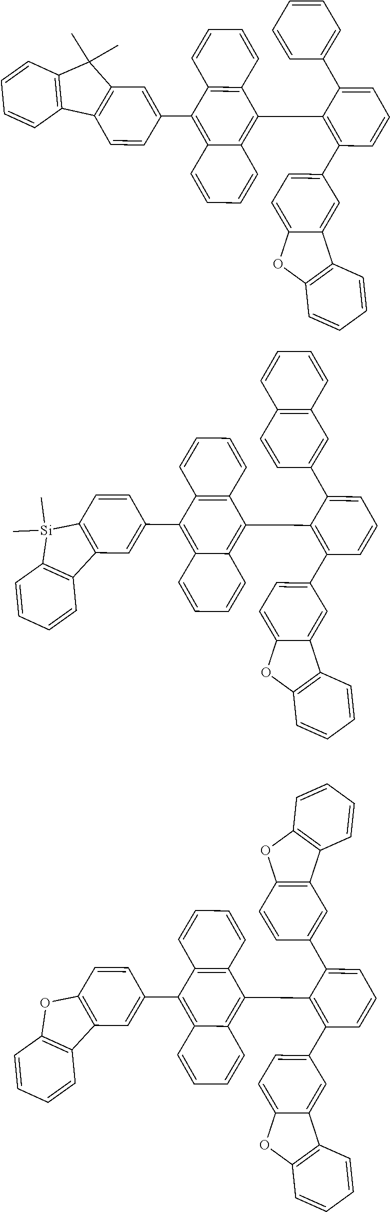

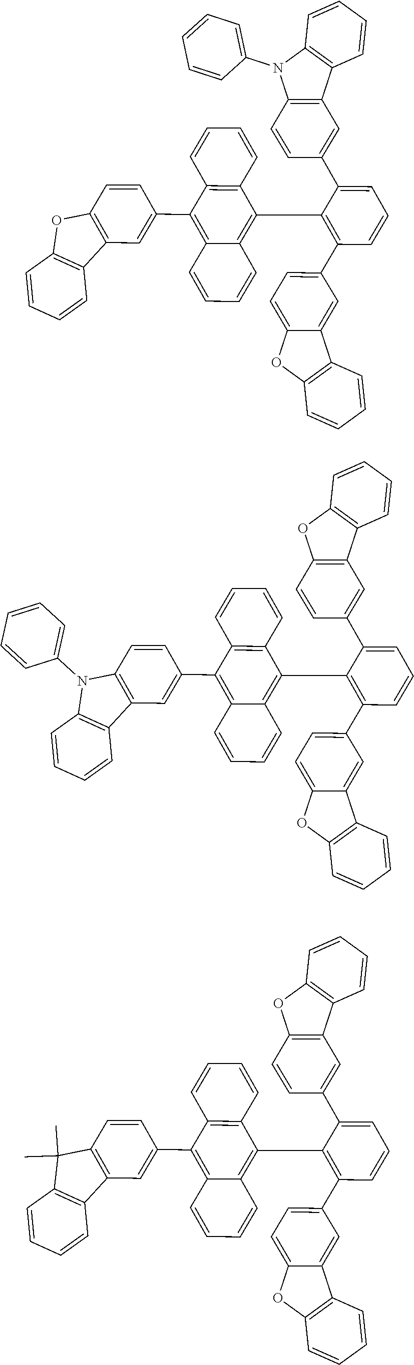

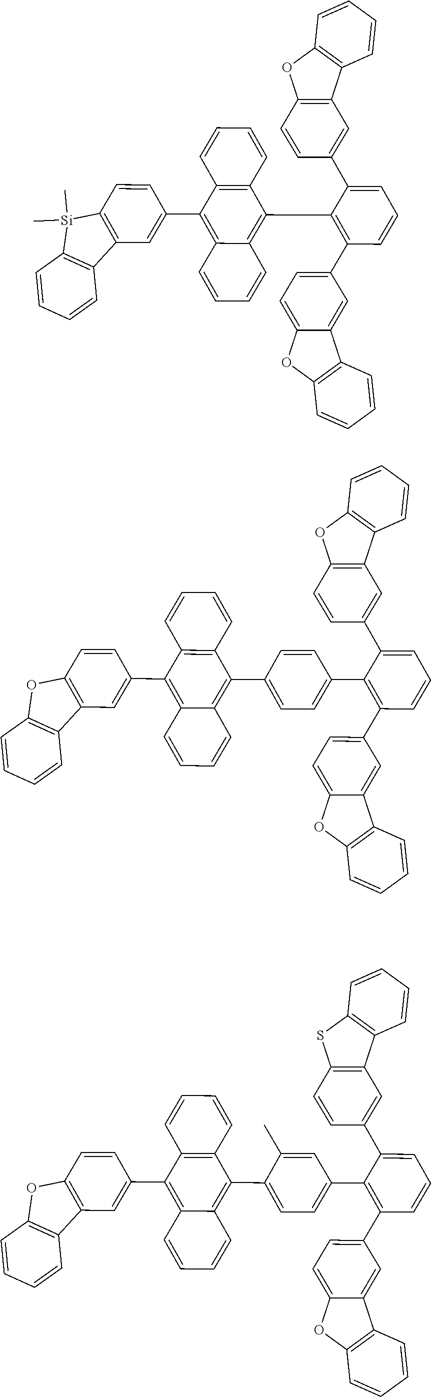 [SMILES: C[Si]1(C)c2ccccc2-c2cc(-c3c4ccccc4c(-c4c(-c5ccc6oc7ccccc7c6c5)cccc4-c4ccc5oc6ccccc6c5c4)c4ccccc34)ccc21 ; c1cc(-c2ccc3oc4ccccc4c3c2)c(-c2ccc(-c3c4ccccc4c(-c4ccc5oc6ccccc6c5c4)c4ccccc34)cc2)c(-c2ccc3oc4ccccc4c3c2)c1 ; Cc1cc(-c2c(-c3ccc4oc5ccccc5c4c3)cccc2-c2ccc3sc4ccccc4c3c2)ccc1-c1c2ccccc2c(-c2ccc3oc4ccccc4c3c2)c2ccccc12]

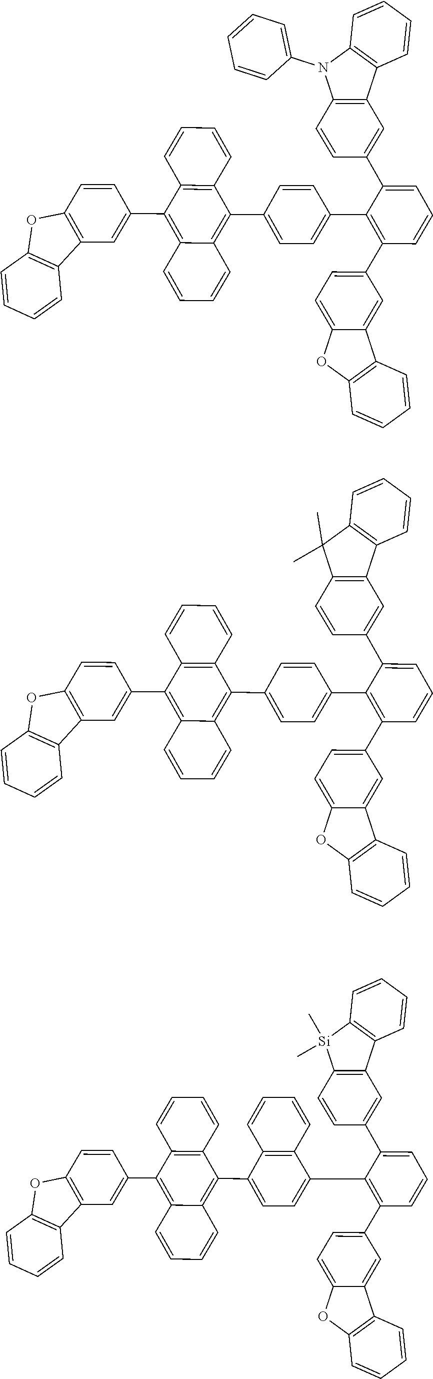 [SMILES: c1ccc(-n2c3ccccc3c3cc(-c4cccc(-c5ccc6oc7ccccc7c6c5)c4-c4ccc(-c5c6ccccc6c(-c6ccc7oc8ccccc8c7c6)c6ccccc56)cc4)ccc32)cc1 ; CC1(C)c2ccccc2-c2cc(-c3cccc(-c4ccc5oc6ccccc6c5c4)c3-c3ccc(-c4c5ccccc5c(-c5ccc6oc7ccccc7c6c5)c5ccccc45)cc3)ccc21 ; C[Si]1(C)c2ccccc2-c2cc(-c3cccc(-c4ccc5oc6ccccc6c5c4)c3-c3ccc(-c4c5ccccc5c(-c5ccc6oc7ccccc7c6c5)c5ccccc45)c4ccccc34)ccc21]

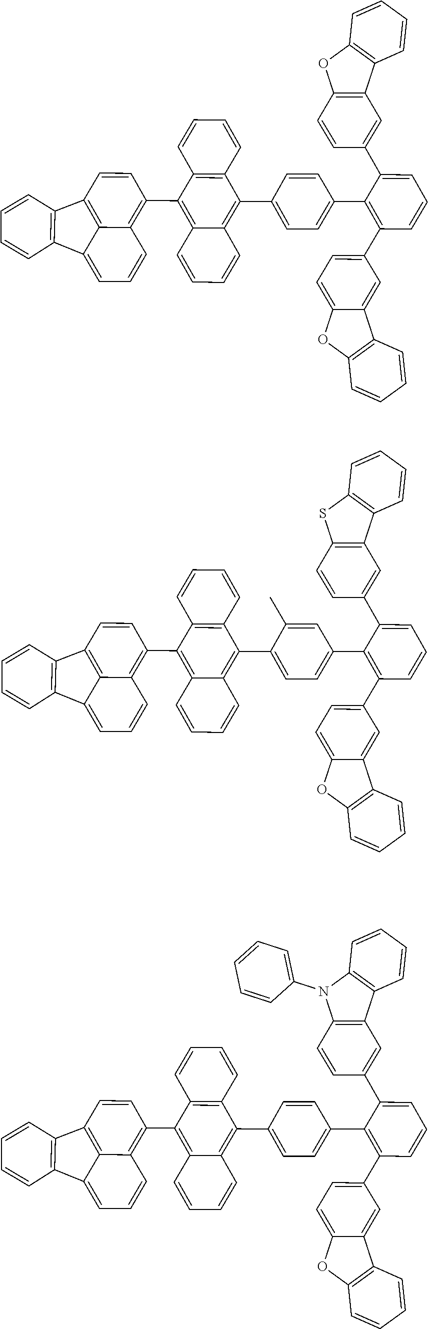 [SMILES: c1ccc2c(c1)-c1cccc3c(-c4c5ccccc5c(-c5ccc(-c6c(-c7ccc8oc9ccccc9c8c7)cccc6-c6ccc7oc8ccccc8c7c6)cc5)c5ccccc45)ccc-2c13 ; Cc1cc(-c2c(-c3ccc4oc5ccccc5c4c3)cccc2-c2ccc3sc4ccccc4c3c2)ccc1-c1c2ccccc2c(-c2ccc3c4c(cccc24)-c2ccccc2-3)c2ccccc12 ; c1ccc(-n2c3ccccc3c3cc(-c4cccc(-c5ccc6oc7ccccc7c6c5)c4-c4ccc(-c5c6ccccc6c(-c6ccc7c8c(cccc68)-c6ccccc6-7)c6ccccc56)cc4)ccc32)cc1]

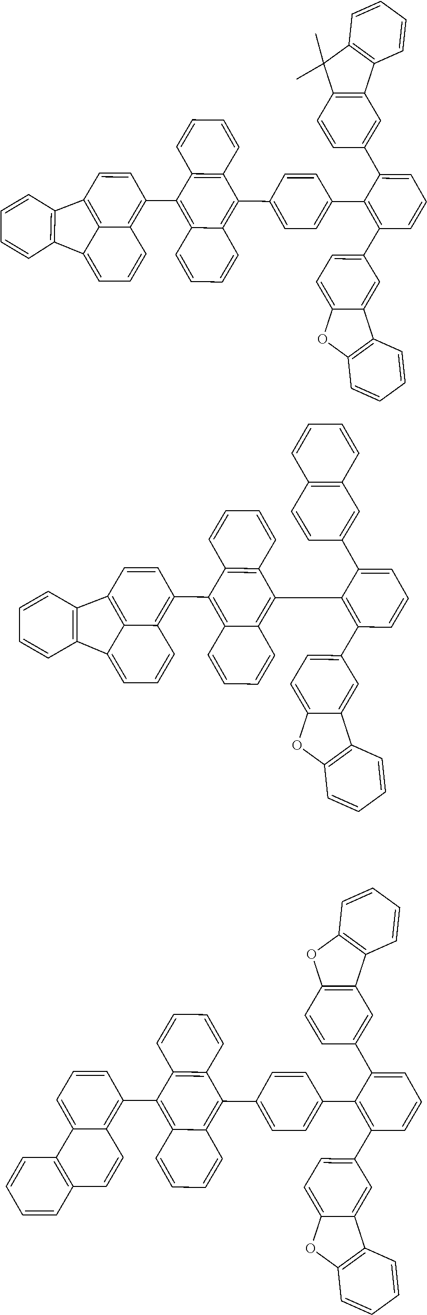

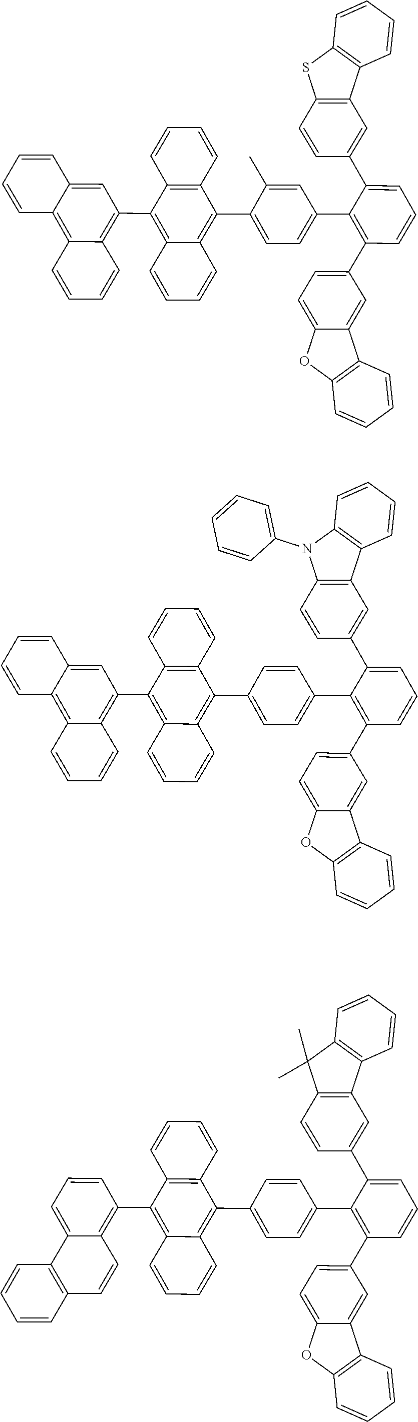 [SMILES: Cc1cc(-c2c(-c3ccc4oc5ccccc5c4c3)cccc2-c2ccc3sc4ccccc4c3c2)ccc1-c1c2ccccc2c(-c2cc3ccccc3c3ccccc23)c2ccccc12 ; c1ccc(-n2c3ccccc3c3cc(-c4cccc(-c5ccc6oc7ccccc7c6c5)c4-c4ccc(-c5c6ccccc6c(-c6cc7ccccc7c7ccccc67)c6ccccc56)cc4)ccc32)cc1 ; CC1(C)c2ccccc2-c2cc(-c3cccc(-c4ccc5oc6ccccc6c5c4)c3-c3ccc(-c4c5ccccc5c(-c5cccc6c5ccc5ccccc56)c5ccccc45)cc3)ccc21]

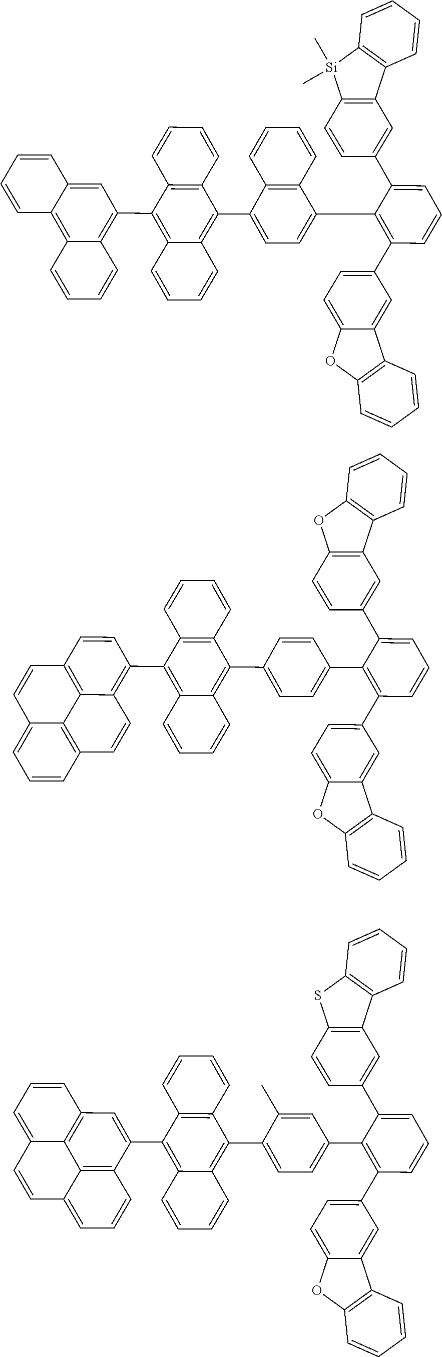

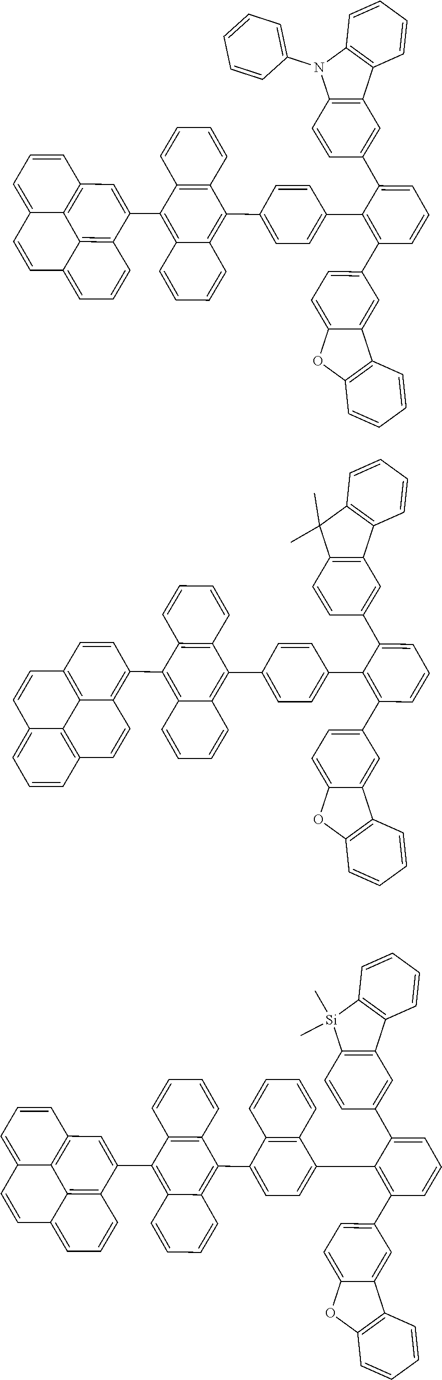

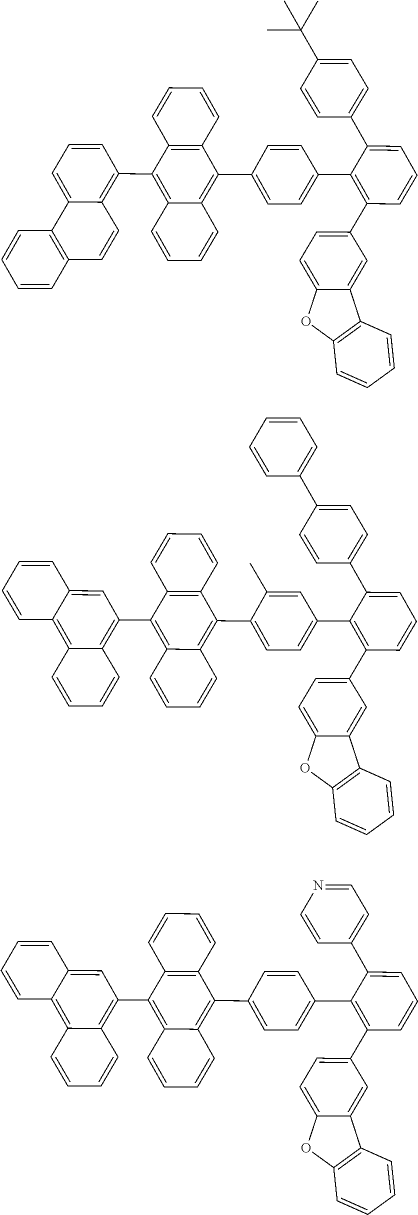 [SMILES: CC(C)(C)c1ccc(-c2cccc(-c3ccc4oc5ccccc5c4c3)c2-c2ccc(-c3c4ccccc4c(-c4cccc5c4ccc4ccccc45)c4ccccc34)cc2)cc1 ; Cc1cc(-c2c(-c3ccc(-c4ccccc4)cc3)cccc2-c2ccc3oc4ccccc4c3c2)ccc1-c1c2ccccc2c(-c2cc3ccccc3c3ccccc23)c2ccccc12 ; c1cc(-c2ccncc2)c(-c2ccc(-c3c4ccccc4c(-c4cc5ccccc5c5ccccc45)c4ccccc34)cc2)c(-c2ccc3oc4ccccc4c3c2)c1]

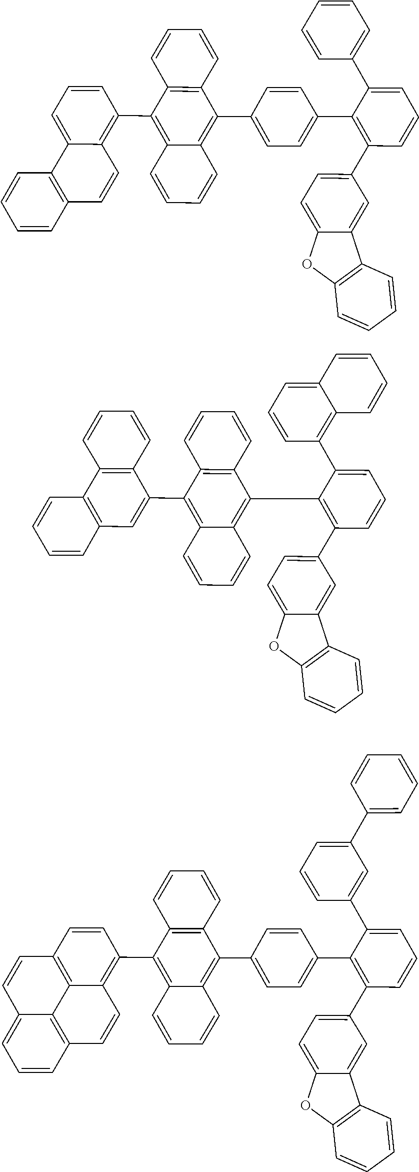

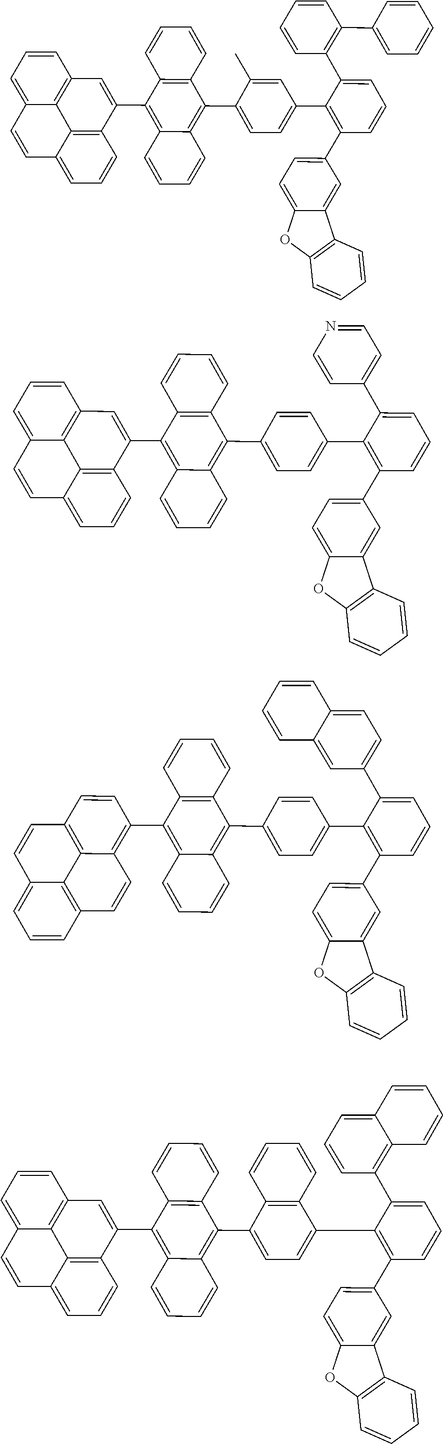

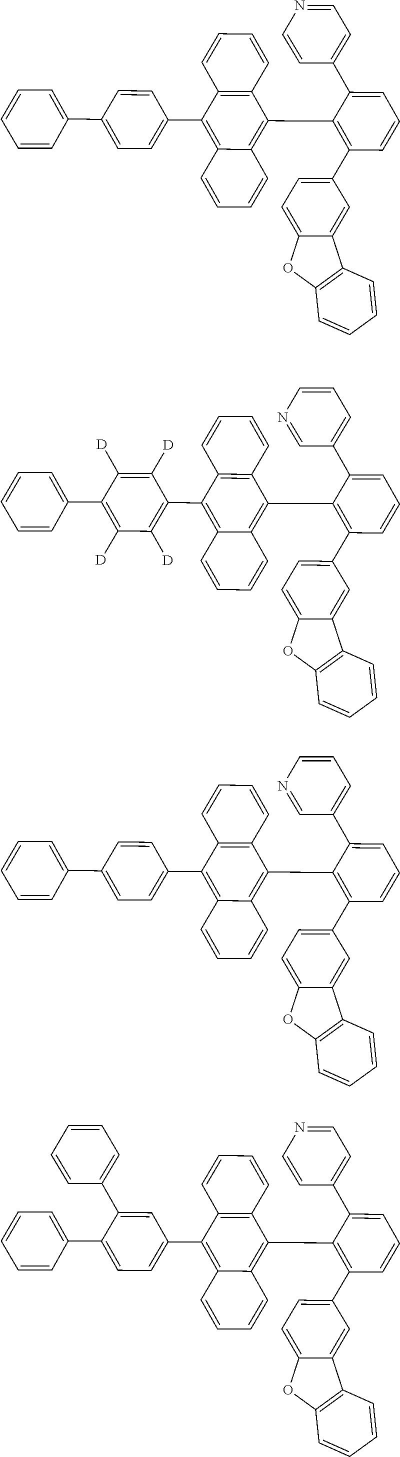

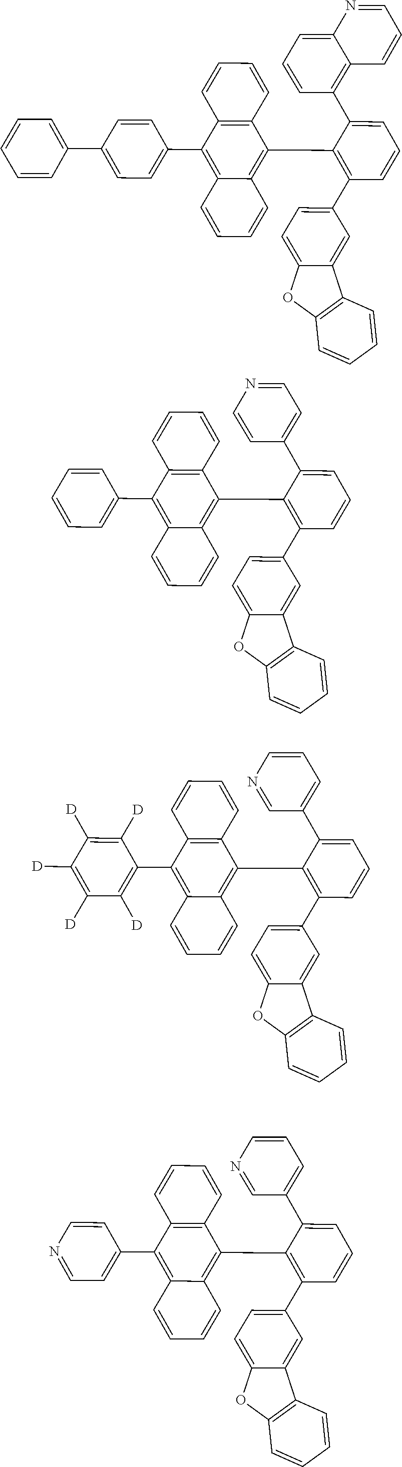

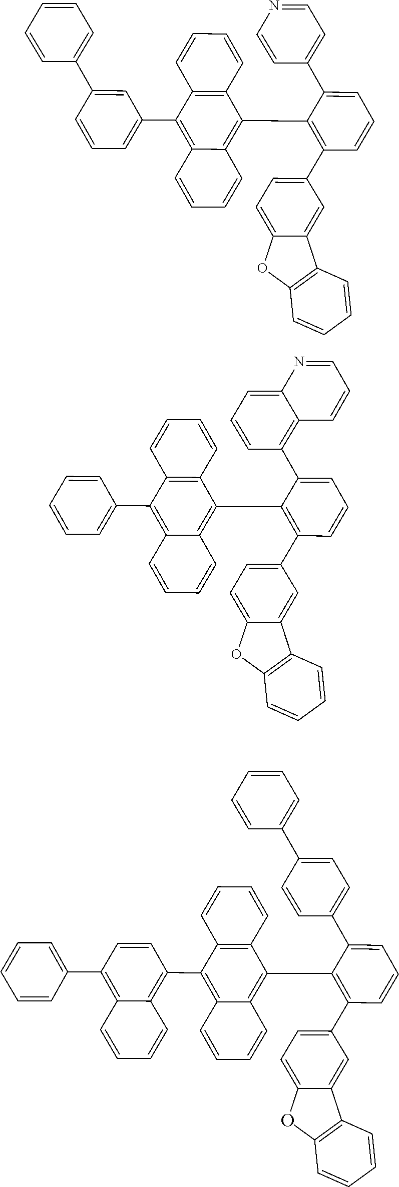

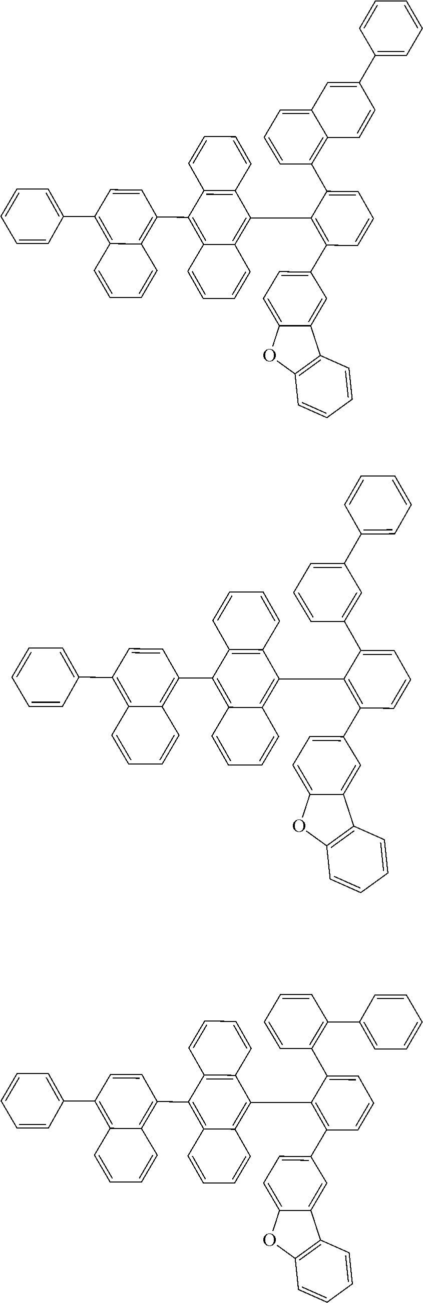 [SMILES: c1ccc(-c2ccc3c(-c4cccc(-c5ccc6oc7ccccc7c6c5)c4-c4c5ccccc5c(-c5ccc(-c6ccccc6)c6ccccc56)c5ccccc45)cccc3c2)cc1 ; c1ccc(-c2cccc(-c3cccc(-c4ccc5oc6ccccc6c5c4)c3-c3c4ccccc4c(-c4ccc(-c5ccccc5)c5ccccc45)c4ccccc34)c2)cc1 ; c1ccc(-c2ccccc2-c2cccc(-c3ccc4oc5ccccc5c4c3)c2-c2c3ccccc3c(-c3ccc(-c4ccccc4)c4ccccc34)c3ccccc23)cc1]

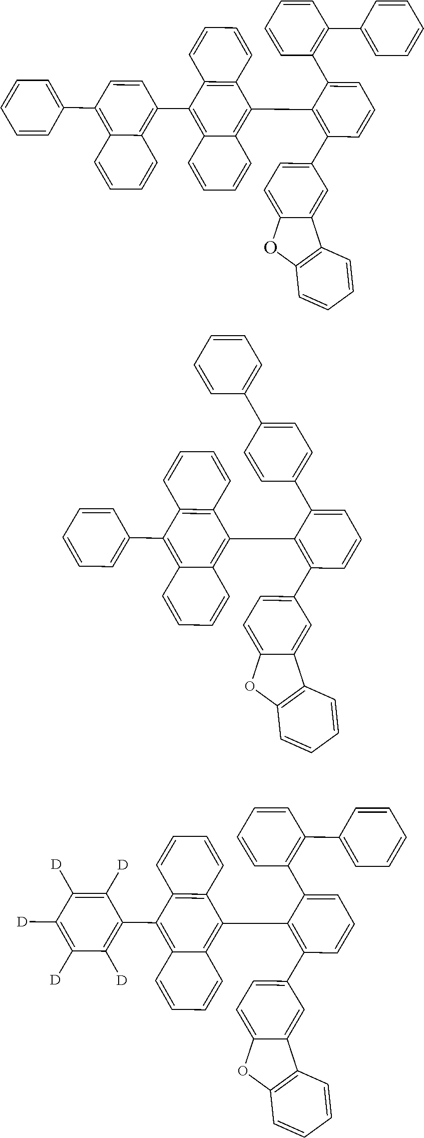 [SMILES: c1ccc(-c2ccccc2-c2cccc(-c3ccc4oc5ccccc5c4c3)c2-c2c3ccccc3c(-c3ccc(-c4ccccc4)c4ccccc34)c3ccccc23)cc1 ; c1ccc(-c2ccc(-c3cccc(-c4ccc5oc6ccccc6c5c4)c3-c3c4ccccc4c(-c4ccccc4)c4ccccc34)cc2)cc1 ; [2H]c1c([2H])c([2H])c(-c2c3ccccc3c(-c3c(-c4ccc5oc6ccccc6c5c4)cccc3-c3ccccc3-c3ccccc3)c3ccccc23)c([2H])c1[2H]]

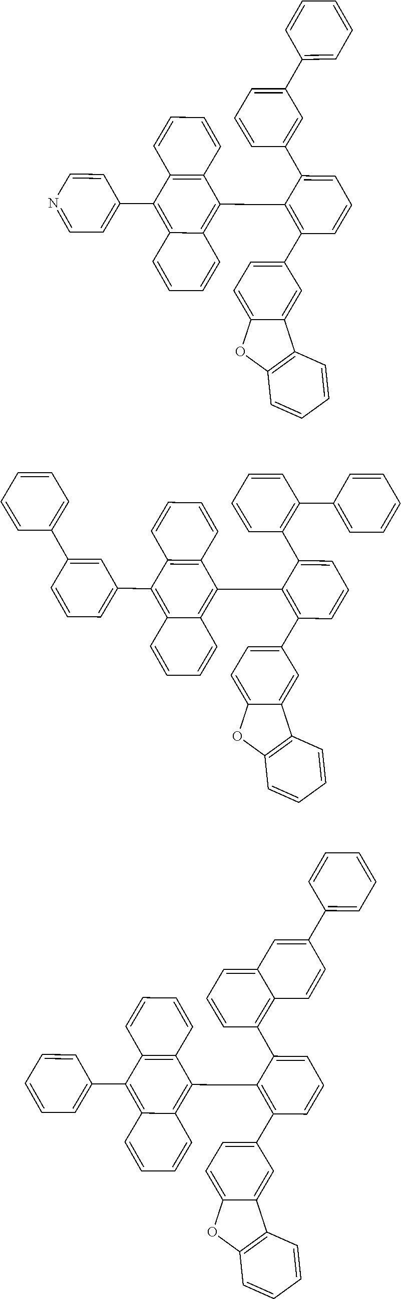 [SMILES: c1ccc(-c2cccc(-c3cccc(-c4ccc5oc6ccccc6c5c4)c3-c3c4ccccc4c(-c4ccncc4)c4ccccc34)c2)cc1 ; c1ccc(-c2cccc(-c3c4ccccc4c(-c4c(-c5ccc6oc7ccccc7c6c5)cccc4-c4ccccc4-c4ccccc4)c4ccccc34)c2)cc1 ; c1ccc(-c2ccc3c(-c4cccc(-c5ccc6oc7ccccc7c6c5)c4-c4c5ccccc5c(-c5ccccc5)c5ccccc45)cccc3c2)cc1]

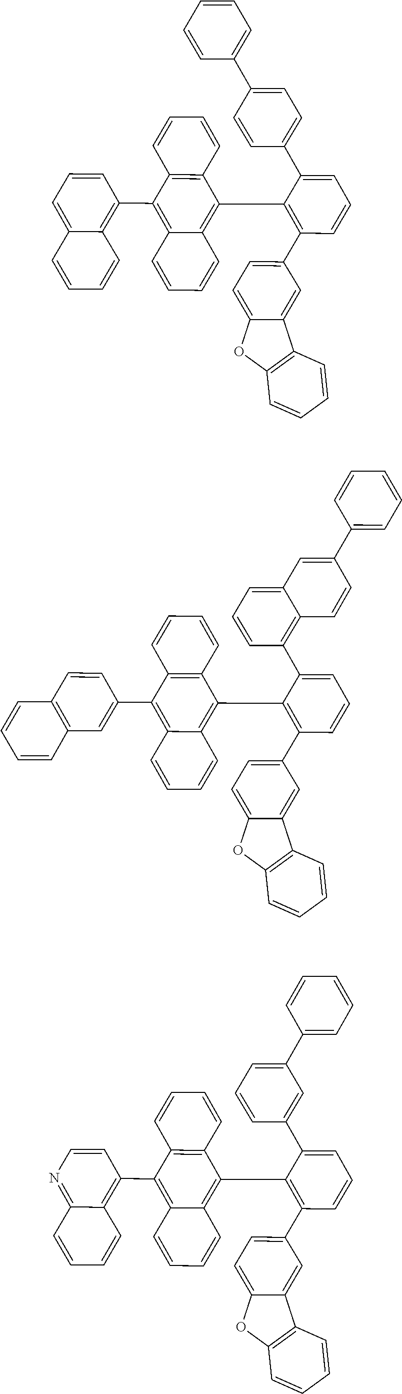 [SMILES: c1ccc(-c2ccc(-c3cccc(-c4ccc5oc6ccccc6c5c4)c3-c3c4ccccc4c(-c4cccc5ccccc45)c4ccccc34)cc2)cc1 ; c1ccc(-c2ccc3c(-c4cccc(-c5ccc6oc7ccccc7c6c5)c4-c4c5ccccc5c(-c5ccc6ccccc6c5)c5ccccc45)cccc3c2)cc1 ; c1ccc(-c2cccc(-c3cccc(-c4ccc5oc6ccccc6c5c4)c3-c3c4ccccc4c(-c4ccnc5ccccc45)c4ccccc34)c2)cc1]

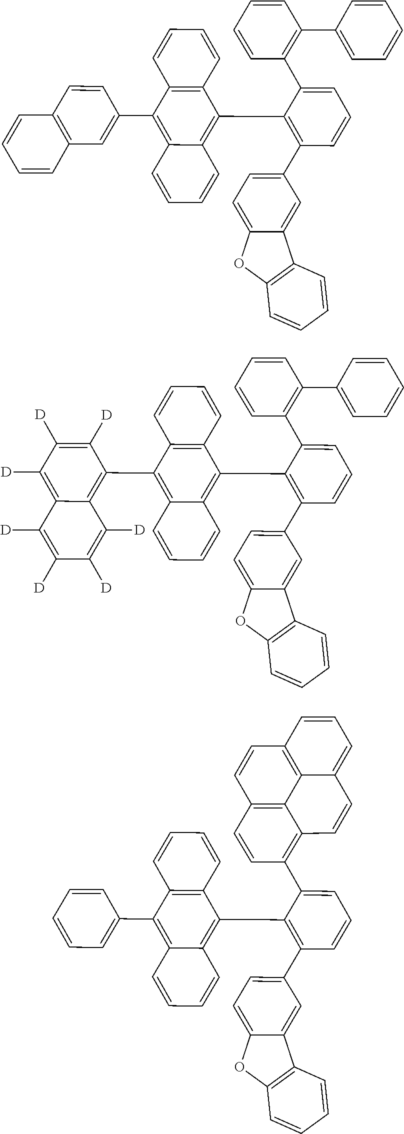

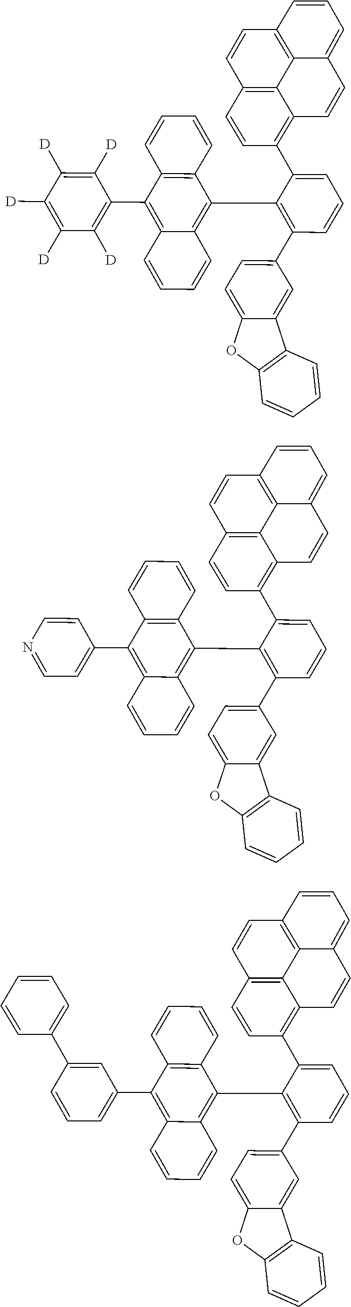

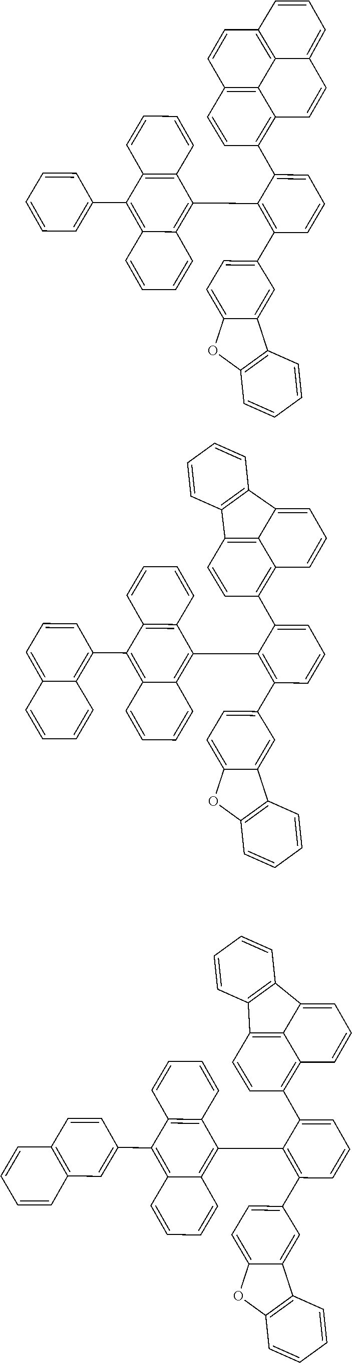 [SMILES: c1ccc(-c2c3ccccc3c(-c3c(-c4ccc5oc6ccccc6c5c4)cccc3-c3ccc4ccc5cccc6ccc3c4c56)c3ccccc23)cc1 ; c1ccc2c(c1)-c1cccc3c(-c4cccc(-c5ccc6oc7ccccc7c6c5)c4-c4c5ccccc5c(-c5cccc6ccccc56)c5ccccc45)ccc-2c13 ; c1ccc2c(c1)-c1cccc3c(-c4cccc(-c5ccc6oc7ccccc7c6c5)c4-c4c5ccccc5c(-c5ccc6ccccc6c5)c5ccccc45)ccc-2c13]

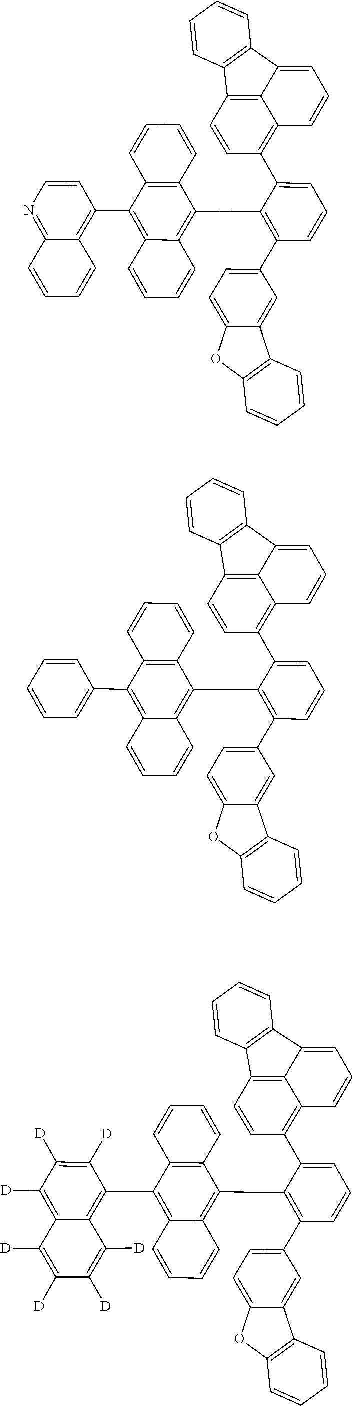 [SMILES: c1ccc2c(c1)-c1cccc3c(-c4cccc(-c5ccc6oc7ccccc7c6c5)c4-c4c5ccccc5c(-c5ccnc6ccccc56)c5ccccc45)ccc-2c13 ; c1ccc(-c2c3ccccc3c(-c3c(-c4ccc5oc6ccccc6c5c4)cccc3-c3ccc4c5c(cccc35)-c3ccccc3-4)c3ccccc23)cc1 ; [2H]c1c([2H])c([2H])c2c(-c3c4ccccc4c(-c4c(-c5ccc6oc7ccccc7c6c5)cccc4-c4ccc5c6c(cccc46)-c4ccccc4-5)c4ccccc34)c([2H])c([2H])c([2H])c2c1[2H]]

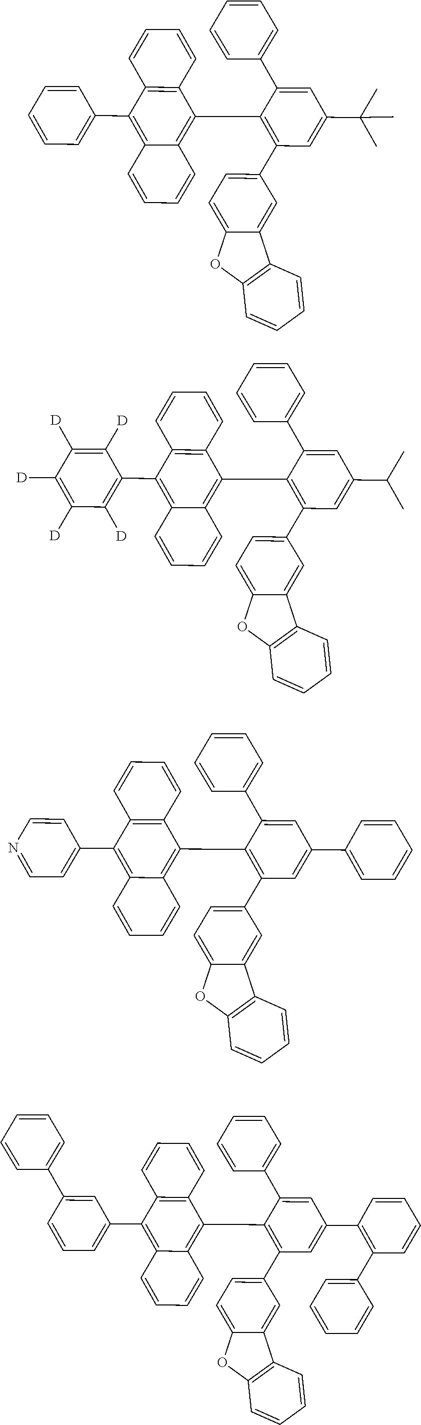

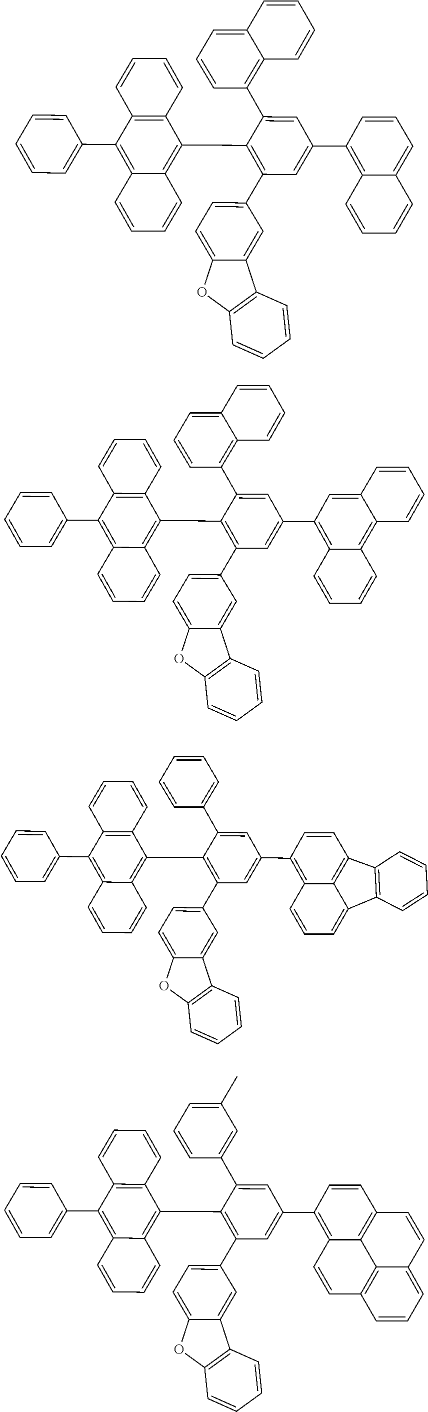

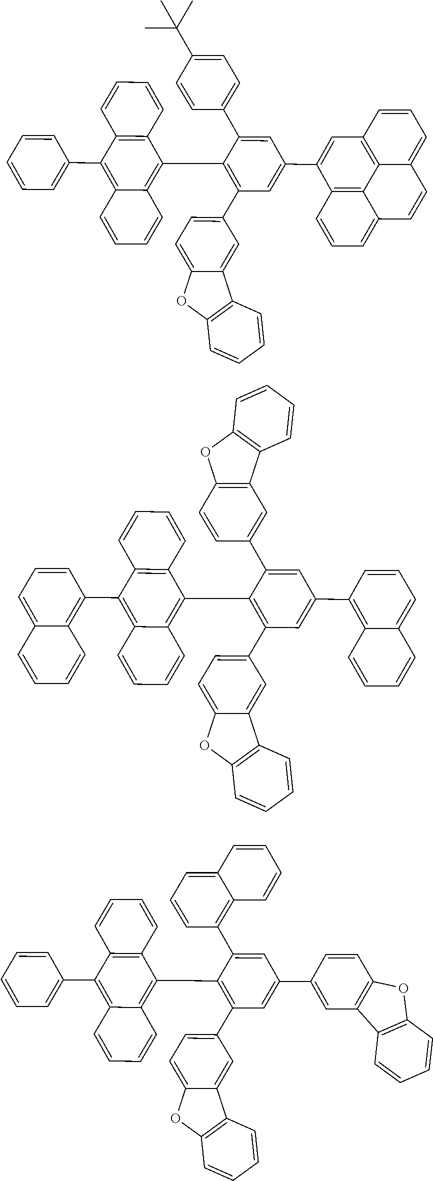

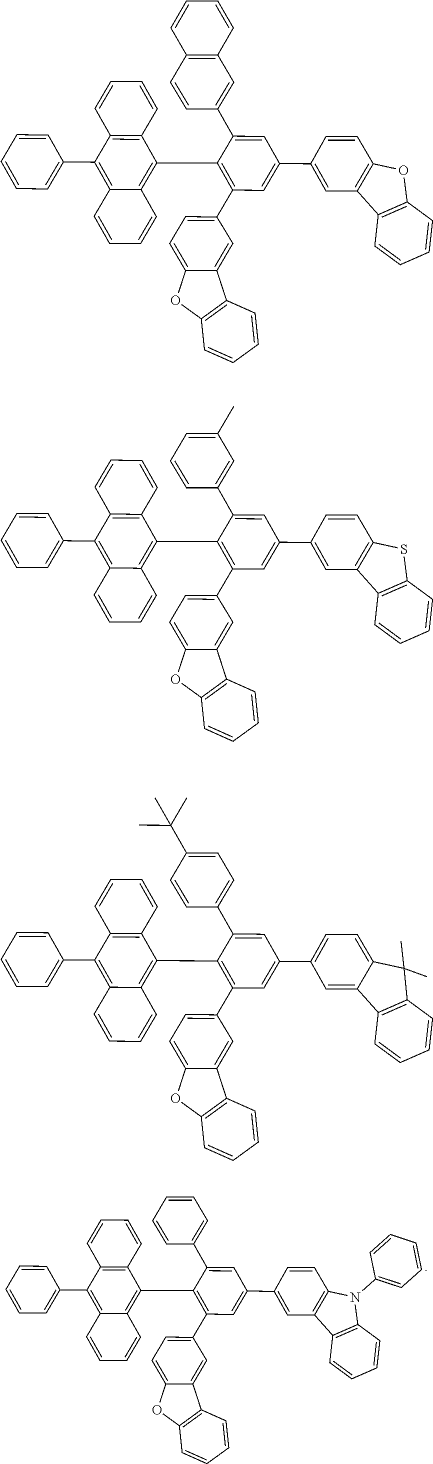 [SMILES: c1ccc(-c2c3ccccc3c(-c3c(-c4ccc5ccccc5c4)cc(-c4ccc5oc6ccccc6c5c4)cc3-c3ccc4oc5ccccc5c4c3)c3ccccc23)cc1 ; Cc1cccc(-c2cc(-c3ccc4sc5ccccc5c4c3)cc(-c3ccc4oc5ccccc5c4c3)c2-c2c3ccccc3c(-c3ccccc3)c3ccccc23)c1 ; CC(C)(C)c1ccc(-c2cc(-c3ccc4c(c3)-c3ccccc3C4(C)C)cc(-c3ccc4oc5ccccc5c4c3)c2-c2c3ccccc3c(-c3ccccc3)c3ccccc23)cc1 ; c1ccc(-c2cc(-c3ccc4c(c3)c3ccccc3n4-c3ccccc3)cc(-c3ccc4oc5ccccc5c4c3)c2-c2c3ccccc3c(-c3ccccc3)c3ccccc23)cc1]

19. A composition, wherein the composition comprises one or more compounds and 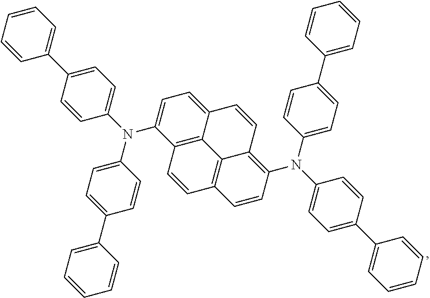 [SMILES: c1ccc(-c2ccc(N(c3ccc(-c4ccccc4)cc3)c3ccc4ccc5c(N(c6ccc(-c7ccccc7)cc6)c6ccc(-c7ccccc7)cc6)ccc6ccc3c4c65)cc2)cc1]
wherein one or more compounds are represented by formula (1): 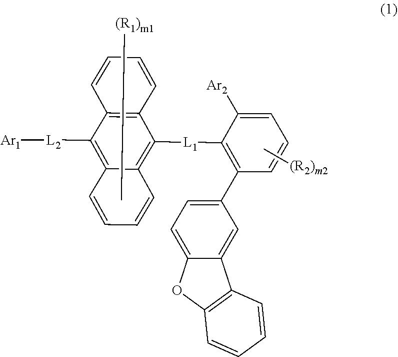
wherein Ar₁ is selected from an aromatic group containing 6 to 18 ring atoms and a heteroaromatic group containing 6 to 13 ring atoms;
Ar₂ is selected from an aromatic group containing 6 to 14 ring atoms and a heteroaromatic group containing 13 to 19 ring atoms;
L₁ and L₂ are each independently selected from a single bond and a group containing 6 ring atoms;
R₁ and R₂ are each independently selected from -D, a C₄ alkyl group, an aromatic group containing 6 to 10 ring atoms, and a heteroaromatic group containing 12 ring atoms;
m1 is 0, 2, or 8; and
m2 is 0 or 1.

20. An organic electronic device, wherein the organic electronic device comprises a first electrode, a second electrode, and an organic functional layer disposed between the first electrode and the second electrode, wherein the organic functional layer comprises one or more compounds represented by formula (1): 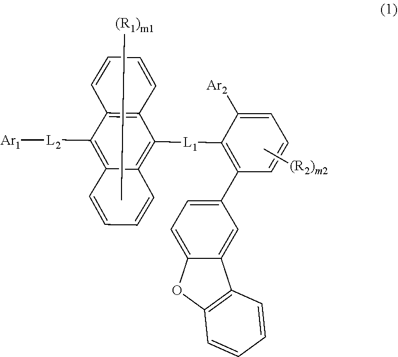
wherein Ar₁ is selected from an aromatic group containing 6 to 18 ring atoms and a heteroaromatic group containing 6 to 13 ring atoms;
Ar₂ is selected from an aromatic group containing 6 to 14 ring atoms and a heteroaromatic group containing 13 to 19 ring atoms;
L₁ and L₂ are each independently selected from a single bond and a group containing 6 ring atoms;
R₁ and R₂ are each independently selected from -D, a C₄ alkyl group, an aromatic group containing 6 to 10 ring atoms, and a heteroaromatic group containing 12 ring atoms;
m1 is 0, 2, or 8; and
m2 is 0 or 1.
